(19) United States
(12) Patent Application Publication
(10) Pub. No.: US 2026/0174867 A1
(43) Pub. Date: Jun. 25, 2026

(54) REDUCTION OF ADIPOSE TISSUE

(71) Applicant: LEIOS THERAPEUTICS, INC.

(72) Inventors: Daniel PIACQUADIO, San Diego, CA (US); John Daniel DOBAK, III, La Jolla, CA (US)

(21) Appl. No.: 19/535,472

(22) Filed: Feb. 10, 2026

Related U.S. Application Data
(63) Continuation of application No. PCT/US2024/058264, filed on Dec. 3, 2024.
(60) Provisional application No. 63/606,082, filed on Dec. 4, 2023.
(60) Provisional application No. 63/618,768, filed on Jan. 8, 2024.
(60) Provisional application No. 63/657,001, filed on Jun. 6, 2024.

(51) Int. Cl. A61K 47/10; A61K 31/573

(52) U.S. Cl. CPC A61K 47/10; A61K 31/573

(57) ABSTRACT

Described herein are compositions, formulations, methods, and systems for reducing regional fat deposits and treating fat-related conditions.

### INCORPORATION BY REFERENCE

This application is a continuation application of International Application No. PCT/US2024/058264, filed Dec. 3, 2024, which claims the benefit of U.S. Provisional Application No. 63/606,082, filed on Dec. 4, 2023; 63/618,768, filed on Jan. 8, 2024; and 63/657,001, filed on Jun. 6, 2024, the entire contents of each of which are incorporated herein by reference.

### BACKGROUND

Procedures for removing fat from the human body have increased in prevalence as the general population has increased in body weight and age. Lipoplasty and other surgical methods for removing fat present significant risks including death, high rates of complications, infection, seromas, skin damage, thromboembolism, or fluid imbalances. Thus, there remains a need for alternative procedures for removal of fat deposits.

### SUMMARY

While pharmaceutical detergent compositions for reduction of adipose tissue, including submental adipose tissue, are utilized, they have failed to achieve widespread adoption in the field. In cases of reduction of submental adipose tissue with detergent compositions, such compositions have failed to achieve a patient significant two grade improvement reduction in submental adipose tissue within a reasonable number of treatment sessions, and induce significant injection related side effects in human subjects. The current standard of care for non-surgical reduction of adipose tissue with injectable pharmaceutical detergent compositions is subcutaneous injection of 1% w/w deoxycholic acid. However, many human subjects undergoing subcutaneous injection of 1% w/w deoxycholic acid for reduction of submental adipose tissue fail to exhibit at least a two grade improvement reduction in submental adipose tissue, which the minimum improvement desired by most subjects to be considered a cosmetically significant result, with less than 19% of subjects exhibiting a two grade improvement or larger reduction in submental adipose tissue. Further, as many as 87% of subjects undergoing subcutaneous injection of 1% w/w deoxycholic acid exhibit an injection related side effect, with up to 42% of said subjects exhibiting an injection related side effect which lasts for more than 30 days. There remains a need in the art for improved methods of reducing adipose tissue, including submental adipose tissue, in human subjects with increased safety and efficacy as compared the current standard of care.

Similar to deoxycholic acid, polidocanol functions as a detergent to induce cytolysis of adipocytes and result in local reduction of fat tissue. Surprisingly and unexpectedly, is shown that the polidocanol pharmaceutical compositions disclosed herein when administered according to the methods described herein result in approximately 80% of human subjects undergoing treatment exhibiting a two grade improvement or larger reduction in submental adipose tissue, with fewer subjects exhibiting any injection related side effect. Subject that do experience an injection related side effect exhibited a side effect of reduced severity and duration, with a significant majority of subjects exhibiting a grade 0 (none) or grade 1 (mild) side effect, and less than 5% of subjects exhibiting an injection related side effect lasting longer than 30 days. Such improved results as to both safety and efficacy with a detergent based active pharmaceutical ingredient when injected subcutaneous for reduction of adipose tissue in human subjects has not been previously observed, represents a significant improvement over the standard of care, and out performs expectations of those of skill in the art in safety and efficacy for such polidocanol detergent based formulations.

Described herein are pharmaceutical compositions, formulations, methods, and systems to reduce regional fat, adipose tissue, adipocyte, or regional or localized adiposity. Described herein, ins some aspects, is a method of improving elimination of subcutaneous adipose tissue in a subject comprising injecting the subject with a pharmaceutical composition comprising 3.0-4.5% w/w polidocanol, wherein the method improves elimination of subcutaneous adipose tissue in the subject by at least 1.5x compared to injection of 1% w/w deoxycholic acid. Described herein, in some aspects, is a method of improving elimination of subcutaneous adipose tissue in a subject comprising injecting the subject with a pharmaceutical composition comprising 3.0-4.5% w/w polidocanol, wherein the method improves elimination of subcutaneous adipose tissue in the subject by at least 1.5× compared to injection of 2% w/w deoxycholic acid. Described herein, in some aspects, is a method of improving elimination of subcutaneous adipose tissue in a subject comprising injecting the subject with a pharmaceutical composition comprising 3.0-4.5% w/w polidocanol, wherein the subject exhibits at least a two grade improvement in submental adipose tissue reduction. Described herein, in some aspects, is a method of eliminating of subcutaneous adipose tissue in a subject comprising injecting the subject with more than an average of more than 30 doses per treatment during a clinical treatment regimen of injection of a pharmaceutical composition comprising 3.0-4.5% w/w polidocanol in a single treatment session. Described herein, in some aspects, is a method of eliminating of subcutaneous adipose tissue in a subject comprising injecting the subject with a plurality of doses of a pharmaceutical composition comprising 3.0-4.5% w/w polidocanol over an area of at least 15 cm². Described herein, in some aspects, is a method of eliminating of subcutaneous adipose tissue in a subject comprising injecting the subject with a pharmaceutical composition comprising 3.0-4.5% w/w polidocanol in a plurality of treatment sessions, each treatment session being more than 23 days apart more than 23 days apart but not more than 60 days apart. Described herein, in some aspects, is a method of eliminating of subcutaneous adipose tissue in a subject comprising injecting the subject with a pharmaceutical composition comprising 3.0-4.5% w/w polidocanol in a plurality of treatment sessions, comprising at administering between two and six treatment sessions to the subject. Described herein, in some aspects, is a method of eliminating of subcutaneous adipose tissue in a subject comprising injecting the subject with a pharmaceutical composition comprising administering at least 2 treatment sessions to the subject, wherein at least one of the second or subsequent treatment(s) sessions comprises injecting the subject with a reduced concentration of polidocanol than was used in the first treatment. Described herein, in some aspects, is a method of reducing an injection related side effect in a subject comprising injecting the subject with a pharmaceutical composition comprising a progressively reduced concentration of polidocanol in a plurality of treatment sessions. Described herein, in some aspects, is a method of reducing an injection related side effect in a subject comprising injecting the subject with a pharmaceutical composition comprising in a plurality of treatment sessions comprising at least one of: a) administering a progressively reduced number of injections of the pharmaceutical composition in a second or subsequent treatment session(s) of the plurality of treatment sessions, or b) administering a reduced volume of injectate of the pharmaceutical composition in the second or subsequent treatment session(s). Described herein, in some aspects, is a method of reducing an injection related side effect in a subject comprising injecting the subject with a pharmaceutical composition comprising 3.0-4.5% w/w polidocanol.

In some embodiments, the subject is human. In some embodiments, the method improves elimination of subcutaneous adipose tissue in the subject by at least 50% compared to injection of 1% w/w deoxycholic acid. In some embodiments, the method improves elimination of subcutaneous adipose tissue in the subject by at least 50% compared to injection of 2% w/w deoxycholic acid. In some embodiments, the subject exhibits at least a two grade improvement in submental adipose tissue reduction. In some embodiments, the method comprises injecting the subject with an average of more than 30 doses per treatment during a clinical treatment regimen of up to six treatments of the pharmaceutical composition. In some embodiments, the method comprises injecting the subject with a plurality of doses of a pharmaceutical composition over an area of at least 15 cm². In some embodiments, the method comprises injecting the subject with a plurality of doses of a pharmaceutical composition over an area of at least 16, 17, 18, 19, 20, 21, 22, 23, 24, 25, 26, 27, 28, 29, 30, 35, or 40 cm². In some embodiments, the area is an effective treatment area. In some embodiments, the method comprises injecting the subject with the pharmaceutical composition in a plurality of treatment sessions, each treatment session being more than 23 days apart but not more than 60 days apart. In some embodiments, the method reduces an injection related side effect in the subject. In some embodiments, the method improves a rate of elimination of subcutaneous adipose tissue in the subject compared to injection of 1% w/w deoxycholic acid. In some embodiments, the method improves elimination of subcutaneous adipose tissue in the subject by at least 1.75, 2, 2.25, 2.5, 2.75, 3, 3.25, 3.5, 3.75, 4, 4.5, or 5× compared to injection of 1% w/w deoxycholic acid. In some embodiments, the method improves elimination of subcutaneous adipose tissue in the subject by at least 1.75, or 2× compared to injection of 2% w/w deoxycholic acid. In some embodiments, the method includes administering at least 2 treatment sessions to the subject, wherein at least one of the second or subsequent treatment session(s) comprises injecting the subject with a reduced concentration of polidocanol than was used in the first treatment. In some embodiments, the method includes administering a plurality of treatment sessions to the subject, wherein a progressively reduced concentration of polidocanol is administered to the subject in the plurality of treatment sessions. In some embodiments, the method includes injecting the subject with a pharmaceutical composition comprising in a plurality of treatment sessions comprising at least one of: a) administering a progressively reduced number of injections of the pharmaceutical composition in a second or subsequent treatment session(s) of the plurality of treatment sessions, or b) administering a reduced volume of injectate of the pharmaceutical composition in the second or subsequent treatment session(s). In some embodiments, a pharmaceutical composition comprising up to 4.5% w/w polidocanol is administered to the subject in a first treatment session of a plurality of treatment sessions, and a pharmaceutical composition comprising less than 4.5% w/w polidocanol is administered to the subject in a subsequent treatment session(s). In some embodiments the pharmaceutical composition comprising up to 4.5% w/w polidocanol is administered to the subject in the first treatment session and a second treatment session of a plurality of treatment sessions. The method of any one of the preceding claims, wherein a pharmaceutical composition comprising 4.5% w/w polidocanol is administered to the subject in a first treatment session of a plurality of treatment sessions, and a pharmaceutical composition comprising less than 4.5% w/w polidocanol is administered to the subject in a subsequent treatment session(s). In some embodiments the pharmaceutical composition comprising 4.5% w/w polidocanol is administered to the subject in the first treatment session and a second treatment session of a plurality of treatment sessions. In some embodiments the pharmaceutical composition comprising less than 4.5% w/w polidocanol comprises up to 3.0% w/w polidocanol. In some embodiments the pharmaceutical composition comprising up to 3.0% w/w polidocanol is administered in a third treatment session and a fourth treatment session, wherein the subsequent treatment session(s) comprises the third treatment session and the fourth treatment session. In some embodiments the pharmaceutical composition comprising less than 4.5% w/w polidocanol comprises 3.0% w/w polidocanol. In some embodiments the pharmaceutical composition comprising 3.0% w/w polidocanol is administered in a third treatment session and a fourth treatment session, wherein the subsequent treatment session(s) comprises the third treatment session and the fourth treatment session. In some embodiments a pharmaceutical composition comprising less than 3.0% w/w polidocanol is administered to the subject in a second subsequent treatment session(s). In some embodiments the pharmaceutical composition comprising less than 3.0% w/w polidocanol comprises up to 2.0% w/w polidocanol. In some embodiments the second subsequent treatment session(s) comprise a fifth treatment session and a sixth treatment session. In some embodiments the pharmaceutical composition comprising up to 2.0% w/w polidocanol are administered to the subject in the fifth treatment session and the sixth treatment session. In some embodiments the pharmaceutical composition comprising less than 3.0% w/w polidocanol comprises 2.0% w/w polidocanol. In some embodiments the pharmaceutical composition comprising 2.0% w/w polidocanol are administered to the subject in the fifth treatment session and the sixth treatment session. In some embodiments the administering the reduced volume of injectate of the pharmaceutical composition in the second or subsequent treatment session(s) comprises at least one of the injections in the second or subsequent treatment session(s) having a reduced injectate volume of individual injections of a plurality of injections than was administered in a first subsequent treatment session. In some embodiments the administering the reduced volume of injectate of the pharmaceutical composition in the second or subsequent treatment session(s) comprises each of the injections in the second or subsequent treatment session(s) having a reduced injectate volume of individual injections of a plurality of injections than was administered in a first subsequent treatment session. In some embodiments, the method reduces a mass of subcutaneous adipose tissue in the subject. In some embodiments, the method reduces a mass of subcutaneous adipose tissue in the subject in a region of the injection by 10, 20, 30, 40, 50, 60, or 75% w/w. In some embodiments, the region is a submental region. In some embodiments, the method reduces a volume of subcutaneous adipose tissue in the subject. In some embodiments, the method reduces a volume of subcutaneous adipose tissue in the subject in a region of the injection by 10, 20, 30, 40, 50, 60, or 75% v/v. In some embodiments, the region is a submental region. In some embodiments, the subject exhibits at least a one grade improvement in submental adipose tissue reduction. In some embodiments, the one grade improvement in submental adipose tissue reduction is exhibited within 12, 11, 10, 9, or 8 weeks following injection with the pharmaceutical composition. In some embodiments, a one grade improvement in submental adipose tissue reduction is exhibited following administering at least three treatment sessions to the subject. In some embodiments, the subject exhibits at least a two grade improvement in submental adipose tissue reduction. In some embodiments, the one grade improvement in submental adipose tissue reduction is exhibited within 24 weeks following injection with the pharmaceutical composition. In some embodiments, the one grade improvement in submental adipose tissue reduction is exhibited following administering at least six treatment sessions to the subject. In some embodiments, the method comprises injecting the subject with up to 50 doses of the pharmaceutical composition. In some embodiments, the method comprises injecting the subject with at least 50 doses of the pharmaceutical composition. In some embodiments, the method comprises injecting the subject with at least 20 and up to 50 doses of the pharmaceutical composition. In some embodiments, the method comprises injecting the subject with an average of at least 30 and up to 50 doses of the pharmaceutical composition. In some embodiments, each dose is not more than 0.2 mL. In some embodiments, each dose is less than 0.2 mL. In some embodiments, the method comprises administering at least three treatment sessions to the subject. In some embodiments, the method comprises administering at least six treatment sessions to the subject. In some embodiments, the method comprises administering between two and six treatment sessions to the subject. In some embodiments, the method comprises administering up to six treatment sessions to the subject. In some embodiments, each treatment session is administered at least 3 weeks apart. In some embodiments, each treatment session is administered more than 30 days apart. In some embodiments, each dose is injected at least 1 cm apart. In some embodiments, each dose is injected up to 1 cm apart. In some embodiments, each dose is injected about 1 cm apart. In some embodiments, a submental region of the subject is treated. In some embodiments, a flank region of the subject is treated. In some embodiments, an abdominal region of the subject is treated. In some embodiments, a lingual region of the subject is treated. In some embodiments, the subject exhibits at least a one grade improvement in submental adipose tissue reduction. In some embodiments, the one grade improvement in submental adipose tissue reduction is exhibited after 2 treatment sessions. In some embodiments, the one grade improvement in submental adipose tissue reduction is exhibited within about 8 weeks. In some embodiments, the subject exhibits at least a two grade improvement in submental adipose tissue reduction. In some embodiments, the two grade improvement in submental adipose tissue reduction is exhibited after 3 treatment sessions. In some embodiments, the two grade improvement in submental adipose tissue reduction is exhibited within about 24 weeks. In some embodiments, the subject exhibits a decrease in convexity, fullness, bulging, bloating, stretching, in a region injected with the pharmaceutical composition. In some embodiments, the subject exhibits an improvement in appearance in a region injected with the pharmaceutical composition. In some embodiments, the pharmaceutical composition comprises about 3.0% w/w polidocanol. In some embodiments, the pharmaceutical composition comprises about 4.5% w/w polidocanol. In some embodiments, the pharmaceutical composition is a subcutaneous injection formulation. In some embodiments, the pharmaceutical composition is an aqueous formulation. In some embodiments, the pharmaceutical composition comprises a cosolvent. In some embodiments, the cosolvent comprises propylene glycol. In some embodiments, the propylene glycol is comprised in an amount of up to 2.25% w/w. In some embodiments, the pharmaceutical composition comprises a buffer. In some embodiments, the pharmaceutical composition comprises a binary buffers system. In some embodiments, the binary buffer system comprises sodium phosphate and potassium phosphate. In some embodiments, the binary buffer system comprises sodium phosphate dibasic dihydrate and potassium phosphate monobasic. In some embodiments, the sodium phosphate dibasic dihydrate is comprised in an amount of up to about 0.25% w/w. In some embodiments, the potassium phosphate monobasic is comprised in an amount of up to about 0.1% w/w. In some embodiments, the pharmaceutical composition comprises an osmolarity of less than about 400 mM/kg. In some embodiments, the pharmaceutical composition comprises an alkaline pH. In some embodiments, the pharmaceutical composition comprises a pH of 7-8. In some embodiments, the pharmaceutical composition comprises a critical micelle concentration of up to 0.10 millimolar. In some embodiments, the pharmaceutical composition comprises a critical micelle concentration of 0.07-0.1 mM. In some embodiments, the injecting is subcutaneous. In some embodiments, the method locally reduces subcutaneous adipose tissue. In some embodiments, the method locally reduces submental adipose tissue. In some embodiments, the comparative injection of 1% w/w deoxycholic acid in a same volume and/or a same spacing of injections. In some embodiments, the comparative injection of 2% w/w deoxycholic acid in a same volume and/or a same spacing of injections. In some embodiments, the reduction in the injection related side effect is exhibited as compared to injection of 1% w/w deoxycholic acid. In some embodiments, the reduction in the injection related side effect is exhibited as compared to injection of 2% w/w deoxycholic acid. In some embodiments, the side effect comprises numbness, induration, paresthesia, nodule, pruritis, or nerve injury. In some embodiments, the side effect comprises Erythema, injection site pain, bruising, edema, or swelling. In some embodiments, the method described herein reduces a severity of the side effect. In some embodiments, the side effect comprises numbness, induration, paresthesia, nodule, pruritis, nerve injury, injection site pain, bruising, edema, or swelling, wherein a 100, 90, 80, 70, 60, 50, 45, 40, 35, 30, 25, 20, 15, 10, or 5% decrease in occurrence of the side effect is observed as compared to injection of 1% w/w deoxycholic acid. In some embodiments, the side effect comprises numbness, induration, paresthesia, nodule, pruritis, nerve injury, injection site pain, bruising, edema, or swelling, wherein a 100, 90, 80, 70, 60, 50, 45, 40, 35, 30, 25, 20, 15, 10, or 5% decrease in occurrence of the side effect is observed as compared to injection of 2% w/w deoxycholic acid. In some embodiments, the comparative injection of 1% w/w deoxycholic acid in a same volume and/or a same spacing of injections, or wherein the comparative injection of 2% w/w deoxycholic acid is in a same volume and/or a same spacing of injections. In some embodiments, reducing the injection related side effect in the subject comprises the subject not exhibiting any injection related side effect after 30 days. In some embodiments, reducing the injection related side effect in the subject comprises less than 40% of subjects exhibiting any injection related side effect. In some embodiments, the reducing the injection related side effect in the subject comprises the subject not exhibiting any injection related side effect after 30 days following injection. In some embodiments, the reducing the injection related side effect in the subject comprises less than 50% of subjects exhibiting an injection related side effect comprising pain, bruising, burning, stinging, erythema, numbness, induration, paresthesia, nodule, pruritis, nerve injury, or combinations thereof. In some embodiments, the reducing the injection related side effect in the subject comprises the subject exhibiting reduced occurrence of the injection related side effect in subsequent treatments when receiving or after receiving a subsequent injection of the pharmaceutical composition. In some embodiments, the reducing the injection related side effect in the subject comprises fewer subjects exhibiting injection related side effect in subsequent treatments when receiving a subsequent injection of the pharmaceutical composition. In some embodiments, the reducing the injection related side effect in the subject comprises the subject not exhibiting a worsening of the injection related side effect following a treatment session. In some embodiments, the reducing the injection related side effect in the subject comprises the subject not exhibiting a worsening of the injection related side effect following receiving a subsequent injection of the pharmaceutical composition. In some embodiments, the reducing the injection related side effect in the subject comprises the injection related side effect resolving in the subject within 30 days. In some embodiments, the reducing the injection related side effect in the subject comprises the injection related side effect resolving in the subject prior to receiving a subsequent injection of the pharmaceutical composition. In some embodiments, the injection related side effect is pain, bruising, or edema. In some embodiments, up to six treatments are administered to the subject. In some embodiments, at least a one grade improvement in submental adipose tissue reduction is exhibited by the subject following the treatment. In some embodiments, at least a two grade improvement in submental adipose tissue reduction is exhibited by the subject following the treatment. In some embodiments, between three and 50 injections of the composition are administered to the subject per treatment session. In some embodiments, an average of 10 to 30 injections of the composition are administered to the subject per treatment session. In some embodiments, an average of at least 10 injections of the composition are administered to the subject per treatment session. In some embodiments, the grade improvement in submental adipose tissue reduction refers to a Patient Submental Fat Scale, Clinical Submental Fat Scale, or a Composite CSFS/PSFS Responder Scale. In some embodiments, each treatment session is administered more than 25, 28, 30, 35, 40, 45, 50, or 55 days apart but not more than 60 days apart. In some embodiments, the grade improvement in submental adipose tissue reduction refers to a Patient Submental Fat Scale, Clinical Submental Fat Scale, or a Composite CSFS/PSFS Responder Scale. Described herein, in some aspects, is a method of improving elimination or reduction of subcutaneous adipose tissue of a subject comprising injecting the subject with a pharmaceutical composition comprising 2.0-4.5% w/w polidocanol, wherein the subject exhibits at least a one grade improvement in flank adipose tissue reduction. Described herein, in some aspects, is a method of improving body contouring of the flank area of a subject comprising injecting the subject with a pharmaceutical composition comprising 2.0-4.5% w/w polidocanol, wherein the subject exhibits at least a one grade improvement in body contouring of the flank area. In some embodiments, the pharmaceutical composition comprises 3.0-4.5% w/w polidocanol. In some embodiments, the pharmaceutical composition is administered by subcutaneous injection. In some embodiments, the subject receives up to 6 injections at one-month intervals. In some embodiments, the one grade improvement in submental adipose tissue reduction in the flank of a subject is exhibited within 12, 11, 10, 9, 8, or 4 weeks following injection with the pharmaceutical composition. In some embodiments, the grade improvement in submental adipose tissue reduction refers to a Patient Global Impression of Change or a Clinical Global Impression of Change.

### BRIEF DESCRIPTION OF THE DRAWINGS

FIG. 1 illustrates the photonumeric guide for assessing varying degrees of submental fat which was used by the “patient” (PSFS) and clinician (CSFS) to assess the effectiveness of polidocanol described herein for eliminating submental adipose tissue in the patient (also referred to as a subject).

FIGS. 2A to 2E illustrate a non-limiting example of submental study. FIG. 2A illustrates co-primary endpoint-Composite CSFS/PSFS responders>/=2. CSFS stands for Clinical Submental Fat Scale and PSFS stands for patient submental fat scale. FIG. 2A also illustrates comparison with Kybella (deoxycholic acid). FIG. 2B illustrates co-primary endpoint-Composite CSFS/PSFS responders>/=1. FIG. 2C illustrates CSFS/PSFS=3-point improvement. FIG. 2D illustrates proportion of responders clinician submental fat scale (ITT). FIG. 2E illustrates proportion of responders patient submental fat scale (ITT). * denotes statistical significance based on Fisher Exact Test. ** denotes average of Refine 1 and 2 trials.

FIG. 3 illustrates 10XB101 showing >2-Fold increase in mean CSFS change at same time point versus Kybella (ITT Analysis).

FIG. 4 illustrates proportion of responders overtime with CSFS≥2 (ITT).

FIG. 5 illustrates greater loss of submental fat during treatment leads to reduced drug volume and injection number.

FIG. 6 illustrates submental fat loss after 12-week post treatment accompanied by decreased CSFS and PSFS.

FIG. 7 illustrates improvements to an injection related side effect(s) when administering polidocanol pharmaceutical formulations according to methods described herein.

FIG. 8 illustrates improvements to an injection related side effect(s) when administering polidocanol pharmaceutical formulations according to methods described herein.

FIGS. 9A and 9B illustrate injection related side effects in subject administered 1% deoxycholic acid.

FIG. 10 is a bar graph illustrating Clinician Global Impression of Change (CGIC) after 4-12 weeks post treatment.

FIG. 11 is a bar graph illustrating Patient Global Impression of Change (PGIC) after 4-12 weeks post treatment.

FIG. 12 illustrates flank fat loss after 4-weeks post treatment accompanied by moderate improved CGIC and PGIC.

FIG. 13 illustrates flank fat loss after 12-weeks post treatment accompanied by much improved CGIC and PGIC.

### DETAILED DESCRIPTION

While pharmaceutical detergent compositions for reduction of adipose tissue, including submental adipose tissue, are utilized, they have failed to achieve widespread adoption in the field. In cases of reduction of submental adipose tissue with detergent compositions, such compositions have failed to achieve a patient significant two grade improvement reduction in submental adipose tissue within a reasonable number of treatment sessions, and induce significant injection related side effects in human subjects. The current standard of care for non-surgical reduction of adipose tissue with injectable pharmaceutical detergent compositions is subcutaneous injection of 1% w/w deoxycholic acid. However, many human subjects undergoing subcutaneous injection of 1% w/w deoxycholic acid for reduction of submental adipose tissue fail to exhibit at least a two grade improvement reduction in submental adipose tissue, which the minimum improvement desired by most subjects to be considered a cosmetically significant result, with less than 19% of subjects exhibiting a two grade improvement or larger reduction in submental adipose tissue. Further, as many as 87% of subjects undergoing subcutaneous injection of 1% w/w deoxycholic acid exhibit an injection related side effect, with up to 42% of said subjects exhibiting an injection related side effect which lasts for more than 30 days. There remains a need in the art for improved methods of reducing adipose tissue, including submental adipose tissue, in human subjects with increased safety and efficacy as compared the current standard of care.

Similar to deoxycholic acid, polidocanol functions as a detergent to induce cytolysis of adipocytes and result in local reduction of fat tissue. Surprisingly and unexpectedly, is shown that the polidocanol pharmaceutical compositions disclosed herein when administered according to the methods described herein result in approximately 80% of human subjects undergoing treatment exhibiting a two grade improvement or larger reduction in submental adipose tissue, with fewer subjects exhibiting any injection related side effect. Subject that do experience an injection related side effect exhibited a side effect of reduced severity and duration, with a significant majority of subjects exhibiting a grade 0 (none) or grade 1 (mild) side effect, and less than 5% of subjects exhibiting an injection related side effect lasting longer than 30 days. Such improved results as to both safety and efficacy with a detergent based active pharmaceutical ingredient when injected subcutaneous for reduction of adipose tissue in human subjects has not been previously observed, represents a significant improvement over the standard of care, and out performs expectations of those of skill in the art in safety and efficacy for such polidocanol detergent based formulations.

Described herein, inter alia, are adipolytic formulations, non-invasive methods and systems, and kits for body contouring, including reducing, emulsifying, and/or eliminating subcutaneous adipose tissue, including fat deposits. These formulations have been shown to be beneficial, for example, for emulsifying and destroying cell membranes of adipose tissue. In some embodiments, the adipolytic formulation or a method of utilizing the adipolytic formulation comprises the use of polidocanol (also referred to as Laureth-9) or polidocanol-like compounds described herein. In some embodiments, the polidocanol or polidocanol-like compound comprise a structure of

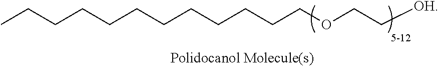

In some embodiments, the polidocanol or polidocanol-like compound consist a structure of

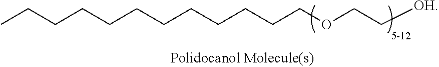

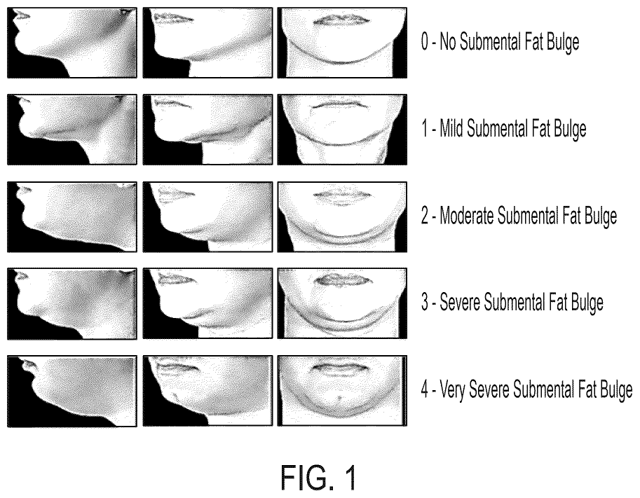

In some aspects, polidocanol functions as a detergent, where detergent effects of polidocanol can induce cytolysis of adipocytes and result in local reduction of fat tissue. Focal reduction of fat tissue is a commonly desired treatment sought by patients. For example, submental fat (SMF) accumulation can lead to loss of definition in the submental area and fullness or convexity of the cervicomental angle. In some embodiments, the effectiveness of polidocanol for reducing or eliminating fat accumulation such as SMF can be determined by examining and scoring the reduction or elimination of such fat accumulation in the treated subjects. For example, FIG. 1 illustrates a visual representation of the scoring for patent submental fat scale (PSFS).

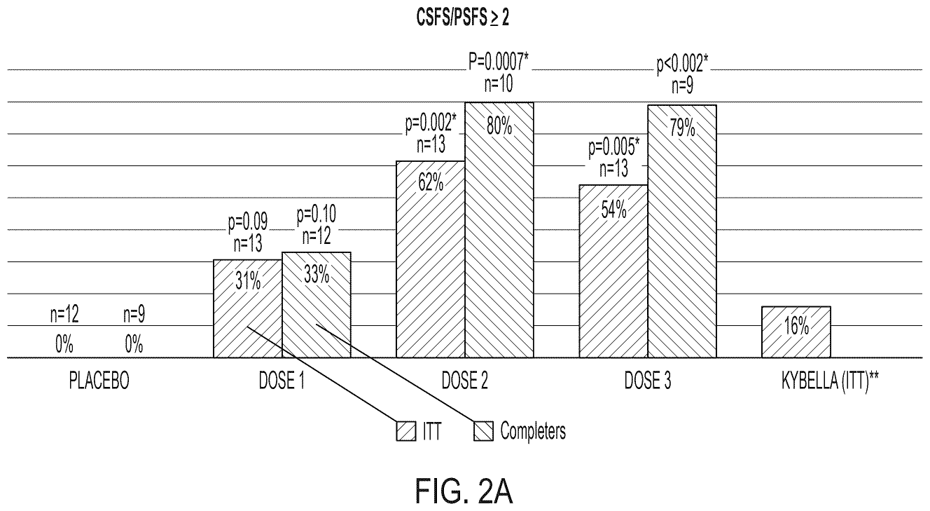

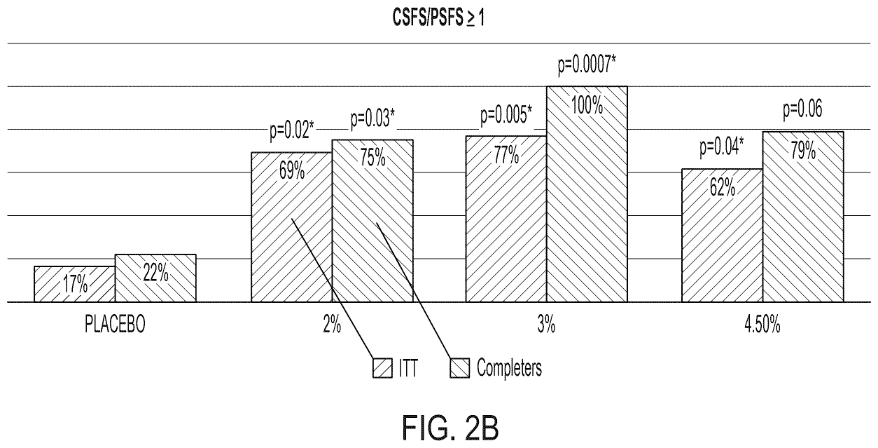

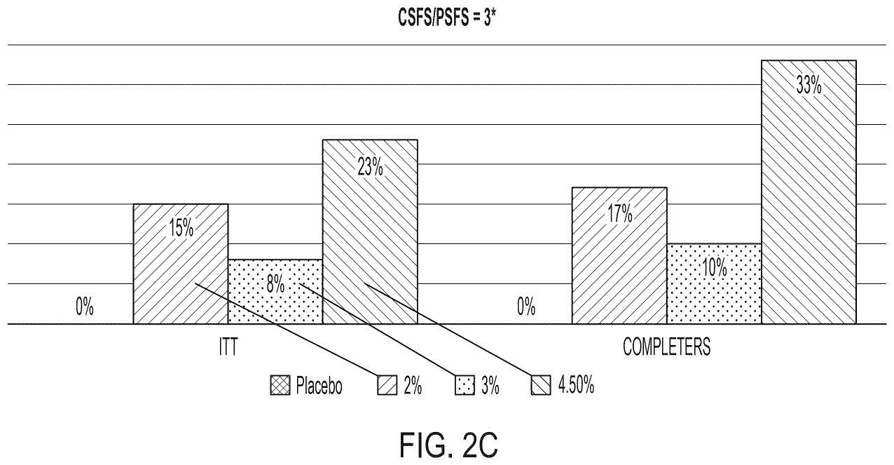

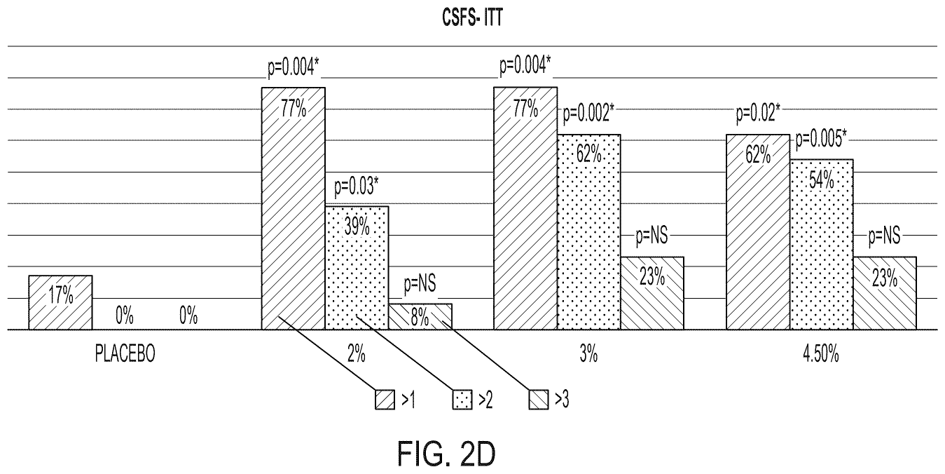

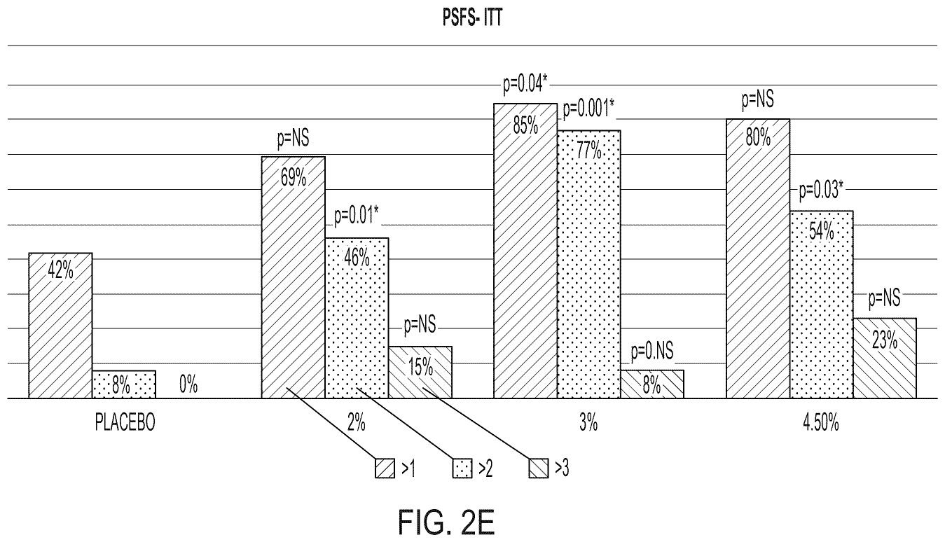

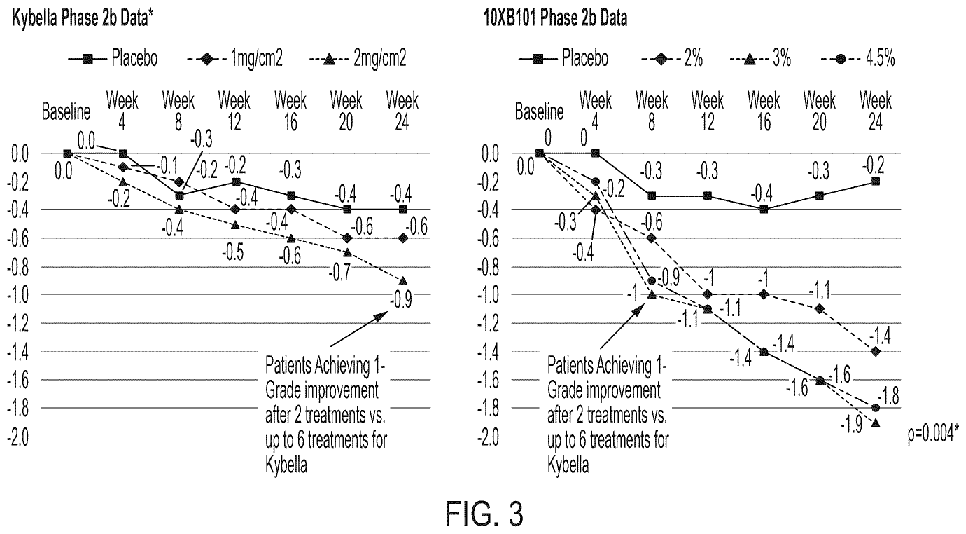

Described herein, in some aspects, is a method of improving elimination of subcutaneous adipose tissue in a subject comprising injecting the subject with a pharmaceutical composition comprising 3.0-4.5% w/w polidocanol. In some embodiments, the improvement of elimination of subcutaneous adipose tissue can be compared to elimination of subcutaneous adipose tissue induced by other agents. In some embodiments, the other agents comprise deoxycholic acid. In some embodiments, the method improves elimination of subcutaneous adipose tissue in the subject by at least 1.5× compared to injection of at least 1% w/w deoxycholic acid. FIG. 2A and FIG. 3 illustrate such improvement over deoxycholic acid. Also described herein, in some aspects, is a method of improving elimination of subcutaneous adipose tissue in a subject comprising injecting the subject with a pharmaceutical composition comprising 3.0-4.5% w/w polidocanol, where the subject exhibits at least a two grade improvement in submental adipose tissue reduction. In some embodiments, the method of eliminating of subcutaneous adipose tissue in a subject comprises injecting the subject with a plurality of doses of a pharmaceutical composition comprising 3.0-4.5% w/w polidocanol in a single treatment session. In some embodiments, the method comprises injecting the subject with a plurality of doses of a pharmaceutical composition comprising 3.0-4.5% w/w polidocanol over an area of at least 15 cm². In some embodiments, the method comprises injecting the subject with a plurality of doses of a pharmaceutical composition over an area of at least 16, 17, 18, 19, 20, 21, 22, 23, 24, 25, 26, 27, 28, 29, 30, 35, or 40 cm². In some embodiments, the method comprises injecting the subject with a pharmaceutical composition comprising 3.0-4.5% w/w polidocanol in a plurality of treatment sessions, where each treatment session is more than 23 days apart but not more than 60 days apart. In some embodiments, the method comprises injecting the subject with a pharmaceutical composition comprising 3.0-4.5% w/w polidocanol in a plurality of treatment sessions, comprising at administering between two and six treatment sessions to the subject. In some embodiments, the method reduces an injection related side effect in a subject.

### Definitions

The term “polidocanol” is used herein in accordance with its plain, ordinary meaning and includes the active pharmaceutical ingredient by the same name. Polidocanol includes a terminal alcohol connected to a hydrophilic polyethoxylate portion (ethylene glycol units) and a non-polar hydrophobic carbon chain. Polidocanol is typically provided as a synthetic mixture of alkyl ethoxylate homologues. The ethoxylate homologues may have an average carbon chain length from about 10 to about 14 (e.g. about 12). The average number of ethoxylate units may be from about 6 to about 12 (e.g. about 6, 7, 8 or 9). The average number of ethoxylate units may be from about 7 to about 11 (e.g. about 6, 7, 8 or 9). In embodiments, the average number of ethoxylate units is about 6. In embodiments, the average number of ethoxylate units is about 7. In embodiments, the average number of ethoxylate units is about 8. In embodiments, the average number of ethoxylate units is about 9. In embodiments, the polidocanol may be the mixture produced by reacting 1 mole of the corresponding C12 alcohol with 9 moles of ethoxide equivalents (e.g. ethylene oxide). In embodiments, the polidocanol is the product of reacting 1 mole of the corresponding C12 alcohol with 9 moles of ethoxide equivalents under basic conditions (e.g. with a metal base such as potassium hydroxide).

The term “average molecular weight” as used herein refers to the weight average molecular weight of the polymer. The average molecular weight is determined by summing the weights of all the chains and then dividing by the total number of chains.

The term “Clinical Submental Fat Scale” (CSFS) as used herein refers to a discrete scale based on the investigator's clinical evaluation of the subject as the subject appears on the day of the evaluation. The CSFS score is based on the investigator's clinical evaluation of the subject, including palpation of the chin and neck area; anterior, oblique, and profile views of the chin and neck; and observation of pronation, supination, and lateral movement of the head determined by an Investigator's selection of a best grade (0 to 4) on the 5-point static scale that best describes the subject's level of SMF according to provided representative pictures (in FIG. 1) and the following scale:

| Score | Description |
|---|---|
| 0 | No Submental Fat Bulge |
| 1 | Mild Submental Fat Bulge |
| 2 | Moderate Submental Fat Bulge |
| 3 | Severe Submental Fat Bulge |
| 4 | Very Severe Submental Fat Bulge |

The term “Patient Submental Fat Scale” (PSFS) as used herein refers to a single question survey of the subject based on how the subject assess their SMF on the day of the evaluation. The subject will position their head in the Frankfort plane and use a mirror to look at the area under their chin to help them answer the survey with instructions provided to train the subject on proper head placement and mirror use during assessment. In the single question survey the subject assess the best grade (0 to 4) on the 5-point static scale that best describes the subject's level of SMF according to provided representative pictures (in FIG. 1), and the following scale:

| Score | Description |
|---|---|
| 0 | No Submental Fat Bulge |
| 1 | Mild Submental Fat Bulge |
| 2 | Moderate Submental Fat Bulge |
| 3 | Severe Submental Fat Bulge |
| 4 | Very Severe Submental Fat Bulge |

The term “Composite CSFS/PSFS Responder Scale” as used herein refers to a “xyz” level of improvement (e.g. ≥1 or ≥2)” refers to a grading assessment where BOTH the CSFS and PSFS values are at or above the designated “xyz” level of grade improvement. For example; at a given visit if the CSFS grade shows a 1 point reduction from baseline and PSFS reflects a 2 point reduction from baseline then the Composite CSFS/PSFS scale meets the ≥1 level of success, but fails to meet the ≥2 level of success.

The term “grade improvement” as used herein (e.g., a two grade improvement, a one grade improvement, etc.), refers to a score change on the PSFS or the CSFS (e.g., a Score 3 to a Score 2 constituting a two grade improvement).

The term “effective treatment area” as used herein refers to the area of excess submental fat that is present compared to “No submental Fat” as defined in the grading scale (FIG. 1) as a CSFS score of zero (0).

The side effect grades as used herein refer to a: grade 0 (none), grade 1 (mild), grade 2 (moderate), or a grade 3 (severe) side effect.

In embodiments, the average molecular weight of the polidocanol (the mixture of alkyl ethoxylates) is about 400 g/mol to about 800 g/mole. In embodiments, the average molecular weight of the polidocanol is about 400 g/mol to about 700 g/mole. In embodiments, the average molecular weight of the polidocanol is about 400 g/mol to about 650 g/mole. In embodiments, the average molecular weight of the polidocanol is about 480 g/mol to about 620 g/mole. In embodiments, the average molecular weight of the polidocanol is about 480 g/mol to about 600 g/mole. In embodiments, the average molecular weight of the polidocanol is about 450 g/mol to about 750 g/mole. In embodiments, the average molecular weight of the polidocanol is about 450 g/mol. In embodiments, the average molecular weight of the polidocanol is about 460 g/mol. In embodiments, the average molecular weight of the polidocanol is about 470 g/mol. In embodiments, the average molecular weight of the polidocanol is about 480 g/mol. In embodiments, the average molecular weight of the polidocanol is about 490 g/mol. In embodiments, the average molecular weight of the polidocanol is about 500 g/mol. In embodiments, the average molecular weight of the polidocanol is about 510 g/mol. In embodiments, the average molecular weight of the polidocanol is about 520 g/mol. In embodiments, the average molecular weight of the polidocanol is about 530 g/mol. In embodiments, the average molecular weight of the polidocanol is about 540 g/mol. In embodiments, the average molecular weight of the polidocanol is about 550 g/mol. In embodiments, the average molecular weight of the polidocanol is about 560 g/mol. In embodiments, the average molecular weight of the polidocanol is about 570 g/mol. In embodiments, the average molecular weight of the polidocanol is about 580 g/mol. In embodiments, the average molecular weight of the polidocanol is about 590 g/mol. In embodiments, the average molecular weight of the polidocanol is about 600 g/mol. In embodiments, the average molecular weight of the polidocanol is about 625 g/mol. In embodiments, the average molecular weight of the polidocanol is about 650 g/mol. In embodiments, the average molecular weight of the polidocanol is about 493 g/mol. In embodiments, the average molecular weight of the polidocanol is 493 g/mol.

In embodiments, the polidocanol contains a mixture of C12 ethoxylates (e.g. with homologues) ranging from 3-18 ethoxide units. In embodiments, the polidocanol contains a mixture of C12 ethoxylates ranging from 1-23 ethoxide units. In embodiments, the polidocanol contains a mixture of C12 ethoxylates ranging from 15-20 ethoxide units. In embodiments, there are approximately 15 ethoxylated C12 molecular species. In embodiments, no one ethoxylated species is present at more than 20% of the total alkyl ethoxylates, in some not more than 15% and in some not more than 10%. In embodiments, the average degree of ethoxylation is 7. In embodiments, the average degree of ethoxylation is 8, 9, 10 or 11. In embodiments, C14 alkyl ethoxylate units are present in the polidocanol mixture. In embodiments, the average ethoxylation is 6, in some it is 7, in others it is 8 and in others it is 9. In embodiments, a polidocanol with a narrow range of ethoxylate units is produced from a reaction involving catalysts, such as metal oxide, that produces ethoxylated alcohols in the range of 6-11. In embodiments, the average molecular weight of the alkyl ethoxylated homologues is approximately 440 g/mole. In embodiments, the average molecular weight of the alkyl ethoxylated homologues is 500 g/mol, in some embodiments 600 g/mole.

A “C₃-C₆ alcohol,” as used herein, refers to a compound having from 3 to 6 carbons and at least one hydroxyl moiety. In embodiments, the C₃-C₆ alcohol contains an alkyl chain of from 3 to 6 carbons, wherein each carbon is substituted with hydrogen or hydroxyl. In embodiments, the C₃-C₆ alcohol contains an alkyl chain of from 3 to 6 carbons, wherein each carbon is substituted with hydrogen or hydroxyl, and the C₃-C₆ alcohol contains 1 to 6 hydroxyl moieties. In embodiments, the C₃-C₆ alcohol contains an alkyl chain of from 3 to 6 carbons, wherein each carbon is substituted with hydrogen or hydroxyl, and the C₃-C₆ alcohol contains 1 to 3 hydroxyl moieties. In embodiments, the C₃-C₆ alcohol contains an alkyl chain of from 3 to 6 carbons, wherein each carbon is substituted with hydrogen or hydroxyl, and the C₃-C₆ alcohol contains 1 or 2 hydroxyl moieties. In embodiments, the C₃-C₆ alcohol contains an alkyl chain of from 3 to 6 carbons, wherein each carbon is substituted with hydrogen or hydroxyl, and the C₃-C₆ alcohol contains 2 hydroxyl moieties. In embodiments, the C₃-C₆ alcohol contains an alkyl chain of 3 carbons (i.e. a C3 alcohol), wherein each carbon is substituted with hydrogen or hydroxyl, and the C₃ alcohol contains 1 to 3 hydroxyl moieties. In embodiments, the C₃-C₆ alcohol contains an alkyl chain of 3 carbons (i.e. a C3 alcohol), wherein each carbon is substituted with hydrogen or hydroxyl, and the C₃ alcohol contains 1 or 2 hydroxyl moieties. In embodiments, the C₃-C₆ alcohol contains an alkyl chain of 3 carbons (i.e. a C3 alcohol), wherein each carbon is substituted with hydrogen or hydroxyl, and the C3 alcohol contains 2 hydroxyl moieties. In embodiments, the C₃-C₆ alcohol is a propane-diol. In embodiments, the C₃-C₆ alcohol is a propane-1,2-diol (propylene glycol).

As used herein, the term “polidocanol-like compound” refers to a compound of Formula I or Formula II described herein, including embodiments thereof.

An “effective amount” is an amount sufficient to accomplish a stated purpose (e.g. achieve the effect for which it is administered, treat a disease, reduce adipose tissue or cells, lyse cells, reduce one or more symptoms of a disease or condition). An example of an “effective amount” is an amount sufficient to contribute to the treatment, prevention, or reduction of a symptom or symptoms of a disease or condition, which could also be referred to as a “therapeutically effective amount.” A “therapeutically effective amount,” as used herein, refers to a sufficient amount of an agent (e.g., polidocanol) or other compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses may be the amount of the composition including a compound as disclosed herein required to provide a clinically significant decrease in disease symptoms without undue adverse side effects. An appropriate “effective amount” in any individual case can be determined using techniques, such as a dose escalation study. The term “therapeutically effective amount” includes, for example, a prophylactically effective amount. An “effective amount” of a compound disclosed herein, such as polidocanol used alone or in combination with other compounds, is an amount effective to achieve a desired pharmacologic effect or therapeutic improvement without undue adverse side effects. It is to be understood that “an effective amount” or “a therapeutically effective amount” can vary from subject to subject, due to variation in metabolism of a compound of Formula I or Formula II as described herein (such as polidocanol), age, weight, general condition of the subject, the condition being treated, the severity of the condition being treated, and the judgment of the prescribing physician. A “cosmetically effective amount” as used herein refers to the amount of a compound sufficient to improve the outward physical appearance of a subject. The outward physical appearance of a subject includes, for example, the reduction of fat deposition in certain regions of the body including, for example, the midsection of the body. In embodiments, a cosmetically effective amount refers to a sufficient amount of an agent (e.g., polidocanol) which will improve the cosmetic appearance at the localized site of treatment. A cosmetically effective amount of polidocanol may be an amount effective to achieve a cosmetically desirable improvement. It is to be understood that a cosmetically effective amount can vary from subject to subject, due to numerous factors including for example age, weight, general condition of the subject, the condition being treated, the severity of the condition being treated, and the judgment of the prescribing physician. For example, a cosmetically effective amount of an agent (e.g., polidocanol) may be an amount capable of improving the cosmetic appearance at the localized site of treatment by reducing the amount of adipose tissue or cells at the localized size or an area adjacent to the localized site of treatment.

In certain embodiments, the phrase “pharmaceutically acceptable salt(s) and other suitable forms”, and similar language as used herein, means those salts, solvates, hydrates, and other suitable forms of compounds described herein that are safe and effective for administration in mammals. Pharmaceutically acceptable salts include salts of acidic or basic groups present in compounds described herein. In certain embodiments, the pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. In certain embodiments, one or more compounds described herein form pharmaceutically acceptable salts with various amino acids. In certain embodiments, the base salts include, but are not limited to, aluminum, calcium, lithium, magnesium, potassium, sodium, zinc, and diethanolamine salts. Pharmaceutically acceptable salts in certain embodiments of the formulations described herein, are as described in Berge et al., J. Pharm. Sci. 66:1-19 (1977), incorporated in entirety by reference herein.

In certain embodiments, the term “cosmetically acceptable salt and other suitable forms” and similar language used herein means any salt, hydrate, solvate, or other suitable form that is cosmetically tolerated if used appropriately for a cosmetic treatment especially if used on or applied to humans and/or mammals. In certain embodiments, these salts include, but are not restricted to the salts used to form base addition salts, either inorganic, such as for example and in a non-limiting sense, lithium, sodium, potassium, calcium, magnesium or aluminum, among others, or organic such as for example and in a non-limiting sense, ethylamine, diethylamine, ethylenediamine, ethanolamine, diethanolamine, arginine, lysine, histidine, or piperazine among others; or acid addition salts, either organic, such as for example and in a non-limiting sense, acetate, citrate, lactate, malonate, maleate, tartrate, fumarate, benzoate, aspartate, glutamate, succinate, oleate, trifluoroacetate, oxalate, pamoate or gluconate among others, or inorganic, such as for example and in a non-limiting sense, chloride, sulfate, borate, or carbonate among others. The cosmetically acceptable salts described herein can be obtained by conventional methods well known in the state of the art as described in Berge et al., J. Pharm. Sci. Id., incorporated in entirety by reference herein.

“Pharmaceutically acceptable excipient” and “pharmaceutically acceptable carrier” refer to a substance that aids the administration of an active agent to a subject and can be included in the compositions described herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, mannitol, gum acacia, calcium phosphate, alginates, tragacanth, calcium silicate, microcrystalline cellulose, cellulose, syrup, and methyl cellulose, colors, and the like. The formulations can additionally include: lubricating agents such as talc, magnesium stearate, and mineral oil; wetting agents; emulsifying and suspending agents; preserving agents such as methyl and propylhydroxy benzoates; sweetening agents; and flavoring agents. The compositions described herein can be formulated so as to provide quick, sustained or delayed release of the active ingredient after administration to the patient by employing procedures known in the art. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

Any suitable pharmaceutically acceptable excipient appropriate for a particular route of administration can be used. Examples of pharmaceutically acceptable carriers include, but are not limited to, buffers, saline, or other aqueous media. The compounds of the invention are preferably soluble in the carrier which is employed for their administration (e.g., subcutaneous). Alternatively, a suspension of the active compound or compounds (e.g., a suspension of microparticles) in a suitable carrier is employed. Some embodiments include any suitable lipophilic carrier, for example, modified oils (e.g., CREMOPHOR® BASF, Germany), soybean oil, polyethylene glycol, derivatized polyethers, combinations thereof, and the like. Some embodiments include a microparticulate and/or a nanoparticulate carrier. Some embodiments include one or more sustained or controlled release carriers or agents, for example, polymer microspheres. Some embodiments include excipients suitable for stable suspensions for micronized particles of polidocanol. In further or additional embodiments, the formulations include an immediate release excipient. In embodiments, the pharmaceutically excipient is not a C₃-C₆ alcohol (e.g. propylene glycol).

The term “fat pad” refers herein to any cushions made of a pocket of fascia and filled with fat deposits (e.g. fatty acids) that are present in humans or mammalians.

As used herein, “administering” means administering by any route, including local, parenteral, by infusion, injection, implantation, or subcutaneous administration. For example, the formulations described herein can be administered by subcutaneous injection or delivery. For example, the formulations described herein can be administered by intravenous injection or delivery. As used herein, a formulation formulated for subcutaneous injection can include sterile isotonic aqueous buffer. Where necessary, the compositions can also include a solubilizing agent.

Embodiments of the composition are formulated for administration by any suitable method, for example, as described in REMINGTON: THE SCIENCE AND PRACTICE OF PHARMACY (21st ed., Lippincott Williams & Wilkins). Exemplary routes of administration include, but are not limited to parenteral, oral, subcutaneous, topical, intramuscular, transdermal, transmucosal, sublingual, intranasal, transvascular, subcutaneous, orbital, or respiratory. In some embodiments, the composition is formulated for injection of an area at which treatment is desired, for example, in a regional fat deposit, such as for example excessive sub-mental adiposity. In some embodiments, provided are methods for subcutaneous delivery as well as compositions and formulations that are formulated to be suitable for subcutaneous delivery.

Injectable formulations are administered using any method known in the art, for example, using a single needle, multiple needles, and/or using a needleless injection device. In some embodiments, a tissue loading dose of the active ingredients formulated in a suitable carrier delivered by injection. In some embodiments, delivery includes single needle injection. In some embodiments, delivery includes injection using a multi-needle array, which, in some embodiments, provides a wide dispersion of the formulation in the target tissue. In some embodiments, formulations are injected in a manner that allows dispersal into the appropriate layer of subcutaneous fat in areas where regional fat exists. In some embodiments, a micropump is used to deliver the injectable formulations.

The formulations described herein may also be administered via a device. A number of devices have been proposed to facilitate self-administration of pharmaceutical formulations. The device typically includes a reservoir containing, for example, pre-loaded with, the pharmaceutical formulation to be administered. For example, a micropump can provide precise subcutaneous administration of small quantities of a liquid pharmaceutical formulation. Such micropumps can be compact and portable. Another type of device useful for subcutaneous delivery or administration of pharmaceutical formulations is often referred to as a patch device or a pump-patch device. Patch devices usually are attached directly to the skin of a patient.

Accordingly, in various embodiments, a device such as a micropump or patch device can include a reservoir containing a pharmaceutical formulation, a subcutaneous injection needle configured for removable insertion into skin of a patient, a micropump having an inlet in fluid communication with the reservoir and an outlet in fluid communication with the subcutaneous injection needle, a control system configured for controlling the micropump to deliver the pharmaceutical formulation from the reservoir to the subcutaneous injection needle, whereby the pharmaceutical formulation is administered subcutaneously to a patient, and a housing for supporting the reservoir, subcutaneous injection needle, micropump and control system, the housing being portable and adapted for contact with the skin of the patient. The pharmaceutical formulation contained within the reservoir can be any of the pharmaceutical formulations of the present teachings.

In certain embodiments, the device can be of a unitary construction. Such devices can be for a single or one-time use. In particular embodiments, the device can be of a multi-piece construction, in such devices, a disposable or a re-sizeable portion or component can be present. For example, a housing defining or including the reservoir can be a disposable or a reusable component of the device, in some embodiments, the disposable or reusable housing defining or including the reservoir can contain a pharmaceutical formulation of the present teachings. In various embodiments, the subcutaneous injection needle can be a disposable component of the device. When administered for the treatment or inhibition of a particular disease state, condition or disorder, it is understood that an effective dosage can vary depending upon many factors such as the particular compound or therapeutic combination utilized, the mode of administration, and severity of the condition being treated, as well as the various physical factors related to the individual being treated. In therapeutic applications, a compound or therapeutic combination of the present teachings can be provided to a patient already suffering from a disorder, for example, subcutaneous adipose tissue, in an amount sufficient to reduce and/or prevent the symptoms of the disorder and its complications. The dosage to be used in the treatment of a specific individual typically must be subjectively determined by the attending physician. The variables involved include the specific condition and its state as well as the size, age and response pattern of the patient.

The term “about” when used in conjunction with a numerical value indicates the value is within 10% of the numerical value. In embodiments, “about” means within 5% of the numerical value. In embodiments, “about” means within 1% of the numerical value. For example, about 1.0% means 0.9% to 1.1%. It will be understood that the indicated value is included as an embodiment within the range specified by the use of “about” referring to the numerical value. In embodiments, “approximately” when applied to a numerical value has the same meaning as “about”.

The term “buffer” is used in accordance with its common meaning within the biological sciences and refers to a solution including a mixture of a weak acid and its conjugate base or a weak base and its conjugate acid. A buffer has the property that the pH of the solution changes very little when a small amount of acid or base is added to it. Buffer solutions are used as a means of keeping pH at a nearly constant value in a wide variety of chemical applications. Examples of suitable buffers include phosphate buffers and those known in the literature (see, for example, Troy, D. B., ed. (2005) Remington: The Science and Practice of Pharmacy, 21st ed., Lippincott Williams & Wilkins).

### Methods of Treatment

In an aspect is provided a method of reducing subcutaneous adipose tissue in a subject in need thereof, the method including administering to the subject a pharmaceutical formulation described herein. Surprisingly and unexpectedly, is shown that the polidocanol pharmaceutical compositions disclosed herein when administered according to the methods described herein result in approximately 80% of human subjects undergoing treatment exhibiting a two grade improvement or larger reduction in submental adipose tissue, with fewer subjects exhibiting any injection related side effect. Subject that do experience an injection related side effect exhibited a side effect of reduced severity and duration, with a significant majority of subjects exhibiting a grade 0 (none) or grade 1 (mild) side effect, and less than 5% of subjects exhibiting an injection related side effect lasting longer than 30 days. Such improved results as to both safety and efficacy with a detergent based active pharmaceutical ingredient when injected subcutaneous for reduction of adipose tissue in human subjects has not been previously observed, represents a significant improvement over the standard of care, and out performs expectations of those of skill in the art in safety and efficacy for such polidocanol detergent based formulations.

In some aspects, the method improves elimination of subcutaneous adipose tissue in the subject by at least 50% compared to injection of 1% w/w deoxycholic acid. In some embodiments, the method improves elimination of subcutaneous adipose tissue in the subject by at least 50% compared to injection of 2% w/w deoxycholic acid. In some embodiments, the subject exhibits at least a two grade improvement in submental adipose tissue reduction. In some embodiments, the method comprises injecting the subject with a plurality of doses of the pharmaceutical composition/In some embodiments, the method comprises injecting the subject with an average of more than 30 doses per treatment during a clinical treatment regimen of up to six treatments of the pharmaceutical composition described here. In some embodiments, the method comprises injecting the subject with a plurality of doses of a pharmaceutical composition over an area of at least 15 cm². In some embodiments, the method comprises injecting the subject with a plurality of doses of a pharmaceutical composition over an area of at least 16, 17, 18, 19, 20, 21, 22, 23, 24, 25, 26, 27, 28, 29, 30, 35, or 40 cm². In some embodiments, the method comprises injecting the subject with the pharmaceutical composition in a plurality of treatment sessions, each treatment session being more than 23 days apart but not more than 60 days apart. In some embodiments, the method reduces an injection related side effect in the subject. In some embodiments, the method improves a rate of elimination of subcutaneous adipose tissue in the subject compared to injection of 1% w/w deoxycholic acid. In some embodiments, the method improves elimination of subcutaneous adipose tissue in the subject by at least 1.75, 2, 2.25, 2.5, 2.75, 3, 3.25, 3.5, 3.75, 4, 4.5, or 5× compared to injection of 1% w/w deoxycholic acid. In some embodiments, the method improves elimination of subcutaneous adipose tissue in the subject by at least 1.75, or 2× compared to injection of 2% w/w deoxycholic acid. In some embodiments, the method reduces a mass of subcutaneous adipose tissue in the subject. In some embodiments, the method reduces a mass of subcutaneous adipose tissue in the subject in a region of the injection by 10, 20, 30, 40, 50, 60, or 75% w/w. In some embodiments, the region is a submental region. In some embodiments, the method reduces a volume of subcutaneous adipose tissue in the subject. In some embodiments, the method reduces a volume of subcutaneous adipose tissue in the subject in a region of the injection by 10, 20, 30, 40, 50, 60, or 75% v/v. In some embodiments, the region is a submental region. In some embodiments, the subject exhibits at least a one grade improvement in submental adipose tissue reduction. In some embodiments, the one grade improvement in submental adipose tissue reduction is exhibited within 12, 11, 10, 9, or 8 weeks following injection with the pharmaceutical composition. In some embodiments, the one grade improvement in submental adipose tissue reduction is exhibited within 12, 11, 10, 9, 8, 7, 6, 5, or 4 weeks following injection with the pharmaceutical composition. In some embodiments, the one grade improvement in submental adipose tissue reduction in the flank of a subject is exhibited within 12, 11, 10, 9, 8, 7, 6, 5, or 4 weeks following injection with the pharmaceutical composition. In some embodiments, the one grade improvement in submental adipose tissue reduction is exhibited following administering at least three treatment sessions to the subject. In some embodiments, the subject exhibits at least a two grade improvement in submental adipose tissue reduction. In some embodiments, the one grade improvement in submental adipose tissue reduction is exhibited within 24 weeks following injection with the pharmaceutical composition. In some embodiments, the one grade improvement in submental adipose tissue reduction is exhibited following administering at least six treatment sessions to the subject. In some embodiments, the method comprises injecting the subject with up to 50 doses of the pharmaceutical composition. In some embodiments, the method comprises injecting the subject with at least 50 doses of the pharmaceutical composition. In some embodiments, the method comprises injecting the subject with at least 20 and up to 50 doses of the pharmaceutical composition. In some embodiments, the method comprises injecting the subject with an average of at least 30 and up to 50 doses of the pharmaceutical composition. In some embodiments, each dose is not more than 0.2 mL. In some embodiments, each dose is less than 0.2 mL. In some embodiments, the method comprises administering at least three treatment sessions to the subject. In some embodiments, the method comprises administering at least six treatment sessions to the subject. In some embodiments, the method comprises administering between two and six treatment sessions to the subject. In some embodiments, the method comprises administering up to six treatment sessions to the subject. In some embodiments, each treatment session is administered at least 3 weeks apart. In some embodiments, each treatment session is administered more than 30 days apart. In some embodiments, each dose is injected at least 1 cm apart. In some embodiments, each dose is injected up to 1 cm apart. In some embodiments, each dose is injected about 1 cm apart. In some embodiments, a submental region of the subject is treated. In some embodiments, a flank region of the subject is treated. In some embodiments, an abdominal region of the subject is treated. In some embodiments, a lingual region of the subject is treated. In some embodiments, the subject exhibits at least a one grade improvement in submental adipose tissue reduction. In some embodiments, a method improves elimination or reduction of subcutaneous adipose tissue of a subject. In some embodiments, the method of improving elimination or reduction of subcutaneous adipose tissue of a subject comprising injecting the subject with a pharmaceutical composition comprising 2.0-4.5% w/w polidocanol, wherein the subject exhibits at least a one grade improvement in flank adipose tissue reduction. In some embodiments, a method improves body contouring of the flank area of a subject. In some embodiments, the method of improving body contouring of the flank area of a subject comprising injecting the subject with a pharmaceutical composition comprising 2.0-4.5% w/w polidocanol, wherein the subject exhibits at least a one grade improvement in body contouring of the flank area. In some embodiments, the one grade improvement in submental adipose tissue reduction is exhibited after 2 treatment sessions. In some embodiments, the one grade improvement in submental adipose tissue reduction is exhibited within about 8 weeks. In some embodiments, the subject exhibits at least a two grade improvement in submental adipose tissue reduction. In some embodiments, the two grade improvement in submental adipose tissue reduction is exhibited after 3 treatment sessions. In some embodiments, the two grade improvement in submental adipose tissue reduction is exhibited within about 24 weeks. In some embodiments, the grade improvement in submental adipose tissue reduction refers to a Patient Submental Fat Scale, Clinical Submental Fat Scale, or a Composite CSFS/PSFS Responder Scale. In some embodiments, the subject exhibits a decrease in convexity, fullness, bulging, bloating, stretching, in a region injected with the pharmaceutical composition. In some embodiments, the subject exhibits an improvement in appearance in a region injected with the pharmaceutical composition. In some embodiments, the pharmaceutical composition comprises about 2.0% w/w polidocanol. In some embodiments, the pharmaceutical composition comprises about 3.0% w/w polidocanol. In some embodiments, the pharmaceutical composition comprises about 4.5% w/w polidocanol. In some embodiments, the pharmaceutical composition is a subcutaneous injection formulation. In some embodiments, the pharmaceutical composition is an aqueous formulation. In some embodiments, the pharmaceutical composition comprises a cosolvent. In some embodiments, the cosolvent comprises propylene glycol. In some embodiments, the propylene glycol is comprised in an amount of up to 2.25% w/w. In some embodiments, the pharmaceutical composition comprises a buffer. In some embodiments, the pharmaceutical composition comprises a binary buffers system. In some embodiments, the binary buffer system comprises sodium phosphate and potassium phosphate. In some embodiments, the binary buffer system comprises sodium phosphate dibasic dihydrate and potassium phosphate monobasic. In some embodiments, the sodium phosphate dibasic dihydrate is comprised in an amount of up to about 0.25% w/w. In some embodiments, the potassium phosphate monobasic is comprised in an amount of up to about 0.1% w/w. In some embodiments, the pharmaceutical composition comprises an osmolarity of less than about 400 mM/kg. In some embodiments, the pharmaceutical composition comprises an alkaline pH. In some embodiments, the pharmaceutical composition comprises a pH of 7-8. In some embodiments, the pharmaceutical composition comprises a critical micelle concentration of up to 0.10 millimolar. In some embodiments, the pharmaceutical composition comprises a critical micelle concentration of 0.07-0.1 mM. In some embodiments, the injecting is subcutaneous. In some embodiments, the method locally reduces subcutaneous adipose tissue. In some embodiments, the method locally reduces submental adipose tissue. In some embodiments, the comparative injection of 1% w/w deoxycholic acid in a same volume and/or a same spacing of injections. In some embodiments, the comparative injection of 2% w/w deoxycholic acid in a same volume and/or a same spacing of injections. In some embodiments, the reduction in the injection related side effect is exhibited as compared to injection of 1% w/w deoxycholic acid. In some embodiments, the reduction in the injection related side effect is exhibited as compared to injection of 2% w/w deoxycholic acid. In some embodiments, the side effect comprises numbness, induration, paresthesia, nodule, pruritis, or nerve injury. In some embodiments, the side effect comprises Erythema, injection site pain, bruising, edema, or swelling. In some embodiments, the method described herein reduces a severity of the side effect. In some embodiments, the side effect comprises numbness, induration, paresthesia, nodule, pruritis, nerve injury, injection site pain, bruising, edema, or swelling, wherein a 100, 90, 80, 70, 60, 50, 45, 40, 35, 30, 25, 20, 15, 10, or 5% decrease in occurrence of the side effect is observed as compared to injection of 1% w/w deoxycholic acid. In some embodiments, the side effect comprises numbness, induration, paresthesia, nodule, pruritis, nerve injury, injection site pain, bruising, edema, or swelling, wherein a 100, 90, 80, 70, 60, 50, 45, 40, 35, 30, 25, 20, 15, 10, or 5% decrease in occurrence of the side effect is observed as compared to injection of 2% w/w deoxycholic acid. In some embodiments, the comparative injection of 1% w/w deoxycholic acid in a same volume and/or a same spacing of injections, or wherein the comparative injection of 2% w/w deoxycholic acid is in a same volume and/or a same spacing of injections.

Described herein, in some aspects, is a method of eliminating of subcutaneous adipose tissue in a subject comprising injecting the subject with a pharmaceutical composition comprising administering at least 2 treatment sessions to the subject, wherein at least one of the second or subsequent treatment(s) sessions comprises injecting the subject with a reduced concentration of polidocanol than was used in the first treatment. Described herein, in some aspects, is a method of reducing an injection related side effect in a subject comprising injecting the subject with a pharmaceutical composition comprising a progressively reduced concentration of polidocanol in a plurality of treatment sessions. Described herein, in some aspects, is a method of reducing an injection related side effect in a subject comprising injecting the subject with a pharmaceutical composition comprising in a plurality of treatment sessions comprising at least one of: a) administering a progressively reduced number of injections of the pharmaceutical composition in a second or subsequent treatment session(s) of the plurality of treatment sessions, or b) administering a reduced volume of injectate of the pharmaceutical composition in the second or subsequent treatment session(s). In some embodiments, the method includes administering at least 2 treatment sessions to the subject, wherein at least one of the second or subsequent treatment session(s) comprises injecting the subject with a reduced concentration of polidocanol than was used in the first treatment. In some embodiments, the method includes administering a plurality of treatment sessions to the subject, wherein a progressively reduced concentration of polidocanol is administered to the subject in the plurality of treatment sessions. In some embodiments, the method includes injecting the subject with a pharmaceutical composition comprising in a plurality of treatment sessions comprising at least one of: a) administering a progressively reduced number of injections of the pharmaceutical composition in a second or subsequent treatment session(s) of the plurality of treatment sessions, or b) administering a reduced volume of injectate of the pharmaceutical composition in the second or subsequent treatment session(s). In some embodiments, a pharmaceutical composition comprising up to 4.5% w/w polidocanol is administered to the subject in a first treatment session of a plurality of treatment sessions, and a pharmaceutical composition comprising less than 4.5% w/w polidocanol is administered to the subject in a subsequent treatment session(s). In some embodiments the pharmaceutical composition comprising up to 4.5% w/w polidocanol is administered to the subject in the first treatment session and a second treatment session of a plurality of treatment sessions. The method of any one of the preceding claims, wherein a pharmaceutical composition comprising 4.5% w/w polidocanol is administered to the subject in a first treatment session of a plurality of treatment sessions, and a pharmaceutical composition comprising less than 4.5% w/w polidocanol is administered to the subject in a subsequent treatment session(s). In some embodiments the pharmaceutical composition comprising 4.5% w/w polidocanol is administered to the subject in the first treatment session and a second treatment session of a plurality of treatment sessions. In some embodiments the pharmaceutical composition comprising less than 4.5% w/w polidocanol comprises up to 3.0% w/w polidocanol. In some embodiments the pharmaceutical composition comprising up to 3.0% w/w polidocanol is administered in a third treatment session and a fourth treatment session, wherein the subsequent treatment session(s) comprises the third treatment session and the fourth treatment session. In some embodiments the pharmaceutical composition comprising less than 4.5% w/w polidocanol comprises 3.0% w/w polidocanol. In some embodiments the pharmaceutical composition comprising 3.0% w/w polidocanol is administered in a third treatment session and a fourth treatment session, wherein the subsequent treatment session(s) comprises the third treatment session and the fourth treatment session. In some embodiments a pharmaceutical composition comprising less than 3.0% w/w polidocanol is administered to the subject in a second subsequent treatment session(s). In some embodiments the pharmaceutical composition comprising less than 3.0% w/w polidocanol comprises up to 2.0% w/w polidocanol. In some embodiments the second subsequent treatment session(s) comprise a fifth treatment session and a sixth treatment session. In some embodiments the pharmaceutical composition comprising up to 2.0% w/w polidocanol are administered to the subject in the fifth treatment session and the sixth treatment session. In some embodiments the pharmaceutical composition comprising less than 3.0% w/w polidocanol comprises 2.0% w/w polidocanol. In some embodiments the pharmaceutical composition comprising 2.0% w/w polidocanol are administered to the subject in the fifth treatment session and the sixth treatment session. In some embodiments the administering the reduced volume of injectate of the pharmaceutical composition in the second or subsequent treatment session(s) comprises at least one of the injections in the second or subsequent treatment session(s) having a reduced injectate volume of individual injections of a plurality of injections than was administered in a first subsequent treatment session. In some embodiments the administering the reduced volume of injectate of the pharmaceutical composition in the second or subsequent treatment session(s) comprises each of the injections in the second or subsequent treatment session(s) having a reduced injectate volume of individual injections of a plurality of injections than was administered in a first subsequent treatment session. In certain aspects, providing a progressively decreasing dosage concentration of polidocanol reduces an injection related side effect by reducing a volume of healthy tissue which is exposed to the detergent agent and reducing mass transfer of polidocanol to healthy tissue surrounding through adipose tissue injection sites, however, it is surprising and unexpected that even reduced concentrations of polidocanol in subsequent treatments facilitate significant apolysis of submental fat tissue, for example, a grade two or larger reduction in submental fat, while also reducing an injection related side effect.

In some embodiments, the method improves elimination of subcutaneous adipose tissue in a subject by injecting the subject with a pharmaceutical composition comprising 3.0-4.5% w/w polidocanol, wherein the method improves elimination of subcutaneous adipose tissue in the subject by at least 1.5× compared to injection of 1% w/w deoxycholic acid. In some embodiments, the method improves elimination of subcutaneous adipose tissue in a subject comprising injecting the subject with a pharmaceutical composition comprising 3.0-4.5% w/w polidocanol, where the method improves elimination of subcutaneous adipose tissue in the subject by at least 1.5× compared to injection of 2% w/w deoxycholic acid. In some embodiments, the method improves elimination of subcutaneous adipose tissue in a subject comprising injecting the subject with a pharmaceutical composition comprising 3.0-4.5% w/w polidocanol, where the subject exhibits at least a two grade improvement in submental adipose tissue reduction. In some embodiments, the method eliminates subcutaneous adipose tissue in a subject by injecting the subject with an average of more than 30 doses per treatment during a clinical treatment regimen of up to six treatments of a pharmaceutical composition comprising 3.0-4.5% w/w polidocanol in a single treatment session. In some embodiments, the method eliminates of subcutaneous adipose tissue in a subject comprising injecting the subject with a plurality of doses of a pharmaceutical composition comprising 3.0-4.5% w/w polidocanol over an area of at least 15 cm². In some embodiments, the method comprises injecting the subject with a plurality of doses of a pharmaceutical composition over an area of at least 16, 17, 18, 19, 20, 21, 22, 23, 24, 25, 26, 27, 28, 29, 30, 35, or 40 cm². In some embodiments, the method eliminates of subcutaneous adipose tissue in a subject by injecting the subject with a pharmaceutical composition comprising 3.0-4.5% w/w polidocanol in a plurality of treatment sessions, each treatment session being more than 23 days apart but not more than 60 days apart. In some embodiments, the method eliminates subcutaneous adipose tissue in a subject by injecting the subject with a pharmaceutical composition comprising 3.0-4.5% w/w polidocanol in a plurality of treatment sessions, comprising at administering between two and six treatment sessions to the subject. In some embodiments, the method reduces injection related side effect in a subject comprising injecting the subject with a pharmaceutical composition comprising 3.0-4.5% w/w polidocanol. In some embodiments, the subject is human. In some embodiments, the method improves elimination of subcutaneous adipose tissue in the subject by at least 50% compared to injection of 1% w/w deoxycholic acid. In some embodiments, the method improves elimination of subcutaneous adipose tissue in the subject by at least 50% compared to injection of 2% w/w deoxycholic acid. FIG. 2A and FIG. 3 illustrate the increased efficacy of adipose tissue removal compared to deoxycholic acid (Kybella). In some embodiments, the subject exhibits at least a two grade improvement in submental adipose tissue reduction. In some embodiments, the method comprises injecting the subject with an average of more than 30 doses per treatment during a clinical treatment regimen of up to six treatments of the pharmaceutical composition. In some embodiments, the method comprises injecting the subject with a plurality of doses of a pharmaceutical composition over an area of at least 15 cm². In some embodiments, the method comprises injecting the subject with a plurality of doses of a pharmaceutical composition over an area of at least 16, 17, 18, 19, 20, 21, 22, 23, 24, 25, 26, 27, 28, 29, 30, 35, or 40 cm². In some embodiments, the area is an effective treatment area. In some embodiments, the method comprises injecting the subject with the pharmaceutical composition in a plurality of treatment sessions, each treatment session being more than 23 days apart but not more than 60 days apart. In some embodiments, the method reduces an injection related side effect in the subject. In some embodiments, the method improves a rate of elimination of subcutaneous adipose tissue in the subject compared to injection of 1% w/w deoxycholic acid. In some embodiments, the method improves elimination of subcutaneous adipose tissue in the subject by at least 1.75, 2, 2.25, 2.5, 2.75, 3, 3.25, 3.5, 3.75, 4, 4.5, or 5× compared to injection of 1% w/w deoxycholic acid. In some embodiments, the method improves elimination of subcutaneous adipose tissue in the subject by at least 1.75, or 2× compared to injection of 2% w/w deoxycholic acid. In some embodiments, the method reduces a mass of subcutaneous adipose tissue in the subject. In some embodiments, the method reduces a mass of subcutaneous adipose tissue in the subject in a region of the injection by 10, 20, 30, 40, 50, 60, or 75% w/w. In some embodiments, the region is a submental region. In some embodiments, the method reduces a volume of subcutaneous adipose tissue in the subject. In some embodiments, the method reduces a volume of subcutaneous adipose tissue in the subject in a region of the injection by 10, 20, 30, 40, 50, 60, or 75% v/v. In some embodiments, the region is a submental region. In some embodiments, the subject exhibits at least a one grade improvement in submental adipose tissue reduction. In some embodiments, the one grade improvement in submental adipose tissue reduction is exhibited within 12, 11, 10, 9, or 8 weeks following injection with the pharmaceutical composition. In some embodiments, the one grade improvement in submental adipose tissue reduction is exhibited following administering at least three treatment sessions to the subject. In some embodiments, the subject exhibits at least a two grade improvement in submental adipose tissue reduction. In some embodiments, the one grade improvement in submental adipose tissue reduction is exhibited within 24 weeks following injection with the pharmaceutical composition. In some embodiments, the one grade improvement in submental adipose tissue reduction is exhibited following administering at least six treatment sessions to the subject. In some embodiments, the method comprises injecting the subject with up to 50 doses of the pharmaceutical composition. In some embodiments, the method comprises injecting the subject with at least 50 doses of the pharmaceutical composition. In some embodiments, the method comprises injecting the subject with at least 20 and up to 50 doses of the pharmaceutical composition. In some embodiments, the method comprises injecting the subject with an average of at least 30 and up to 50 doses of the pharmaceutical composition. In some embodiments, each dose is not more than 0.2 mL. In some embodiments, each dose is less than 0.2 mL. In some embodiments, the method comprises administering at least three treatment sessions to the subject. In some embodiments, the method comprises administering at least six treatment sessions to the subject. In some embodiments, the method comprises administering between two and six treatment sessions to the subject. In some embodiments, the method comprises administering up to six treatment sessions to the subject. In some embodiments, each treatment session is administered at least 3 weeks apart. In some embodiments, each treatment session is administered more than 30 days apart. In some embodiments, each dose is injected at least 1 cm apart. In some embodiments, each dose is injected up to 1 cm apart. In some embodiments, each dose is injected about 1 cm apart. In some embodiments, a submental region of the subject is treated. In some embodiments, a flank region of the subject is treated. In some embodiments, an abdominal region of the subject is treated. In some embodiments, a lingual region of the subject is treated. In some embodiments, the subject exhibits at least a one grade improvement in submental adipose tissue reduction. In some embodiments, the one grade improvement in submental adipose tissue reduction is exhibited after 2 treatment sessions. In some embodiments, the one grade improvement in submental adipose tissue reduction is exhibited within about 8 weeks. In some embodiments, the subject exhibits at least a two grade improvement in submental adipose tissue reduction. In some embodiments, the two grade improvement in submental adipose tissue reduction is exhibited after 3 treatment sessions. In some embodiments, the two grade improvement in submental adipose tissue reduction is exhibited within about 24 weeks. In some embodiments, the subject exhibits a decrease in convexity, fullness, bulging, bloating, stretching, in a region injected with the pharmaceutical composition. Tables 9-12 illustrate the decreased adverse events stemmed from the pharmaceutical composition treatment. In some embodiments, the subject exhibits an improvement in appearance in a region injected with the pharmaceutical composition. In some embodiments, the pharmaceutical composition comprises about 3.0% w/w polidocanol. In some embodiments, the pharmaceutical composition comprises about 4.5% w/w polidocanol. In some embodiments, the pharmaceutical composition is a subcutaneous injection formulation. In some embodiments, the pharmaceutical composition is an aqueous formulation. In some embodiments, the pharmaceutical composition comprises a cosolvent. In some embodiments, the cosolvent comprises propylene glycol. In some embodiments, the propylene glycol is comprised in an amount of up to 2.25% w/w. In some embodiments, the pharmaceutical composition comprises a buffer. In some embodiments, the pharmaceutical composition comprises a binary buffers system. In some embodiments, the binary buffer system comprises sodium phosphate and potassium phosphate. In some embodiments, the binary buffer system comprises sodium phosphate dibasic dihydrate and potassium phosphate monobasic. In some embodiments, the sodium phosphate dibasic dihydrate is comprised in an amount of up to about 0.25% w/w. In some embodiments, the potassium phosphate monobasic is comprised in an amount of up to about 0.1% w/w. In some embodiments, the pharmaceutical composition comprises an osmolarity of less than about 400 mM/kg. In some embodiments, the pharmaceutical composition comprises an alkaline pH. In some embodiments, the pharmaceutical composition comprises a pH of 7-8. In some embodiments, the pharmaceutical composition comprises a critical micelle concentration of up to 0.10 millimolar. In some embodiments, the pharmaceutical composition comprises a critical micelle concentration of 0.07-0.1 mM. In some embodiments, the injecting is subcutaneous. In some embodiments, the method locally reduces subcutaneous adipose tissue. In some embodiments, the method locally reduces submental adipose tissue. In some embodiments, the comparative injection of 1% w/w deoxycholic acid in a same volume and/or a same spacing of injections. In some embodiments, the comparative injection of 2% w/w deoxycholic acid in a same volume and/or a same spacing of injections. In some embodiments, the reduction in the injection related side effect is exhibited as compared to injection of 1% w/w deoxycholic acid. In some embodiments, the reduction in the injection related side effect is exhibited as compared to injection of 2% w/w deoxycholic acid. In some embodiments, the side effect comprises numbness, induration, paresthesia, nodule, pruritis, or nerve injury. In some embodiments, the side effect comprises Erythema, injection site pain, bruising, edema, or swelling. In some embodiments, the method described herein reduces a severity of the side effect. In some embodiments, the side effect comprises numbness, induration, paresthesia, nodule, pruritis, nerve injury, injection site pain, bruising, edema, or swelling, wherein a 100, 90, 80, 70, 60, 50, 45, 40, 35, 30, 25, 20, 15, 10, or 5% decrease in occurrence of the side effect is observed as compared to injection of 1% w/w deoxycholic acid. In some embodiments, the side effect comprises numbness, induration, paresthesia, nodule, pruritis, nerve injury, injection site pain, bruising, edema, or swelling, wherein a 100, 90, 80, 70, 60, 50, 45, 40, 35, 30, 25, 20, 15, 10, or 5% decrease in occurrence of the side effect is observed as compared to injection of 2% w/w deoxycholic acid. In some embodiments, the comparative injection of 1% w/w deoxycholic acid in a same volume and/or a same spacing of injections, or wherein the comparative injection of 2% w/w deoxycholic acid is in a same volume and/or a same spacing of injections.

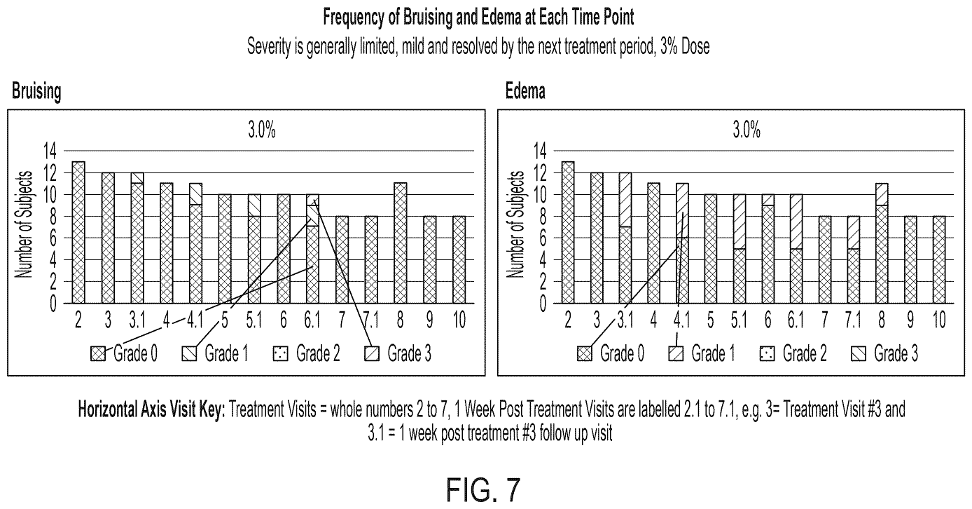

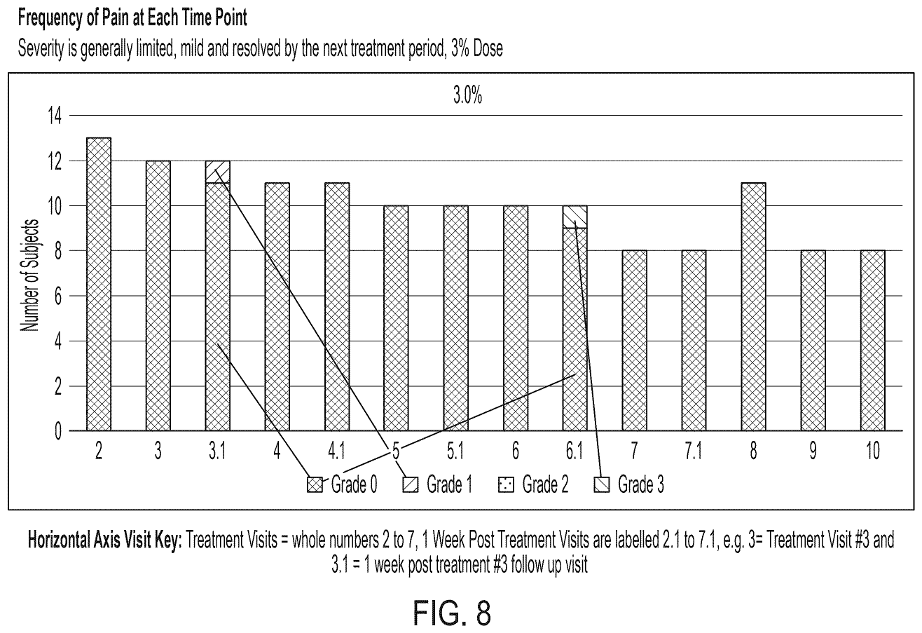

FIG. 7 illustrates improvements to an injection related side effect(s) when administering polidocanol pharmaceutical formulations according to methods described herein. In FIG. 7, the number of subjects exhibiting a side effect of bruising or edema is shown, along with the grade of the side effect. In FIG. 7, 13 subjects total exhibited bruising, with almost all subjects exhibiting a grade 0 (none) bruising, with only 2 subjects exhibiting grade 1 (mild) bruising, no subjects exhibiting grade 2 bruising, and only 1 subject exhibiting grade 3 bruising. In FIG. 7, 13 subjects total exhibited edema, with most subjects exhibiting a grade 0 (none) edema, with only 5 subjects exhibiting grade 1 (mild) bruising, no subjects exhibiting grade 2 or grade 3 edema. In FIG. 7, it is further shown that fewer subjects exhibit bruising or edema as the trial continued and further treatments were provided at visits 3, 4, 5, and 6. In FIG. 7, it is further shown that a side effect of bruising or edema, if present, generally did not worsen following the treatment and it is further shown that incidences of bruising or edema, if present, generally resolved by the next treatment dose. FIG. 8 illustrates improvements to an injection related side effect(s), specifically pain, when administering polidocanol pharmaceutical formulations according to methods described herein. In FIG. 8, it is shown that the frequency of pain was generally mild, with the vast majority of subjects exhibiting grade 0 (none) pain, and it is further shown that incidences of pain generally resolved by the next treatment dose. In some embodiments, the reducing the injection related side effect in the subject comprises the subject not exhibiting any injection related side effect after 30 days following injection. In some embodiments, the reducing the injection related side effect in the subject comprises less than 50% of subjects exhibiting an injection related side effect comprising pain, bruising, burning, stinging, erythema, numbness, induration, paresthesia, nodule, pruritis, nerve injury, or combinations thereof. In some embodiments, the reducing the injection related side effect in the subject comprises the subject exhibiting reduced occurrence of the injection related side effect in subsequent treatments when receiving or after receiving a subsequent injection of the pharmaceutical composition. In some embodiments, the reducing the injection related side effect in the subject comprises fewer subjects exhibiting injection related side effect in subsequent treatments when receiving a subsequent injection of the pharmaceutical composition. In some embodiments, the reducing the injection related side effect in the subject comprises the subject not exhibiting a worsening of the injection related side effect following a treatment session. In some embodiments, the reducing the injection related side effect in the subject comprises the subject not exhibiting a worsening of the injection related side effect following receiving a subsequent injection of the pharmaceutical composition. In some embodiments, the reducing the injection related side effect in the subject comprises the injection related side effect resolving in the subject within 30 days. In some embodiments, the reducing the injection related side effect in the subject comprises the injection related side effect resolving in the subject prior to receiving a subsequent injection of the pharmaceutical composition. In some embodiments, the injection related side effect is pain, bruising, or edema. In some embodiments, up to six treatments are administered to the subject. In some embodiments, at least a one grade improvement in submental adipose tissue reduction is exhibited by the subject following the treatment. In some embodiments, at least a two grade improvement in submental adipose tissue reduction is exhibited by the subject following the treatment. In some embodiments, between three and 50 injections of the composition are administered to the subject per treatment session. In some embodiments, an average of 10 to 30 injections of the composition are administered to the subject per treatment session. In some embodiments, an average of at least 10 injections of the composition are administered to the subject per treatment session. In some embodiments, each treatment session is administered more than 25, 28, 30, 35, 40, 45, 50, or 55 days apart but not more than 60 days apart.

In embodiments, reducing subcutaneous adipose tissue includes destroying fat cells (e.g., through apoptosis or necrosis or cell lysis induced directly by the pharmaceutical formulation or killing fat cells). In embodiments, reducing subcutaneous adipose tissue includes contouring an area of a subject (e.g., contouring a subcutaneous tissue(s) of the subject). In embodiments, contouring includes reducing the size of the area. In embodiments, reducing subcutaneous adipose tissue includes reducing the size (e.g., volume or mass) of the subcutaneous adipose tissue. In embodiments, reducing subcutaneous adipose tissue includes reducing the weight of the subcutaneous adipose tissue. In embodiments, reducing subcutaneous adipose tissue includes reducing the physical integrity (e.g., rigidity, shape, firmness, solidity, or density) of the subcutaneous adipose tissue. In embodiments, reducing subcutaneous adipose tissue includes reducing the subcutaneous adipose tissue in direct contact with the pharmaceutical formulation. In embodiments, reducing subcutaneous adipose tissue includes reducing the fat cells in direct contact with the pharmaceutical formulation. In embodiments, reducing subcutaneous adipose tissue includes reducing the subcutaneous adipose tissue into which the pharmaceutical formulation is administered. In embodiments, reducing subcutaneous adipose tissue includes reducing the subcutaneous adipose tissue substantially connected to the subcutaneous adipose tissue into which the pharmaceutical formulation is administered. In embodiments, reducing subcutaneous adipose tissue includes reducing the subcutaneous adipose tissue connected to the subcutaneous adipose tissue into which the pharmaceutical formulation is administered.

In embodiments, reducing subcutaneous adipose tissue consists essentially of destroying fat cells (e.g., through apoptosis or necrosis or cell lysis induced directly by the pharmaceutical formulation or killing fat cells). In embodiments, reducing subcutaneous adipose tissue consists essentially of contouring an area of a subject (e.g., contouring a subcutaneous tissue(s) of the subject). In embodiments, contouring consists essentially of reducing the size of the area. In embodiments, reducing subcutaneous adipose tissue consists essentially of reducing the size (e.g., volume or mass) of the subcutaneous adipose tissue. In embodiments, reducing subcutaneous adipose tissue consists essentially of reducing the weight of the subcutaneous adipose tissue. In embodiments, reducing subcutaneous adipose tissue consists essentially of reducing the physical integrity (e.g., rigidity, shape, firmness, solidity, or density) of the subcutaneous adipose tissue. In embodiments, reducing subcutaneous adipose tissue consists essentially of reducing the subcutaneous adipose tissue in direct contact with the pharmaceutical formulation. In embodiments, reducing subcutaneous adipose tissue consists essentially of reducing the fat cells in direct contact with the pharmaceutical formulation. In embodiments, reducing subcutaneous adipose tissue consists essentially of reducing the subcutaneous adipose tissue into which the pharmaceutical formulation is administered. In embodiments, reducing subcutaneous adipose tissue consists essentially of reducing the subcutaneous adipose tissue substantially connected to the subcutaneous adipose tissue into which the pharmaceutical formulation is administered. In embodiments, reducing subcutaneous adipose tissue consists essentially of reducing the subcutaneous adipose tissue connected to the subcutaneous adipose tissue into which the pharmaceutical formulation is administered.

In embodiments, reducing subcutaneous adipose tissue consists of destroying fat cells (e.g., through apoptosis or necrosis or cell lysis induced directly by the pharmaceutical formulation or killing fat cells). In embodiments, reducing subcutaneous adipose tissue consists of contouring an area of a subject (e.g., contouring a subcutaneous tissue(s) of the subject). In embodiments, contouring consists of reducing the size of the area. In embodiments, reducing subcutaneous adipose tissue consists of reducing the size (e.g., volume or mass) of the subcutaneous adipose tissue. In embodiments, reducing subcutaneous adipose tissue consists of reducing the weight of the subcutaneous adipose tissue. In embodiments, reducing subcutaneous adipose tissue consists of reducing the physical integrity (e.g., rigidity, shape, firmness, solidity, or density) of the subcutaneous adipose tissue. In embodiments, reducing subcutaneous adipose tissue consists of reducing the subcutaneous adipose tissue in direct contact with the pharmaceutical formulation. In embodiments, reducing subcutaneous adipose tissue consists of reducing the fat cells in direct contact with the pharmaceutical formulation. In embodiments, reducing subcutaneous adipose tissue consists of reducing the subcutaneous adipose tissue into which the pharmaceutical formulation is administered. In embodiments, reducing subcutaneous adipose tissue consists of reducing the subcutaneous adipose tissue substantially connected to the subcutaneous adipose tissue into which the pharmaceutical formulation is administered. In embodiments, reducing subcutaneous adipose tissue consists of reducing the subcutaneous adipose tissue connected to the subcutaneous adipose tissue into which the pharmaceutical formulation is administered.

In embodiments, reducing subcutaneous adipose tissue is reduction by greater than about 1% (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%). In embodiments, reducing subcutaneous adipose tissue is reduction by less than about 1% (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%). In embodiments, reducing subcutaneous adipose tissue is reduction by about 1% (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%)

In embodiments, reducing subcutaneous adipose tissue is reduction by greater than 1% (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%). In embodiments, reducing subcutaneous adipose tissue is reduction by less than 1% (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%). In embodiments, reducing subcutaneous adipose tissue is reduction by 1% (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%).

In embodiments, the pharmaceutical formulation is administered within a plurality of treatment sessions. In embodiments, each treatment session is spaced by at least 14 days. In embodiments, each treatment session is separated from another treatment session by at least 7 days. In embodiments, each treatment session is separated from another treatment session by at least 8 days. In embodiments, each treatment session is separated from another treatment session by at least 9 days. In embodiments, each treatment session is separated from another treatment session by at least 10 days. In embodiments, each treatment session is separated from another treatment session by at least 11 days. In embodiments, each treatment session is separated from another treatment session by at least 12 days. In embodiments, each treatment session is separated from another treatment session by at least 13 days. In embodiments, each treatment session is separated from another treatment session by at least 14 days. In embodiments, each treatment session is separated from another treatment session by at least 15 days. In embodiments, each treatment session is separated from another treatment session by at least 16 days. In embodiments, each treatment session is separated from another treatment session by at least 17 days. In embodiments, each treatment session is separated from another treatment session by at least 18 days. In embodiments, each treatment session is separated from another treatment session by at least 19 days. In embodiments, each treatment session is separated from another treatment session by at least 20 days. In embodiments, each treatment session is separated from another treatment session by at least 21 days. In embodiments, each treatment session is separated from another treatment session by at least 22 days. In embodiments, each treatment session is separated from another treatment session by at least 23 days. In embodiments, each treatment session is separated from another treatment session by at least 24 days. In embodiments, each treatment session is separated from another treatment session by at least 25 days. In embodiments, each treatment session is separated from another treatment session by at least 26 days. In embodiments, each treatment session is separated from another treatment session by at least 27 days. In embodiments, each treatment session is separated from another treatment session by at least 28 days. In embodiments, each treatment session is separated from another treatment session by at least 29 days. In embodiments, each treatment session is separated from another treatment session by at least 30 days. In embodiments, each treatment session is separated from another treatment session by at least 31 days. In embodiments, each treatment session is separated from another treatment session by at least four weeks. In embodiments, each treatment session is separated from another treatment session by at least five weeks. In embodiments, each treatment session is separated from another treatment session by at least six weeks. In embodiments, each treatment session is separated from another treatment session by at least seven weeks. In embodiments, each treatment session is separated from another treatment session by at least eight weeks. In embodiments, each treatment session is separated from another treatment session by at least one month. In embodiments, each treatment session is separated from another treatment session by at least two months. In embodiments, each treatment session is separated from another treatment session by at least three months. In embodiments, each treatment session is separated from another treatment session by at least four months. In embodiments, each treatment session is separated from another treatment session by at least five months. In embodiments, each treatment session is separated from another treatment session by at least six months. In embodiments, each treatment session is separated from another treatment session by at least nine months. In embodiments, each treatment session is separated from another treatment session by at least one year.

In embodiments, the pharmaceutical formulation is administered within a plurality of treatment sessions. In embodiments, each treatment session is spaced by about 14 days. In embodiments, each treatment session is separated from another treatment session by about 7 days. In embodiments, each treatment session is separated from another treatment session by about 8 days. In embodiments, each treatment session is separated from another treatment session by about 9 days. In embodiments, each treatment session is separated from another treatment session by about 10 days. In embodiments, each treatment session is separated from another treatment session by about 11 days. In embodiments, each treatment session is separated from another treatment session by about 12 days. In embodiments, each treatment session is separated from another treatment session by about 13 days. In embodiments, each treatment session is separated from another treatment session by about 14 days. In embodiments, each treatment session is separated from another treatment session by about 15 days. In embodiments, each treatment session is separated from another treatment session by about 16 days. In embodiments, each treatment session is separated from another treatment session by about 17 days. In embodiments, each treatment session is separated from another treatment session by about 18 days. In embodiments, each treatment session is separated from another treatment session by about 19 days. In embodiments, each treatment session is separated from another treatment session by about 20 days. In embodiments, each treatment session is separated from another treatment session by about 21 days. In embodiments, each treatment session is separated from another treatment session by about 22 days. In embodiments, each treatment session is separated from another treatment session by about 23 days. In embodiments, each treatment session is separated from another treatment session by about 24 days. In embodiments, each treatment session is separated from another treatment session by about 25 days. In embodiments, each treatment session is separated from another treatment session by about 26 days. In embodiments, each treatment session is separated from another treatment session by about 27 days. In embodiments, each treatment session is separated from another treatment session by about 28 days. In embodiments, each treatment session is separated from another treatment session by about 29 days. In embodiments, each treatment session is separated from another treatment session by about 30 days. In embodiments, each treatment session is separated from another treatment session by about 31 days. In embodiments, each treatment session is separated from another treatment session by about four weeks. In embodiments, each treatment session is separated from another treatment session by about five weeks. In embodiments, each treatment session is separated from another treatment session by about six weeks. In embodiments, each treatment session is separated from another treatment session by about seven weeks. In embodiments, each treatment session is separated from another treatment session by about eight weeks. In embodiments, each treatment session is separated from another treatment session by about one month. In embodiments, each treatment session is separated from another treatment session by about two months. In embodiments, each treatment session is separated from another treatment session by about three months. In embodiments, each treatment session is separated from another treatment session by about four months. In embodiments, each treatment session is separated from another treatment session by about five months. In embodiments, each treatment session is separated from another treatment session by about six months. In embodiments, each treatment session is separated from another treatment session by about nine months. In embodiments, each treatment session is separated from another treatment session by about one year.

In embodiments, each treatment session includes administering a plurality of subcutaneous injections of the pharmaceutical formulation into adipose tissue of the subject. In embodiments, the plurality of subcutaneous injections is about 2. In embodiments, the plurality of subcutaneous injections is about 3. In embodiments, the plurality of subcutaneous injections is about 4. In embodiments, the plurality of subcutaneous injections is about 5. In embodiments, the plurality of subcutaneous injections is about 6. In embodiments, the plurality of subcutaneous injections is about 7. In embodiments, the plurality of subcutaneous injections is about 8. In embodiments, the plurality of subcutaneous injections is about 9. In embodiments, the plurality of subcutaneous injections is about 10. In embodiments, the plurality of subcutaneous injections is about 15. In embodiments, the plurality of subcutaneous injections is about 20. In embodiments, the plurality of subcutaneous injections is about 25. In embodiments, the plurality of subcutaneous injections is about 30.

In embodiments, each treatment session includes administering a plurality of subcutaneous injections of the pharmaceutical formulation into adipose tissue of the subject. In embodiments, the plurality of subcutaneous injections is 2. In embodiments, the plurality of subcutaneous injections is 3. In embodiments, the plurality of subcutaneous injections is 4. In embodiments, the plurality of subcutaneous injections is 5. In embodiments, the plurality of subcutaneous injections is 6. In embodiments, the plurality of subcutaneous injections is 7. In embodiments, the plurality of subcutaneous injections is 8. In embodiments, the plurality of subcutaneous injections is 9. In embodiments, the plurality of subcutaneous injections is 10. In embodiments, the plurality of subcutaneous injections is 15. In embodiments, the plurality of subcutaneous injections is 20. In embodiments, the plurality of subcutaneous injections is 25. In embodiments, the plurality of subcutaneous injections is 30. In embodiments, the plurality of subcutaneous injections is about 2, 3, 4, 5, 6, 7, 8, 9, 10, 11, 12, 13, 14, 15, 16, 17, 18, 19, 20, 21, 22, 23, 24, 25, 26, 27, 28, 29, or 30. In embodiments, the plurality of subcutaneous injections is 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In embodiments, the administrating is subcutaneously administering. In embodiments, the method locally reduces subcutaneous adipose tissue. In embodiments, the administering includes a plurality of subcutaneous injections of the pharmaceutical formulation into adipose tissue of said subject. In embodiments, the plurality of subcutaneous injections is about 2, 3, 4, 5, 6, 7, 8, 9, 10, 11, 12, 13, 14, 15, 16, 17, 18, 19, 20, 21, 22, 23, 24, 25, 26, 27, 28, 29, or 30. In embodiments, the plurality of subcutaneous injections is 2, 3, 4, 5, 6, 7, 8, 9, 10, 11, 12, 13, 14, 15, 16, 17, 18, 19, 20, 21, 22, 23, 24, 25, 26, 27, 28, 29, or 30. In embodiments, the plurality of subcutaneous injections are spaced about 0.1 cm apart. In embodiments, the plurality of subcutaneous injections are spaced about 0.2 cm apart. In embodiments, the plurality of subcutaneous injections are spaced about 0.3 cm apart. In embodiments, the plurality of subcutaneous injections are spaced about 0.4 cm apart. In embodiments, the plurality of subcutaneous injections are spaced about 0.5 cm apart. In embodiments, the plurality of subcutaneous injections are spaced about 0.6 cm apart. In embodiments, the plurality of subcutaneous injections are spaced about 0.7 cm apart. In embodiments, the plurality of subcutaneous injections are spaced about 0.8 cm apart. In embodiments, the plurality of subcutaneous injections are spaced about 0.9 cm apart. In embodiments, the plurality of subcutaneous injections are spaced about 1.0 cm apart. In embodiments, the plurality of subcutaneous injections are spaced about 1.5 cm apart. In embodiments, the plurality of subcutaneous injections are spaced about 2.0 cm apart. In embodiments, the plurality of subcutaneous injections are spaced about 2.5 cm apart. In embodiments, the plurality of subcutaneous injections are spaced about 3.0 cm apart. In embodiments, the plurality of subcutaneous injections are spaced about 3.5 cm apart. In embodiments, the plurality of subcutaneous injections are spaced about 4.0 cm apart. In embodiments, the plurality of subcutaneous injections are spaced about 4.5 cm apart. In embodiments, the plurality of subcutaneous injections are spaced about 5.0 cm apart. In embodiments, the plurality of subcutaneous injections are spaced about 10.0 cm apart.

In embodiments, the plurality of subcutaneous injections are spaced 0.1 cm apart. In embodiments, the plurality of subcutaneous injections are spaced 0.2 cm apart. In embodiments, the plurality of subcutaneous injections are spaced 0.3 cm apart. In embodiments, the plurality of subcutaneous injections are spaced 0.4 cm apart. In embodiments, the plurality of subcutaneous injections are spaced 0.5 cm apart. In embodiments, the plurality of subcutaneous injections are spaced 0.6 cm apart. In embodiments, the plurality of subcutaneous injections are spaced 0.7 cm apart. In embodiments, the plurality of subcutaneous injections are spaced 0.8 cm apart. In embodiments, the plurality of subcutaneous injections are spaced 0.9 cm apart. In embodiments, the plurality of subcutaneous injections are spaced 1.0 cm apart. In embodiments, the plurality of subcutaneous injections are spaced 1.5 cm apart. In embodiments, the plurality of subcutaneous injections are spaced 2.0 cm apart. In embodiments, the plurality of subcutaneous injections are spaced 2.5 cm apart. In embodiments, the plurality of subcutaneous injections are spaced 3.0 cm apart. In embodiments, the plurality of subcutaneous injections are spaced 3.5 cm apart. In embodiments, the plurality of subcutaneous injections are spaced 4.0 cm apart. In embodiments, the plurality of subcutaneous injections are spaced 4.5 cm apart. In embodiments, the plurality of subcutaneous injections are spaced 5.0 cm apart. In embodiments, the plurality of subcutaneous injections are spaced 10.0 cm apart.

In embodiments, the plurality of subcutaneous injections are spaced an average of about 0.1 cm apart. In embodiments, the plurality of subcutaneous injections are spaced an average of about 0.2 cm apart. In embodiments, the plurality of subcutaneous injections are spaced an average of about 0.3 cm apart. In embodiments, the plurality of subcutaneous injections are spaced an average of about 0.4 cm apart. In embodiments, the plurality of subcutaneous injections are spaced an average of about 0.5 cm apart. In embodiments, the plurality of subcutaneous injections are spaced an average of about 0.6 cm apart. In embodiments, the plurality of subcutaneous injections are spaced an average of about 0.7 cm apart. In embodiments, the plurality of subcutaneous injections are spaced an average of about 0.8 cm apart. In embodiments, the plurality of subcutaneous injections are spaced an average of about 0.9 cm apart. In embodiments, the plurality of subcutaneous injections are spaced an average of about 1.0 cm apart. In embodiments, the plurality of subcutaneous injections are spaced an average of about 1.5 cm apart. In embodiments, the plurality of subcutaneous injections are spaced an average of about 2.0 cm apart. In embodiments, the plurality of subcutaneous injections are spaced an average of about 2.5 cm apart. In embodiments, the plurality of subcutaneous injections are spaced an average of about 3.0 cm apart. In embodiments, the plurality of subcutaneous injections are spaced an average of about 3.5 cm apart. In embodiments, the plurality of subcutaneous injections are spaced an average of about 4.0 cm apart. In embodiments, the plurality of subcutaneous injections are spaced an average of about 4.5 cm apart. In embodiments, the plurality of subcutaneous injections are spaced an average of about 5.0 cm apart. In embodiments, the plurality of subcutaneous injections are spaced an average of about 10.0 cm apart.

In embodiments, the plurality of subcutaneous injections are spaced an average of 0.1 cm apart. In embodiments, the plurality of subcutaneous injections are spaced an average of 0.2 cm apart. In embodiments, the plurality of subcutaneous injections are spaced an average of 0.3 cm apart. In embodiments, the plurality of subcutaneous injections are spaced an average of 0.4 cm apart. In embodiments, the plurality of subcutaneous injections are spaced an average of 0.5 cm apart. In embodiments, the plurality of subcutaneous injections are spaced an average of 0.6 cm apart. In embodiments, the plurality of subcutaneous injections are spaced an average of 0.7 cm apart. In embodiments, the plurality of subcutaneous injections are spaced an average of 0.8 cm apart. In embodiments, the plurality of subcutaneous injections are spaced an average of 0.9 cm apart. In embodiments, the plurality of subcutaneous injections are spaced an average of 1.0 cm apart. In embodiments, the plurality of subcutaneous injections are spaced an average of 1.5 cm apart. In embodiments, the plurality of subcutaneous injections are spaced an average of 2.0 cm apart. In embodiments, the plurality of subcutaneous injections are spaced an average of 2.5 cm apart. In embodiments, the plurality of subcutaneous injections are spaced an average of 3.0 cm apart. In embodiments, the plurality of subcutaneous injections are spaced an average of 3.5 cm apart. In embodiments, the plurality of subcutaneous injections are spaced an average of 4.0 cm apart. In embodiments, the plurality of subcutaneous injections are spaced an average of 4.5 cm apart. In embodiments, the plurality of subcutaneous injections are spaced an average of 5.0 cm apart. In embodiments, the plurality of subcutaneous injections are spaced an average of 10.0 cm apart. In embodiments, the method locally reduces submental adipose tissue.

In embodiments, the pharmaceutical formulation is administered in a volume of from about 0.1 cc to about 5.0 cc. In embodiments, the pharmaceutical formulation is administered in a volume of from about 1.0 cc to about 2.0 cc. In embodiments, the pharmaceutical formulation is administered in a volume of from about 0.1 cc to about 0.5 cc. In embodiments, the pharmaceutical formulation is administered in a volume of from about 0.2 cc to about 0.5 cc. In embodiments, the pharmaceutical formulation is administered in a volume of from about 0.3 cc to about 0.5 cc. In embodiments, the pharmaceutical formulation is administered in a volume of from about 0.4 cc to about 0.5 cc. In embodiments, the pharmaceutical formulation is administered in a volume of from about 0.1 cc to about 0.4 cc. In embodiments, the pharmaceutical formulation is administered in a volume of from about 0.1 cc to about 0.3 cc. In embodiments, the pharmaceutical formulation is administered in a volume of from about 0.1 cc to about 0.2 cc. In embodiments, the pharmaceutical formulation is administered in a volume of from about 0.1 cc to about 0.6 cc. In embodiments, the pharmaceutical formulation is administered in a volume of from about 0.1 cc to about 0.7 cc. In embodiments, the pharmaceutical formulation is administered in a volume of from about 0.1 cc to about 0.8 cc. In embodiments, the pharmaceutical formulation is administered in a volume of from about 0.1 cc to about 0.9 cc. In embodiments, the pharmaceutical formulation is administered in a volume of from about 0.1 cc to about 1.0 cc. In embodiments, the pharmaceutical formulation is administered in a volume of from about 0.3 cc to about 0.4 cc. In embodiments, the pharmaceutical formulation is administered in a volume of from about 0.2 cc to about 0.3 cc. In embodiments, the pharmaceutical formulation is administered in a volume of from about 0.2 cc to about 0.4 cc. In embodiments, the pharmaceutical formulation is administered in a volume of from about 0.2 cc to about 0.5 cc. In embodiments, the pharmaceutical formulation is administered in a volume of from about 0.2 cc to about 0.6 cc. In embodiments, the pharmaceutical formulation is administered in a volume of from about 0.2 cc to about 0.7 cc. In embodiments, the pharmaceutical formulation is administered in a volume of from about 0.2 cc to about 0.8 cc. In embodiments, the pharmaceutical formulation is administered in a volume of from about 0.2 cc to about 0.9 cc. In embodiments, the pharmaceutical formulation is administered in a volume of from about 0.2 cc to about 1.0 cc.

In embodiments, the pharmaceutical formulation is administered in a volume of about 0.1 cc. In embodiments, the pharmaceutical formulation is administered in a volume of about 0.2 cc. In embodiments, the pharmaceutical formulation is administered in a volume of about 0.3 cc. In embodiments, the pharmaceutical formulation is administered in a volume of about 0.4 cc. In embodiments, the pharmaceutical formulation is administered in a volume of about 0.5 cc. In embodiments, the pharmaceutical formulation is administered in a volume of about 0.6 cc. In embodiments, the pharmaceutical formulation is administered in a volume of about 0.7 cc. In embodiments, the pharmaceutical formulation is administered in a volume of about 0.8 cc. In embodiments, the pharmaceutical formulation is administered in a volume of about 0.9 cc. In embodiments, the pharmaceutical formulation is administered in a volume of about 1.0 cc. In embodiments, the pharmaceutical formulation is administered in a volume of about 2.0 cc. In embodiments, the pharmaceutical formulation is administered in a volume of about 3.0 cc. In embodiments, the pharmaceutical formulation is administered in a volume of about 4.0 cc. In embodiments, the pharmaceutical formulation is administered in a volume of about 5.0 cc.

In embodiments, the pharmaceutical formulation is administered in a volume of from 1.0 cc to 2.0 cc. In embodiments, the pharmaceutical formulation is administered in a volume of from 0.1 cc to 0.5 cc. In embodiments, the pharmaceutical formulation is administered in a volume of from 0.2 cc to 0.5 cc. In embodiments, the pharmaceutical formulation is administered in a volume of from 0.3 cc to 0.5 cc. In embodiments, the pharmaceutical formulation is administered in a volume of from 0.4 cc to 0.5 cc. In embodiments, the pharmaceutical formulation is administered in a volume of from 0.1 cc to 0.4 cc. In embodiments, the pharmaceutical formulation is administered in a volume of from 0.1 cc to 0.3 cc. In embodiments, the pharmaceutical formulation is administered in a volume of from 0.1 cc to 0.2 cc. In embodiments, the pharmaceutical formulation is administered in a volume of from 0.1 cc to 0.6 cc. In embodiments, the pharmaceutical formulation is administered in a volume of from 0.1 cc to 0.7 cc. In embodiments, the pharmaceutical formulation is administered in a volume of from 0.1 cc to 0.8 cc. In embodiments, the pharmaceutical formulation is administered in a volume of from 0.1 cc to 0.9 cc. In embodiments, the pharmaceutical formulation is administered in a volume of from 0.1 cc to 1.0 cc.

In embodiments, the pharmaceutical formulation is administered in a volume of from 0.3 cc to 0.4 cc. In embodiments, the pharmaceutical formulation is administered in a volume of from 0.2 cc to 0.3 cc. In embodiments, the pharmaceutical formulation is administered in a volume of from 0.2 cc to 0.4 cc. In embodiments, the pharmaceutical formulation is administered in a volume of from 0.2 cc to 0.5 cc. In embodiments, the pharmaceutical formulation is administered in a volume of from 0.2 cc to 0.6 cc. In embodiments, the pharmaceutical formulation is administered in a volume of from 0.2 cc to 0.7 cc. In embodiments, the pharmaceutical formulation is administered in a volume of from 0.2 cc to 0.8 cc. In embodiments, the pharmaceutical formulation is administered in a volume of from 0.2 cc to 0.9 cc. In embodiments, the pharmaceutical formulation is administered in a volume of from 0.2 cc to 1.0 cc.

In embodiments, the pharmaceutical formulation is administered in a volume of 0.1 cc. In embodiments, the pharmaceutical formulation is administered in a volume of 0.2 cc. In embodiments, the pharmaceutical formulation is administered in a volume of 0.3 cc. In embodiments, the pharmaceutical formulation is administered in a volume of 0.4 cc. In embodiments, the pharmaceutical formulation is administered in a volume of 0.5 cc. In embodiments, the pharmaceutical formulation is administered in a volume of 0.6 cc. In embodiments, the pharmaceutical formulation is administered in a volume of 0.7 cc. In embodiments, the pharmaceutical formulation is administered in a volume of 0.8 cc. In embodiments, the pharmaceutical formulation is administered in a volume of 0.9 cc. In embodiments, the pharmaceutical formulation is administered in a volume of 1.0 cc. In embodiments, the pharmaceutical formulation is administered in a volume of 2.0 cc.

In embodiments, each individual administration (e.g., individual injection) of the pharmaceutical formulation is administered in a volume of from about 0.1 cc to about 5.0 cc. In embodiments, each individual administration (e.g., individual injection) of the pharmaceutical formulation is administered in a volume of from about 1.0 cc to about 2.0 cc. In embodiments, each individual administration (e.g., individual injection) of the pharmaceutical formulation is administered in a volume of from about 0.1 cc to about 0.5 cc. In embodiments, each individual administration (e.g., individual injection) of the pharmaceutical formulation is administered in a volume of from about 0.2 cc to about 0.5 cc. In embodiments, each individual administration (e.g., individual injection) of the pharmaceutical formulation is administered in a volume of from about 0.3 cc to about 0.5 cc. In embodiments, each individual administration (e.g., individual injection) of the pharmaceutical formulation is administered in a volume of from about 0.4 cc to about 0.5 cc. In embodiments, each individual administration (e.g., individual injection) of the pharmaceutical formulation is administered in a volume of from about 0.1 cc to about 0.4 cc. In embodiments, each individual administration (e.g., individual injection) of the pharmaceutical formulation is administered in a volume of from about 0.1 cc to about 0.3 cc. In embodiments, each individual administration (e.g., individual injection) of the pharmaceutical formulation is administered in a volume of from about 0.1 cc to about 0.2 cc. In embodiments, each individual administration (e.g., individual injection) of the pharmaceutical formulation is administered in a volume of from about 0.1 cc to about 0.6 cc. In embodiments, each individual administration (e.g., individual injection) of the pharmaceutical formulation is administered in a volume of from about 0.1 cc to about 0.7 cc. In embodiments, each individual administration (e.g., individual injection) of the pharmaceutical formulation is administered in a volume of from about 0.1 cc to about 0.8 cc. In embodiments, each individual administration (e.g., individual injection) of the pharmaceutical formulation is administered in a volume of from about 0.1 cc to about 0.9 cc. In embodiments, each individual administration (e.g., individual injection) of the pharmaceutical formulation is administered in a volume of from about 0.1 cc to about 1.0 cc. In embodiments, each individual administration (e.g., individual injection) of the pharmaceutical formulation is administered in a volume of from about 0.3 cc to about 0.4 cc. In embodiments, each individual administration (e.g., individual injection) of the pharmaceutical formulation is administered in a volume of from about 0.2 cc to about 0.3 cc. In embodiments, each individual administration (e.g., individual injection) of the pharmaceutical formulation is administered in a volume of from about 0.2 cc to about 0.4 cc. In embodiments, each individual administration (e.g., individual injection) of the pharmaceutical formulation is administered in a volume of from about 0.2 cc to about 0.5 cc. In embodiments, each individual administration (e.g., individual injection) of the pharmaceutical formulation is administered in a volume of from about 0.2 cc to about 0.6 cc. In embodiments, each individual administration (e.g., individual injection) of the pharmaceutical formulation is administered in a volume of from about 0.2 cc to about 0.7 cc. In embodiments, each individual administration (e.g., individual injection) of the pharmaceutical formulation is administered in a volume of from about 0.2 cc to about 0.8 cc. In embodiments, each individual administration (e.g., individual injection) of the pharmaceutical formulation is administered in a volume of from about 0.2 cc to about 0.9 cc. In embodiments, each individual administration (e.g., individual injection) of the pharmaceutical formulation is administered in a volume of from about 0.2 cc to about 1.0 cc.

In embodiments, each individual administration (e.g., individual injection) of the pharmaceutical formulation is administered in a volume of about 0.1 cc. In embodiments, each individual administration (e.g., individual injection) of the pharmaceutical formulation is administered in a volume of about 0.2 cc. In embodiments, each individual administration (e.g., individual injection) of the pharmaceutical formulation is administered in a volume of about 0.3 cc. In embodiments, each individual administration (e.g., individual injection) of the pharmaceutical formulation is administered in a volume of about 0.4 cc. In embodiments, each individual administration (e.g., individual injection) of the pharmaceutical formulation is administered in a volume of about 0.5 cc. In embodiments, each individual administration (e.g., individual injection) of the pharmaceutical formulation is administered in a volume of about 0.6 cc. In embodiments, each individual administration (e.g., individual injection) of the pharmaceutical formulation is administered in a volume of about 0.7 cc. In embodiments, each individual administration (e.g., individual injection) of the pharmaceutical formulation is administered in a volume of about 0.8 cc. In embodiments, each individual administration (e.g., individual injection) of the pharmaceutical formulation is administered in a volume of about 0.9 cc. In embodiments, each individual administration (e.g., individual injection) of the pharmaceutical formulation is administered in a volume of about 1.0 cc. In embodiments, each individual administration (e.g., individual injection) of the pharmaceutical formulation is administered in a volume of about 2.0 cc. In embodiments, each individual administration (e.g., individual injection) of the pharmaceutical formulation is administered in a volume of about 3.0 cc. In embodiments, each individual administration (e.g., individual injection) of the pharmaceutical formulation is administered in a volume of about 4.0 cc. In embodiments, each individual administration (e.g., individual injection) of the pharmaceutical formulation is administered in a volume of about 5.0 cc.

In embodiments, each individual administration (e.g., individual injection) of the pharmaceutical formulation is administered in a volume of from 0.1 cc to 0.5 cc. In embodiments, each individual administration (e.g., individual injection) of the pharmaceutical formulation is administered in a volume of from 0.2 cc to 0.5 cc. In embodiments, each individual administration (e.g., individual injection) of the pharmaceutical formulation is administered in a volume of from 0.3 cc to 0.5 cc. In embodiments, each individual administration (e.g., individual injection) of the pharmaceutical formulation is administered in a volume of from 0.4 cc to 0.5 cc. In embodiments, each individual administration (e.g., individual injection) of the pharmaceutical formulation is administered in a volume of from 0.1 cc to 0.4 cc. In embodiments, each individual administration (e.g., individual injection) of the pharmaceutical formulation is administered in a volume of from 0.1 cc to 0.3 cc. In embodiments, each individual administration (e.g., individual injection) of the pharmaceutical formulation is administered in a volume of from 0.1 cc to 0.2 cc. In embodiments, each individual administration (e.g., individual injection) of the pharmaceutical formulation is administered in a volume of from 0.1 cc to 0.6 cc. In embodiments, each individual administration (e.g., individual injection) of the pharmaceutical formulation is administered in a volume of from 0.1 cc to 0.7 cc. In embodiments, each individual administration (e.g., individual injection) of the pharmaceutical formulation is administered in a volume of from 0.1 cc to 0.8 cc. In embodiments, each individual administration (e.g., individual injection) of the pharmaceutical formulation is administered in a volume of from 0.1 cc to 0.9 cc. In embodiments, each individual administration (e.g., individual injection) of the pharmaceutical formulation is administered in a volume of from 0.1 cc to 1.0 cc. In embodiments, each individual administration (e.g., individual injection) of the pharmaceutical formulation is administered in a volume of from 0.3 cc to 0.4 cc. In embodiments, each individual administration (e.g., individual injection) of the pharmaceutical formulation is administered in a volume of from 0.2 cc to 0.3 cc. In embodiments, each individual administration (e.g., individual injection) of the pharmaceutical formulation is administered in a volume of from 0.2 cc to 0.4 cc. In embodiments, each individual administration (e.g., individual injection) of the pharmaceutical formulation is administered in a volume of from 0.2 cc to 0.5 cc. In embodiments, each individual administration (e.g., individual injection) of the pharmaceutical formulation is administered in a volume of from 0.2 cc to 0.6 cc. In embodiments, each individual administration (e.g., individual injection) of the pharmaceutical formulation is administered in a volume of from 0.2 cc to 0.7 cc. In embodiments, each individual administration (e.g., individual injection) of the pharmaceutical formulation is administered in a volume of from 0.2 cc to 0.8 cc. In embodiments, each individual administration (e.g., individual injection) of the pharmaceutical formulation is administered in a volume of from 0.2 cc to 0.9 cc. In embodiments, each individual administration (e.g., individual injection) of the pharmaceutical formulation is administered in a volume of from 0.2 cc to 1.0 cc.

In embodiments, each individual administration (e.g., individual injection) of the pharmaceutical formulation is administered in a volume of 0.1 cc. In embodiments, each individual administration (e.g., individual injection) of the pharmaceutical formulation is administered in a volume of 0.2 cc. In embodiments, each individual administration (e.g., individual injection) of the pharmaceutical formulation is administered in a volume of 0.3 cc. In embodiments, each individual administration (e.g., individual injection) of the pharmaceutical formulation is administered in a volume of 0.4 cc. In embodiments, each individual administration (e.g., individual injection) of the pharmaceutical formulation is administered in a volume of 0.5 cc. In embodiments, each individual administration (e.g., individual injection) of the pharmaceutical formulation is administered in a volume of 0.6 cc. In embodiments, each individual administration (e.g., individual injection) of the pharmaceutical formulation is administered in a volume of 0.7 cc. In embodiments, each individual administration (e.g., individual injection) of the pharmaceutical formulation is administered in a volume of 0.8 cc. In embodiments, each individual administration (e.g., individual injection) of the pharmaceutical formulation is administered in a volume of 0.9 cc. In embodiments, each individual administration (e.g., individual injection) of the pharmaceutical formulation is administered in a volume of 1.0 cc.

In embodiments, the pharmaceutical composition is supplied in a volume of about 0.1 cc to about 20.0 cc. In embodiments, the pharmaceutical composition is supplied in a volume of about 0.1 cc to about 15.0 cc. In embodiments, the pharmaceutical composition is supplied in a volume of about 0.1 cc to about 10.0 cc. In embodiments, the pharmaceutical composition is supplied in a volume of about 1.0 cc to about 20.0 cc. In embodiments, the pharmaceutical composition is supplied in a volume of about 1.0 cc to about 15.0 cc. In embodiments, the pharmaceutical composition is supplied in a volume of about 1.0 cc to about 10.0 cc. In embodiments, the pharmaceutical composition is supplied in a volume of about 0.1 cc to about 20.0 cc. In embodiments, the pharmaceutical composition is supplied in a volume of about 5.0 cc. In embodiments, the pharmaceutical composition is supplied in a volume of about 6.0 cc. In embodiments, the pharmaceutical composition is supplied in a volume of about 7.0 cc. In embodiments, the pharmaceutical composition is supplied in a volume of about 8.0 cc. In embodiments, the pharmaceutical composition is supplied in a volume of about 9.0 cc. In embodiments, the pharmaceutical composition is supplied in a volume of about 10.0 cc. In embodiments, the pharmaceutical composition is supplied in a volume of about 11.0 cc. In embodiments, the pharmaceutical composition is supplied in a volume of about 12.0 cc. In embodiments, the pharmaceutical composition is supplied in a volume of about 13.0 cc. In embodiments, the pharmaceutical composition is supplied in a volume of about 14.0 cc. In embodiments, the pharmaceutical composition is supplied in a volume of about 15.0 cc. In embodiments, the pharmaceutical composition is supplied in a volume of about 16.0 cc. In embodiments, the pharmaceutical composition is supplied in a volume of about 17.0 cc. In embodiments, the pharmaceutical composition is supplied in a volume of about 18.0 cc. In embodiments, the pharmaceutical composition is supplied in a volume of about 19.0 cc. In embodiments, the pharmaceutical composition is supplied in a volume of about 20.0 cc.

In embodiments of the method, the pharmaceutical formulation includes polidocanol. In embodiments of the method, the pharmaceutical formulation consists primarily of polidocanol. In embodiments of the method, the pharmaceutical formulation consists of polidocanol. In embodiments of the method, the pharmaceutical formulation includes a co-solvent. In embodiments the co-solvent is a C₃-C₆ alcohol as described herein. In embodiments the co-solvent is propylene glycol. In embodiments, the co-solvent reduces polidocanol crystallization (e.g., relative to control, for example relative to the absence of the co-solvent). In embodiments, the co-solvent increases polidocanol solubilization (e.g., relative to control, for example relative to the absence of the co-solvent). In embodiments, the co-solvent increases the uniformity of reduction of subcutaneous adipose tissue by polidocanol in the affected area (e.g., relative to control, for example relative to the absence of the co-solvent). In embodiments, the co-solvent decreases patchiness of subcutaneous adipose tissue reduction by polidocanol in the affected area (e.g., relative to control, for example relative to the absence of the co-solvent). In embodiments, the co-solvent increases evenness of subcutaneous adipose tissue reduction by polidocanol in the affected area (e.g., relative to control, for example relative to the absence of the co-solvent).

In embodiments, the co-solvent increases the uniformity of reduction of subcutaneous adipose tissue by the pharmaceutical formulation in the affected area (e.g., relative to control, for example relative to the absence of the co-solvent). In embodiments, the co-solvent decreases patchiness of subcutaneous adipose tissue reduction by the pharmaceutical formulation in the affected area (e.g., relative to control, for example relative to the absence of the co-solvent). In embodiments, the co-solvent increases evenness of subcutaneous adipose tissue reduction by the pharmaceutical formulation in the affected area (e.g., relative to control, for example relative to the absence of the co-solvent).

In embodiments, the co-solvent increases the uniformity of necrosis of adipocytes (fat cells) by polidocanol in the affected area (e.g., relative to control, for example relative to the absence of the co-solvent). In embodiments, the co-solvent increases the uniformity of adipocyte (fat cell) destruction (e.g., by apoptosis, necrosis, or lysis) by polidocanol in the affected area (e.g., relative to control, for example relative to the absence of the co-solvent). In embodiments, the co-solvent increases evenness of adipocyte (fat cell) necrosis by polidocanol in the affected area (e.g., relative to control, for example relative to the absence of the co-solvent). In embodiments, the co-solvent increases evenness of adipocyte (fat cell) destruction (e.g., by apoptosis, necrosis, or lysis) by polidocanol in the affected area (e.g., relative to control, for example relative to the absence of the co-solvent).

In embodiments, the co-solvent increases the uniformity of necrosis of adipocytes (fat cells) by the pharmaceutical formulation in the affected area (e.g., relative to control, for example relative to the absence of the co-solvent). In embodiments, the co-solvent increases the uniformity of adipocyte (fat cell) destruction (e.g., by apoptosis, necrosis, or lysis) by the pharmaceutical formulation in the affected area (e.g., relative to control, for example relative to the absence of the co-solvent). In embodiments, the co-solvent increases evenness of adipocyte (fat cell) necrosis by the pharmaceutical formulation in the affected area (e.g., relative to control, for example relative to the absence of the co-solvent). In embodiments, the co-solvent increases evenness of adipocyte (fat cell) destruction (e.g., by apoptosis, necrosis, or lysis) by the pharmaceutical formulation in the affected area (e.g., relative to control, for example relative to the absence of the co-solvent).

In as aspect is provided a method of reducing subcutaneous adipose tissue in a subject in need thereof, the method including administering to the subject a pharmaceutical formulation including polidocanol and a co-solvent, where the pharmaceutical formulation is administered within a plurality of treatment sessions, wherein each treatment session is spaced by at least 14 days.

In embodiments, each treatment session is spaced by at least 28 days.

In an aspect, provided herein are one or more methods described herein to reduce fat deposits under the eye, chin, or arm, as well as the buttock, calf, back, thigh, ankle, or stomach of a mammal. In another embodiment, the methods described herein reduce specific types of fat deposits such as eyelid fat herniation, lipomas, lipodystrophy, buffalo hump lipodystrophy, or fat deposits associated with cellulite. In another embodiment, the method reduces fat associated with fat redistribution syndrome, eyelid fat herniation, lipomas, Dercum's disease, lipodystrophy, buffalo hump lipodystrophy, dorsocervical fat, visceral adiposity, breast enlargement, hyperadiposity, diffused body fat around trunk and arms, and fat deposits associated with cellulite.

Provided herein are methods of selective, ablative and/or non-ablative fat reduction in an individual in need, the method including: administering to the individual an effective amount of a formulation described herein. Also provided herein are methods of treating a lipoma in an individual, the methods including subcutaneously administering or providing to the individual a formulation described herein. Described herein are adipolytic compositions and formulations, non-invasive methods and systems, and kits for body contouring, including reducing, emulsifying, and/or eliminating subcutaneous adipose tissue, including fat deposits.

In an aspect, provided herein is a method for increasing muscle mass in a subject in need thereof, including administering to the subject a sustained release or rapid release formulation described herein.

In an aspect is provided a method for local fat reduction is provided.

The method may include administering an effective amount of polidocanol. The polidocanol may contain a mixture of C12 alkyl ethoxylate homologues wherein at least about 10% (e.g. at least about 15%) of the mixture of C12 alkyl ethoxylate homologues has an HLB from about 10 to about 15. The polidocanol may be provided in a formulation with a co-solvent, for example propylene glycol. The polidocanol may contain a mixture of C12 alkyl ethoxylate homologues wherein at least 10% (e.g. at least 15%) of the mixture of C12 alkyl ethoxylate homologues has an HLB from 10 to 15. The polidocanol may be provided in a formulation with a co-solvent, for example propylene glycol.

In embodiments, the polidocanol provided herein is chromatographically pure. In other embodiments, the polidocanol is not chromatographically pure.

The method may include administering an effective amount of polidocanol (e.g. subcutaneously). The polidocanol may be at a concentration greater than 0.1% and less than 1.5%. The polidocanol may be formulated with a co-solvent such as propylene glycol. In embodiments, the administration frequency is not less than 14 days (e.g., not less than 28 days).

The polidocanol may be at a concentration less than about 1.5%. The volume may be less than about 0.5 cc per injection. The polidocanol may be at a concentration less than 1.5%. The volume may be less than 0.5 cc per injection. The polidocanol may be formulated with a co-solvent such as propylene glycol. In embodiments, the administration frequency is not less than 14 days (e.g., not less than 28 days).

In embodiments, substantial fat destruction of an inguinal fat deposit is accomplished using a concentration of polidocanol less than about 2.0%. In embodiments, substantial fat destruction of the inguinal fat deposit is accomplished using a concentration of polidocanol less than about 1.75%. In embodiments, substantial fat destruction of the inguinal fat deposit is accomplished using a concentration of polidocanol less than about 1.5%. In embodiments, substantial fat destruction of the inguinal fat deposit is accomplished using a concentration of polidocanol less than about 1.4%. In embodiments, substantial fat destruction of the inguinal fat deposit is accomplished using a concentration of polidocanol less than about 1.3%. In embodiments, substantial fat destruction of the inguinal fat deposit is accomplished using a concentration of polidocanol less than about 1.25%. In embodiments, substantial fat destruction of the inguinal fat deposit is accomplished using a concentration of polidocanol less than about 1.2%. In embodiments, substantial fat destruction of the inguinal fat deposit is accomplished using a concentration of polidocanol less than about 1.1%. In embodiments, substantial fat destruction of the inguinal fat deposit is accomplished using a concentration of polidocanol less than about 1.0%. In embodiments, substantial fat destruction of the inguinal fat deposit is accomplished using a concentration of polidocanol less than about 0.9%. In embodiments, substantial fat destruction of the inguinal fat deposit is accomplished using a concentration of polidocanol less than about 0.8%. In embodiments, substantial fat destruction of the inguinal fat deposit is accomplished using a concentration of polidocanol less than about 0.7%. In embodiments, substantial fat destruction of the inguinal fat deposit is accomplished using a concentration of polidocanol less than about 0.6%.

In embodiments, substantial fat destruction of an inguinal fat deposit is accomplished using a concentration of polidocanol less than 2.0%. In embodiments, substantial fat destruction of the inguinal fat deposit is accomplished using a concentration of polidocanol less than 1.75%. In embodiments, substantial fat destruction of the inguinal fat deposit is accomplished using a concentration of polidocanol less than 1.5%. In embodiments, substantial fat destruction of the inguinal fat deposit is accomplished using a concentration of polidocanol less than 1.4%. In embodiments, substantial fat destruction of the inguinal fat deposit is accomplished using a concentration of polidocanol less than 1.3%. In embodiments, substantial fat destruction of the inguinal fat deposit is accomplished using a concentration of polidocanol less than 1.25%. In embodiments, substantial fat destruction of the inguinal fat deposit is accomplished using a concentration of polidocanol less than 1.2%. In embodiments, substantial fat destruction of the inguinal fat deposit is accomplished using a concentration of polidocanol less than 1.1%. In embodiments, substantial fat destruction of the inguinal fat deposit is accomplished using a concentration of polidocanol less than 1.0%. In embodiments, substantial fat destruction of the inguinal fat deposit is accomplished using a concentration of polidocanol less than 0.9%. In embodiments, substantial fat destruction of the inguinal fat deposit is accomplished using a concentration of polidocanol less than 0.8%. In embodiments, substantial fat destruction of the inguinal fat deposit is accomplished using a concentration of polidocanol less than 0.7%. In embodiments, substantial fat destruction of the inguinal fat deposit is accomplished using a concentration of polidocanol less than 0.6%.

In embodiments, substantial fat destruction of the inguinal fat deposit is accomplished using a concentration of polidocanol from about 0.1% to about 1.5%. In embodiments, substantial fat destruction of the inguinal fat deposit is accomplished using a concentration of polidocanol from about 0.2% to about 1.5%. In embodiments, substantial fat destruction of the inguinal fat deposit is accomplished using a concentration of polidocanol from about 0.3% to about 1.5%. In embodiments, substantial fat destruction of the inguinal fat deposit is accomplished using a concentration of polidocanol from about 0.4% to about 1.5%. In embodiments, substantial fat destruction of the inguinal fat deposit is accomplished using a concentration of polidocanol from about 0.5% to about 1.5%.

in embodiments, substantial fat destruction of the inguinal fat deposit is accomplished using a concentration of polidocanol from 0.1% to 1.5%. In embodiments, substantial fat destruction of the inguinal fat deposit is accomplished using a concentration of polidocanol from 0.2% to 1.5%. In embodiments, substantial fat destruction of the inguinal fat deposit is accomplished using a concentration of polidocanol from 0.3% to 1.5%. In embodiments, substantial fat destruction of the inguinal fat deposit is accomplished using a concentration of polidocanol from 0.4% to 1.5%. In embodiments, substantial fat destruction of the inguinal fat deposit is accomplished using a concentration of polidocanol from 0.5% to 1.5%. In embodiments, substantial fat destruction of the inguinal fat deposit is accomplished using a concentration of polidocanol from about 0.1% to about 1.25%. In embodiments, substantial fat destruction of the inguinal fat deposit is accomplished using a concentration of polidocanol from about 0.2% to about 1.25%. In embodiments, substantial fat destruction of the inguinal fat deposit is accomplished using a concentration of polidocanol from about 0.3% to about 1.25%. In embodiments, substantial fat destruction of the inguinal fat deposit is accomplished using a concentration of polidocanol from about 0.4% to about 1.25%. In embodiments, substantial fat destruction of the inguinal fat deposit is accomplished using a concentration of polidocanol from about 0.5% to about 1.25%.

In embodiments, substantial fat destruction of the inguinal fat deposit is accomplished using a concentration of polidocanol from 0.1% to 1.25%. In embodiments, substantial fat destruction of the inguinal fat deposit is accomplished using a concentration of polidocanol from 0.2% to 1.25%. In embodiments, substantial fat destruction of the inguinal fat deposit is accomplished using a concentration of polidocanol from 0.3% to 1.25%. In embodiments, substantial fat destruction of the inguinal fat deposit is accomplished using a concentration of polidocanol from 0.4% to 1.25%. In embodiments, substantial fat destruction of the inguinal fat deposit is accomplished using a concentration of polidocanol from 0.5% to 1.25%.

In embodiments, substantial fat destruction of the inguinal fat deposit is accomplished using a concentration of polidocanol from about 0.1% to about 1.0%. In embodiments, substantial fat destruction of the inguinal fat deposit is accomplished using a concentration of polidocanol from about 0.2% to about 1.0%. In embodiments, substantial fat destruction of the inguinal fat deposit is accomplished using a concentration of polidocanol from about 0.3% to about 1.0%. In embodiments, substantial fat destruction of the inguinal fat deposit is accomplished using a concentration of polidocanol from about 0.4% to about 1.0%. In embodiments, substantial fat destruction of the inguinal fat deposit is accomplished using a concentration of polidocanol from about 0.5% to about 1.0%.

In embodiments, substantial fat destruction of the inguinal fat deposit is accomplished using a concentration of polidocanol from 0.1% to 1.0%. In embodiments, substantial fat destruction of the inguinal fat deposit is accomplished using a concentration of polidocanol from 0.2% to 1.0%. In embodiments, substantial fat destruction of the inguinal fat deposit is accomplished using a concentration of polidocanol from 0.3% to 1.0%. In embodiments, substantial fat destruction of the inguinal fat deposit is accomplished using a concentration of polidocanol from 0.4% to 1.0%. In embodiments, substantial fat destruction of the inguinal fat deposit is accomplished using a concentration of polidocanol from 0.5% to 1.0%.

In embodiments, the polidocanol is the product of reacting 1 mole of the corresponding C12 alcohol with 9 moles of ethoxide equivalents under basic conditions (e.g. with a metal base such as potassium hydroxide). In related embodiments, the average number of ethoxylate units of the polidocanol is about 6. In related embodiments, the average number of ethoxylate units of the polidocanol is about 7. In related embodiments, the average number of ethoxylate units of the polidocanol is about 8. In related embodiments, the average number of ethoxylate units of the polidocanol is about 9. In related embodiments, the average number of ethoxylate units of the polidocanol is about 10. In related embodiments, the average molecular weight of the polidocanol is about 480 g/mol to about 620 g/mole. In related embodiments, the average molecular weight of the polidocanol is about 494 g/mol.

The method may include injecting an effective amount of polidocanol (e.g. in an aqueous formulation) into subcutaneous fat of a subject in need thereof. In embodiments, the polidocanol is administered less than once per day. In embodiments, the polidocanol is administered about once per week. In embodiments, the polidocanol is administered about once every 14 days (e.g. twice per month). In embodiments, the polidocanol is administered about once every 28 days (e.g. once per month). In embodiments, the polidocanol is administered once per week. In embodiments, the polidocanol is administered once every 14 days (e.g. twice per month). In embodiments, the polidocanol is administered once every 28 days (e.g. once per month). The injections may occur at no less than 14 day intervals (e.g., no less than 28 day intervals). In some embodiments, the subject receives up to 6 injections at one-month intervals. In some embodiments, the subject receives up to 5 injections at one-month intervals. In some embodiments, the subject receives up to 4 injections at one-month intervals. In some embodiments, the subject receives up to 3 injections at one-month intervals. In some embodiments, the subject receives up to 2 injections at one-month intervals. In some embodiments, the subject receives one injection. In embodiments, the polidocanol is provided in a composition described herein (e.g. with a co-solvent such as propylene glycol). In embodiments, the administration of polidocanol is provided in a manner that selectively destroys adipose cells relative to skin cells (e.g. dermal cells). In embodiments, the polidocanol is present in an amount of about 0.1%-10% w/w or about 0.1%-5% w/w. In some embodiments, the polidocanol is present in an amount of about 0.1% w/w to about 10% w/w. In some embodiments, the polidocanol is present in an amount of about 0.1% w/w to about 0.2% w/w, about 0.1% w/w to about 0.5% w/w, about 0.1% w/w to about 1% w/w, about 0.1% w/w to about 2% w/w, about 0.1% w/w to about 3% w/w, about 0.1% w/w to about 4% w/w, about 0.1% w/w to about 4.5% w/w, about 0.1% w/w to about 5% w/w, about 0.1% w/w to about 6% w/w, about 0.1% w/w to about 8% w/w, about 0.1% w/w to about 10% w/w, about 0.2% w/w to about 0.5% w/w, about 0.2% w/w to about 1% w/w, about 0.2% w/w to about 2% w/w, about 0.2% w/w to about 3% w/w, about 0.2% w/w to about 4% w/w, about 0.2% w/w to about 4.5% w/w, about 0.2% w/w to about 5% w/w, about 0.2% w/w to about 6% w/w, about 0.2% w/w to about 8% w/w, about 0.2% w/w to about 10% w/w, about 0.5% w/w to about 1% w/w, about 0.5% w/w to about 2% w/w, about 0.5% w/w to about 3% w/w, about 0.5% w/w to about 4% w/w, about 0.5% w/w to about 4.5% w/w, about 0.5% w/w to about 5% w/w, about 0.5% w/w to about 6% w/w, about 0.5% w/w to about 8% w/w, about 0.5% w/w to about 10% w/w, about 1% w/w to about 2% w/w, about 1% w/w to about 3% w/w, about 1% w/w to about 4% w/w, about 1% w/w to about 4.5% w/w, about 1% w/w to about 5% w/w, about 1% w/w to about 6% w/w, about 1% w/w to about 8% w/w, about 1% w/w to about 10% w/w, about 2% w/w to about 3% w/w, about 2% w/w to about 4% w/w, about 2% w/w to about 4.5% w/w, about 2% w/w to about 5% w/w, about 2% w/w to about 6% w/w, about 2% w/w to about 8% w/w, about 2% w/w to about 10% w/w, about 3% w/w to about 4% w/w, about 3% w/w to about 4.5% w/w, about 3% w/w to about 5% w/w, about 3% w/w to about 6% w/w, about 3% w/w to about 8% w/w, about 3% w/w to about 10% w/w, about 4% w/w to about 4.5% w/w, about 4% w/w to about 5% w/w, about 4% w/w to about 6% w/w, about 4% w/w to about 8% w/w, about 4% w/w to about 10% w/w, about 4.5% w/w to about 5% w/w, about 4.5% w/w to about 6% w/w, about 4.5% w/w to about 8% w/w, about 4.5% w/w to about 10% w/w, about 5% w/w to about 6% w/w, about 5% w/w to about 8% w/w, about 5% w/w to about 10% w/w, about 6% w/w to about 8% w/w, about 6% w/w to about 10% w/w, or about 8% w/w to about 10% w/w. In some embodiments, the polidocanol is present in an amount of about 0.1% w/w, about 0.2% w/w, about 0.5% w/w, about 1% w/w, about 2% w/w, about 3% w/w, about 4% w/w, about 4.5% w/w, about 5% w/w, about 6% w/w, about 8% w/w, or about 10% w/w. In some embodiments, the polidocanol is present in an amount of at least about 0.1% w/w, about 0.2% w/w, about 0.5% w/w, about 1% w/w, about 2% w/w, about 3% w/w, about 4% w/w, about 4.5% w/w, about 5% w/w, about 6% w/w, or about 8% w/w. In some embodiments, the polidocanol is present in an amount of at most about 0.2% w/w, about 0.5% w/w, about 1% w/w, about 2% w/w, about 3% w/w, about 4% w/w, about 4.5% w/w, about 5% w/w, about 6% w/w, about 8% w/w, or about 10% w/w. In some embodiments, the polidocanol is present in an amount of about 2.0% w/w. In some embodiments, the polidocanol is present in an amount of about 3.0% w/w. In some embodiments, the polidocanol is present in an amount of about 4.5% w/w.

In an aspect is provided a method for treating adipose tissue including administering to a human or animal subject by subcutaneous injection a formulation including an effective amount, for example a therapeutically or cosmetically effective amount, of polidocanol and an alcohol having 3 to 6 carbon atoms (e.g., from about 0.5% w/w to about 10% w/w). In some embodiments, the alcohol having 3 to 6 carbon atoms is present in an amount of from about 1% w/w to about 5% w/w. In some embodiments, the alcohol having 3 to 6 carbon atoms is present in an amount of approximately 5% w/w. In some embodiments, the method includes treating adipose tissue including administering to a human or animal subject by subcutaneous injection a formulation including polidocanol in an amount that is greater than 1.0% w/w and from about 0.5% w/w to about 10% w/w of an alcohol having 3 to 6 carbon atoms. In some embodiments, the method includes treating adipose tissue including administering to a human or animal subject by subcutaneous injection a formulation including polidocanol in an amount from about 0.1% w/w to about 20.0% w/w and from about 0.5% w/w to about 10% w/w of an alcohol having 3 to 6 carbon atoms. In some embodiments, the method includes treating adipose tissue including administering to a human or animal subject by subcutaneous injection a formulation including polidocanol in an amount from about 0.5% w/w to about 10.0% w/w and from about 0.5% w/w to about 10% w/w of an alcohol having 3 to 6 carbon atoms. In some embodiments, the method includes treating adipose tissue including administering to a human or animal subject by subcutaneous injection a formulation including polidocanol in an amount from about 0.1% w/w to about 2.0% w/w and from about 0.5% w/w to about 10% w/w of an alcohol having 3 to 6 carbon atoms. In some embodiments, the method includes treating adipose tissue including administering to a human or animal subject by subcutaneous injection a formulation including polidocanol in an amount of about 0.5% w/w, 1.25%, or 2.0% w/w and from about 0.5% w/w to about 10% w/w of an alcohol having 3 to 6 carbon atoms. In some embodiments, the formulation has a pH of from about 6 to 9. In some embodiments, the formulation has a pH of from about 7 to 8.

In an aspect is provided a method for treating adipose tissue including administering to a human or animal subject by subcutaneous injection a formulation including an effective amount, for example a therapeutically or cosmetically effective amount, of polidocanol and an alcohol having 3 to 6 carbon atoms (e.g., from 0.5% w/w to 10% w/w). In some embodiments, the alcohol having 3 to 6 carbon atoms is present in an amount of from 1% w/w to 5% w/w. In some embodiments, the alcohol having 3 to 6 carbon atoms is present in an amount of approximately 5% w/w. In some embodiments, the method includes treating adipose tissue including administering to a human or animal subject by subcutaneous injection a formulation including polidocanol in an amount that is greater than 1.0% w/w and from 0.5% w/w to 10% w/w of an alcohol having 3 to 6 carbon atoms. In some embodiments, the method includes treating adipose tissue including administering to a human or animal subject by subcutaneous injection a formulation including polidocanol in an amount from 0.1% w/w to 20.0% w/w and from 0.5% w/w to 10% w/w of an alcohol having 3 to 6 carbon atoms. In some embodiments, the method includes treating adipose tissue including administering to a human or animal subject by subcutaneous injection a formulation including polidocanol in an amount from 0.5% w/w to 10.0% w/w and from 0.5% w/w to 10% w/w of an alcohol having 3 to 6 carbon atoms. In some embodiments, the method includes treating adipose tissue including administering to a human or animal subject by subcutaneous injection a formulation including polidocanol in an amount from 0.1% w/w to 2.0% w/w and from 0.5% w/w to 10% w/w of an alcohol having 3 to 6 carbon atoms. In some embodiments, the method includes treating adipose tissue including administering to a human or animal subject by subcutaneous injection a formulation including polidocanol in an amount of 0.5% w/w, 1.25%, or 2.0% w/w and from 0.5% w/w to 10% w/w of an alcohol having 3 to 6 carbon atoms. In some embodiments, the formulation has a pH of from 6 to 9. In some embodiments, the formulation has a pH of from 7 to 8.

In an aspect is provided a method for the reduction and/or prevention of subcutaneous adipose tissue including administering to a human or animal subject by subcutaneous injection polidocanol (e.g., wherein said administering is performed no more than once every 28 days). The polidocanol may be provided in an aqueous formulation. The formulation may be any appropriate composition described herein, including a composition with a co-solvent such as propylene glycol.

In as aspect is provided a method for the reduction and/or prevention of subcutaneous adipose tissue. In some embodiments, the method includes administering to a human or animal subject by subcutaneous injection a formulation including polidocanol in an amount of from about 0.5% w/w to 2.0% w/w and propylene glycol in an amount of from about 1% w/w to 5% w/w, wherein the formulation does not contain polyethylene glycol or poloxamers. In some embodiments, the method includes administering to a human or animal subject by subcutaneous injection a formulation including polidocanol in an amount of from about 0.5% w/w to 2.0% w/w and glycerine in an amount of from about 1% w/w to 5% w/w, wherein the formulation does not contain polyethylene glycol or poloxamers. In some embodiments, the method includes administering to a human or animal subject by subcutaneous injection a formulation including polidocanol in an amount of from 0.5% w/w to 2.0% w/w and propylene glycol in an amount of from 1% w/w to 5% w/w, wherein the formulation does not contain polyethylene glycol or poloxamers. In some embodiments, the method includes administering to a human or animal subject by subcutaneous injection a formulation including polidocanol in an amount of from 0.5% w/w to 2.0% w/w and glycerine in an amount of from 1% w/w to 5% w/w, wherein the formulation does not contain polyethylene glycol or poloxamers.

In as aspect is provided a method for treating adipose tissue including administering a formulations or composition described herein. In some embodiments, described herein are methods for treating adipose tissue including administering to a human or animal subject in need by subcutaneous injection a formulation including an effective amount, including for example a therapeutically or cosmetically effective amount, of polidocanol (about 0.5% w/w to about 10% w/w). In some embodiments, described herein are methods for treating adipose tissue including administering to a human or animal subject in need by subcutaneous injection a formulation including an effective amount, including for example a therapeutically or cosmetically effective amount, of polidocanol and from about 0.5% w/w to about 10% w/w of glycerine. In some embodiments, the methods for treating adipose tissue include administering to a human or animal subject in need by subcutaneous injection a formulation including an effective amount, including for example a therapeutically or cosmetically effective amount, of polidocanol and from about 0.5% w/w to about 10% w/w of propylene glycol wherein the formulation does not contain ethanol or an ether. In some embodiments, the methods for treating adipose tissue include administering to a human or animal subject in need by subcutaneous injection a formulation including an effective amount, including for example a therapeutically or cosmetically effective amount, of polidocanol and from about 0.5% w/w to about 10% w/w of glycerine wherein the formulation does not contain ethanol or an ether. In some embodiments, the methods for treating adipose tissue include administering to a human or animal subject in need by subcutaneous injection a formulation including an effective amount, including for example a therapeutically or cosmetically effective amount, of polidocanol and from about 0.5% w/w to about 10% w/w of propylene glycol wherein the formulation does not contain aliphatic polyethers, such as polyethylene glycol or poloxamers. In some embodiments, the methods for treating adipose tissue include administering to a human or animal subject in need by subcutaneous injection a formulation including an effective amount, including for example a therapeutically or cosmetically effective amount, of polidocanol and from about 0.5% w/w to about 10% w/w of glycerine wherein the formulation does not contain aliphatic polyethers, such as polyethylene glycol or poloxamers. In some embodiments, the formulations do not contain ethanol or an ether or aliphatic polyethers such as polyethylene glycol or poloxamers. In some embodiments, described herein are methods for treating adipose tissue including administering to a human or animal subject in need by subcutaneous injection a formulation including an effective amount, including for example a therapeutically or cosmetically effective amount, of polidocanol and from 0.5% w/w to 10% w/w of propylene glycol. In some embodiments, described herein are methods for treating adipose tissue including administering to a human or animal subject in need by subcutaneous injection a formulation including an effective amount, including for example a therapeutically or cosmetically effective amount, of polidocanol and from 0.5% w/w to 10% w/w of glycerine. In some embodiments, the methods for treating adipose tissue include administering to a human or animal subject in need by subcutaneous injection a formulation including an effective amount, including for example a therapeutically or cosmetically effective amount, of polidocanol and from 0.5% w/w to 10% w/w of propylene glycol wherein the formulation does not contain ethanol or an ether. In some embodiments, the methods for treating adipose tissue include administering to a human or animal subject in need by subcutaneous injection a formulation including an effective amount, including for example a therapeutically or cosmetically effective amount, of polidocanol and from 0.5% w/w to 10% w/w of glycerine wherein the formulation does not contain ethanol or an ether. In some embodiments, the methods for treating adipose tissue include administering to a human or animal subject in need by subcutaneous injection a formulation including an effective amount, including for example a therapeutically or cosmetically effective amount, of polidocanol and from 0.5% w/w to 10% w/w of propylene glycol wherein the formulation does not contain aliphatic polyethers, such as polyethylene glycol or poloxamers. In some embodiments, the methods for treating adipose tissue include administering to a human or animal subject in need by subcutaneous injection a formulation including an effective amount, including for example a therapeutically or cosmetically effective amount, of polidocanol and from 0.5% w/w to 10% w/w of glycerine wherein the formulation does not contain aliphatic polyethers, such as polyethylene glycol or poloxamers. In some embodiments, the formulations do not contain ethanol or an ether or aliphatic polyethers such as polyethylene glycol or poloxamers.

In an aspect is provided a cosmetic or therapeutic method including subcutaneously administering or providing to a human a formulation including a compound of Formula I or Formula II or a pharmaceutically acceptable or cosmetically acceptable salt or other form suitable for administration and a liquid carrier that is formulated for injection into a layer of subcutaneous fat in or to a human in need. In an embodiment, a formulation including polidocanol of Formula II is administered to the human to treat a disease selected from one or more of: fat tissue, abdominal fat accumulation, regional adiposity, excess sub-mental fat, and exophthalmos due to thyroid eye disease. In certain embodiments, the formulation is provided to the human to affect a shape, contour, or appearance of the human body. In an embodiment, the shape, contour, or appearance is in a region of the body (e.g., the abdominal region or eye region of the human). In certain other embodiments, the formulation is administered or provided to the human subcutaneously as an abdominal, peri-orbital, intra-orbital, or sub-mental injection. In an embodiment, a formulation described herein is administered or provided to the human subcutaneously to an abdominal region, an ophthalmic region, or a sub-mental region. In certain embodiments of the cosmetic and/or therapeutic methods described herein, the formulation is administered or provided to the human in the inside region of the knees, the middle to upper area of the upper arm (including the tricep area), the sub-mental area (including the area under the chin, for example the wattle (which is understood to refer to the fleshy fold of skin in the sub-mental area of the human)), the abdomen, the hips, the inner thigh, the outer thigh, the buttocks, the lower back, the upper back, or the chest.

In as aspect is provided a method for treating fat accumulation including administering an injectable formulation including a compound of Formula I or Formula II or a pharmaceutically acceptable or cosmetically acceptable salt or other suitable form thereof and a liquid carrier that is formulated for injection into a layer of subcutaneous fat in a human in need. In certain embodiments is a method for treating a fat accumulation including administering an injectable formulation including polidocanol, or a polidocanol-like compound of Formula I or Formula II or a pharmaceutically acceptable or cosmetically acceptable salt or other suitable form thereof and a liquid carrier formulated for injection into a layer of subcutaneous fat in a human in need. Also provided are methods of treating regional adipose tissue including administering an injectable formulation including at least one compound of Formula I or Formula II or pharmaceutically acceptable or cosmetically acceptable salt or other suitable form, or combinations thereof and a liquid carrier formulated for injection into a layer of subcutaneous fat in a human in need. Also provided are methods of treating regional adipose tissue including administering an injectable formulation including polidocanol of Formula II or a pharmaceutically acceptable or cosmetically acceptable salt or other suitable form thereof and a liquid carrier formulated for injection into a layer of subcutaneous fat in a human in need. Also provided are methods of treating regional adiposity including administering an injectable formulation including polidocanol of Formula II or a pharmaceutically acceptable or cosmetically acceptable salt or other suitable form thereof and a liquid carrier formulated for injection into a layer of subcutaneous fat in a human in need.

In as aspect. provided is a method including administering an injectable formulation including at least one of a compound of Formula I or Formula II or a pharmaceutically acceptable or cosmetically acceptable salt or other suitable form thereof, or combinations thereof and a liquid carrier formulated for injection into a layer of subcutaneous fat in a human in need, wherein administration results in emulsifying, necrosis, lysing or destroying one or more adipose cells. In an embodiment, provided is a method including administering an injectable formulation including polidocanol of Formula II or a pharmaceutically acceptable or cosmetically acceptable salt or other suitable form thereof and a liquid carrier formulated for injection into a layer of subcutaneous fat in a human in need, wherein the administration results in emulsifying, necrosis, lysing or destroying one or more adipose cells. In certain embodiments, the methods described herein further result in an accompanying inflammatory reaction that at least partially removes or decreases destroyed or lysed adipose cells.

Provided herein is a method including administering an injectable formulation including a compound of Formula I or Formula II such as polidocanol or a pharmaceutically acceptable or cosmetically acceptable salt or other suitable form thereof and a liquid carrier formulated for injection into a layer of subcutaneous fat in a human in need, wherein said administration results in selectively emulsifying, necrotizing, lysing or destroying one or more adipose cells while, in certain situations, leaving surrounding tissue largely unaffected.

In certain embodiments, provided is a formulation or composition including a compound of Formula I or Formula II (e.g., polidocanol) that provides one or more of the following: (a) a critical micelle concentration (“CMC”) of less than about 5 millimolar; (b) an HLB value of from about 10 to about 15; and (c) is non-ionic. In some embodiments, these formulations are used for contacting fat tissue, abdominal fat accumulation, regional adiposity, excess sub-mental fat, and exophthalmos (e.g., due to thyroid eye disease), and/or for emulsifying, necrotizing, lysing or destroying one or more adipose cells while, in certain situations, leaving surrounding tissue largely unaffected (e.g., unlysed or less lysed in comparison to the lysis of fat cells (e.g., percentage of lysed cells, number of lysed cells, degree of lysis of a tissue or cell population).

In certain embodiments, the compound is polidocanol. Also provided herein is a method of administration to subcutaneous tissue including contacting the subcutaneous tissue with a pharmaceutically effective amount of polidocanol, provided that the polidocanol is present in an amount of about 0.1%-10% w/w or about 0.1%-10.0% w/w. In embodiments, the polidocanol is present in an amount of about 0.1%-10% w/w or about 0.1%-5% w/w. In some embodiments, the polidocanol is present in an amount of about 0.1% w/w to about 10% w/w. In some embodiments, the polidocanol is present in an amount of about 0.1% w/w to about 0.2% w/w, about 0.1% w/w to about 0.5% w/w, about 0.1% w/w to about 1% w/w, about 0.1% w/w to about 2% w/w, about 0.1% w/w to about 3% w/w, about 0.1% w/w to about 4% w/w, about 0.1% w/w to about 4.5% w/w, about 0.1% w/w to about 5% w/w, about 0.1% w/w to about 6% w/w, about 0.1% w/w to about 8% w/w, about 0.1% w/w to about 10% w/w, about 0.2% w/w to about 0.5% w/w, about 0.2% w/w to about 1% w/w, about 0.2% w/w to about 2% w/w, about 0.2% w/w to about 3% w/w, about 0.2% w/w to about 4% w/w, about 0.2% w/w to about 4.5% w/w, about 0.2% w/w to about 5% w/w, about 0.2% w/w to about 6% w/w, about 0.2% w/w to about 8% w/w, about 0.2% w/w to about 10% w/w, about 0.5% w/w to about 1% w/w, about 0.5% w/w to about 2% w/w, about 0.5% w/w to about 3% w/w, about 0.5% w/w to about 4% w/w, about 0.5% w/w to about 4.5% w/w, about 0.5% w/w to about 5% w/w, about 0.5% w/w to about 6% w/w, about 0.5% w/w to about 8% w/w, about 0.5% w/w to about 10% w/w, about 1% w/w to about 2% w/w, about 1% w/w to about 3% w/w, about 1% w/w to about 4% w/w, about 1% w/w to about 4.5% w/w, about 1% w/w to about 5% w/w, about 1% w/w to about 6% w/w, about 1% w/w to about 8% w/w, about 1% w/w to about 10% w/w, about 2% w/w to about 3% w/w, about 2% w/w to about 4% w/w, about 2% w/w to about 4.5% w/w, about 2% w/w to about 5% w/w, about 2% w/w to about 6% w/w, about 2% w/w to about 8% w/w, about 2% w/w to about 10% w/w, about 3% w/w to about 4% w/w, about 3% w/w to about 4.5% w/w, about 3% w/w to about 5% w/w, about 3% w/w to about 6% w/w, about 3% w/w to about 8% w/w, about 3% w/w to about 10% w/w, about 4% w/w to about 4.5% w/w, about 4% w/w to about 5% w/w, about 4% w/w to about 6% w/w, about 4% w/w to about 8% w/w, about 4% w/w to about 10% w/w, about 4.5% w/w to about 5% w/w, about 4.5% w/w to about 6% w/w, about 4.5% w/w to about 8% w/w, about 4.5% w/w to about 10% w/w, about 5% w/w to about 6% w/w, about 5% w/w to about 8% w/w, about 5% w/w to about 10% w/w, about 6% w/w to about 8% w/w, about 6% w/w to about 10% w/w, or about 8% w/w to about 10% w/w. In some embodiments, the polidocanol is present in an amount of about 0.1% w/w, about 0.2% w/w, about 0.5% w/w, about 1% w/w, about 2% w/w, about 3% w/w, about 4% w/w, about 4.5% w/w, about 5% w/w, about 6% w/w, about 8% w/w, or about 10% w/w. In some embodiments, the polidocanol is present in an amount of at least about 0.1% w/w, about 0.2% w/w, about 0.5% w/w, about 1% w/w, about 2% w/w, about 3% w/w, about 4% w/w, about 4.5% w/w, about 5% w/w, about 6% w/w, or about 8% w/w. In some embodiments, the polidocanol is present in an amount of at most about 0.2% w/w, about 0.5% w/w, about 1% w/w, about 2% w/w, about 3% w/w, about 4% w/w, about 4.5% w/w, about 5% w/w, about 6% w/w, about 8% w/w, or about 10% w/w. In some embodiments, the polidocanol is present in an amount of about 2.0% w/w. In some embodiments, the polidocanol is present in an amount of about 3.0% w/w. In some embodiments, the polidocanol is present in an amount of about 4.5% w/w. In as aspect is provided a method of treating lipoma in an individual, including subcutaneously administering or providing to the individual a formulation described herein. In certain embodiments, the method of treating lipoma includes administering to the individual an effective amount, including for example a therapeutically or cosmetically effective amount, of a formulation including polidocanol or a polidocanol-like compound of Formula II, or a pharmaceutically acceptable or cosmetically acceptable salt or other suitable form thereof; and a liquid carrier; wherein the polidocanol or a pharmaceutically acceptable or cosmetically acceptable salt or other suitable form thereof and the liquid carrier are formulated for injection into a layer of subcutaneous fat in the individual in need thereof.

In another aspect, provided herein are methods and formulations that facilitate dispersal of compound of Formula I or Formula II such as polidocanol and salts and other suitable forms thereof into a layer of subcutaneous fat at a regional fat site selected from one or more of the following: a sub-mental region, an abdominal region, a waist, a hip, a lateral buttock, a thigh, a peri-orbital region, an intra-orbital region, and intramuscular region. In certain embodiments is a formulation for the treatment of one or more of: abdominal fat accumulation, regional adiposity, excessive adiposity in the sub-mental region, and exophthalmos caused by thyroid eye disease. In certain embodiments are provided formulations to affect a shape, contour, or appearance of the human body.

In an aspect, provided herein is a method of treating regional adipose tissue, regional adiposity, or regional fat accumulation in an individual, said method including administering to the individual an effective amount of a formulation including: at least one of a compound of Formula I or Formula II such as polidocanol or a pharmaceutically acceptable or cosmetically acceptable salt or other suitable form thereof; and a liquid carrier; wherein the compound of Formula I or Formula II or a pharmaceutically acceptable or cosmetically acceptable salt or other suitable form thereof and the liquid carrier are formulated for injection into a layer of subcutaneous fat in the individual in need thereof.

In as aspect is provided a method of selective, ablative and/or non-ablative fat reduction in an individual in need, said method including administering to said individual an effective amount of a formulation described herein. In as aspect is provided a method of treating a lipoma in an individual, said methods including subcutaneously administering or providing to the individual a formulation described herein.

In another aspect provided herein is a method for increasing muscle mass in a subject in need thereof, including administering to the subject a sustained release or rapid release or immediate release formulation described herein.

In as aspect is provided a method to reduce fat deposits adjacent (e.g., under, over, lateral to, near, forming part of) the eye, chin including the sub-mental region, arm, buttock, calf, back, thigh, ankle, or stomach of a mammal in need thereof. In another embodiment, the methods described herein reduce specific types of fat deposits such as eyelid fat herniation, lipomas, lipodystrophy, buffalo hump lipodystrophy, or fat deposits associated with cellulite.

In yet another aspect, provided is a cosmetic or therapeutic method including subcutaneously administering or providing to a human, including to the subcutaneous tissue of a human, a formulation for treating regional adipose tissue, regional adiposity, or regional fat accumulation including: an effective amount of a compound of Formula I or II such as polidocanol or a pharmaceutically acceptable or cosmetically acceptable salt or other suitable form thereof; and a liquid carrier; the polidocanol or a pharmaceutically acceptable or cosmetically acceptable salt or other suitable form thereof and the liquid carrier formulated for injection into a layer of subcutaneous fat in a human in need. In an embodiment, provided is a method wherein the formulation described herein is administered to the human to treat an indication selected from one or more of: abdominal adiposity, excessive sub-mental adiposity, regional adiposity, and exophthalmos due to thyroid eye disease. In one embodiment, the formulation is provided to the human by subcutaneous injection. In another embodiment, the formulation is provided to the human by transdermal application to the other skin of a patient. In some embodiments, provided is a cosmetic method wherein a formulation described herein is provided to the human to affect a shape, contour, or appearance of the human body. In certain embodiments, the shape, contour, or appearance is in a region of the body (e.g., the abdominal region, sub-mental region, or eye region of the human). In certain embodiments, a formulation described herein is administered or provided to the human subcutaneously as a peri-orbital, intra-orbital, or sub-mental injection. In an embodiment, a formulation described herein is administered or provided to the human subcutaneously to an abdominal region, an ophthalmic region, or a sub-mental region. In certain embodiments, a formulation described herein is administered or provided to the human in the inside region of the knees, the middle to upper area of the upper arm (including the triceps' area), the sub-mental area (including the area under the chin, for example the wattle (which is understood to refer to the fleshy fold of skin in the sub-mental area of the human)), the abdomen, the hips, the inner thigh, the outer thigh, the buttocks, the lower back, the upper back, or the chest.

In an additional aspect, provided herein is a method for treating a fat accumulation including administering an injectable formulation described herein. In an embodiment, provided is a method for treating regional adipose tissue, said method including the step of administering an injectable formulation including at least one compound of Formula II described herein. In certain embodiments, provided is a method for treating regional adiposity including administering an injectable formulation including polidocanol described herein.

In another aspect, provided herein is a method including administering an injectable formulation including a compound of Formula I or Formula II described herein, that results in lysing or destroying one or more adipose cells.

In an aspect, provided is a method including administering an injectable formulation including polidocanol described herein, that results in selectively lysing or destroying one or more adipose cells while in certain applications leaving surrounding tissue largely unaffected.

In an aspect, provided herein is a method of treating regional adipose tissue, regional adiposity, or regional fat accumulation in an individual, said method including administering to the individual an effective amount of a formulation including: a compound of Formula I or Formula II such as polidocanol or a pharmaceutically acceptable or cosmetically acceptable salt or other suitable form thereof; and a liquid carrier; wherein the compound of Formula I or Formula II or a pharmaceutically acceptable or cosmetically acceptable salt or other suitable form thereof and the liquid carrier are formulated for injection into a layer of subcutaneous fat in the individual in need thereof.

In an aspect, provided herein is a method of selective, ablative and/or non-ablative fat reduction in an individual in need, said method including: administering to said individual an effective amount of a formulation including a compound of Formula I or Formula II such as polidocanol or a pharmaceutically acceptable or cosmetically acceptable salt or other suitable form thereof; and a liquid carrier; wherein the compound of Formula I or Formula II or a pharmaceutically acceptable or cosmetically acceptable salt or other suitable form thereof and the liquid carrier are formulated for injection into a layer of subcutaneous fat in the individual.

In certain embodiments, the formulations described herein are administered to an individual to treat an indication selected from one or more of: abdominal adiposity, regional adiposity, excessive sub-mental adipose tissue which often presents as a double chin, and exophthalmos due to thyroid eye disease. In certain embodiments, provided are formulations to affect a shape, contour, or appearance of the human body. In some embodiments are provided cosmetic methods and formulations wherein the formulation is administered or provided to the human subcutaneously as a peri-orbital, intra-orbital, or sub-mental injection. In certain embodiments, the formulations described herein are administered or provided to the individual subcutaneously to an abdominal region, an ophthalmic region, or a sub-mental region. In further or additional embodiments, the formulations described here are administered to the head region of a patient, including for example to address a need in the forehead area of the patient, a brow lift, a Crow's lift, a peri-orbital depression, a cheek enhancement, a nasal labial fold (also referred to as “smile lines”), the glabella, lip enhancement, or sub-mental fat.

Multiple injections over a region are applied to achieve a pharmaceutical or cosmetic effect. These injections may be spaced from 0.1 to up to 5 cm apart. A small volume per injection, less than 0.5 mL, may require spacing 1.0 cm or less, with larger volumes per injection allowing for larger spacing, such that 1.0 mL to 2.0 mL per injection may allow spacing of 1 cm or more and reduce the number of injections required to treat an area. Improving the cosmetic effect and reducing side effects may also be achieved by using larger per injection volume and a lower concentration (w/w) of polidocanol, a polidocanol like compound or a cosmetically acceptable salt. In certain embodiments, a 0.1-1.0% (w/w) with a 1 mL to per injection volume is provided to the desired cosmetic effect with fewer side effects.

In some embodiments, more than one treatment session of a certain region spaced 1-8 weeks apart is provided to achieve the desired pharmaceutical or cosmetic effect. In certain embodiments, the treatment is provided less frequently than once per day. In one treatment embodiment, five to twenty 0.1 to 1.0 mL injections spaced 0.1 cm to 1.0 cm apart of 0.1% to 10.0% weight/weight (as used herein, “w/w”), including about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, or about 10.0% of polidocanol, a polidocanol like, or cosmetically or pharmaceutically acceptable salt or other suitable form thereof, is injected into the sub-mental (under chin) region of a patient. In one treatment embodiment, five to twenty 0.1 to 1.0 mL injections spaced 0.1 cm to 1.0 cm apart of 0.1% to 10.0% weight/weight (as used herein, “w/w”), including 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, or 10.0% of polidocanol, a polidocanol like, or cosmetically or pharmaceutically acceptable salt or other suitable form thereof, is injected into the sub-mental (under chin) region of a patient. As an example, the treatment is repeated up to 6 times at two to four week intervals until the desired cosmetic effect is achieved. In another treatment embodiment, ten 0.2 mL injections spaced 0.5 cm apart of about 0.5%, about 1.0%, about 3.0%, about 5.0%, or about 10.0% weight/weight of polidocanol, or a polidocanol like compound, or cosmetically or pharmaceutically acceptable salts thereof, are injected into the sub-mental (under chin) region of a patient. In one embodiment, the treatment is repeated 4 times at 4 week intervals until the desired cosmetic effect is achieved. In some embodiments, the subject receives up to 6 injections at one-month intervals. In another treatment embodiment, ten 0.2 mL injections spaced 1.0 cm apart of 1.0% weight/weight of polidocanol, a polidocanol like compound, or cosmetically or pharmaceutically acceptable salt or other suitable form thereof, is injected into the sub-mental (under chin) region of a patient. In one treatment embodiment, the treatment is repeated 4 times at 4 week intervals until the desired cosmetic effect is achieved. In still another treatment embodiment, ten 0.4 mL injections spaced 1.0 cm apart of 0.5% weight/weight of polidocanol, a polidocanol like compound, or cosmetically or pharmaceutically acceptable salt or other suitable form thereof, is injected into the sub-mental (under chin) region of a patient. For example, the treatment is repeated 4 times at 4 week intervals until the desired cosmetic effect is achieved. In another treatment embodiment, up to twenty 0.2 mL injections spaced 1.0 cm apart of 1.0% weight/weight of polidocanol, a polidocanol like compound, or cosmetically or pharmaceutically acceptable salt or other suitable form thereof, is injected into the sub-mental (under chin) region of a patient. The treatment is repeated up to 4 times at 4-week intervals until the desired cosmetic effect is achieved. The treatment is repeated up to 4 times at 4-week intervals until the desired cosmetic effect is achieved. In another still another treatment embodiment, up to twenty 0.2 mL injections spaced 1.0 cm apart of 0.5% weight/weight of polidocanol, a polidocanol like compound, or cosmetically or pharmaceutically acceptable salt or other suitable form thereof, is injected into the sub-mental (under chin) region of a patient. The treatment is repeated 4 times at 4 week intervals until the desired cosmetic effect is achieved.

For example, reductions in inguinal fat pad mass of 15% were found at day 28 after a single treatment of polidocanol at doses of 0.5% and 1.25%. Higher concentrations (i.e., 2.0%) were shown to yield slightly less (i.e., 10%) fat pad mass reductions. Thus, in embodiments, the polidocanol is administered approximately once per month and/or at concentrations less than about 2.0%. The treatment may employ about 0.1 to 0.3 mL (e.g. 0.2 mL) of a polidocanol at a concentration provided herein. In embodiments, the polidocanol is a concentration of from about 0.5% and 1.25%. The administration may be via injection. In embodiments, the injection is at a spacing of about 0.2 to 0.8 cm apart across the inguinal fat pad. Thus, in embodiments, the polidocanol is administered approximately once per month and/or at concentrations less than 2.0%. The treatment may employ 0.1 to 0.3 mL (e.g., 0.2 mL) of a polidocanol at a concentration provided herein. In embodiments, the polidocanol is a concentration of from 0.5% and 1.25%. The administration may be via injection. In embodiments, the injection is at a spacing of 0.2 to 0.8 cm apart across the inguinal fat pad. In embodiments, the injection is at a spacing of about 0.5 cm apart across the inguinal fat pad. In addition, externally visible reduction in inguinal fat pad fullness was observed resulting in enhancement of the inguinal crease consistent with a benefit of visible changes in body contour.

In an aspect, provided herein is a method of treating regional adipose tissue, regional adiposity, sub-mental adiposity, or regional fat accumulation in an individual, said method including administering to the individual an effective amount of a formulation including: at least one of a compound of Formula I or Formula II such as polidocanol or a pharmaceutically acceptable or cosmetically acceptable salt or other suitable form thereof; and a liquid carrier; wherein the compound of Formula I or Formula II or a pharmaceutically acceptable or cosmetically acceptable salt or other suitable form thereof and the liquid carrier are formulated for injection into a layer of subcutaneous fat in the individual in need thereof.

In some embodiments, in addition to treating a subject with any of the compositions described herein, a physician or other authorized medical caregiver prescribes a liposuction procedure to a subject, or performs a liposuction procedure on the subject to further reduce regional fat deposits. In some embodiments, a liposuction procedure is performed on a subject to whom has been administered a composition including a therapeutically effective amount of a compound of Formula I or Formula II such as polidocanol or a pharmaceutically acceptable or cosmetically acceptable salt or other suitable form thereof. Without wishing to be bound by theory, administering any of the pharmaceutical and cosmetic compositions described herein to a subject prior to liposuction is likely to increase the efficacy of the liposuction procedure. In embodiments, the method does not include administration of a lipase. In embodiments, the method does not include administration of a colipase. In embodiments, the method includes a pharmaceutical formulation as described herein (including in an aspect, embodiment, claim, example, table, or figure). In embodiments, the method includes an injectable formulation as described herein (including in an aspect, embodiment, claim, example, table, or figure). In embodiments, the method includes a formulation as described herein (including in an aspect, embodiment, claim, example, table, or figure). In embodiments, the method includes a composition (e.g., a pharmaceutical composition, injectable composition) as described herein.

### Compositions

In an aspect is provided a pharmaceutical formulation including polidocanol in an amount from about 0.5% w/w to about 10.0% w/w. In an aspect is provided a pharmaceutical formulation including polidocanol in an amount from about 0.1% w/w to about 10% w/w/In embodiments, the polidocanol is present in an amount of about 0.1%-10% w/w or about 0.1%-5% w/w. In some embodiments, the polidocanol is present in an amount of about 0.1% w/w to about 10% w/w. In some embodiments, the polidocanol is present in an amount of about 0.1% w/w to about 0.2% w/w, about 0.1% w/w to about 0.5% w/w, about 0.1% w/w to about 1% w/w, about 0.1% w/w to about 2% w/w, about 0.1% w/w to about 3% w/w, about 0.1% w/w to about 4% w/w, about 0.1% w/w to about 4.5% w/w, about 0.1% w/w to about 5% w/w, about 0.1% w/w to about 6% w/w, about 0.1% w/w to about 8% w/w, about 0.1% w/w to about 10% w/w, about 0.2% w/w to about 0.5% w/w, about 0.2% w/w to about 1% w/w, about 0.2% w/w to about 2% w/w, about 0.2% w/w to about 3% w/w, about 0.2% w/w to about 4% w/w, about 0.2% w/w to about 4.5% w/w, about 0.2% w/w to about 5% w/w, about 0.2% w/w to about 6% w/w, about 0.2% w/w to about 8% w/w, about 0.2% w/w to about 10% w/w, about 0.5% w/w to about 1% w/w, about 0.5% w/w to about 2% w/w, about 0.5% w/w to about 3% w/w, about 0.5% w/w to about 4% w/w, about 0.5% w/w to about 4.5% w/w, about 0.5% w/w to about 5% w/w, about 0.5% w/w to about 6% w/w, about 0.5% w/w to about 8% w/w, about 0.5% w/w to about 10% w/w, about 1% w/w to about 2% w/w, about 1% w/w to about 3% w/w, about 1% w/w to about 4% w/w, about 1% w/w to about 4.5% w/w, about 1% w/w to about 5% w/w, about 1% w/w to about 6% w/w, about 1% w/w to about 8% w/w, about 1% w/w to about 10% w/w, about 2% w/w to about 3% w/w, about 2% w/w to about 4% w/w, about 2% w/w to about 4.5% w/w, about 2% w/w to about 5% w/w, about 2% w/w to about 6% w/w, about 2% w/w to about 8% w/w, about 2% w/w to about 10% w/w, about 3% w/w to about 4% w/w, about 3% w/w to about 4.5% w/w, about 3% w/w to about 5% w/w, about 3% w/w to about 6% w/w, about 3% w/w to about 8% w/w, about 3% w/w to about 10% w/w, about 4% w/w to about 4.5% w/w, about 4% w/w to about 5% w/w, about 4% w/w to about 6% w/w, about 4% w/w to about 8% w/w, about 4% w/w to about 10% w/w, about 4.5% w/w to about 5% w/w, about 4.5% w/w to about 6% w/w, about 4.5% w/w to about 8% w/w, about 4.5% w/w to about 10% w/w, about 5% w/w to about 6% w/w, about 5% w/w to about 8% w/w, about 5% w/w to about 10% w/w, about 6% w/w to about 8% w/w, about 6% w/w to about 10% w/w, or about 8% w/w to about 10% w/w. In some embodiments, the polidocanol is present in an amount of about 0.1% w/w, about 0.2% w/w, about 0.5% w/w, about 1% w/w, about 2% w/w, about 3% w/w, about 4% w/w, about 4.5% w/w, about 5% w/w, about 6% w/w, about 8% w/w, or about 10% w/w. In some embodiments, the polidocanol is present in an amount of at least about 0.1% w/w, about 0.2% w/w, about 0.5% w/w, about 1% w/w, about 2% w/w, about 3% w/w, about 4% w/w, about 4.5% w/w, about 5% w/w, about 6% w/w, or about 8% w/w. In some embodiments, the polidocanol is present in an amount of at most about 0.2% w/w, about 0.5% w/w, about 1% w/w, about 2% w/w, about 3% w/w, about 4% w/w, about 4.5% w/w, about 5% w/w, about 6% w/w, about 8% w/w, or about 10% w/w. In some embodiments, the pharmaceutical formulation comprises at least 3.0% w/w of polidocanol. In some embodiments, the pharmaceutical formulation comprises up to 4.5% w/w of polidocanol.

In some embodiments, the pharmaceutical formulation consists of about 2.0% w/w of polidocanol. In some embodiments, the pharmaceutical formulation consists of about 3.0% w/w of polidocanol. In some embodiments, the pharmaceutical formulation consists of about 4.5% w/w of polidocanol.

In embodiments, the pharmaceutical formulation consists essentially of polidocanol in an amount from about 0.5% w/w to about 10.0% w/w; and a C₃-C₆ alcohol in an amount from about 0.5% w/w to about 10% w/w. The term “consisting essentially of” in this context refers to a pharmaceutical formulation comprising polidocanol, a C₃-C₆ alcohol and no other component that is independently capable of reducing fat according to the methods described herein.

In embodiments, the pharmaceutical formulations described herein do not include a beta-2-adrenergic receptor agonist (e.g. bambuterol, bitolterol, broxaterol, carbuterol, carmoterol, clenbuterol, ibuterol, sulfonterol, isoproterenol, trimetoquinol, formoterol, desformoterol, hexoprenaline, ibuterol, indacaterol, isoetharine, isoprenaline, isoproterenol, levalbuterol, metaproterenol, picumeterol, pirbuterol, procaterol, reproterol, rimiterol, salbutamol, salmeterol; sulfonterol, terbutaline, trimetoquinol, tulobuterol, TA-2005 (8-hydroxy-5-((1R)-1-hydroxy-2-(N-((1R)-2-(4-methoxyphenyl)-1-methylethyl-) amino)ethyl)-carbostyril hydrochloride), QAB-149 (Novartis), TA-2005, GSK-159797, or GSK-642444, or a salt, optical isomer, racemate, solvate, or polymorph thereof) or a pharmaceutically acceptable or cosmetically acceptable salt thereof.

In embodiments, the pharmaceutical formulations described herein do not include a ketotifen or analog of ketotifen or a pharmaceutically acceptable or cosmetically acceptable salt thereof. In embodiments, the pharmaceutical formulations described herein do not include a thyroid hormone (e.g. thyroxine (T4) or triiodothyronine (T3)) a pharmaceutically acceptable or cosmetically acceptable salt thereof. In embodiments, the pharmaceutical formulations described herein do not include a natriuretic peptide (e.g. atrial natriuretic peptide (ANP), brain natriuretic peptide (BNP), C-type natriuretic peptide, and dendroaspis natriuretic peptide, natriuretic peptide receptor A (NPrA), natriuretic peptide receptor (NPrB)) a pharmaceutically acceptable or cosmetically acceptable salt thereof. In embodiments, the pharmaceutical formulations described herein do not include 1, 25-dihydroxy Vitamin D3 or its analogues a pharmaceutically acceptable or cosmetically acceptable salt thereof.

In embodiments, the pharmaceutical formulations described herein do not include glycerol. In embodiments, the pharmaceutical formulations described herein do not include polyethylene glycol. In embodiments, the pharmaceutical formulations described are not topical compositions (such as a topical gel composition). In embodiments, the pharmaceutical formulations described herein do not include hydroxypropyl cellulose. In embodiments, the pharmaceutical formulations described herein do not include isopropyl myristate.

In embodiments, the pharmaceutical formulations described herein are not oral compositions. In embodiments, the pharmaceutical formulations described herein do not include starch. In embodiments, the pharmaceutical formulations described herein do not include gelatin. In embodiments, the pharmaceutical formulations described herein do not include hydroxypropylmethylcellulose.

In embodiments, the pharmaceutical formulations described herein are not an emulsion. In embodiments, the pharmaceutical formulations described herein are not a micro-emulsion. In embodiments, the pharmaceutical formulations described herein do not include a lipophilic substance (e.g. natural oils, esters of middle-chain alkyl acids with glycols, octyl-dodecanol, silicon oils, paraffins and vitamins). In embodiments, the pharmaceutical formulations described herein do not include a compound having an HLB of 6 or less. In embodiments, the pharmaceutical formulations described herein do not include a compound having an HLB of 5 or less. In embodiments, the pharmaceutical formulations described herein do not include a compound having an HLB of 4 or less. In embodiments, the pharmaceutical formulations described herein do not include a compound having an HLB of 3 or less. In embodiments, the pharmaceutical formulations described herein do not include a compound having an HLB of 7 or less. In embodiments, the pharmaceutical formulations described herein do not include a compound having an HLB of 8 or less.

In embodiments, the C₃-C₆ alcohol is propylene glycol. In embodiments, the C₃-C₆ alcohol (e.g., propylene glycol) is present in an amount from about 2.0% w/w to about 5.0% w/w. In embodiments, the C₃-C₆ alcohol (e.g., propylene glycol) is present in an amount from about 2.2% w/w to about 5.0% w/w. In embodiments, the C₃-C₆ alcohol (e.g., propylene glycol) is present in an amount from about 2.4% w/w to about 5.0% w/w. In embodiments, the C₃-C₆ alcohol (e.g., propylene glycol) is present in an amount from about 2.6% w/w to about 5.0% w/w. In embodiments, the C₃-C₆ alcohol (e.g., propylene glycol) is present in an amount from about 2.8% w/w to about 5.0% w/w. In embodiments, the C₃-C₆ alcohol (e.g., propylene glycol) is present in an amount from about 3.0% w/w to about 5.0% w/w. In embodiments, the C₃-C₆ alcohol (e.g., propylene glycol) is present in an amount from about 3.2% w/w to about 5.0% w/w. In embodiments, the C₃-C₆ alcohol (e.g., propylene glycol) is present in an amount from about 3.4% w/w to about 5.0% w/w. In embodiments, the C₃-C₆ alcohol (e.g., propylene glycol) is present in an amount from about 3.6% w/w to about 5.0% w/w. In embodiments, the C₃-C₆ alcohol (e.g., propylene glycol) is present in an amount from about 3.8% w/w to about 5.0% w/w. In embodiments, the C₃-C₆ alcohol (e.g., propylene glycol) is present in an amount from about 4.0% w/w to about 5.0% w/w. In embodiments, the C₃-C₆ alcohol (e.g., propylene glycol) is present in an amount from about 4.2% w/w to about 5.0% w/w. In embodiments, the C₃-C₆ alcohol (e.g., propylene glycol) is present in an amount from about 4.4% w/w to about 5.0% w/w.

In embodiments, the pharmaceutical formulation is a subcutaneous injection formulation. In embodiments, the pharmaceutical formulation consists of a subcutaneous injection formulation. In embodiments, the pharmaceutical formulation consists essentially of a subcutaneous injection formulation. In embodiments, the pharmaceutical formulation includes a subcutaneous injection formulation. In embodiments, the pharmaceutical formulation is an aqueous formulation. In embodiments, the pharmaceutical formulation is not a gel (e.g., at room temperature, at physiological temperatures, at normal human temperature, at about 37 degrees Celsius (“deg C.”), at 37 deg C., at about 35 deg. C. to about 41.5 deg. C., at 35 deg C. to 41.5 deg C., at about 36.5 deg. C. to 37.5 deg C., at 36.5 deg. C. to 37.5 deg. C.).

In embodiments, the pharmaceutical formulation has an osmolality of less than about 400 milliosmoles/kg. In embodiments, the pharmaceutical formulation has an osmolality of less than about 380 milliosmoles/kg. In embodiments, the pharmaceutical formulation has an osmolality of less than about 360 milliosmoles/kg. In embodiments, the pharmaceutical formulation has an osmolality of less than about 340 milliosmoles/kg. In embodiments, the pharmaceutical formulation has an osmolality of less than about 320 milliosmoles/kg. In embodiments, the pharmaceutical formulation has an osmolality of less than about 300 milliosmoles/kg. In embodiments, the pharmaceutical formulation has an osmolality of less than about 280 milliosmoles/kg. In embodiments, the pharmaceutical formulation has an osmolality of less than about 260 milliosmoles/kg. In embodiments, the pharmaceutical formulation has an osmolality of less than about 240 milliosmoles/kg.

In embodiments, the pharmaceutical formulation has an osmolality of less than 400 milliosmoles/kg. In embodiments, the pharmaceutical formulation has an osmolality of less than 380 milliosmoles/kg. In embodiments, the pharmaceutical formulation has an osmolality of less than 360 milliosmoles/kg. In embodiments, the pharmaceutical formulation has an osmolality of less than 340 milliosmoles/kg. In embodiments, the pharmaceutical formulation has an osmolality of less than 320 milliosmoles/kg. In embodiments, the pharmaceutical formulation has an osmolality of less than 300 milliosmoles/kg. In embodiments, the pharmaceutical formulation has an osmolality of less than 280 milliosmoles/kg. In embodiments, the pharmaceutical formulation has an osmolality of less than 260 milliosmoles/kg. In embodiments, the pharmaceutical formulation has an osmolality of less than 240 milliosmoles/kg.

In embodiments, the pharmaceutical formulation has a pH of about 7 to about 8. In embodiments, the pharmaceutical formulation has a pH of about 7.1 to about 8. In embodiments, the pharmaceutical formulation has a pH of about 7.2 to about 8. In embodiments, the pharmaceutical formulation has a pH of about 7.3 to about 8. In embodiments, the pharmaceutical formulation has a pH of about 7.4 to about 8. In embodiments, the pharmaceutical formulation has a pH of about 7.5 to about 8. In embodiments, the pharmaceutical formulation has a pH of about 7.6 to about 8. In embodiments, the pharmaceutical formulation has a pH of about 7.7 to about 8. In embodiments, the pharmaceutical formulation has a pH of about 7.8 to about 8. In embodiments, the pharmaceutical formulation has a pH of about 7.9 to about 8. In embodiments, the pharmaceutical formulation has a pH of about 7 to about 7.9. In embodiments, the pharmaceutical formulation has a pH of about 7 to about 7.8. In embodiments, the pharmaceutical formulation has a pH of about 7 to about 7.7. In embodiments, the pharmaceutical formulation has a pH of about 7 to about 7.6. In embodiments, the pharmaceutical formulation has a pH of about 7 to about 7.5. In embodiments, the pharmaceutical formulation has a pH of about 7 to about 7.4. In embodiments, the pharmaceutical formulation has a pH of about 7 to about 7.3. In embodiments, the pharmaceutical formulation has a pH of about 7 to about 7.2. In embodiments, the pharmaceutical formulation has a pH of about 7 to about 7.1. In embodiments, the pharmaceutical formulation has a pH of about 7.1 to about 7.9. In embodiments, the pharmaceutical formulation has a pH of about 7.2 to about 7.8. In embodiments, the pharmaceutical formulation has a pH of about 7.3 to about 7.7. In embodiments, the pharmaceutical formulation has a pH of about 7.4 to about 7.6.

In embodiments, the pharmaceutical formulation has a pH of 7 to 8. In embodiments, the pharmaceutical formulation has a pH of 7.1 to 8. In embodiments, the pharmaceutical formulation has a pH of 7.2 to 8. In embodiments, the pharmaceutical formulation has a pH of 7.3 to 8. In embodiments, the pharmaceutical formulation has a pH of 7.4 to 8. In embodiments, the pharmaceutical formulation has a pH of 7.5 to 8. In embodiments, the pharmaceutical formulation has a pH of 7.6 to 8. In embodiments, the pharmaceutical formulation has a pH of 7.7 to 8. In embodiments, the pharmaceutical formulation has a pH of 7.8 to 8. In embodiments, the pharmaceutical formulation has a pH of 7.9 to 8. In embodiments, the pharmaceutical formulation has a pH of 7 to 7.9. In embodiments, the pharmaceutical formulation has a pH of 7 to 7.8. In embodiments, the pharmaceutical formulation has a pH of 7 to 7.7. In embodiments, the pharmaceutical formulation has a pH of 7 to 7.6. In embodiments, the pharmaceutical formulation has a pH of 7 to 7.5. In embodiments, the pharmaceutical formulation has a pH of 7 to 7.4. In embodiments, the pharmaceutical formulation has a pH of 7 to 7.3. In embodiments, the pharmaceutical formulation has a pH of 7 to 7.2. In embodiments, the pharmaceutical formulation has a pH of 7 to 7.1. In embodiments, the pharmaceutical formulation has a pH of 7.1 to 7.9. In embodiments, the pharmaceutical formulation has a pH of 7.2 to 7.8. In embodiments, the pharmaceutical formulation has a pH of 7.3 to 7.7. In embodiments, the pharmaceutical formulation has a pH of 7.4 to 7.6.

In embodiments, the pharmaceutical formulation has a pH of about 7.0. In embodiments, the pharmaceutical formulation has a pH of about 7.1. In embodiments, the pharmaceutical formulation has a pH of about 7.2. In embodiments, the pharmaceutical formulation has a pH of about 7.3. In embodiments, the pharmaceutical formulation has a pH of about 7.4. In embodiments, the pharmaceutical formulation has a pH of about 7.5. In embodiments, the pharmaceutical formulation has a pH of about 7.6. In embodiments, the pharmaceutical formulation has a pH of about 7.7. In embodiments, the pharmaceutical formulation has a pH of about 7.8. In embodiments, the pharmaceutical formulation has a pH of about 7.9. In embodiments, the pharmaceutical formulation has a pH of about 8.0.

In embodiments, the pharmaceutical formulation has a pH of 7.0. In embodiments, the pharmaceutical formulation has a pH of 7.1. In embodiments, the pharmaceutical formulation has a pH of 7.2. In embodiments, the pharmaceutical formulation has a pH of 7.3. In embodiments, the pharmaceutical formulation has a pH of 7.4. In embodiments, the pharmaceutical formulation has a pH of 7.5. In embodiments, the pharmaceutical formulation has a pH of 7.6. In embodiments, the pharmaceutical formulation has a pH of 7.7. In embodiments, the pharmaceutical formulation has a pH of 7.8. In embodiments, the pharmaceutical formulation has a pH of 7.9. In embodiments, the pharmaceutical formulation has a pH of 8.0.

In embodiments, the pharmaceutical formulation has a volume from about 0.2 cc to about 0.5 cc. In embodiments, the pharmaceutical formulation has a volume from about 0.25 cc to about 0.5 cc. In embodiments, the pharmaceutical formulation has a volume from about 0.3 cc to about 0.5 cc. In embodiments, the pharmaceutical formulation has a volume from about 0.35 cc to about 0.5 cc. In embodiments, the pharmaceutical formulation has a volume from about 0.4 cc to about 0.5 cc. In embodiments, the pharmaceutical formulation has a volume from about 0.45 cc to about 0.5 cc. In embodiments, the pharmaceutical formulation has a volume from about 0.2 cc to about 0.45 cc. In embodiments, the pharmaceutical formulation has a volume from about 0.2 cc to about 0.4 cc. In embodiments, the pharmaceutical formulation has a volume from about 0.2 cc to about 0.35 cc. In embodiments, the pharmaceutical formulation has a volume from about 0.2 cc to about 0.3 cc. In embodiments, the pharmaceutical formulation has a volume from about 0.2 cc to about 0.25 cc.

In embodiments, the pharmaceutical formulation has a volume from 0.2 cc to 0.5 cc. In embodiments, the pharmaceutical formulation has a volume from 0.25 cc to 0.5 cc. In embodiments, the pharmaceutical formulation has a volume from 0.3 cc to 0.5 cc. In embodiments, the pharmaceutical formulation has a volume from 0.35 cc to 0.5 cc. In embodiments, the pharmaceutical formulation has a volume from 0.4 cc to 0.5 cc. In embodiments, the pharmaceutical formulation has a volume from 0.45 cc to 0.5 cc. In embodiments, the pharmaceutical formulation has a volume from 0.2 cc to 0.45 cc. In embodiments, the pharmaceutical formulation has a volume from 0.2 cc to 0.4 cc. In embodiments, the pharmaceutical formulation has a volume from 0.2 cc to 0.35 cc. In embodiments, the pharmaceutical formulation has a volume from 0.2 cc to 0.3 cc. In embodiments, the pharmaceutical formulation has a volume from 0.2 cc to 0.25 cc.

In embodiments, the pharmaceutical formulation has a volume of about 0.20 cc. In embodiments, the pharmaceutical formulation has a volume of about 0.25 cc. In embodiments, the pharmaceutical formulation has a volume of about 0.30 cc. In embodiments, the pharmaceutical formulation has a volume of about 0.35 cc. In embodiments, the pharmaceutical formulation has a volume of about 0.40 cc. In embodiments, the pharmaceutical formulation has a volume of about 0.45 cc. In embodiments, the pharmaceutical formulation has a volume of about 0.50 cc.

In embodiments, the pharmaceutical formulation has a volume of 0.20 cc. In embodiments, the pharmaceutical formulation has a volume of 0.25 cc. In embodiments, the pharmaceutical formulation has a volume of 0.30 cc. In embodiments, the pharmaceutical formulation has a volume of 0.35 cc. In embodiments, the pharmaceutical formulation has a volume of 0.40 cc. In embodiments, the pharmaceutical formulation has a volume of 0.45 cc. In embodiments, the pharmaceutical formulation has a volume of 0.50 cc.

In embodiments, the pharmaceutical formulation further includes a buffer. In embodiments, the buffer is phosphate buffer (e.g., with Na⁺, K⁺, or both cations).

In embodiments, the pharmaceutical formulation includes water, polidocanol, buffer, propylene glycol, chloride and a monovalent metal ion selected from sodium, potassium or a mixture of sodium and potassium. In embodiments, the pharmaceutical formulation consists essentially of water, polidocanol, buffer, propylene glycol, chloride and a monovalent metal ion selected from sodium, potassium or a mixture of sodium and potassium. In embodiments, the pharmaceutical formulation consists of water, polidocanol, buffer, propylene glycol, chloride and a monovalent metal ion selected from sodium, potassium or a mixture of sodium and potassium.

In embodiments, the pharmaceutical formulation includes one or more of: buffers, diluents, lubricating agents, solubilizers, solvents; surfactants, penetration enhancers, polymers, dispersion agents, wetting agents, emulsifying and suspending agents, or preserving agents. Examples of dispersion agents include, but are not limited to, hyaluronidase and collagenase. In some embodiments, the dispersion agents, such as collagenase, are administered prior to the administration of the pharmaceutical formulation described herein.

In embodiments, the pharmaceutical formulation includes one or more of: acetylene glycol, ethylene glycol, diethylene glycol, triethylene glycol, propylene glycol, tetraethylene glycol, polyethylene glycol, dipropylene glycol, polypropylene glycol, hexylene glycol, thiodiglycol, glycerin, or 1,2,6-hexanetriol.

In embodiments, the pharmaceutical formulation does not include a lipase or colipase.

In embodiments, the pharmaceutical formulation includes one or more of: anti-microbial agents, vasoconstrictors, anti-thrombotic agents, anti-coagulation agents, suds-depressants, anti-inflammatory agents, analgesics, dispersion agents, anti-dispersion agents, penetration enhancers, steroids, tranquilizers, muscle relaxants, or anti-diarrhea agents.

In embodiments, the pH of the pharmaceutical formulation may be maintained with the use of a buffer. Various buffers are known in the art and it is contemplated that any buffer having buffering capacity at the desired pH can be used in the formulations disclosed herein. In embodiments, the buffer is a phosphate buffer.

In embodiments, the water employed in the pharmaceutical formulation is sterile water. In still a further embodiment, a pharmaceutical formulation may include a preserving agent. In embodiments, the preserving agent in a pharmaceutical formulation described herein is benzyl alcohol. In embodiments, an effective amount of benzyl alcohol is about 0.9% w/w benzyl alcohol. In embodiments, an effective amount of benzyl alcohol is 0.9% w/w benzyl alcohol.

In an embodiment, the pharmaceutical formulation is split into a plurality of individual smaller pharmaceutical formulations which are separately administered to the fat cells. For example, the pharmaceutical formulation may be split into 5, 10, 15, 20, 25 or 30 separate pharmaceutical formulations and, in some cases, up to 50 separate pharmaceutical formulations. In embodiments, each such smaller pharmaceutical formulation is itself a pharmaceutical formulation and may have a volume described herein.

Provided herein is a solution to an unmet and long-felt need in the cosmetic and aesthetic industry for an emulsifying detergent formulation for reducing fat deposits that overcomes the shortcomings and failures of previous attempts in the field. In particular, provided herein are injectable formulations for treating or contacting fat cells, regional adipose tissue, regional adiposity, or regional fat accumulation. In embodiments, the formulations described herein include an effective amount, including for example a therapeutically or cosmetically effective amount, of polidocanol, polidocanol-like compound, or a pharmaceutically acceptable or cosmetically acceptable salt or other suitable form thereof; and a liquid carrier including aqueous carriers; wherein the polidocanol, polidocanol-like compound and the liquid carrier are formulated for injection into a layer of fat, including subcutaneous fat, in the individual in need thereof. In embodiment, the formulations do not include a lipase. In embodiment, the formulations do not include a colipase.

In embodiments, the polidocanol produced herein results in a mixture of unbranched, saturated C12 primary alcohol ethoxylate homologues having a range of ethoxide units. For example, HPLC characterization of embodiments of product polidocanol synthesized herein results in approximately 15 peaks associated with varying numbers of ethoxide units. In the polidocanol product, for example, no single peak makes up more than 10% of the total mixture. The average ethoxylation number of the polidocanol characterized is 6-7 and the average molecular weight is approximately 493 g/mol.

In embodiments, potassium hydroxide is used as the catalyst to produce polidocanol from ethylene oxide (e.g. 9 moles) and dodecanol (e.g. 1 mole).

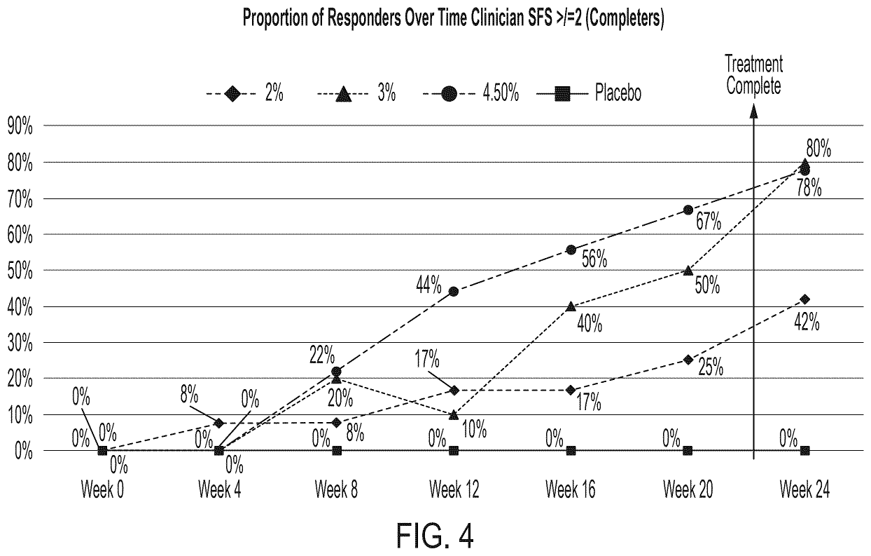

Polidocanol products with different ranges or percentages of ethoxylate homologues may be produced using different catalysts (e.g. different metal oxide catalysts). See e.g. Van OS, N. M., Nonionic Surfactants—Organic Chemistry, Marcel Dekker Inc., New York, 1998, p. 101 at FIGS. 4A-4C. In embodiments, the polidocanol includes fewer than 15 major peaks (e.g. 10 or less) as shown by HPLC characterization. The polidocanol may include about 7 to about 11 ethoxylate units.

In embodiments, the product polidocanol includes alkyl ethoxylate homologue mixtures containing a high proportion of detergent/surfactant homologues per gram of mixture produced (e.g., by the methods provided herein).

In embodiments, polidocanol is formulated with a co-solvent. The co-solvent may be propylene glycol.

In embodiments, the intravascular formulation of polidocanol contains ethanol at approximately 5% to improve formulation stability (particularly at lower temperatures) and to reduce foaming. Ethanol is suitable for intravascular administration due to its rapid dilution by the blood. However, for subcutaneous administration, where the injected formulation resides longer at the administration site, ethanol is not ideal. Ethanol is irritating when administered subcutaneously and can cause burning, swelling, redness, and skin discoloration secondary to inflammation. In addition, ethanol is generally toxic to a variety of tissues and may limit the selective effect of polidocanol on subcutaneous fat tissue, resulting in more complications.

### Formulations Including Compounds of Formula I or Formula II

In an aspect is provided an injectable formulation for treating fat, regional fat, adipose tissue, regional adiposity, or regional fat accumulation including: an effective amount, including for example a therapeutically or cosmetically effective amount, of the compound of Formula I:

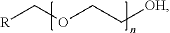

or a pharmaceutically acceptable or cosmetically acceptable salt or other suitable form thereof, and wherein n=2 to 55; R is a saturated, linear C₇-C₂₄ hydrocarbon (alkyl), or an unsaturated, linear C₇-C₂₄ hydrocarbon; and a liquid carrier. In some embodiments, the Formula I comprises a structure of:

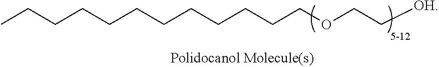

In embodiments, the compound of Formula I, or a pharmaceutically acceptable or cosmetically acceptable salt or other suitable form thereof and the liquid carrier are formulated for injection into a layer of subcutaneous fat in a human in need. In embodiments, the injectable formulation is formulated for subcutaneous injection.

In embodiments, R is a saturated, linear C₇-C₂₄ hydrocarbon. In some embodiments, R is a saturated, linear C₇-C₁₆ hydrocarbon, and in some embodiments R is a saturated, linear C₇-C₁₂ hydrocarbon. In some embodiments, R is a saturated, linear C₁₂-C₁₈ hydrocarbon. In an embodiment, R is a saturated, linear C₁₈-C₂₄ hydrocarbon. In certain embodiments, R is a saturated, linear C₇-C₂₄ hydrocarbon. In some embodiments, R is a saturated, linear C₇-C₁₂ hydrocarbon. In some embodiments, R is a saturated, linear C₁₂-C₁₈ hydrocarbon. In an embodiment, R is a saturated, linear C₁₈-C₂₄ hydrocarbon.

In embodiments, R is an unsaturated, linear C₇-C₂₄ hydrocarbon. In some embodiments, R is an unsaturated, linear C₇-C₁₂ hydrocarbon. In an embodiment, R is an unsaturated, linear C₁₂-C₁₈ hydrocarbon. In some embodiments, R is an unsaturated, linear C₁₈-C₂₄ hydrocarbon. In certain embodiments, the unsaturated, linear hydrocarbon includes at least one alkene moiety. In some embodiments, the unsaturated, linear hydrocarbon includes at least one alkene moiety of cis configuration. In an embodiment, R is an unsaturated, linear C₇-C₂₄ hydrocarbon. In some embodiments, R is an unsaturated, linear C₇-C₁₂ hydrocarbon. In an embodiment, R is an unsaturated, linear C₁₂-C₁₈ hydrocarbon. In some embodiments, R is an unsaturated, linear C₁₈-C₂₄ hydrocarbon. In certain embodiments, the unsaturated, linear hydrocarbon includes at least one alkene moiety. In certain embodiments, the unsaturated, linear hydrocarbon includes at least one alkene moiety of trans configuration. In some embodiments, the unsaturated, linear hydrocarbon includes at least one alkyne moiety.

In embodiments, the injectable formulation is for treating fat. In embodiments, the injectable formulation is for treating regional fat. In embodiments, the injectable formulation is for treating adipose tissue. In embodiments, the injectable formulation is for treating regional adiposity. In embodiments, the injectable formulation is for treating regional fat accumulation.

In embodiments, the injectable formulation includes an effective amount, including for example a therapeutically or cosmetically effective amount, of the compound of Formula I

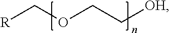

wherein R is a saturated or unsaturated, linear C₁₁ or C₁₂ hydrocarbon. In certain embodiments, the unsaturated, linear hydrocarbon includes at least one alkene moiety. In some embodiments, the unsaturated, linear hydrocarbon includes at least one alkene moiety of cis configuration. In some embodiments, the unsaturated, linear hydrocarbon includes at least one alkene moiety of trans configuration. In certain embodiments, the unsaturated, linear hydrocarbon includes at least one alkyne moiety.

In embodiments, the injectable formulation includes an effective amount, including for example a therapeutically or cosmetically effective amount, of the compound of Formula I, wherein n is 5 to 32. In some embodiments, n is 7 to 21. In certain embodiments, n is 7. In some embodiments, n is 9. In an embodiment, n is 11. In embodiments, n is 5 to 32. In certain embodiments, n is 7 to 21. In a specific embodiment, R is a saturated linear C₁₂ hydrocarbon and n is 9.

Provided below is the chemical structure of Formula II, wherein n is 2 to 55 (e.g. approximately 9).

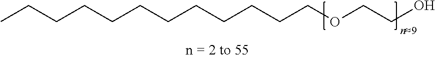

In some embodiments, the Formula II comprises a structure of:

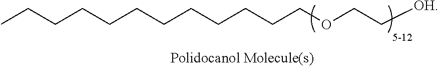

In certain embodiments of the formulations described herein, provided is a compound of Formula II wherein n is from 2 to 55. Where n is approximately 9, the compound of Formula II may be referred to herein as a polidocanol. In some embodiments, n is selected from the range of 5 to 32 (e.g. 9). In certain embodiments, n is selected from the range of 7 to 21. In embodiments, n is 7 to 15. In embodiments, n is 7 to 14. In certain embodiments, n=7. In certain embodiments, n=8. In certain embodiments, n=9. In certain embodiments, n=10. In certain embodiments, n=11. In certain embodiments, n=12.

In an aspect, provided herein are injectable formulations for treating or contacting fat tissue, regional adipose tissue, regional adiposity, or regional fat accumulation, the formulations provide an effective amount, including for example a therapeutically or cosmetically effective amount, of a compound of Formula II or a pharmaceutically acceptable or cosmetically acceptable salt or other suitable form thereof, and a liquid carrier.

In embodiments, the compound or a pharmaceutically acceptable or cosmetically acceptable salt or other suitable form thereof and the liquid carrier are formulated for injection into a layer of subcutaneous fat in a human in need. In embodiments, the injectable formulation is formulated for subcutaneous injection.

In embodiments, the polidocanol is present in an amount of about 0.1%-10% w/w or about 0.1%-5% w/w. In embodiments, the polidocanol is present in an amount of about 0.1%-10% w/w or about 0.1%-5% w/w. In some embodiments, the polidocanol is present in an amount of about 0.1% w/w to about 10% w/w. In some embodiments, the polidocanol is present in an amount of about 0.1% w/w to about 0.2% w/w, about 0.1% w/w to about 0.5% w/w, about 0.1% w/w to about 1% w/w, about 0.1% w/w to about 2% w/w, about 0.1% w/w to about 3% w/w, about 0.1% w/w to about 4% w/w, about 0.1% w/w to about 4.5% w/w, about 0.1% w/w to about 5% w/w, about 0.1% w/w to about 6% w/w, about 0.1% w/w to about 8% w/w, about 0.1% w/w to about 10% w/w, about 0.2% w/w to about 0.5% w/w, about 0.2% w/w to about 1% w/w, about 0.2% w/w to about 2% w/w, about 0.2% w/w to about 3% w/w, about 0.2% w/w to about 4% w/w, about 0.2% w/w to about 4.5% w/w, about 0.2% w/w to about 5% w/w, about 0.2% w/w to about 6% w/w, about 0.2% w/w to about 8% w/w, about 0.2% w/w to about 10% w/w, about 0.5% w/w to about 1% w/w, about 0.5% w/w to about 2% w/w, about 0.5% w/w to about 3% w/w, about 0.5% w/w to about 4% w/w, about 0.5% w/w to about 4.5% w/w, about 0.5% w/w to about 5% w/w, about 0.5% w/w to about 6% w/w, about 0.5% w/w to about 8% w/w, about 0.5% w/w to about 10% w/w, about 1% w/w to about 2% w/w, about 1% w/w to about 3% w/w, about 1% w/w to about 4% w/w, about 1% w/w to about 4.5% w/w, about 1% w/w to about 5% w/w, about 1% w/w to about 6% w/w, about 1% w/w to about 8% w/w, about 1% w/w to about 10% w/w, about 2% w/w to about 3% w/w, about 2% w/w to about 4% w/w, about 2% w/w to about 4.5% w/w, about 2% w/w to about 5% w/w, about 2% w/w to about 6% w/w, about 2% w/w to about 8% w/w, about 2% w/w to about 10% w/w, about 3% w/w to about 4% w/w, about 3% w/w to about 4.5% w/w, about 3% w/w to about 5% w/w, about 3% w/w to about 6% w/w, about 3% w/w to about 8% w/w, about 3% w/w to about 10% w/w, about 4% w/w to about 4.5% w/w, about 4% w/w to about 5% w/w, about 4% w/w to about 6% w/w, about 4% w/w to about 8% w/w, about 4% w/w to about 10% w/w, about 4.5% w/w to about 5% w/w, about 4.5% w/w to about 6% w/w, about 4.5% w/w to about 8% w/w, about 4.5% w/w to about 10% w/w, about 5% w/w to about 6% w/w, about 5% w/w to about 8% w/w, about 5% w/w to about 10% w/w, about 6% w/w to about 8% w/w, about 6% w/w to about 10% w/w, or about 8% w/w to about 10% w/w. In some embodiments, the polidocanol is present in an amount of about 0.1% w/w, about 0.2% w/w, about 0.5% w/w, about 1% w/w, about 2% w/w, about 3% w/w, about 4% w/w, about 4.5% w/w, about 5% w/w, about 6% w/w, about 8% w/w, or about 10% w/w. In some embodiments, the polidocanol is present in an amount of at least about 0.1% w/w, about 0.2% w/w, about 0.5% w/w, about 1% w/w, about 2% w/w, about 3% w/w, about 4% w/w, about 4.5% w/w, about 5% w/w, about 6% w/w, or about 8% w/w. In some embodiments, the polidocanol is present in an amount of at most about 0.2% w/w, about 0.5% w/w, about 1% w/w, about 2% w/w, about 3% w/w, about 4% w/w, about 4.5% w/w, about 5% w/w, about 6% w/w, about 8% w/w, or about 10% w/w.

In embodiments, the compound of Formula II is present in an amount of about 0.1%-10% w/w or about 0.1%-5% w/w. In some embodiments, the compound of Formula II is present in an amount of about 0.5%-3% w/w, or less than about 0.5% w/w, or less than about 1% w/w, or less than about 2% w/w, or less than about 3% w/w, or less than about 4% w/w, or less than about 5% w/w, or less than about 10% w/w. In further or additional embodiments, the compound of Formula II is present in an amount that is greater than 1% w/w, greater than 1.5% w/w, greater than 2% w/w, greater than 2.5% w/w, greater than 3% w/w, greater than 4% w/w, greater than 5% w/w, greater than 6% w/w, or is about 10% w/w. In embodiments, the compound of Formula II is present in an amount between about 0.75% and about 1.50% w/w. In embodiments, the compound of Formula II is present in an amount between about 0.80% and about 1.45% w/w. In embodiments, the compound of Formula II is present in an amount between about 0.85% and about 1.40% w/w. In embodiments, the compound of Formula II is present in an amount between about 0.90% and about 1.35% w/w. In embodiments, the compound of Formula II is present in an amount between about 0.95% and about 1.30% w/w. In embodiments, the compound of Formula II is present in an amount between about 1.00% and about 1.25% w/w. In embodiments, the compound of Formula II is present in an amount between about 1.05% and about 1.20% w/w. In embodiments, the compound of Formula II is present in an amount between about 1.10% and about 1.15% w/w. In embodiments, the compound of Formula II is present in an amount between about 1.00% and about 1.30% w/w.

In embodiments, the compound of Formula II is present in an amount between about 1.00% and about 1.35% w/w. In embodiments, the compound of Formula II is present in an amount between about 1.00% and about 1.40% w/w. In embodiments, the compound of Formula II is present in an amount between about 1.00% and about 1.45% w/w. In embodiments, the compound of Formula II is present in an amount between about 1.00% and about 1.50% w/w. In embodiments, the compound of Formula II is present in an amount of about 1.00% w/w. In embodiments, the compound of Formula II is present in an amount of about 1.05% w/w. In embodiments, the compound of Formula II is present in an amount of about 1.10% w/w. In embodiments, the compound of Formula II is present in an amount of about 1.15% w/w. In embodiments, the compound of Formula II is present in an amount of about 1.20% w/w. In embodiments, the compound of Formula II is present in an amount of about 1.25% w/w. In embodiments, the compound of Formula II is present in an amount of about 1.30% w/w. In embodiments, the compound of Formula II is present in an amount of about 1.35% w/w. In embodiments, the compound of Formula II is present in an amount of about 1.40% w/w. In embodiments, the compound of Formula II is present in an amount of about 1.45% w/w. In embodiments, the compound of Formula II is present in an amount of about 1.50% w/w. In embodiments, the compound of Formula II is present in an amount between 0.75% and 1.50% w/w. In embodiments, the compound of Formula II is present in an amount between 0.80% and 1.45% w/w. In embodiments, the compound of Formula II is present in an amount between 0.85% and 1.40% w/w. In embodiments, the compound of Formula II is present in an amount between 0.90% and 1.35% w/w. In embodiments, the compound of Formula II is present in an amount between 0.95% and 1.30% w/w. In embodiments, the compound of Formula II is present in an amount between 1.00% and 1.25% w/w. In embodiments, the compound of Formula II is present in an amount between 1.05% and 1.20% w/w. In embodiments, the compound of Formula II is present in an amount between 1.10% and 1.15% w/w. In embodiments, the compound of Formula II is present in an amount between 1.00% and 1.30% w/w. In embodiments, the compound of Formula II is present in an amount between 1.00% and 1.35% w/w. In embodiments, the compound of Formula II is present in an amount between 1.00% and 1.40% w/w. In embodiments, the compound of Formula II is present in an amount between 1.00% and 1.45% w/w. In embodiments, the compound of Formula II is present in an amount between 1.00% and 1.50% w/w. In embodiments, the compound of Formula II is present in an amount of about 1.00% w/w. In embodiments, the compound of Formula II is present in an amount of about 1.05% w/w. In embodiments, the compound of Formula II is present in an amount of about 1.10% w/w. In embodiments, the compound of Formula II is present in an amount of about 1.15% w/w. In embodiments, the compound of Formula II is present in an amount of about 1.20% w/w. In embodiments, the compound of Formula II is present in an amount of about 1.25% w/w. In embodiments, the compound of Formula II is present in an amount of about 1.30% w/w. In embodiments, the compound of Formula II is present in an amount of about 1.35% w/w. In embodiments, the compound of Formula II is present in an amount of about 1.40% w/w. In embodiments, the compound of Formula II is present in an amount of about 1.45% w/w. In embodiments, the compound of Formula II is present in an amount of about 1.50% w/w.

In embodiments, the compound of Formula I is present in an amount of about 0.1%-10% w/w or about 0.1%-5% w/w. In some embodiments, the compound of Formula I is present in an amount of about 0.5%-3% w/w, or less than about 0.5% w/w, or less than about 1% w/w, or less than about 2% w/w, or less than about 3% w/w, or less than about 4% w/w, or less than about 5% w/w, or less than about 10% w/w. In further or additional embodiments, the compound of Formula I is present in an amount that is greater than 1% w/w, greater than 1.5% w/w, greater than 2% w/w, greater than 2.5% w/w, greater than 3% w/w, greater than 4% w/w, greater than 5% w/w, greater than 6% w/w, or is about 10% w/w. In embodiments, the compound of Formula I is present in an amount between about 0.75% and about 1.50% w/w. In embodiments, the compound of Formula I is present in an amount between about 0.80% and about 1.45% w/w. In embodiments, the compound of Formula I is present in an amount between about 0.85% and about 1.40% w/w. In embodiments, the compound of Formula I is present in an amount between about 0.90% and about 1.35% w/w. In embodiments, the compound of Formula I is present in an amount between about 0.95% and about 1.30% w/w. In embodiments, the compound of Formula I is present in an amount between about 1.00% and about 1.25% w/w. In embodiments, the compound of Formula I is present in an amount between about 1.05% and about 1.20% w/w. In embodiments, the compound of Formula I is present in an amount between about 1.10% and about 1.15% w/w. In embodiments, the compound of Formula I is present in an amount between about 1.00% and about 1.30% w/w. In embodiments, the compound of Formula I is present in an amount between about 1.00% and about 1.35% w/w. In embodiments, the compound of Formula I is present in an amount between about 1.00% and about 1.40% w/w. In embodiments, the compound of Formula I is present in an amount between about 1.00% and about 1.45% w/w. In embodiments, the compound of Formula I is present in an amount between about 1.00% and about 1.50% w/w. In embodiments, the compound of Formula I is present in an amount of about 1.00% w/w. In embodiments, the compound of Formula I is present in an amount of about 1.05% w/w. In embodiments, the compound of Formula I is present in an amount of about 1.10% w/w. In embodiments, the compound of Formula I is present in an amount of about 1.15% w/w. In embodiments, the compound of Formula I is present in an amount of about 1.20% w/w. In embodiments, the compound of Formula I is present in an amount of about 1.25% w/w. In embodiments, the compound of Formula I is present in an amount of about 1.30% w/w. In embodiments, the compound of Formula I is present in an amount of about 1.35% w/w. In embodiments, the compound of Formula I is present in an amount of about 1.40% w/w. In embodiments, the compound of Formula I is present in an amount of about 1.45% w/w. In embodiments, the compound of Formula I is present in an amount of about 1.50% w/w. In embodiments, the compound of Formula I is present in an amount between 0.75% and 1.50% w/w. In embodiments, the compound of Formula I is present in an amount between 0.80% and 1.45% w/w. In embodiments, the compound of Formula I is present in an amount between 0.85% and 1.40% w/w. In embodiments, the compound of Formula I is present in an amount between 0.90% and 1.35% w/w. In embodiments, the compound of Formula I is present in an amount between 0.95% and 1.30% w/w. In embodiments, the compound of Formula I is present in an amount between 1.00% and 1.25% w/w. In embodiments, the compound of Formula I is present in an amount between 1.05% and 1.20% w/w. In embodiments, the compound of Formula I is present in an amount between 1.10% and 1.15% w/w. In embodiments, the compound of Formula I is present in an amount between 1.00% and 1.30% w/w. In embodiments, the compound of Formula I is present in an amount between 1.00% and 1.35% w/w. In embodiments, the compound of Formula I is present in an amount between 1.00% and 1.40% w/w. In embodiments, the compound of Formula I is present in an amount between 1.00% and 1.45% w/w. In embodiments, the compound of Formula I is present in an amount between 1.00% and 1.50% w/w. In embodiments, the compound of Formula I is present in an amount of about 1.00% w/w. In embodiments, the compound of Formula I is present in an amount of about 1.05% w/w. In embodiments, the compound of Formula I is present in an amount of about 1.10% w/w. In embodiments, the compound of Formula I is present in an amount of about 1.15% w/w. In embodiments, the compound of Formula I is present in an amount of about 1.20% w/w. In embodiments, the compound of Formula I is present in an amount of about 1.25% w/w. In embodiments, the compound of Formula I is present in an amount of about 1.30% w/w. In embodiments, the compound of Formula I is present in an amount of about 1.35% w/w. In embodiments, the compound of Formula I is present in an amount of about 1.40% w/w. In embodiments, the compound of Formula I is present in an amount of about 1.45% w/w. In embodiments, the compound of Formula I is present in an amount of about 1.50% w/w.

While not wishing to be bound by theory, in embodiments, polidocanol and related compounds of Formula I or Formula II preferentially lyse fat cells while leaving surrounding tissue largely unaffected (e.g., unlysed or less lysed in comparison to the lysis of fat cells (e.g., percentage of lysed cells, number of lysed cells, degree of lysis of a tissue or cell population)). Accordingly, in some embodiments, the formulations described herein provide selective reduction (e.g., reduction of fat cells or tissue in a greater amount than adjacent non-fat cells or tissue) of regional and/or subcutaneous accumulations of adipose tissue and adipocytes, including cellulite, through subcutaneous administration of polidocanol, or polidocanol-like compounds of Formula I or Formula II. In some embodiments, the compositions described herein are useful for treating cellulitic fat accumulation and/or lipomas.

In certain embodiments, provided is a formulation or composition including a compound of Formula I or Formula II (e.g., polidocanol) that provides one or more of the following: (a) a critical micelle concentration (“CMC”) of less than about 5 millimolar; (b) an HLB value of from about 10 to about 15; and (c) is non-ionic. In some embodiments, these formulations are used for contacting fat tissue, abdominal fat accumulation, regional adiposity, excess sub-mental fat, and exophthalmos (e.g., due to thyroid eye disease), and/or for emulsifying, necrotizing, lysing or destroying one or more adipose cells while, in certain situations, leaving surrounding tissue largely unaffected (e.g., unlysed or less lysed in comparison to the lysis of fat cells (e.g., percentage of lysed cells, number of lysed cells, degree of lysis of a tissue or cell population). In certain embodiments, the compound is polidocanol.

In embodiments, the polidocanol is present in an amount of about 0.1%-10% w/w or about 0.1%-5% w/w. In some embodiments, the polidocanol is present in an amount of about 0.1% w/w to about 10% w/w. In some embodiments, the polidocanol is present in an amount of about 0.1% w/w to about 0.2% w/w, about 0.1% w/w to about 0.5% w/w, about 0.1% w/w to about 1% w/w, about 0.1% w/w to about 2% w/w, about 0.1% w/w to about 3% w/w, about 0.1% w/w to about 4% w/w, about 0.1% w/w to about 4.5% w/w, about 0.1% w/w to about 5% w/w, about 0.1% w/w to about 6% w/w, about 0.1% w/w to about 8% w/w, about 0.1% w/w to about 10% w/w, about 0.2% w/w to about 0.5% w/w, about 0.2% w/w to about 1% w/w, about 0.2% w/w to about 2% w/w, about 0.2% w/w to about 3% w/w, about 0.2% w/w to about 4% w/w, about 0.2% w/w to about 4.5% w/w, about 0.2% w/w to about 5% w/w, about 0.2% w/w to about 6% w/w, about 0.2% w/w to about 8% w/w, about 0.2% w/w to about 10% w/w, about 0.5% w/w to about 1% w/w, about 0.5% w/w to about 2% w/w, about 0.5% w/w to about 3% w/w, about 0.5% w/w to about 4% w/w, about 0.5% w/w to about 4.5% w/w, about 0.5% w/w to about 5% w/w, about 0.5% w/w to about 6% w/w, about 0.5% w/w to about 8% w/w, about 0.5% w/w to about 10% w/w, about 1% w/w to about 2% w/w, about 1% w/w to about 3% w/w, about 1% w/w to about 4% w/w, about 1% w/w to about 4.5% w/w, about 1% w/w to about 5% w/w, about 1% w/w to about 6% w/w, about 1% w/w to about 8% w/w, about 1% w/w to about 10% w/w, about 2% w/w to about 3% w/w, about 2% w/w to about 4% w/w, about 2% w/w to about 4.5% w/w, about 2% w/w to about 5% w/w, about 2% w/w to about 6% w/w, about 2% w/w to about 8% w/w, about 2% w/w to about 10% w/w, about 3% w/w to about 4% w/w, about 3% w/w to about 4.5% w/w, about 3% w/w to about 5% w/w, about 3% w/w to about 6% w/w, about 3% w/w to about 8% w/w, about 3% w/w to about 10% w/w, about 4% w/w to about 4.5% w/w, about 4% w/w to about 5% w/w, about 4% w/w to about 6% w/w, about 4% w/w to about 8% w/w, about 4% w/w to about 10% w/w, about 4.5% w/w to about 5% w/w, about 4.5% w/w to about 6% w/w, about 4.5% w/w to about 8% w/w, about 4.5% w/w to about 10% w/w, about 5% w/w to about 6% w/w, about 5% w/w to about 8% w/w, about 5% w/w to about 10% w/w, about 6% w/w to about 8% w/w, about 6% w/w to about 10% w/w, or about 8% w/w to about 10% w/w. In some embodiments, the polidocanol is present in an amount of about 0.1% w/w, about 0.2% w/w, about 0.5% w/w, about 1% w/w, about 2% w/w, about 3% w/w, about 4% w/w, about 4.5% w/w, about 5% w/w, about 6% w/w, about 8% w/w, or about 10% w/w. In some embodiments, the polidocanol is present in an amount of at least about 0.1% w/w, about 0.2% w/w, about 0.5% w/w, about 1% w/w, about 2% w/w, about 3% w/w, about 4% w/w, about 4.5% w/w, about 5% w/w, about 6% w/w, or about 8% w/w. In some embodiments, the polidocanol is present in an amount of at most about 0.2% w/w, about 0.5% w/w, about 1% w/w, about 2% w/w, about 3% w/w, about 4% w/w, about 4.5% w/w, about 5% w/w, about 6% w/w, about 8% w/w, or about 10% w/w.

### Critical Micelle Concentration (“CMC”)

The critical micelle concentration refers to the concentration of surfactants above which micelles form and all additional surfactants added to the system will form micelles. In some embodiments, provided is a formulation including a compound of Formula I or Formula II (e.g., polidocanol) that provides a critical micelle concentration (“CMC”) of less than 5 millimolar, less about 4 millimolar, less than about 3 millimolar, less than about 2 millimolar, less than about 1 millimolar, less than about 0.9 millimolar, less than about 0.7 millimolar, less than about 0.5 millimolar, or less than about 0.1 millimolar. In some embodiments, the CMC is from about 0.01 to about 10 millimolar. In some embodiments, the CMC is from 0.5 to about 1.0. In further or additional embodiments, the CMC is from 0.7 to 0.8. In some embodiments, provided is formulation that is used for contacting fat tissue, abdominal fat accumulation, regional adiposity, excess sub-mental fat, and exophthalmos (e.g., due to thyroid eye disease), and/or for emulsifying, necrotizing, lysing or destroying one or more adipose cells while, in certain situations, leaving surrounding tissue largely unaffected (e.g., unlysed or less lysed in comparison to the lysis of fat cells (e.g., percentage of lysed cells, number of lysed cells, degree of lysis of a tissue or cell population). In certain embodiments, the compound is polidocanol. In some embodiments, the formulations provided herein form a micelle after administration to tissue, including subcutaneous adiposity tissue. In some embodiments, the concentration of the polidocanol in the formulation must be above the critical micelle concentration of the formulation to allow for polidocanol micelle formulation, in order to emulsify the subcutaneous adipose tissue with polidocanol micelles.

### Hydrophilic-Lipophilic Balance (“HLB”)

In some embodiments, provided is a formulation including a polidocanol of Formula I or Formula II (e.g., polidocanol) that has an HLB value of from about 10 to about 15. Changing the nature of the hydrophobic hydrocarbon alkyl tail of a compound of Formulas I or II such as increasing or decreasing the length or the degree of saturation can affect the HLB. The length of the hydrophilic head (e.g., a polyether) of a compound of Formulas I or II can also affect the HLB. For example, too many polyethers can cause the compound to be too water soluble and act like ionic detergent and have unwanted effects on healthy tissue (e.g., adjacent, surrounding, non-fat tissue). On the other hand, too few polyether groups the compound will render the compound not soluble or immiscible (e.g., crystallization, aggregation) in aqueous solution causing formulation and manufacture complications (e.g., non-uniform formulation), and in turn decreased effectiveness (e.g., non-uniform adipose reduction over an area or non-uniform reduction of adipocytes over an area, patchiness of fat reduction). Thus, the disclosed n values of the compounds of Formulas I and II as described herein are desirable.

In some embodiments, provided is a formulation including a compound of Formula I or Formula II (e.g., polidocanol) that has an HLB value of from about 11-14, of about 12-13, of about 12.5-13.5, of about 11, of about 12, about 13, of about 14, or of about 15. In some embodiments, these formulations are used for contacting fat tissue, abdominal fat accumulation, regional adiposity, excess sub-mental fat, and exophthalmos (e.g., due to thyroid eye disease), and/or for emulsifying, necrotizing, lysing or destroying one or more adipose cells while, in certain applications, leaving surrounding tissue largely unaffected (e.g., unlysed or less lysed in comparison to the lysis of fat cells (e.g., percentage of lysed cells, number of lysed cells, degree of lysis of a tissue or cell population). In certain embodiments, the compound is polidocanol.

As described above, the polidocanol may be a mixture of C₁₂ alkyl ethoxylate homologues. In embodiments, about 10% to about 90% of the mixture has a HLB from about 10 to about 15. In embodiments, about 20% to about 80% of the mixture has a HLB from about 10 to about 15. In embodiments, about 30% to about 70% of the mixture has a HLB from about 10 to about 15. In embodiments, about 40% to about 60% of the mixture has a HLB from about 10 to about 15. In embodiments, about 50% of the mixture has a HLB from about 10 to about 15. In embodiments, about 10% to about 20% of the mixture has a HLB from about 10 to about 15. In embodiments, about 15% of the mixture has an HLB from about 10 to about 15. In embodiments, about 5% to about 20% of the mixture has an HLB below about 10. In embodiments, about 10% to about 20% of the mixture has an HLB below 10. In embodiments, about 10% of the mixture has an HLB below about 10. In embodiments, about 20% of the mixture has an HLB below about 10. In embodiments, about 20% of the mixture has an HLB below about 10. In embodiments, about 1% to about 20% of the mixture has an HLB above about 10. In embodiments, about 1% to about 15% of the mixture has an HLB above 10. In embodiments, about 1% to about 10% of the mixture has an HLB above about 10. In embodiments, about 1% to about 5% of the mixture has an HLB above about 10. In embodiments, about 10% of the mixture has an HLB above about 10.

As described above, the polidocanol may a mixture of C12 alkyl ethoxylate homologues. In embodiments, 10% to 90% of the mixture has a HLB from 10 to 15. In embodiments, 20% to 80% of the mixture has a HLB from 10 to 15. In embodiments, 30% to 70% of the mixture has a HLB from 10 to 15. In embodiments, 40% to 60% of the mixture has a HLB from 10 to 15. In embodiments, 50% of the mixture has a HLB from 10 to 15. In embodiments, 10% to 20% of the mixture has a HLB from 10 to 15. In embodiments, 15% of the mixture has an HLB from 10 to 15. In embodiments, 5% to 20% of the mixture has an HLB below 10. In embodiments, 10% to 20% of the mixture has an HLB below 10. In embodiments, 10% of the mixture has an HLB below 10. In embodiments, 20% of the mixture has an HLB below 10. In embodiments, 20% of the mixture has an HLB below 10. In embodiments, 1% to 20% of the mixture has an HLB above 10. In embodiments, 1% to 15% of the mixture has an HLB above 10. In embodiments, 1% to 10% of the mixture has an HLB above 10. In embodiments, 1% to 5% of the mixture has an HLB above 10. In embodiments, 10% of the mixture has an HLB above 10.

### Non-Ionic Compounds/Safety Profile for Human Administration

In some embodiments, the formulation includes a compound of Formula I or Formula II that is non-ionic. Such compounds tend to not denature non-targeted tissue, including proteins, upon administration, which leads to a better safety and tolerability profile. In addition, these compounds have natural anesthetic properties which can reduce injection pain and improve tolerability. In some embodiments, these formulations including the non-ionic detergent are used for contacting fat tissue, abdominal fat accumulation, regional adiposity, excess sub-mental fat, and exophthalmos (e.g., due to thyroid eye disease), and/or for emulsifying, necrotizing, lysing or destroying one or more adipose cells while in specific applications leaving surrounding tissue largely unaffected (e.g., unlysed or less lysed in comparison to the lysis of fat cells (e.g., percentage of lysed cells, number of lysed cells, degree of lysis of a tissue or cell population). In certain embodiments, the compound is polidocanol.

In an exemplary embodiment, a formulation includes from about 0.1 mg to about 100 mg (e.g., about 0.5, 0.7 mg, 1 mg, 1.5 mg, 2.0 mg, 2.5 mg, 3 mg, 3.5 mg, 4 mg, 4.5 mg, 5 mg, 5.5 mg, 6 mg, 6.5 mg, 7 mg, 8 mg, 9 mg, 10 mg, 15 mg, 20 mg, 25 mg, 50 mg, 75 mg, or 100 mg, or any other amount from about 0.1 mg to about 100 mg) of a compound of Formula I or Formula II such as polidocanol. The amount of active ingredient in the formulation depends on the period of administration prescribed (including about daily, about every 3 days to about 12 months, e.g., 4 days, 5 days, 7 days, 10 days, 1 month, 45 days, 2 months, 3 months, 6 months, 8 months, 9 months, or any other release period from about daily to about 12 months). In an exemplary embodiment, a formulation includes from 0.1 mg to 100 mg (e.g., 0.5, 0.7 mg, 1 mg, 1.5 mg, 2.0 mg, 2.5 mg, 3 mg, 3.5 mg, 4 mg, 4.5 mg, 5 mg, 5.5 mg, 6 mg, 6.5 mg, 7 mg, 8 mg, 9 mg, 10 mg, 15 mg, 20 mg, 25 mg, 50 mg, 75 mg, or 100 mg, or any other amount from 0.1 mg to 100 mg) of a compound of Formula I or Formula II such as polidocanol. The amount of active ingredient in the formulation depends on the period of administration prescribed (including daily every 3 days to 12 months, e.g., 4 days, 5 days, 7 days, 10 days, 1 month, 45 days, 2 months, 3 months, 6 months, 8 months, 9 months, or any other release period from daily to 12 months). The amount of active ingredient in the formulation depends on the number of individual administrations (e.g., injections spread over an area) that collectively are a single treatment (e.g., one total administration to the subject at one time).

### Co-Solvent and Additives

It has been discovered herein, inter alia, that a C₃-C₆ alcohol such as propylene glycol is non-irritating when injected with polidocanol. Thus, in embodiments, a C₃-C₆ alcohol such as propylene glycol is administered as a co-solvent in the polidocanol compositions described herein (e.g., in a non-irritating amount).

It has further been found herein that the use of a C₃-C₆ alcohol such as propylene glycol as a co-solvent provides a reduced amount of patchy and incomplete fat tissue necrosis relative to the absence of co-solvent (e.g., propylene glycol). Thus, in embodiments, the C₃-C₆ alcohol such as propylene glycol is provided (e.g., in a composition including an active ingredient such as polidocanol) in an amount effective to reduce the amount of patchy or incomplete fat tissue necrosis relative to the absence of the C₃-C₆ alcohol such as propylene glycol.

It has been further discovered herein that a C₃-C₆ alcohol such as propylene glycol may reduce the tendency of polidocanol to solidify or crystallize upon injection into a subject relative to the absence of co-solvent (e.g., propylene glycol). In embodiments, a C₃-C₆ alcohol such as propylene glycol is provided (e.g., in a composition including an active ingredient such as polidocanol) in an amount effective to reduce the amount of crystallization or solidification of polidocanol in the subject relative to the absence of the C₃-C₆ alcohol such as propylene glycol.

It has been further discovered herein that a C₃-C₆ alcohol such as propylene glycol may reduce foam formation in the polidocanol compositions provided herein and/or increase the stability of the polidocanol compositions provided herein. In embodiments, a C₃-C₆ alcohol such as propylene glycol is provided in an amount effective to reduce the amount of foam formation in the polidocanol compositions relative to the absence of the C₃-C₆ alcohol such as propylene glycol. In embodiments, a C₃-C₆ alcohol such as propylene glycol is provided in an amount effective to increase the stability of the polidocanol compositions relative to the absence of the C₃-C₆ alcohol such as propylene glycol.

In embodiments, 2.5% of a C₃-C₆ alcohol such as propylene glycol is provided (e.g., in a composition including an active ingredient such as polidocanol).

Also provided herein are polidocanol compositions having an osmolality greater than about 600 osm. In embodiments, the polidocanol composition may include a buffer, such as a phosphate buffer. The phosphate buffer may be provided in concentration of 1% (w/w) within the polidocanol composition (e.g. see Tables 2-6 below). The composition may further include a C₃-C₆ alcohol such as propylene glycol (e.g. in a concentration of about 2.5% or 5%). In embodiments, the amount of the C₃-C₆ alcohol such as propylene glycol is about 2.5% and the amount of polidocanol is about 2.0% or less, wherein the composition is isotonic (e.g. greater than 600 osm). In embodiments provided herein containing a C₃-C₆ alcohol the C₃-C₆ alcohol may be glycerin.

It has been discovered herein that a C₃-C₆ alcohol such as propylene glycol is non-irritating when injected with a compound of Formula II. Thus, in embodiments, a C₃-C₆ alcohol such as propylene glycol is administered as a co-solvent in the compound of Formula II compositions described herein (e.g., in a non-irritating amount). It has further been found herein that the use of a C₃-C₆ alcohol such as propylene glycol as a co-solvent provides a reduced amount of patchy and incomplete fat tissue necrosis relative to the absence of co-solvent (e.g., propylene glycol). Thus, in embodiments, a C₃-C₆ alcohol such as propylene glycol is provided (e.g., in a composition including an active ingredient such as a compound of Formula II) in an amount effective to reduce the amount of patchy or incomplete fat tissue necrosis relative to the absence of the C₃-C₆ alcohol such as propylene glycol. It has been further discovered herein that a C₃-C₆ alcohol such as propylene glycol may reduce the tendency of compound of Formula II to solidify or crystallize upon injection into a subject relative to the absence of co-solvent (e.g., propylene glycol). In embodiments, a C₃-C₆ alcohol such as propylene glycol is provided (e.g., in a composition including an active ingredient such as a compound of Formula II) in an amount effective to reduce the amount of crystallization or solidification of a compound of Formula II in the subject relative to the absence of the C₃-C₆ alcohol such as propylene glycol. It has been further discovered herein that a C₃-C₆ alcohol such as propylene glycol may reduce foam formation in the compound of Formula II compositions provided herein and/or increase the stability of the compound of Formula II compositions provided herein. In embodiments, a C₃-C₆ alcohol such as propylene glycol is provided in an amount effective to reduce the amount of foam formation in the compound of Formula II compositions relative to the absence of the C₃-C₆ alcohol such as propylene glycol. In embodiments, a C₃-C₆ alcohol such as propylene glycol is provided in an amount effective to increase the stability of the compound of Formula II compositions relative to the absence of the C₃-C₆ alcohol such as propylene glycol. In embodiments, 2.5% of a C₃-C₆ alcohol such as propylene glycol is provided (e.g., in a composition including an active ingredient such as compound of Formula II). Also provided herein are compound of Formula II compositions having an osmolality greater than about 600 osm. In embodiments, the compound of Formula II composition may include a buffer, such as a phosphate buffer. The phosphate buffer may be provided in concentration of 1% (w/w) within the polidocanol composition (e.g. see Tables 2-6 below). The composition may further include a C₃-C₆ alcohol such as propylene glycol (e.g. in a concentration of about 2.5% or 5%). In embodiments, the amount of a C₃-C₆ alcohol such as propylene glycol is about 2.5% and the amount of compound of Formula II is about 2.0% or less, wherein the composition is isotonic (e.g. greater than 600 osm). In embodiments, the a C₃-C₆ alcohol is glycerin.

It has been discovered herein that a C₃-C₆ alcohol such as propylene glycol is non-irritating when injected with a compound of Formula I. Thus, in embodiments, a C₃-C₆ alcohol such as propylene glycol is administered as a co-solvent in a compound of Formula I compositions described herein (e.g., in a non-irritating amount). It has further been found herein that the use of a C₃-C₆ alcohol such as propylene glycol as a co-solvent provides a reduced amount of patchy and incomplete fat tissue necrosis relative to the absence of co-solvent (e.g., propylene glycol). Thus, in embodiments, a C₃-C₆ alcohol such as propylene glycol is provided (e.g., in a composition including an active ingredient such as a compound of Formula I) in an amount effective to reduce the amount of patchy or incomplete fat tissue necrosis relative to the absence of the C₃-C₆ alcohol such as propylene glycol. It has been further discovered herein that a C₃-C₆ alcohol such as propylene glycol may reduce the tendency of a compound of Formula I to solidify or crystallize upon injection into a subject relative to the absence of co-solvent (e.g., propylene glycol). In embodiments, a C₃-C₆ alcohol such as propylene glycol is provided (e.g., in a composition including an active ingredient such as a compound of Formula I) in an amount effective to reduce the amount of crystallization or solidification of a compound of Formula I in the subject relative to the absence of the C₃-C₆ alcohol such as propylene glycol. It has been further discovered herein that a C₃-C₆ alcohol such as propylene glycol may reduce foam formation in the compound of Formula I compositions provided herein and/or increase the stability of the compound of Formula I compositions provided herein. In embodiments, a C₃-C₆ alcohol such as propylene glycol is provided in an amount effective to reduce the amount of foam formation in the compound of Formula I compositions relative to the absence of the C₃-C₆ alcohol such as propylene glycol. In embodiments, a C₃-C₆ alcohol such as propylene glycol is provided in an amount effective to increase the stability of the compound of Formula I compositions relative to the absence of the C₃-C₆ alcohol such as propylene glycol. In embodiments, 2.5% of a C₃-C₆ alcohol such as propylene glycol is provided (e.g., in a composition including an active ingredient such as a compound of Formula I). Also provided herein are compound of Formula I compositions having an osmolality greater than about 600 osm. In embodiments, the compound of Formula I composition may include a buffer, such as a phosphate buffer. The phosphate buffer may be provided in concentration of 1% (w/w) within a C₃-C₆ alcohol such as the polidocanol composition (e.g. see Tables 2-6 below). The composition may further include a C₃-C₆ alcohol such as propylene glycol (e.g. in a concentration of about 2.5% or 5%). In embodiments, the amount of a C₃-C₆ alcohol such as propylene glycol is about 2.5% and the amount of a compound of Formula I is about 2.0% or less, wherein the composition is isotonic (e.g. greater than 600 osm). In embodiments, the C₃-C₆ alcohol is glycerin.

### Formulation Carriers

In some embodiments, the liquid carrier is a lipophilic liquid carrier. In certain embodiments, the liquid carrier is aqueous. In an embodiment, a formulation including a compound of formula I or II (e.g., polidocanol) described herein allows dispersal of the compound or salt other suitable form thereof into the layer of subcutaneous fat at a regional fat site selected from one or more of the following: a sub-mental region, an abdominal region, a waist, a hip, a lateral buttock, a thigh, a peri-orbital region, an intra-orbital region, and intramuscular region, and combinations thereof.

In certain embodiments, provided are formulations for treating regional adipose tissue, regional adiposity, or regional fat accumulation. In certain embodiments, the formulations include an effective amount of at least one compound of Formula I or Formula II (e.g., polidocanol) or a pharmaceutically acceptable or cosmetically acceptable salt and other suitable form thereof; and a liquid carrier; wherein the compound of Formula I or Formula II (e.g., polidocanol) or a pharmaceutically acceptable or cosmetically acceptable salt or others suitable form thereof and the liquid carrier formulated for injection into a layer of subcutaneous fat in the individual in need thereof. In certain embodiments, the compound of Formula II is polidocanol. In some embodiments, the formulation is stable for a period of at least 6 months at a temperature of about 0° C. to about 50° C. In certain embodiments, provided is a formulation described herein, wherein the compound of formula I or II is presented as a salt or other suitable form for administration to a human.

In certain embodiments, provided is a formulation wherein the compound of Formula I or Formula II such as polidocanol or a pharmaceutically acceptable or cosmetically acceptable salt or other suitable form thereof is present in an amount that is equal to or less than about 20% weight/weight (w/w). In certain embodiments, the compound of Formula I or Formula II such as polidocanol or a pharmaceutically acceptable or cosmetically acceptable salt or other suitable form thereof is present in an amount that is equal to or more than about 0.1% w/w to an amount that is equal to or less than about 10% w/w. In another embodiment, the compound or a pharmaceutically acceptable or cosmetically acceptable salt or other suitable form thereof is present in an amount that is equal to or more than about 0.2% w/w to an amount that is equal to or less than about 8% w/w. Also provided are embodiments of the formulations described herein wherein the compound of Formula I or Formula II such as polidocanol or a pharmaceutically acceptable or cosmetically acceptable salt or other suitable form thereof is present in an amount that is equal to or more than about 0.3% w/w to an amount that is equal to or less than about 6% w/w. In an embodiment, a compound or a pharmaceutically acceptable or cosmetically acceptable salt or other suitable form thereof is present in an amount that is equal to or more than about 0.4% w/w to an amount that is equal to or less than about 4% w/w. In some embodiments, a compound of Formula I or Formula II such as polidocanol or a pharmaceutically acceptable or cosmetically acceptable salt or other suitable form thereof is present in an amount that is equal to or more than about 0.5% w/w to an amount that is equal to or less than about 3% w/w. In a further embodiment of the formulations described herein, a compound of Formula I or Formula II or a pharmaceutically acceptable or cosmetically acceptable salt or other suitable form thereof is present in an amount that is equal to about 3% w/w. In an embodiment, a compound of Formula I or Formula II or a pharmaceutically acceptable or cosmetically acceptable salt or other suitable form thereof is present in an amount that is equal to about 0.5% w/w. Also provided are formulations wherein a compound of Formula I or Formula II or a pharmaceutically acceptable or cosmetically acceptable salt or other suitable form thereof is present in an amount that is equal to about 1% w/w. In certain embodiments, the compound of Formula II is polidocanol or a polidocanol-like compound.

In certain embodiments, the polidocanol or a pharmaceutically acceptable or cosmetically acceptable salt or other suitable form thereof is present in an amount that is equal to or less than about 10% weight/weight (w/w). In certain embodiments the polidocanol or a pharmaceutically acceptable or cosmetically acceptable salt or other suitable form thereof is present in an amount that is equal to or more than about 0.1% w/w to an amount that is equal to or less than about 10% w/w. In some other embodiments the polidocanol or a pharmaceutically acceptable or cosmetically acceptable salt or other suitable form thereof is present in an amount that is equal to or more than about 0.2% w/w to an amount that is equal to or less than about 8% w/w. In certain embodiments, the polidocanol or a pharmaceutically acceptable or cosmetically acceptable salt or other suitable form thereof is present in an amount that is equal to or more than about 0.3% w/w to an amount that is equal to or less than about 6% w/w. In some other embodiments the polidocanol or a pharmaceutically acceptable or cosmetically acceptable salt or other suitable form thereof is present in an amount that is equal to or more than about 0.4% w/w to an amount that is equal to or less than about 4% w/w. In select embodiments, the polidocanol or a pharmaceutically acceptable or cosmetically acceptable salt or other suitable form thereof is present in an amount that is equal to or more than about 0.5% w/w to an amount that is equal to or less than about 3% w/w. In some other embodiments the polidocanol or a pharmaceutically acceptable or cosmetically acceptable salt or other suitable form thereof is present in an amount that is equal to about 3% w/w. In certain further embodiments, the polidocanol or a pharmaceutically acceptable or cosmetically acceptable salt or other suitable form thereof is present in an amount that is equal to about 0.5% w/w. In some other embodiments, the polidocanol or a pharmaceutically acceptable or cosmetically acceptable salt or other suitable form thereof is present in an amount that is equal to about 1% w/w.

In certain embodiments, provided is a formulation wherein the compound of Formula I or Formula II (e.g., polidocanol), or a pharmaceutically acceptable or cosmetically acceptable salt thereof, is present in an amount that is equal to or less than about 20% weight/weight (w/w). In embodiment, the compound, or pharmaceutically acceptable or cosmetically acceptable salt thereof, is between about 0.1% w/w and about 10% w/w. In embodiment, the compound, or pharmaceutically acceptable or cosmetically acceptable salt thereof, is between about 0.2% w/w to about 8% w/w. In embodiment, the compound, or pharmaceutically acceptable or cosmetically acceptable salt thereof, is between about 0.3% w/w to about 6% w/w. In embodiment, the compound, or pharmaceutically acceptable or cosmetically acceptable salt thereof, is between about 0.4% w/w to about 4% w/w. In embodiment, the compound, or pharmaceutically acceptable or cosmetically acceptable salt thereof, is between about 0.5% w/w to about 3% w/w. In embodiment, the compound, or pharmaceutically acceptable or cosmetically acceptable salt thereof, is about 3% w/w. In embodiment, the compound, or pharmaceutically acceptable or cosmetically acceptable salt thereof, is about 0.5% w/w. In embodiment, the compound, or pharmaceutically acceptable or cosmetically acceptable salt thereof, is about 1% w/w. In certain embodiments, the compound is polidocanol or a polidocanol-like compound.

In certain embodiments, the polidocanol, or a pharmaceutically acceptable or cosmetically acceptable salt, thereof is less than about 10% weight/weight (w/w). In certain embodiments, the polidocanol, or a pharmaceutically acceptable or cosmetically acceptable salt, thereof is between about 0.1% w/w to about 10% w/w. In certain embodiments, the polidocanol, or a pharmaceutically acceptable or cosmetically acceptable salt, thereof is between about 0.2% w/w to about 8% w/w. In certain embodiments, the polidocanol, or a pharmaceutically acceptable or cosmetically acceptable salt, thereof is between about 0.3% w/w to about 6% w/w. In certain embodiments, the polidocanol, or a pharmaceutically acceptable or cosmetically acceptable salt, thereof is between about 0.4% w/w to about 4% w/w. In certain embodiments, the polidocanol, or a pharmaceutically acceptable or cosmetically acceptable salt, thereof is between about 0.5% w/w to about 3% w/w. In certain embodiments, the polidocanol, or a pharmaceutically acceptable or cosmetically acceptable salt, thereof is about 3% w/w. In certain embodiments, the polidocanol, or a pharmaceutically acceptable or cosmetically acceptable salt, thereof is about 0.5% w/w. In certain embodiments, the polidocanol, or a pharmaceutically acceptable or cosmetically acceptable salt, thereof is about 1% w/w. In certain embodiments, provided is a formulation wherein the compound of Formula I or Formula II (e.g., polidocanol), or a pharmaceutically acceptable or cosmetically acceptable salt thereof, is present in an amount that is equal to or less than 20% weight/weight (w/w). In embodiment, the compound, or pharmaceutically acceptable or cosmetically acceptable salt thereof, is between 0.1% w/w and 10% w/w. In embodiment, the compound, or pharmaceutically acceptable or cosmetically acceptable salt thereof, is between 0.2% w/w to 8% w/w. In embodiment, the compound, or pharmaceutically acceptable or cosmetically acceptable salt thereof, is between 0.3% w/w to 6% w/w. In embodiment, the compound, or pharmaceutically acceptable or cosmetically acceptable salt thereof, is between 0.4% w/w to 4% w/w. In embodiment, the compound, or pharmaceutically acceptable or cosmetically acceptable salt thereof, is between 0.5% w/w to 3% w/w. In embodiment, the compound, or pharmaceutically acceptable or cosmetically acceptable salt thereof, is 3% w/w. In embodiment, the compound, or pharmaceutically acceptable or cosmetically acceptable salt thereof, is 0.5% w/w. In embodiment, the compound, or pharmaceutically acceptable or cosmetically acceptable salt thereof, is 1% w/w. In certain embodiments, the compound is polidocanol or a polidocanol-like compound. In embodiments, the polidocanol is present in an amount of about 0.1%-10% w/w or about 0.1%-5% w/w. In some embodiments, the polidocanol is present in an amount of about 0.1% w/w to about 10% w/w. In some embodiments, the polidocanol is present in an amount of about 0.1% w/w to about 0.2% w/w, about 0.1% w/w to about 0.5% w/w, about 0.1% w/w to about 1% w/w, about 0.1% w/w to about 2% w/w, about 0.1% w/w to about 3% w/w, about 0.1% w/w to about 4% w/w, about 0.1% w/w to about 4.5% w/w, about 0.1% w/w to about 5% w/w, about 0.1% w/w to about 6% w/w, about 0.1% w/w to about 8% w/w, about 0.1% w/w to about 10% w/w, about 0.2% w/w to about 0.5% w/w, about 0.2% w/w to about 1% w/w, about 0.2% w/w to about 2% w/w, about 0.2% w/w to about 3% w/w, about 0.2% w/w to about 4% w/w, about 0.2% w/w to about 4.5% w/w, about 0.2% w/w to about 5% w/w, about 0.2% w/w to about 6% w/w, about 0.2% w/w to about 8% w/w, about 0.2% w/w to about 10% w/w, about 0.5% w/w to about 1% w/w, about 0.5% w/w to about 2% w/w, about 0.5% w/w to about 3% w/w, about 0.5% w/w to about 4% w/w, about 0.5% w/w to about 4.5% w/w, about 0.5% w/w to about 5% w/w, about 0.5% w/w to about 6% w/w, about 0.5% w/w to about 8% w/w, about 0.5% w/w to about 10% w/w, about 1% w/w to about 2% w/w, about 1% w/w to about 3% w/w, about 1% w/w to about 4% w/w, about 1% w/w to about 4.5% w/w, about 1% w/w to about 5% w/w, about 1% w/w to about 6% w/w, about 1% w/w to about 8% w/w, about 1% w/w to about 10% w/w, about 2% w/w to about 3% w/w, about 2% w/w to about 4% w/w, about 2% w/w to about 4.5% w/w, about 2% w/w to about 5% w/w, about 2% w/w to about 6% w/w, about 2% w/w to about 8% w/w, about 2% w/w to about 10% w/w, about 3% w/w to about 4% w/w, about 3% w/w to about 4.5% w/w, about 3% w/w to about 5% w/w, about 3% w/w to about 6% w/w, about 3% w/w to about 8% w/w, about 3% w/w to about 10% w/w, about 4% w/w to about 4.5% w/w, about 4% w/w to about 5% w/w, about 4% w/w to about 6% w/w, about 4% w/w to about 8% w/w, about 4% w/w to about 10% w/w, about 4.5% w/w to about 5% w/w, about 4.5% w/w to about 6% w/w, about 4.5% w/w to about 8% w/w, about 4.5% w/w to about 10% w/w, about 5% w/w to about 6% w/w, about 5% w/w to about 8% w/w, about 5% w/w to about 10% w/w, about 6% w/w to about 8% w/w, about 6% w/w to about 10% w/w, or about 8% w/w to about 10% w/w. In some embodiments, the polidocanol is present in an amount of about 0.1% w/w, about 0.2% w/w, about 0.5% w/w, about 1% w/w, about 2% w/w, about 3% w/w, about 4% w/w, about 4.5% w/w, about 5% w/w, about 6% w/w, about 8% w/w, or about 10% w/w. In some embodiments, the polidocanol is present in an amount of at least about 0.1% w/w, about 0.2% w/w, about 0.5% w/w, about 1% w/w, about 2% w/w, about 3% w/w, about 4% w/w, about 4.5% w/w, about 5% w/w, about 6% w/w, or about 8% w/w. In some embodiments, the polidocanol is present in an amount of at most about 0.2% w/w, about 0.5% w/w, about 1% w/w, about 2% w/w, about 3% w/w, about 4% w/w, about 4.5% w/w, about 5% w/w, about 6% w/w, about 8% w/w, or about 10% w/w.

In certain embodiments, the polidocanol, or a pharmaceutically acceptable or cosmetically acceptable salt, thereof is less than 10% weight/weight (w/w). In certain embodiments, the polidocanol, or a pharmaceutically acceptable or cosmetically acceptable salt, thereof is between 0.1% w/w to 10% w/w. In certain embodiments, the polidocanol, or a pharmaceutically acceptable or cosmetically acceptable salt, thereof is between 0.2% w/w to 8% w/w. In certain embodiments, the polidocanol, or a pharmaceutically acceptable or cosmetically acceptable salt, thereof is between 0.3% w/w to 6% w/w. In certain embodiments, the polidocanol, or a pharmaceutically acceptable or cosmetically acceptable salt, thereof is between 0.4% w/w to 4% w/w. In certain embodiments, the polidocanol, or a pharmaceutically acceptable or cosmetically acceptable salt, thereof is between 0.5% w/w to 3% w/w. In certain embodiments, the polidocanol, or a pharmaceutically acceptable or cosmetically acceptable salt, thereof is 3% w/w. In certain embodiments, the polidocanol, or a pharmaceutically acceptable or cosmetically acceptable salt, thereof is 0.5% w/w. In certain embodiments, the polidocanol, or a pharmaceutically acceptable or cosmetically acceptable salt, thereof is 1% w/w.

Described herein is the determination that safety and tolerability in connection with the reduction and emulsification of fat tissue in human is improved by a formulation including an effective amount, for example a therapeutically or cosmetically effective amount, of polidocanol and from about 0.5% to about 10% of an alcohol having 3 to 6 carbon atoms. Also identified are advantages to formulations including less than 2.0% polidocanol (e.g. from about 0.1% to about 1.5% or about 0.5% to about 1.25% or less than about 1%). Described herein is the determination that safety and tolerability in connection with the reduction and emulsification of fat tissue in human is improved by a formulation including an effective amount, for example a therapeutically or cosmetically effective amount, of polidocanol and from 0.5% to 10% of an alcohol having 3 to 6 carbon atoms. Also identified are advantages to formulations including less than 2.0% polidocanol (e.g. from 0.1% to 1.5% or 0.5% to 1.25% or less than 1%). Also identified are advantages to administration of polidocanol formulations described herein not more than once every 14 days (e.g., not more than 28 days (e.g. approximately monthly administration)).

In some embodiments, described herein are formulations including an effective amount, for example a therapeutically or cosmetically effective amount, of polidocanol and from about 0.5% w/w to about 10% w/w of propylene glycol. In some embodiments, described herein are formulations including an effective amount of polidocanol and from about 0.5% w/w to about 10% w/w of glycerine. Propylene glycol and glycerine are small, water soluble, hydroxylated organic molecules, which ensure formulation stability and safety when used with polidocanol. Both propylene glycol and glycerine are nearly non-toxic and non-irritating and suitable for subcutaneous injection. In some embodiments, the formulation includes an effective amount, example a therapeutically or cosmetically effective amount, of polidocanol and 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, or 10% w/w propylene glycol and does not contain ethanol or an ether. In some embodiments, the formulation includes an effective amount, for example a therapeutically or cosmetically effective amount, of polidocanol and 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, or 10% w/w glycerine and does not contain ethanol or an ether. In some embodiments, the formulation includes an effective amount, for example a therapeutically or cosmetically effective amount, of polidocanol and from about 0.5% to about 10% w/w of propylene glycol and does not contain aliphatic polyethers. In some embodiments, the formulation includes an effective amount, for example a therapeutically or cosmetically effective amount, of polidocanol and from about 0.5% to about 10% w/w of glycerine and does not contain aliphatic polyethers. In some embodiments, the formulation includes an effective amount, for example a therapeutically or cosmetically effective amount, of polidocanol and from about 0.5% to about 10% w/w of propylene glycol and does not contain polyethylene glycol. In some embodiments, the formulation includes an effective amount, for example a therapeutically or cosmetically effective amount, of polidocanol and from about 0.5% to about 10% w/w of glycerine and does not contain polyethylene glycol. In some embodiments, the formulation includes an effective amount, for example a therapeutically or cosmetically effective amount, of polidocanol and from about 0.5% to about 10% w/w of propylene glycol and does not contain poloxamers. In some embodiments, the formulation includes an effective amount, for example a therapeutically or cosmetically effective amount, of polidocanol and from about 0.5% to about 10% w/w of glycerine and does not contain poloxamers. In some embodiments, the formulation is for subcutaneous injection. In some embodiments, the formulation is for injection into the submental region of a human in need. In some embodiments, the formulation is for injection into a fat pad.

In some embodiments, the formulation includes an effective amount, for example a therapeutically or cosmetically effective amount, of polidocanol and from about 1.0% to about 5.0% w/w of an alcohol having 3 to 6 carbon atoms. In some embodiments, the formulation includes an effective amount, for example a therapeutically or cosmetically effective amount, of polidocanol and approximately 5.0% w/w of an alcohol having 3 to 6 carbon atoms. In some embodiments, the formulation includes polidocanol in an amount that is greater than 1.0% w/w and from about 1.0% to about 5.0% w/w of an alcohol having 3 to 6 carbon atoms. In some embodiments, the formulation includes polidocanol in an amount that is greater than 1.0% w/w and approximately 5.0% w/w of an alcohol having 3 to 6 carbon atoms. In some embodiments, the formulation includes polidocanol in an amount that is greater than 1.0% w/w and from about 1.0% to about 5.0% w/w of an alcohol having 3 to 6 carbon atoms and does not contain polyethylene glycol. In some embodiments, the formulation includes polidocanol in an amount that is greater than 1.0% w/w and from about 1.0% to about 5.0% w/w of an alcohol having 3 to 6 carbon atoms and does not contain ethanol or an ether. In some embodiments, the formulation includes polidocanol in an amount that is greater than 1.0% w/w and from about 1.0% to about 5.0% w/w of an alcohol having 3 to 6 carbon atoms and does not contain aliphatic polyethers. In some embodiments, the formulation includes polidocanol in an amount that is greater than 1.0% w/w and from about 1.0% to about 5.0% w/w of an alcohol having 3 to 6 carbon atoms and does not contain poloxamers.

In embodiments, the formulation includes polidocanol in an amount from about 0.1% w/w to about 20.0% w/w and from about 1.0% to about 5.0% w/w of an alcohol having 3 to 6 carbon atoms. In embodiments, the formulation includes polidocanol in an amount from about 0.1% w/w to about 20.0% w/w and about 5.0% w/w of an alcohol having 3 to 6 carbon atoms. In some embodiments, the formulation includes polidocanol in an amount from about 0.1% w/w to about 20.0% w/w and from about 1.0% to about 5.0% w/w of an alcohol having 3 to 6 carbon atoms and does not include polyethylene glycol. In some embodiments, the formulation includes polidocanol in an amount from about 0.1% w/w to about 20.0% w/w and from about 1.0% to about 5.0% w/w of an alcohol having 3 to 6 carbon atoms and does not include ethanol or an ether. In some embodiments, the formulation includes polidocanol in an amount from about 0.1% w/w to about 20.0% w/w and from about 1.0% to about 5.0% w/w of an alcohol having 3 to 6 carbon atoms and does not include aliphatic polyethers. In some embodiments, the formulation includes polidocanol in an amount from about 0.1% w/w to about 20.0% w/w and from about 1.0% to about 5.0% w/w of an alcohol having 3 to 6 carbon atoms and does not include poloxamers.

In some embodiments, the formulation includes polidocanol in an amount from about 0.5% w/w to about 10.0% w/w and from about 1.0% to about 5.0% w/w of an alcohol having 3 to 6 carbon atoms. In some embodiments, the formulation includes polidocanol in an amount from about 0.5% w/w to about 10.0% w/w and approximately 5.0% w/w of an alcohol having 3 to 6 carbon atoms. In some embodiments, the formulation includes polidocanol in an amount from about 0.5% w/w to about 10.0% w/w and from about 1.0% to about 5.0% w/w of an alcohol having 3 to 6 carbon atoms and does not include polyethylene glycol. In some embodiments, the formulation includes polidocanol in an amount from about 0.5% w/w to about 10.0% w/w and from about 1.0% to about 5.0% w/w of an alcohol having 3 to 6 carbon atoms and does not include ethanol or an ether. In some embodiments, the formulation includes polidocanol in an amount from about 0.5% w/w to about 10.0% w/w and from about 1.0% to about 5.0% w/w of an alcohol having 3 to 6 carbon atoms and does not include aliphatic polyethers. In some embodiments, the formulation includes polidocanol in an amount from about 0.5% w/w to about 10.0% w/w and from about 1.0% to about 5.0% w/w of an alcohol having 3 to 6 carbon atoms and does not include poloxamers.

In some embodiments, the formulation includes polidocanol in an amount from about 0.5% w/w to about 2.0% w/w and from about 1.0% to about 5.0% w/w of an alcohol having 3 to 6 carbon atoms. In some embodiments, the formulation includes polidocanol in an amount from about 0.5% w/w to about 2.0% w/w and approximately 5.0% w/w of an alcohol having 3 to 6 carbon atoms. In some embodiments, the formulation includes polidocanol in an amount from about 0.5% w/w to about 2.0% w/w and from about 1.0% to about 5.0% w/w of an alcohol having 3 to 6 carbon atoms and does not include polyethylene glycol. In some embodiments, the formulation includes polidocanol in an amount from about 0.5% w/w to about 2.0% w/w and from about 1.0% to about 5.0% w/w of an alcohol having 3 to 6 carbon atoms and does not include ethanol or an ether. In some embodiments, the formulation includes polidocanol in an amount from about 0.5% w/w to about 2.0% w/w and from about 1.0% to about 5.0% w/w of an alcohol having 3 to 6 carbon atoms and does not include aliphatic polyethers. In some embodiments, the formulation includes polidocanol in an amount from about 0.5% w/w to about 2.0% w/w and from about 1.0% to about 5.0% w/w of an alcohol having 3 to 6 carbon atoms and does not include poloxamers.

In some embodiments, the formulation includes polidocanol in an amount of about 0.5% w/w and from about 1.0% to about 5.0% w/w of an alcohol having 3 to 6 carbon atoms. In some embodiments, the formulation includes polidocanol in an amount of about 0.5% w/w and approximately 5.0% w/w of an alcohol having 3 to 6 carbon atoms. In some embodiments, the formulation includes polidocanol in an amount of about 0.5% w/w and from about 1.0% to about 5.0% w/w of an alcohol having 3 to 6 carbon atoms and does not include polyethylene glycol. In some embodiments, the formulation includes polidocanol in an amount of about 0.5% w/w and from about 1.0% to about 5.0% w/w of an alcohol having 3 to 6 carbon atoms and does not include ethanol or an ether. In some embodiments, the formulation includes polidocanol in an amount of about 0.5% w/w and from about 1.0% to about 5.0% w/w of an alcohol having 3 to 6 carbon atoms and does not include aliphatic polyethers. In some embodiments, the formulation includes polidocanol in an amount of about 0.5% w/w and from about 1.0% to about 5.0% w/w of an alcohol having 3 to 6 carbon atoms and does not include poloxamers.

In some embodiments, the formulation includes polidocanol in an amount of about 1.25% w/w and from about 1.0% to about 5.0% w/w of an alcohol having 3 to 6 carbon atoms. In some embodiments, the formulation includes polidocanol in an amount of about 1.25% w/w and approximately 5.0% w/w of an alcohol having 3 to 6 carbon atoms. In some embodiments, the formulation includes polidocanol in an amount of about 1.25% w/w and from about 1.0% to about 5.0% w/w of an alcohol having 3 to 6 carbon atoms and does not include polyethylene glycol. In some embodiments, the formulation includes polidocanol in an amount of about 1.25% w/w and from about 1.0% to about 5.0% w/w of an alcohol having 3 to 6 carbon atoms and does not include ethanol or an ether. In some embodiments, the formulation includes polidocanol in an amount of about 1.25% w/w and from about 1.0% to about 5.0% w/w of an alcohol having 3 to 6 carbon atoms and does not include aliphatic polyethers. In some embodiments, the formulation includes polidocanol in an amount of about 1.25% w/w and from about 1.0% to about 5.0% w/w of an alcohol having 3 to 6 carbon atoms and does not include poloxamers.

In some embodiments, the formulation includes polidocanol in an amount of about 2.0% w/w and from about 1.0% to about 5.0% w/w of an alcohol having 3 to 6 carbon atoms. In some embodiments, the formulation includes polidocanol in an amount of about 2.0% w/w and approximately 5.0% w/w of an alcohol having 3 to 6 carbon atoms. In some embodiments, the formulation includes polidocanol in an amount of about 2.0% w/w and from about 1.0% to about 5.0% w/w of an alcohol having 3 to 6 carbon atoms and does not include polyethylene glycol. In some embodiments, the formulation includes polidocanol in an amount of about 2.0% w/w and from about 1.0% to about 5.0% w/w of an alcohol having 3 to 6 carbon atoms and does not include ethanol or an ether. In some embodiments, the formulation includes polidocanol in an amount of about 2.0% w/w and from about 1.0% to about 5.0% w/w of an alcohol having 3 to 6 carbon atoms and does not include aliphatic polyethers. In some embodiments, the formulation includes polidocanol in an amount of about 2.0% w/w and from about 1.0% to about 5.0% w/w of an alcohol having 3 to 6 carbon atoms and does not include poloxamers.

In some embodiments, described herein are formulations including an effective amount, for example a therapeutically or cosmetically effective amount, of polidocanol and from 0.5% w/w to 10% w/w of propylene glycol. In some embodiments, described herein are formulations including an effective amount of polidocanol and from 0.5% w/w to 10% w/w of glycerine. Propylene glycol and glycerine are small, water soluble, hydroxylated organic molecules, which ensure formulation stability and safety when used with polidocanol. Both propylene glycol and glycerine are nearly non-toxic and non-irritating and suitable for subcutaneous injection. In some embodiments, the formulation includes an effective amount, example a therapeutically or cosmetically effective amount, of polidocanol and 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, or 10% w/w propylene glycol and does not contain ethanol or an ether. In some embodiments, the formulation includes an effective amount, for example a therapeutically or cosmetically effective amount, of polidocanol and 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, or 10% w/w glycerine and does not contain ethanol or an ether. In some embodiments, the formulation includes an effective amount, for example a therapeutically or cosmetically effective amount, of polidocanol and from 0.5% to 10% w/w of propylene glycol and does not contain aliphatic polyethers. In some embodiments, the formulation includes an effective amount, for example a therapeutically or cosmetically effective amount, of polidocanol and from 0.5% to 10% w/w of glycerine and does not contain aliphatic polyethers. In some embodiments, the formulation includes an effective amount, for example a therapeutically or cosmetically effective amount, of polidocanol and from 0.5% to 10% w/w of propylene glycol and does not contain polyethylene glycol. In some embodiments, the formulation includes an effective amount, for example a therapeutically or cosmetically effective amount, of polidocanol and from 0.5% to 10% w/w of glycerine and does not contain polyethylene glycol. In some embodiments, the formulation includes an effective amount, for example a therapeutically or cosmetically effective amount, of polidocanol and from 0.5% to 10% w/w of propylene glycol and does not contain poloxamers. In some embodiments, the formulation includes an effective amount, for example a therapeutically or cosmetically effective amount, of polidocanol and from 0.5% to 10% w/w of glycerine and does not contain poloxamers. In some embodiments, the formulation is for subcutaneous injection. In some embodiments, the formulation is for injection into the submental region of a human in need. In some embodiments, the formulation is for injection into a fat pad.

In some embodiments, the formulation includes an effective amount, for example a therapeutically or cosmetically effective amount, of polidocanol and from 1.0% to 5.0% w/w of an alcohol having 3 to 6 carbon atoms. In some embodiments, the formulation includes an effective amount, for example a therapeutically or cosmetically effective amount, of polidocanol and 5.0% w/w of an alcohol having 3 to 6 carbon atoms. In some embodiments, the formulation includes polidocanol in an amount that is greater than 1.0% w/w and from 1.0% to 5.0% w/w of an alcohol having 3 to 6 carbon atoms. In some embodiments, the formulation includes polidocanol in an amount that is greater than 1.0% w/w and 5.0% w/w of an alcohol having 3 to 6 carbon atoms. In some embodiments, the formulation includes polidocanol in an amount that is greater than 1.0% w/w and from 1.0% to 5.0% w/w of an alcohol having 3 to 6 carbon atoms and does not contain polyethylene glycol. In some embodiments, the formulation includes polidocanol in an amount that is greater than 1.0% w/w and from 1.0% to 5.0% w/w of an alcohol having 3 to 6 carbon atoms and does not contain ethanol or an ether. In some embodiments, the formulation includes polidocanol in an amount that is greater than 1.0% w/w and from 1.0% to 5.0% w/w of an alcohol having 3 to 6 carbon atoms and does not contain aliphatic polyethers. In some embodiments, the formulation includes polidocanol in an amount that is greater than 1.0% w/w and from 1.0% to 5.0% w/w of an alcohol having 3 to 6 carbon atoms and does not contain poloxamers.

In embodiments, the formulation includes polidocanol in an amount from 0.1% w/w to 20.0% w/w and from 1.0% to 5.0% w/w of an alcohol having 3 to 6 carbon atoms. In embodiments, the formulation includes polidocanol in an amount from 0.1% w/w to 20.0% w/w and 5.0% w/w of an alcohol having 3 to 6 carbon atoms. In some embodiments, the formulation includes polidocanol in an amount from 0.1% w/w to 20.0% w/w and from 1.0% to 5.0% w/w of an alcohol having 3 to 6 carbon atoms and does not include polyethylene glycol. In some embodiments, the formulation includes polidocanol in an amount from 0.1% w/w to 20.0% w/w and from 1.0% to 5.0% w/w of an alcohol having 3 to 6 carbon atoms and does not include ethanol or an ether. In some embodiments, the formulation includes polidocanol in an amount from 0.1% w/w to 20.0% w/w and from 1.0% to 5.0% w/w of an alcohol having 3 to 6 carbon atoms and does not include aliphatic polyethers. In some embodiments, the formulation includes polidocanol in an amount from 0.1% w/w to 20.0% w/w and from 1.0% to 5.0% w/w of an alcohol having 3 to 6 carbon atoms and does not include poloxamers.

In some embodiments, the formulation includes polidocanol in an amount from 0.5% w/w to 10.0% w/w and from 1.0% to 5.0% w/w of an alcohol having 3 to 6 carbon atoms. In some embodiments, the formulation includes polidocanol in an amount from 0.5% w/w to 10.0% w/w and 5.0% w/w of an alcohol having 3 to 6 carbon atoms. In some embodiments, the formulation includes polidocanol in an amount from 0.5% w/w to 10.0% w/w and from 1.0% to 5.0% w/w of an alcohol having 3 to 6 carbon atoms and does not include polyethylene glycol. In some embodiments, the formulation includes polidocanol in an amount from 0.5% w/w to 10.0% w/w and from 1.0% to 5.0% w/w of an alcohol having 3 to 6 carbon atoms and does not include ethanol or an ether. In some embodiments, the formulation includes polidocanol in an amount from 0.5% w/w to 10.0% w/w and from 1.0% to 5.0% w/w of an alcohol having 3 to 6 carbon atoms and does not include aliphatic polyethers. In some embodiments, the formulation includes polidocanol in an amount from 0.5% w/w to 10.0% w/w and from 1.0% to 5.0% w/w of an alcohol having 3 to 6 carbon atoms and does not include poloxamers.

In some embodiments, the formulation includes polidocanol in an amount from 0.5% w/w to 2.0% w/w and from 1.0% to 5.0% w/w of an alcohol having 3 to 6 carbon atoms. In some embodiments, the formulation includes polidocanol in an amount from 0.5% w/w to 2.0% w/w and 5.0% w/w of an alcohol having 3 to 6 carbon atoms. In some embodiments, the formulation includes polidocanol in an amount from 0.5% w/w to 2.0% w/w and from 1.0% to 5.0% w/w of an alcohol having 3 to 6 carbon atoms and does not include polyethylene glycol. In some embodiments, the formulation includes polidocanol in an amount from 0.5% w/w to 2.0% w/w and from 1.0% to 5.0% w/w of an alcohol having 3 to 6 carbon atoms and does not include ethanol or an ether. In some embodiments, the formulation includes polidocanol in an amount from 0.5% w/w to 2.0% w/w and from 1.0% to 5.0% w/w of an alcohol having 3 to 6 carbon atoms and does not include aliphatic polyethers. In some embodiments, the formulation includes polidocanol in an amount from 0.5% w/w to 2.0% w/w and from 1.0% to 5.0% w/w of an alcohol having 3 to 6 carbon atoms and does not include poloxamers.

In some embodiments, the formulation includes polidocanol in an amount of 0.5% w/w and from 1.0% to 5.0% w/w of an alcohol having 3 to 6 carbon atoms. In some embodiments, the formulation includes polidocanol in an amount of 0.5% w/w and 5.0% w/w of an alcohol having 3 to 6 carbon atoms. In some embodiments, the formulation includes polidocanol in an amount of 0.5% w/w and from 1.0% to 5.0% w/w of an alcohol having 3 to 6 carbon atoms and does not include polyethylene glycol. In some embodiments, the formulation includes polidocanol in an amount of 0.5% w/w and from 1.0% to 5.0% w/w of an alcohol having 3 to 6 carbon atoms and does not include ethanol or an ether. In some embodiments, the formulation includes polidocanol in an amount of 0.5% w/w and from 1.0% to 5.0% w/w of an alcohol having 3 to 6 carbon atoms and does not include aliphatic polyethers. In some embodiments, the formulation includes polidocanol in an amount of 0.5% w/w and from 1.0% to 5.0% w/w of an alcohol having 3 to 6 carbon atoms and does not include poloxamers.

In some embodiments, the formulation includes polidocanol in an amount of 1.25% w/w and from 1.0% to 5.0% w/w of an alcohol having 3 to 6 carbon atoms. In some embodiments, the formulation includes polidocanol in an amount of 1.25% w/w and 5.0% w/w of an alcohol having 3 to 6 carbon atoms. In some embodiments, the formulation includes polidocanol in an amount of 1.25% w/w and from 1.0% to 5.0% w/w of an alcohol having 3 to 6 carbon atoms and does not include polyethylene glycol. In some embodiments, the formulation includes polidocanol in an amount of 1.25% w/w and from 1.0% to 5.0% w/w of an alcohol having 3 to 6 carbon atoms and does not include ethanol or an ether. In some embodiments, the formulation includes polidocanol in an amount of 1.25% w/w and from 1.0% to 5.0% w/w of an alcohol having 3 to 6 carbon atoms and does not include aliphatic polyethers. In some embodiments, the formulation includes polidocanol in an amount of 1.25% w/w and from 1.0% to 5.0% w/w of an alcohol having 3 to 6 carbon atoms and does not include poloxamers.

In some embodiments, the formulation includes polidocanol in an amount of 2.0% w/w and from 1.0% to 5.0% w/w of an alcohol having 3 to 6 carbon atoms. In some embodiments, the formulation includes polidocanol in an amount of 2.0% w/w and 5.0% w/w of an alcohol having 3 to 6 carbon atoms. In some embodiments, the formulation includes polidocanol in an amount of 2.0% w/w and from 1.0% to 5.0% w/w of an alcohol having 3 to 6 carbon atoms and does not include polyethylene glycol. In some embodiments, the formulation includes polidocanol in an amount of 2.0% w/w and from 1.0% to 5.0% w/w of an alcohol having 3 to 6 carbon atoms and does not include ethanol or an ether. In some embodiments, the formulation includes polidocanol in an amount of 2.0% w/w and from 1.0% to 5.0% w/w of an alcohol having 3 to 6 carbon atoms and does not include aliphatic polyethers. In some embodiments, the formulation includes polidocanol in an amount of 2.0% w/w and from 1.0% to 5.0% w/w of an alcohol having 3 to 6 carbon atoms and does not include poloxamers.

In some embodiments the osmolality of the formulation is less than 700 milliosmoles. In some embodiments the osmolality of the formulation is less than 400 milliosmoles. In some embodiments the formulation has 5% propylene glycol and less than 700 milliosmoles. In some embodiments the formulation has 2.5% propylene glycol and less than 400 milliosmoles. In some embodiments the osmolality of the formulation is less than 700 milliosmoles/kg. In some embodiments the osmolality of the formulation is less than 400/kg milliosmoles. In some embodiments the formulation has 5% propylene glycol and less than 700 milliosmoles/kg. In some embodiments the formulation has 2.5% propylene glycol and less than 400 milliosmoles/kg.

In some embodiments, the formulation has a pH from about 6 to about 9. In some embodiments, the formulation has a pH of about 7 to about 8. In some embodiments, the formulation includes polidocanol in about 0.5% w/w and about 5.0% propylene glycol and does not include polyethylene glycol or poloxamers and has a pH of about 7 to about 8 and is formulated for subcutaneous injection into the submental region of a human in need. In some embodiments, the formulation includes polidocanol in about 0.5% w/w and about 5.0% glycerine and does not include polyethylene glycol or poloxamers and has a pH of about 7 to about 8 and is formulated for subcutaneous injection into the submental region of a human in need. In some embodiments, the formulation includes polidocanol in an amount of about 1.25% w/w and about 5.0% propylene glycol and does not include polyethylene glycol or poloxamers and has a pH of about 7 to about 8 and is formulated for subcutaneous injection into the submental region of a human in need. In some embodiments, the formulation includes polidocanol in an amount of about 1.25% w/w and about 5.0% glycerine and does not include polyethylene glycol or poloxamers and has a pH of about 7 to about 8 and is formulated for subcutaneous injection into the submental region of a human in need. In some embodiments, the formulation includes polidocanol in an amount of about 2.0% w/w and about 5.0% propylene glycol and does not include polyethylene glycol or poloxamers and has a pH of about 7 to about 8 and is formulated for subcutaneous injection into the submental region of a human in need. In some embodiments, the formulation includes polidocanol in an amount of about 2.0% w/w and about 5.0% glycerine and does not include polyethylene glycol or poloxamers and has a pH of about 7 to about 8 and is formulated for subcutaneous injection into the submental region of a human in need. In some embodiments, the formulation has a pH from 6 to 9. In some embodiments, the formulation has a pH of 7 to 8. In some embodiments, the formulation includes polidocanol in 0.5% w/w and 5.0% propylene glycol and does not include polyethylene glycol or poloxamers and has a pH of 7 to 8 and is formulated for subcutaneous injection into the submental region of a human in need. In some embodiments, the formulation includes polidocanol in 0.5% w/w and 5.0% glycerine and does not include polyethylene glycol or poloxamers and has a pH of 7 to 8 and is formulated for subcutaneous injection into the submental region of a human in need. In some embodiments, the formulation includes polidocanol in an amount of 1.25% w/w and 5.0% propylene glycol and does not include polyethylene glycol or poloxamers and has a pH of 7 to 8 and is formulated for subcutaneous injection into the submental region of a human in need. In some embodiments, the formulation includes polidocanol in an amount of 1.25% w/w and 5.0% glycerine and does not include polyethylene glycol or poloxamers and has a pH of 7 to 8 and is formulated for subcutaneous injection into the submental region of a human in need. In some embodiments, the formulation includes polidocanol in an amount of 2.0% w/wand 5.0% propylene glycol and does not include polyethylene glycol or poloxamers and has a pH of 7 to 8 and is formulated for subcutaneous injection into the submental region of a human in need. In some embodiments, the formulation includes polidocanol in an amount of 2.0% w/w and 5.0% glycerine and does not include polyethylene glycol or poloxamers and has a pH of 7 to 8 and is formulated for subcutaneous injection into the submental region of a human in need.

### Injection Columns

In some embodiments, the formulations described herein are injectable by means of subcutaneous injection. Subcutaneous administration has the benefit of local, targeted administration. Unlike other systemic modes of administration, subcutaneous administration results in a localized distribution of the active agents. In certain embodiments, these injectable formulations include a compound of Formula I or Formula II (e.g., polidocanol) or a pharmaceutically acceptable or cosmetically acceptable salt or other suitable form thereof in an injection volume that is from about 0.01 mL to about 100 mL, and each volume in between this range, and further including ranges from about 0.01 mL to about 100 mL, 0.5 mL to about 50 mL, from about 1 mL to about 25 mL, from about 2 mL to about 20 mL, from about 3 mL to about 10 mL.

In some embodiments, the injectable formulation includes a compound of Formula I or Formula II (e.g., polidocanol) or a pharmaceutically acceptable or cosmetically acceptable salt or other suitable form thereof in an injection volume that is equal to or less than about 2 mL. In some other embodiments the compound of Formula I or Formula II or a pharmaceutically acceptable or cosmetically acceptable salt or other suitable form thereof injection volume is equal to or more than about 0.05 mL to an injection volume that is equal to or less than about 2 mL. In certain other embodiments, the compound of Formula I or Formula II or a pharmaceutically acceptable or cosmetically acceptable salt or other suitable form thereof injection volume is equal to or more than about 0.1 mL to an injection volume that is equal to or less than about 1.8 mL. In some embodiments, the compound of Formula I or Formula II or a pharmaceutically acceptable or cosmetically acceptable salt or other suitable form thereof injection volume is equal to or more than about 0.2 mL to an injection volume that is equal to or less than about 1.6 mL. In certain embodiments, the compound of Formula I or Formula II or a pharmaceutically acceptable or cosmetically acceptable salt or other suitable form thereof injection volume is equal to or more than about 0.3 mL to an injection volume that is equal to or less than about 1.4 mL. In select embodiments, the compound of Formula I or Formula II or a pharmaceutically acceptable or cosmetically acceptable salt or other suitable form thereof injection volume is equal to or more than about 0.4 mL to an injection volume that is equal to or less than about 1.2 mL. In some embodiments, the compound of Formula I or Formula II or a pharmaceutically acceptable or cosmetically acceptable salt or other suitable form thereof injection volume is equal to or more than about 0.5 mL to an injection volume that is equal to or less than about 1 mL. In some embodiments, a compound of Formula I or Formula II or a pharmaceutically acceptable or cosmetically acceptable salt or other suitable form thereof injection volume is equal to about 0.1 mL. In some further embodiments is provided a compound of Formula I or Formula II or a pharmaceutically acceptable or cosmetically acceptable salt or other suitable form thereof injection volume is equal to about 0.2 mL. In certain embodiments, a compound of Formula I or Formula II or a pharmaceutically acceptable or cosmetically acceptable salt or other suitable form thereof injection volume is equal to about 0.5 mL. In select embodiments, a compound of Formula I or Formula II or a pharmaceutically acceptable or cosmetically acceptable salt or other suitable form thereof injection volume is equal to about 1 mL. In certain embodiments, a compound of Formula I or Formula II or a pharmaceutically acceptable or cosmetically acceptable salt or other suitable form thereof injection volume is equal to about 2 mL.

In an aspect, provided is an injectable formulation for treating regional adipose tissue, regional adiposity, or regional fat accumulation including: an effective amount of at least one of a compound of Formula I or Formula II or a pharmaceutically acceptable or cosmetically acceptable salt or other suitable form thereof; and a liquid carrier; the compound of Formula I or Formula II or a pharmaceutically acceptable or cosmetically acceptable salt or other suitable form thereof and the liquid carrier formulated for injection into a layer of subcutaneous fat in a human in need.

In certain embodiments provided is a formulation including a compound of Formula I or Formula II, including polidocanol and polidocanol-like compounds, or a pharmaceutically acceptable or cosmetically acceptable salt or other suitable form thereof wherein the injection volume is equal to or less than about 2 mL. In certain other embodiments, the injection volume is equal to or more than about 0.05 mL to an injection volume that is equal to or less than about 2 mL. In an embodiment, the injection volume is equal to or more than about 0.1 mL to an injection volume that is equal to or less than about 1.8 mL. In some embodiments, the injection volume is equal to or more than about 0.2 mL to an injection volume that is equal to or less than about 1.6 mL. In an embodiment, the injection volume is equal to or more than about 0.3 mL to an injection volume that is equal to or less than about 1.4 mL. Also provided are embodiments wherein injection volume is equal to or more than about 0.4 mL to an injection volume that is equal to or less than about 1.2 mL. In an embodiment, the injection volume is equal to or more than about 0.5 mL to an injection volume that is equal to or less than about 1 mL. In one embodiment is a formulation, including a compound of Formula II such as polidocanol, or a pharmaceutically acceptable or cosmetically acceptable salt or others suitable form thereof wherein the injection volume is equal to about 0.1 mL. In certain embodiments, the injection volume is equal to about 0.2 mL. In an embodiment, the injection volume is equal to about 0.5 mL. In some embodiments, the injection volume is equal to about 1 mL. In an embodiment, the injection volume is equal to about 2 mL.

In certain embodiments, greater than 1% mg/mL of a compound of Formula I or Formula II, including polidocanol and polidocanol-like compounds, or a pharmaceutically acceptable or cosmetically acceptable salt or other suitable form thereof is provided as a sterile aqueous solution of at least 5 mL to provide adequate coverage area upon administration to an animal, including a human. In certain embodiments, less than 0.5 mL of the solution is injected in a plurality of locations into subcutaneous fat. For example, in one embodiment, the plurality of injections are spaced approximately from about 1 to 5 centimeters apart and form a grid like pattern over the fat treatment area, such as the submentum. In another embodiment, at least greater than 50 mg of a compound of Formula I or Formula II, including polidocanol and polidocanol-like compounds, or a pharmaceutically acceptable or cosmetically acceptable salt or other suitable form thereof in 5 mL water for injection with up to 10% (v/v) of an alcohol having 3 to 6 carbon atoms adjusted to a pH of 6.5-8.0 using disodium hydrogen phosphate dehydrate, potassium dihydrogen phosphate, or other suitable buffering agent. In further or additional embodiments, an alcohol having 3 to 6 carbon atoms is replaced at 0.1-10% (v/v) with other solubilizers such as glycerin or cyclodextrins to improve injection tolerability. Alternative pH buffering agents may include citric acid, sodium bicarbonate, sodium phosphate, and the like. In some embodiments, the formulation includes a compound that is suitable for adjusting pH of the formulation as a solution to provide greater stability of the formulation during storage and prior to administration to a patient. For example, in certain embodiments the pH is adjusted to be in the range of from about 4-12, or from about 5-11, or from about 6-10, from about 6 to 8.5, from about 6.5-8, or from about 7-9.

In some embodiments, the formulations described herein are injectable by means of subcutaneous injection. Subcutaneous administration has the benefit of local, targeted administration. Unlike other systemic modes of administration, subcutaneous administration results in a localized distribution of the active agents. In certain embodiments, these injectable formulations include a compound of Formula I or Formula II (e.g., polidocanol) or a pharmaceutically acceptable or cosmetically acceptable salt or other suitable form thereof in an injection volume that is from about 0.01 mL to about 100 mL, and each volume in between this range, and further including ranges from about 0.01 mL to about 100 mL, 0.5 mL to about 50 mL, from about 1 mL to about 25 mL, from about 2 mL to about 20 mL, from about 3 mL to about 10 mL.

In some embodiments, the injectable formulation includes a compound of Formula I or Formula II (e.g., polidocanol) or a pharmaceutically acceptable or cosmetically acceptable salt or other suitable form thereof in an injection volume that is equal to or less than about 2 mL. In embodiments, the injection volume is about 0.05 mL to about 2 mL. In embodiments, the injection volume is about 0.1 mL to about 1.8 mL. In embodiments, the injection volume is about 0.2 mL to about 1.6 mL. In embodiments, the injection volume is about 0.3 mL to about 1.4 mL. In embodiments, the injection volume is about 0.4 mL to about 1.2 mL. In embodiments, the injection volume is about 0.5 mL to about 1 mL. In embodiments, the injection volume is about 0.1 mL. In embodiments, the injection volume is about 0.2 mL. In embodiments, the injection volume is about 0.5 mL. In embodiments, the injection volume is about 1 mL. In embodiments, the injection volume is about 2 mL.

In an aspect, provided is an injectable formulation for treating regional adipose tissue, regional adiposity, or regional fat accumulation including: an effective amount of at least one of a compound of Formula I or Formula II (e.g., polidocanol) or a pharmaceutically acceptable or cosmetically acceptable salt or other suitable form thereof; and a liquid carrier; the compound of Formula I or Formula II (e.g., polidocanol) or a pharmaceutically acceptable or cosmetically acceptable salt or other suitable form thereof and the liquid carrier formulated for injection into a layer of subcutaneous fat in a human in need.

In embodiments, the injection volume is less than about 2 mL. In embodiments, the injection volume is about 0.05 mL to about 2 mL. In embodiments, the injection volume is about 0.1 mL to about 1.8 mL. In embodiments, the injection volume is about 0.2 mL to about 1.6 mL. In embodiments, the injection volume is about 0.3 mL to about 1.4 mL. In embodiments, the injection volume is about 0.4 mL to about 1.2 mL. In embodiments, the injection volume is about 0.5 mL to about 1 mL. In embodiments, the injection volume is about 0.1 mL. In embodiments, the injection volume is about 0.2 mL. In embodiments, the injection volume is about 0.5 mL. In embodiments, the injection volume is about 1 mL. In embodiments, the injection volume is about 2 mL.

In certain embodiments, greater than 1% mg/mL of a compound of Formula I or Formula II, including polidocanol and polidocanol-like compounds, or a pharmaceutically acceptable or cosmetically acceptable salt or other suitable form thereof is provided as a sterile aqueous solution of at least 5 mL to provide adequate coverage area upon administration to an animal, including a human. In certain embodiments, less than 0.5 mL of the solution is injected in a plurality of locations into subcutaneous fat. For example, in one embodiment, the plurality of injections are spaced approximately from about 1 to 5 centimeters apart and form a grid like pattern over the fat treatment area, such as the submentum. In another embodiment, at least greater than 50 mg of a compound of Formula I or Formula II, including polidocanol and polidocanol-like compounds, or a pharmaceutically acceptable or cosmetically acceptable salt or other suitable form thereof in 5 mL water for injection with up to 10% (v/v) of an alcohol having 3 to 6 carbon atoms adjusted to a pH of 6.5-8.0 using disodium hydrogen phosphate dehydrate, potassium dihydrogen phosphate, or other suitable buffering agent. In further or additional embodiments, an alcohol having 3 to 6 carbon atoms is replaced at 0.1-10% (v/v) with other solubilizes such as PEG 300, PEG 400, Polysorbate (TWEEN®) 80, Glycerin, cyclodextrins, or CREMOPHOR® to improve injection tolerability. Alternative pH buffering agents may include citric acid, sodium bicarbonate, sodium phosphate, and the like. In some embodiments, the formulation includes a compound that is suitable for adjusting pH of the formulation as a solution to provide greater stability of the formulation during storage and prior to administration to a patient. For example, in certain embodiments the pH is adjusted to be in the range of from about 4-12, or from about 5-11, or from about 6-10, from about 6 to 8.5, from about 6.5-8, or from about 7-9.

In some embodiments, the formulations described herein are injectable by means of subcutaneous injection. Subcutaneous administration has the benefit of local, targeted administration. Unlike other systemic modes of administration, subcutaneous administration results in a localized distribution of the active agents. In certain embodiments, these injectable formulations include a compound of Formula I or Formula II (e.g., polidocanol) or a pharmaceutically acceptable or cosmetically acceptable salt or other suitable form thereof in an injection volume that is from 0.01 mL to 100 mL, and each volume in between this range, and further including ranges from 0.01 mL to 100 mL, 0.5 mL to 50 mL, from 1 mL to 25 mL, from 2 mL to 20 mL, from 3 mL to 10 mL.

In some embodiments, the injectable formulation includes a compound of Formula I or Formula II (e.g., polidocanol) or a pharmaceutically acceptable or cosmetically acceptable salt or other suitable form thereof in an injection volume that is equal to or less than 2 mL. In embodiments, the injection volume is 0.05 mL to 2 mL. In embodiments, the injection volume is 0.1 mL to 1.8 mL. In embodiments, the injection volume is 0.2 mL to 1.6 mL. In embodiments, the injection volume is 0.3 mL to 1.4 mL. In embodiments, the injection volume is 0.4 mL to 1.2 mL. In embodiments, the injection volume is 0.5 mL to 1 mL. In embodiments, the injection volume is 0.1 mL. In embodiments, the injection volume is 0.2 mL. In embodiments, the injection volume is 0.5 mL. In embodiments, the injection volume is 1 mL. In embodiments, the injection volume is 2 mL.

In an aspect, provided is an injectable formulation for treating regional adipose tissue, regional adiposity, or regional fat accumulation including: an effective amount of at least one of a compound of Formula I or Formula II (e.g., polidocanol) or a pharmaceutically acceptable or cosmetically acceptable salt or other suitable form thereof; and a liquid carrier; the compound of Formula I or Formula II (e.g., polidocanol) or a pharmaceutically acceptable or cosmetically acceptable salt or other suitable form thereof and the liquid carrier formulated for injection into a layer of subcutaneous fat in a human in need.

In embodiments, the injection volume is less than 2 mL. In embodiments, the injection volume is 0.05 mL to 2 mL. In embodiments, the injection volume is 0.1 mL to 1.8 mL. In embodiments, the injection volume is 0.2 mL to 1.6 mL. In embodiments, the injection volume is 0.3 mL to 1.4 mL. In embodiments, the injection volume is 0.4 mL to 1.2 mL. In embodiments, the injection volume is 0.5 mL to 1 mL. In embodiments, the injection volume is 0.1 mL. In embodiments, the injection volume is 0.2 mL. In embodiments, the injection volume is 0.5 mL. In embodiments, the injection volume is 1 mL. In embodiments, the injection volume is 2 mL.

In certain embodiments, greater than 1% mg/mL of a compound of Formula I or Formula II, including polidocanol and polidocanol-like compounds, or a pharmaceutically acceptable or cosmetically acceptable salt or other suitable form thereof is provided as a sterile aqueous solution of at least 5 mL to provide adequate coverage area upon administration to an animal, including a human. In certain embodiments, less than 0.5 mL of the solution is injected in a plurality of locations into subcutaneous fat. For example, in one embodiment, the plurality of injections are spaced from 1 to 5 centimeters apart and form a grid like pattern over the fat treatment area, such as the submentum. In another embodiment, at least greater than 50 mg of a compound of Formula I or Formula II, including polidocanol and polidocanol-like compounds, or a pharmaceutically acceptable or cosmetically acceptable salt or other suitable form thereof in 5 mL water for injection with up to 10% (v/v) of an alcohol having 3 to 6 carbon atoms adjusted to a pH of 6.5-8.0 using disodium hydrogen phosphate dehydrate, potassium dihydrogen phosphate, or other suitable buffering agent. In further or additional embodiments, an alcohol having 3 to 6 carbon atoms is replaced at 0.1-10% (v/v) with other solubilizes such as PEG 300, PEG 400, Polysorbate (TWEEN®) 80, Glycerin, cyclodextrins, or CREMOPHOR® to improve injection tolerability. Alternative pH buffering agents may include citric acid, sodium bicarbonate, sodium phosphate, and the like.

In some embodiments, the formulation includes a compound that is suitable for adjusting pH of the formulation as a solution to provide greater stability of the formulation during storage and prior to administration to a patient. For example, in certain embodiments the pH is adjusted to be in the range of from 4-12, or from 5-11, or from 6-10, from 6 to 8.5, from 6.5-8, or from 7-9.

### Treatment Sites of Compounds of Formula I and II

In certain embodiments, the formulations described herein are administered or provided to the individual in the inside region of the knees, the middle to upper area of the upper arm (including the tricep area), the sub-mental area, the abdomen, the hips, the inner thigh, the outer thigh, the buttocks, the lower back, the upper back, or the chest.

In certain embodiments, the formulation is an extended release formulation. In certain other embodiments, the formulation is a rapid-release or immediate release formulation. In some embodiments, the therapeutically effective amount of the polidocanol is released for about 12 hours to about 45 days (e.g., about 3 days to about 10 days).

In some embodiments, the therapeutically effective amount of the polidocanol is released for 12 hours to 45 days (e.g., 3 days to 10 days).

In another aspect, provided herein are methods and formulations that facilitate dispersal of polidocanol, or a polidocanol-like compound of Formula II and salts and other suitable forms thereof into a layer of subcutaneous fat at a regional fat site selected from one or more of the following: a sub-mental region, an abdominal region, a waist, a hip, a lateral buttock, a thigh, a peri-orbital region and intramuscular region. In certain embodiments provided is a formulation for the treatment of one or more of: abdominal fat accumulation, regional adiposity, excessive adiposity in the sub-mental region, and exophthalmos caused by thyroid eye disease. In certain embodiments are provided formulations to affect a shape, contour, or appearance of the human body.

In some embodiments, the subject to be treated is provided a non-sustained release formulation. In some embodiments, the non-sustained release formulation, after a single dose, provides activity of the compound of Formula II such as polidocanol for a duration from about 4 hours to about 24 hours, e.g., about 6 hours, 8 hours, 10 hours, 12 hours, 16 hours, 18 hours, 21 hours, or any other duration of polidocanol from about four hours to about 24 hours. In some embodiments, the subject to be treated is provided a non-sustained release formulation. In some embodiments, the non-sustained release formulation, after a single dose, provides activity of the compound of Formula II such as polidocanol for a duration from 4 hours to 24 hours, e.g., 6 hours, 8 hours, 10 hours, 12 hours, 16 hours, 18 hours, 21 hours, or any other duration of polidocanol from four hours to 24 hours.

In another aspect provided herein are methods and formulations that facilitate dispersal of polidocanol, polidocanol-like compounds, and salts and other suitable forms thereof into a layer of subcutaneous fat at a regional fat site selected from one or more of the following: a sub-mental region, an abdominal region, a waist, a hip, a lateral buttock, a thigh, a peri-orbital region, an intra-orbital region, and intramuscular region, and combinations thereof. In certain embodiments, provided is a formulation for the treatment of one or more of: abdominal fat accumulation, regional adiposity, exophthalmos caused by thyroid eye disease, and excess adipose or fat tissue in the sub-mental region, which commonly presents as a double chin. In certain embodiments provided are formulations to affect a shape, contour, or appearance of the human body.

In embodiments, a pharmaceutical formulation as described herein (including in an aspect, embodiment, claim, example, table, or figure) is used in a method described herein (including in an aspect, embodiment, claim, example, table, or figure). In embodiments, an injectable formulation as described herein (including in an aspect, embodiment, claim, example, table, or figure) is used in a method described herein (including in an aspect, embodiment, claim, example, table, or figure). In embodiments, a formulation as described herein as described herein (including in an aspect, embodiment, claim, example, table, or figure) is used in a method described herein (including in an aspect, embodiment, claim, example, table, or figure). In embodiments, a composition (e.g., pharmaceutical composition, injectable composition) as described herein as described herein (including in an aspect, embodiment, claim, example, table, or figure) is used in a method described herein (including in an aspect, embodiment, claim, example, table, or figure).

### Kits

In an aspect is provided a kit, including: a cosmetically or therapeutically effective amount, including for example a therapeutically or cosmetically effective amount, of a compound of Formula I or Formula II (e.g., polidocanol) or a pharmaceutically acceptable or cosmetically acceptable salt or other suitable form thereof; an injector; and instructions for use. In certain embodiments of the kit, the compound of Formula I or Formula II (e.g., polidocanol) or a pharmaceutically acceptable or cosmetically acceptable salt or other suitable form thereof is in an aqueous form. In certain embodiments of the kit, the polidocanol or a pharmaceutically acceptable or cosmetically acceptable salt or other suitable form thereof is in an aqueous form. In certain embodiments of the kit, the compound of Formula I or Formula II (e.g., polidocanol) or a pharmaceutically acceptable or cosmetically acceptable salt or other suitable form thereof is in crystalline phase. In certain other embodiments, the compound of Formula I or Formula II (e.g., polidocanol) or a pharmaceutically acceptable or cosmetically acceptable salt or other suitable form thereof is in an amorphous phase. In an embodiment, the compound of Formula I or Formula II (e.g., polidocanol) or a pharmaceutically acceptable or cosmetically acceptable salt or other suitable form thereof is in a semi-crystalline phase. In certain embodiments of the kit, the compound of Formula I or Formula II (e.g., polidocanol) or a pharmaceutically acceptable or cosmetically acceptable salt or other suitable form thereof is in a semi-amorphous phase. In some embodiments, a compound of Formula I or Formula II (e.g., polidocanol) or a pharmaceutically acceptable or cosmetically acceptable salt or other suitable form thereof is in a crystalline or amorphous form. In some embodiments of the kit described herein, the compound of Formula II is polidocanol. In certain embodiments of the kit, the injector contains a needle, is needleless, or includes a subcutaneous applicator. In embodiments, the polidocanol is present in an amount of about 0.1%-10% w/w or about 0.1%-5% w/w. In some embodiments, the polidocanol is present in an amount of about 0.1% w/w to about 10% w/w. In some embodiments, the polidocanol is present in an amount of about 0.1% w/w to about 0.2% w/w, about 0.1% w/w to about 0.5% w/w, about 0.1% w/w to about 1% w/w, about 0.1% w/w to about 2% w/w, about 0.1% w/w to about 3% w/w, about 0.1% w/w to about 4% w/w, about 0.1% w/w to about 4.5% w/w, about 0.1% w/w to about 5% w/w, about 0.1% w/w to about 6% w/w, about 0.1% w/w to about 8% w/w, about 0.1% w/w to about 10% w/w, about 0.2% w/w to about 0.5% w/w, about 0.2% w/w to about 1% w/w, about 0.2% w/w to about 2% w/w, about 0.2% w/w to about 3% w/w, about 0.2% w/w to about 4% w/w, about 0.2% w/w to about 4.5% w/w, about 0.2% w/w to about 5% w/w, about 0.2% w/w to about 6% w/w, about 0.2% w/w to about 8% w/w, about 0.2% w/w to about 10% w/w, about 0.5% w/w to about 1% w/w, about 0.5% w/w to about 2% w/w, about 0.5% w/w to about 3% w/w, about 0.5% w/w to about 4% w/w, about 0.5% w/w to about 4.5% w/w, about 0.5% w/w to about 5% w/w, about 0.5% w/w to about 6% w/w, about 0.5% w/w to about 8% w/w, about 0.5% w/w to about 10% w/w, about 1% w/w to about 2% w/w, about 1% w/w to about 3% w/w, about 1% w/w to about 4% w/w, about 1% w/w to about 4.5% w/w, about 1% w/w to about 5% w/w, about 1% w/w to about 6% w/w, about 1% w/w to about 8% w/w, about 1% w/w to about 10% w/w, about 2% w/w to about 3% w/w, about 2% w/w to about 4% w/w, about 2% w/w to about 4.5% w/w, about 2% w/w to about 5% w/w, about 2% w/w to about 6% w/w, about 2% w/w to about 8% w/w, about 2% w/w to about 10% w/w, about 3% w/w to about 4% w/w, about 3% w/w to about 4.5% w/w, about 3% w/w to about 5% w/w, about 3% w/w to about 6% w/w, about 3% w/w to about 8% w/w, about 3% w/w to about 10% w/w, about 4% w/w to about 4.5% w/w, about 4% w/w to about 5% w/w, about 4% w/w to about 6% w/w, about 4% w/w to about 8% w/w, about 4% w/w to about 10% w/w, about 4.5% w/w to about 5% w/w, about 4.5% w/w to about 6% w/w, about 4.5% w/w to about 8% w/w, about 4.5% w/w to about 10% w/w, about 5% w/w to about 6% w/w, about 5% w/w to about 8% w/w, about 5% w/w to about 10% w/w, about 6% w/w to about 8% w/w, about 6% w/w to about 10% w/w, or about 8% w/w to about 10% w/w. In some embodiments, the polidocanol is present in an amount of about 0.1% w/w, about 0.2% w/w, about 0.5% w/w, about 1% w/w, about 2% w/w, about 3% w/w, about 4% w/w, about 4.5% w/w, about 5% w/w, about 6% w/w, about 8% w/w, or about 10% w/w. In some embodiments, the polidocanol is present in an amount of at least about 0.1% w/w, about 0.2% w/w, about 0.5% w/w, about 1% w/w, about 2% w/w, about 3% w/w, about 4% w/w, about 4.5% w/w, about 5% w/w, about 6% w/w, or about 8% w/w. In some embodiments, the polidocanol is present in an amount of at most about 0.2% w/w, about 0.5% w/w, about 1% w/w, about 2% w/w, about 3% w/w, about 4% w/w, about 4.5% w/w, about 5% w/w, about 6% w/w, about 8% w/w, or about 10% w/w. In some embodiments, the polidocanol is present in an amount of about 2.0% w/w. In some embodiments, the polidocanol is present in an amount of about 3.0% w/w. In some embodiments, the polidocanol is present in an amount of about 4.5% w/w.

In an aspect is provided a kit for treating adipose tissue including the described formulations and instructions for use. In some embodiments, described herein are kits for treating adipose tissue including a formulation including an effective amount, for example a therapeutically or cosmetically effective amount, of polidocanol and propylene glycol (e.g., from about 0.5% w/w to about 10% w/w) and instructions for use. In some embodiments, described herein are kits for treating adipose tissue including a formulation including an effective amount, for example a therapeutically or cosmetically effective amount, of polidocanol and glycerine (e.g., from about 0.5% w/w to about 10% w/w) and instructions for use. In some embodiments, the kits for treating adipose tissue include a formulation including an effective amount, for example a therapeutically or cosmetically effective amount, of polidocanol and propylene glycol (from about 0.5% w/w to about 10% w/w) wherein the formulation does not contain ethanol or an ether. In some embodiments, the kits for treating adipose tissue include a formulation including an effective amount, for example a therapeutically or cosmetically effective amount of polidocanol and glycerine (from about 0.5% w/w to about 10% w/w) wherein the formulation does not contain ethanol or an ether. In some embodiments, the kits for treating adipose tissue include a formulation including an effective amount, including for example a therapeutically or cosmetically effective amount, of polidocanol and propylene glycol (e.g., from about 0.5% w/w to about 10% w/w) wherein the formulation does not contain aliphatic polyethers, such as polyethylene glycol or poloxamers. In some embodiments, the kits for treating adipose tissue include a formulation including an effective amount, including for example a therapeutically or cosmetically effective amount, of polidocanol and glycerine (e.g., from about 0.5% w/w to about 10% w/w) wherein the formulation does not contain aliphatic polyethers, such as polyethylene glycol or poloxamers. In some embodiments, the formulations do not contain ethanol or an ether or aliphatic polyethers such as polyethylene glycol or poloxamers.

In an aspect is provided a kit for treating adipose tissue including the described formulations and instructions for use. In some embodiments, described herein are kits for treating adipose tissue including a formulation including an effective amount, for example a therapeutically or cosmetically effective amount, of polidocanol and propylene glycol (e.g., from 0.5% w/w to 10% w/w) and instructions for use. In some embodiments, described herein are kits for treating adipose tissue including a formulation including an effective amount, for example a therapeutically or cosmetically effective amount, of polidocanol and glycerine (e.g., from 0.5% w/w to 10% w/w) and instructions for use. In some embodiments, the kits for treating adipose tissue include a formulation including an effective amount, for example a therapeutically or cosmetically effective amount, of polidocanol and propylene glycol (from 0.5% w/w to 10% w/w) wherein the formulation does not contain ethanol or an ether. In some embodiments, the kits for treating adipose tissue include a formulation including an effective amount, for example a therapeutically or cosmetically effective amount of polidocanol and glycerine (from 0.5% w/w to 10% w/w) wherein the formulation does not contain ethanol or an ether. In some embodiments, the kits for treating adipose tissue include a formulation including an effective amount, including for example a therapeutically or cosmetically effective amount, of polidocanol and propylene glycol (e.g., from 0.5% w/w to 10% w/w) wherein the formulation does not contain aliphatic polyethers, such as polyethylene glycol or poloxamers. In some embodiments, the kits for treating adipose tissue include a formulation including an effective amount, including for example a therapeutically or cosmetically effective amount, of polidocanol and glycerine (e.g., from 0.5% w/w to 10% w/w) wherein the formulation does not contain aliphatic polyethers, such as polyethylene glycol or poloxamers. In some embodiments, the formulations do not contain ethanol or an ether or aliphatic polyethers such as polyethylene glycol or poloxamers.

In an aspect is provided a kit for treating adipose tissue including a formulation including an effective amount, for example a therapeutically or cosmetically effective amount, of polidocanol and an alcohol having 3 to 6 carbon atoms (e.g., from about 0.5% w/w to about 10% w/w) and instructions for use. In some embodiments, the alcohol having 3 to 6 carbon atoms is present in an amount of from about 1% w/w to about 5% w/w. In some embodiments, the alcohol having 3 to 6 carbon atoms is present in an amount of approximately 5% w/w. In some embodiments, the kit includes a formulation including polidocanol in an amount that is greater than 1.0% w/w and from about 0.5% w/w to about 10% w/w of an alcohol having 3 to 6 carbon atoms. In some embodiments, the kit includes a formulation including polidocanol in an amount from about 0.1% w/w to about 20.0% w/w and from about 0.5% w/w to about 10% w/w of an alcohol having 3 to 6 carbon atoms. In some embodiments, the kit includes a formulation including polidocanol in an amount from about 0.5% w/w to about 10.0% w/w and from about 0.5% w/w to about 10% w/w of an alcohol having 3 to 6 carbon atoms. In some embodiments, the kit includes a formulation including polidocanol in an amount from about 0.1% w/w to about 2.0% w/w and from about 0.5% w/w to about 10% w/w of an alcohol having 3 to 6 carbon atoms. In some embodiments, the kit includes a formulation including polidocanol in an amount of about 0.5% w/w, 1.25%, or 2.0% w/w and from about 0.5% w/w to about 10% w/w of an alcohol having 3 to 6 carbon atoms. In some embodiments, the formulation has a pH of from about 6 to 9. In some embodiments, the formulation has a pH of from about 7 to 8.

In an aspect is provided a kit for treating adipose tissue including a formulation including an effective amount, for example a therapeutically or cosmetically effective amount, of polidocanol and an alcohol having 3 to 6 carbon atoms (e.g., from 0.5% w/w to 10% w/w) and instructions for use. In some embodiments, the alcohol having 3 to 6 carbon atoms is present in an amount of from 1% w/w to 5% w/w. In some embodiments, the alcohol having 3 to 6 carbon atoms is present in an amount of 5% w/w. In some embodiments, the kit includes a formulation including polidocanol in an amount that is greater than 1.0% w/w and from 0.5% w/w to 10% w/w of an alcohol having 3 to 6 carbon atoms. In some embodiments, the kit includes a formulation including polidocanol in an amount from 0.1% w/w to 20.0% w/w and from 0.5% w/w to 10% w/w of an alcohol having 3 to 6 carbon atoms. In some embodiments, the kit includes a formulation including polidocanol in an amount from 0.5% w/w to 10.0% w/w and from 0.5% w/w to 10% w/w of an alcohol having 3 to 6 carbon atoms. In some embodiments, the kit includes a formulation including polidocanol in an amount from 0.1% w/w to 2.0% w/w and from 0.5% w/w to 10% w/w of an alcohol having 3 to 6 carbon atoms. In some embodiments, the kit includes a formulation including polidocanol in an amount of 0.5% w/w, 1.25%, or 2.0% w/w and from 0.5% w/w to 10% w/w of an alcohol having 3 to 6 carbon atoms. In some embodiments, the formulation has a pH of from 6 to 9. In some embodiments, the formulation has a pH of from 7 to 8.

In an aspect, provided herein is a kit, said kit including: a cosmetically or therapeutically effective amount of at least one of a compound of Formula I or Formula II such as polidocanol or a pharmaceutically acceptable or cosmetically acceptable salt or other suitable form thereof; an injector; and instructions for use. In certain embodiments, the kit includes polidocanol or a pharmaceutically acceptable or cosmetically acceptable salt or other suitable form thereof in an aqueous form. In some embodiments, the polidocanol or a pharmaceutically acceptable or cosmetically acceptable salt or other suitable form thereof is in crystalline phase. In an embodiment, the polidocanol or a pharmaceutically acceptable or cosmetically acceptable salt or other suitable form thereof is in an amorphous phase. In a certain embodiment, the polidocanol or a pharmaceutically acceptable or cosmetically acceptable salt or other suitable form thereof is in a semi-crystalline phase. In certain embodiments, the polidocanol or a pharmaceutically acceptable or cosmetically acceptable salt or other suitable form thereof is in a semi-amorphous phase. In an embodiment, the polidocanol or a pharmaceutically acceptable or cosmetically acceptable salt or other suitable form thereof is in a crystalline or amorphous form. In certain embodiments of the kit, the injector contains a needle, is needleless, or includes a subcutaneous applicator.

In certain embodiments, provided herein are kits, including: a cosmetically or therapeutically effective amount of polidocanol, a compound of Formula I, a compound of Formula II, a polidocanol-like compound of Formula II or a pharmaceutically acceptable or cosmetically acceptable salt or other suitable form thereof; an injector; and instructions for use. In embodiments, the kit does not include a lipase. In embodiments, the kit does not include a colipase.

While certain embodiments have been described, these embodiments have been presented by way of example only, and are not intended to limit the scope of the disclosure. The formulations, methods, and systems described herein may be embodied in a variety of other forms. Furthermore, various omissions, substitutions and changes in the form of the formulations, methods, and systems described herein may be made without departing from the spirit of this disclosure. The accompanying claims and their equivalents are intended to cover such forms or modifications.

In embodiments, the kit includes a pharmaceutical formulation as described herein (including in an aspect, embodiment, claim, example, table, or figure). In embodiments, the kit includes an injectable formulation as described herein (including in an aspect, embodiment, claim, example, table, or figure). In embodiments, the kit includes a formulation as described herein (including in an aspect, embodiment, claim, example, table, or figure). In embodiments, the kit includes a composition (e.g., pharmaceutical composition, injectable composition) as described herein (including in an aspect, embodiment, claim, example, table, or figure).

### EXAMPLES

### Example 1. a Multi-Center, Randomized, Double-Blind, Vehicle-Controlled Study of the Safety and Efficacy if Polidocanol (10XB-101) in Adult Subjects with Submental Fat

This example characterized the safety and efficacy of repeated treatment sessions of 10XB-101 with that of the vehicle control in adult subjects with localized subcutaneous fat in the submental area. After enrollment in the study, each subject was randomized (1:1:1:1) to one of the following four treatment groups. An unblinded pharmacist at each site diluted the 10XB-101 solution, 6.0% at treatment visits to achieve the concentration required for each active treatment group or use the correct strength for the assigned treatment group to prepare the test article for injection. Each subject remained in the same treatment group and receive the same corresponding dose concentration for the entire study period. Treatment group A were subjects treated with 2.0% polidocanol. Treatment group B would be subjects treated with 3.0% polidocanol. Treatment group C were subjects treated with 4.5% polidocanol. Treatment group D were subjects treated with vehicle control. Each subject could receive up to 6 treatments over 20 weeks, per protocol, with the test article they were randomized to at Baseline. The treatment duration was approximately 12 months for an individual subject with male or female subjects 18 to 65 years of age with excessive submental fat (SMF). Approximately 52 subjects (13 per treatment group) would be enrolled at three sites. Inclusion criteria for this study included: a male or non-pregnant female 18 to 65 years old; subject had provided written informed consent; females must be post-menopausal, surgically sterile, or use an effective method of birth control; women of childbearing potential (WOCBP) must have a negative urine pregnancy test (UPT) at visit 2/baseline; subject had a score of 3 on the Clinician Submental Fat Scale (CSFS) at Visit 2/Baseline; subject had unwanted submental fat and a score of 3 on the Patient Submental Fat Scale (PSFS) at Visit 2/Baseline; subject had a stable body weight for the past 6 months prior to Visit 2/Baseline; subject agreed to not change dietary or exercise practices or start a weight reduction regimen during the course of the study; subject was willing to undergo test article injections as directed, comply with study instructions, and commit to all follow-up visits for the duration of the study; or subject, in the investigator's opinion, was in good general health and free of any disease state or physical condition that might impair evaluation of the test article injection sites or exposes the subject to an unacceptable risk by study participation. Exclusion criteria for this study included subject was a subject who was pregnant, lactating, or was planning to become pregnant during the study; subject had loose skin (skin laxity) in the submental area, diffuse SMF, or prominent platysma bands at rest that may interfere with evaluation of localized fat, in the opinion of the investigator; subject had a body mass index>40 kg/m²; subject has had any prior treatment for SMF (e.g., deoxycholic acid, cryotherapy, liposuction, surgery); subject had laser, chemical peel, or dermal filler treatments in the submental area within 12 months prior to Visit 2/Baseline; subject had botulinum toxin treatments in the submental area within 6 months prior to Visit 2/Baseline; subject had any scars, facial hair, tattoos, or other features that may interfere with the evaluation of localized fat, in the opinion in the investigator; subject had any medical condition that affects clotting and/or platelet function (e.g., thromboembolic disease, clotting factor deficiencies such as hemophilia); subject had a clinically significant abnormal coagulation test result at Visit 1/Screening; subject is taking any medications that affect clotting and/or platelet function within 7 days prior to Visit 2/Baseline (this included, but was not limited to, nonsteroidal anti-inflammatory medications such as low dose aspirin for prophylaxis, heparin such as low molecular weight heparin, coumadin, clopidogrel, and factor Xa agents such as apixaban. Subjects may not discontinue chronically administered anti-thrombotic medications in order to qualify for the study); subject was immunocompromised, in the opinion of the investigator, based on their medical condition (e.g., human immunodeficiency virus positive, malignancy), medication use, or other factors; subject had any clinically significant medical abnormality or chronic disease of the cardiovascular, gastrointestinal, respiratory (e.g., chronic obstructive pulmonary disease), hepatic, or renal systems in the opinion of the investigator; subject was already enrolled in an investigational drug, biologic, or device study; subject had used an investigational drug, investigational biologic, or investigational device treatment within 30 days prior to Visit 2/Baseline; subject had a history of allergy or sensitivity to polidocanol or any of the other ingredients in the test articles; subject was known to be noncompliant or is unlikely to comply with the requirements of the study protocol (e.g., due to alcoholism, drug dependency, mental incapacity) in the opinion of the investigator; or Subject had history or current symptoms of dysphagia.

Test articles for this study included: 10XB-101 (polidocanol) solution for injection, 2.0%; 10XB-101 (polidocanol) solution for injection, 3.0%; 10XB-101 (polidocanol) solution for injection, 4.5%; 10XB-101 (polidocanol) solution for injection, 6.0%; and vehicle solution for injection. 10XB-101 (polidocanol) solution for subcutaneous injection is a sterile, aqueous, preservative-free injectable solution with polidocanol as the active pharmaceutical ingredient. 10XB-101 injection was manufactured in a current Good Manufacturing Practice-compliant facility and tested prior to release to ensure that each clinical batch meets defined quality specifications. Table 1 illustrates the test article name, active ingredient, and other ingredients. Tables 2-4 illustrate non-limiting examples of the 10XB-101 (polidocanol) solution.

TABLE 1
Test article composition

| | Active | |
|---|---|---|
| Test article name | ingredient | Other ingredients |
| 10XB-101 (polidocanol) | Polidocanol | Propylene glycol, sodium phosphate |
| Solution for Injection, 2.0% | | dibasic dihydrate, monobasic |
| | | potassium phosphate, and water for |
| | | injection |
| 10XB-101 (polidocanol) | Polidocanol | Propylene glycol, sodium phosphate |
| Solution for Injection, 3.0% | | dibasic dihydrate, monobasic |
| | | potassium phosphate, and water for |
| | | injection |
| 10XB-101 (polidocanol) | Polidocanol | Propylene glycol, sodium phosphate |
| Solution for Injection, 4.5% | | dibasic dihydrate, monobasic |
| | | potassium phosphate, and water for |
| | | injection |
| 10XB-101 (polidocanol) | Polidocanol | Propylene glycol, sodium phosphate |
| Solution for Injection, 6.0% | | dibasic dihydrate, monobasic |
| | | potassium phosphate, and water for |
| | | injection |
| Vehicle Solution for | — | Propylene glycol, sodium phosphate |
| Injection | | dibasic dihydrate, monobasic |
| | | potassium phosphate, and water for |
| | | injection |

TABLE 2
Composition of 10XB-101 (Polidocanol) Injection Drug Product, 2%

| | Unit Formula | | Composition | | |
|---|---|---|---|---|---|
| Ingredient | (per mL) | | (% w/w) | IID Limit | IID Routes |
| Polidocanol | 20.0 | mg | 2.0 | N/A | N/A |
| Propylene Glycol | 21.0 | mg | 2.1 | 1.4% w/w; 5% w/w | SQ; IV |
| Sodium Phosphate | 2.4 | mg | 0.24 | 0.14% w/w; 55 mg | SQ; IV |
| Dibasic Dihydrate | | | | | |
| Potassium | 0.85 | mg | 0.085 | 8 mg; 1.36% w/w | SQ; IV |
| Phosphate | | | | | |
| Monobasic | | | | | |
| Water for Injection | 955.75 | mg | 95.575 | N/A | N/A |

IID = Inactive ingredient database,
N/A = not applicable,
SQ = subcutaneous,
IV = intravenous

TABLE 3
Composition of 10XB-101 (Polidocanol) Injection Drug Product, 3%

| | Unit Formula | | Composition | | |
|---|---|---|---|---|---|
| Ingredient | (per mL) | | (% w/w) | IID Limit | IID Routes |
| Polidocanol | 30.0 | mg | 3.0 | N/A | N/A |
| Propylene Glycol | 21.0 | mg | 2.1 | 1.4% w/w; 5% w/w | SQ; IV |
| Sodium Phosphate | 2.4 | mg | 0.24 | 0.14% w/w; 55 mg | SQ; IV |
| Dibasic Dihydrate | | | | | |
| Potassium | 0.85 | mg | 0.085 | 8 mg; 1.36% w/w | SQ; IV |
| Phosphate | | | | | |
| Monobasic | | | | | |
| Water for Injection | 945.75 | mg | 94.575 | N/A | N/A |

IID = Inactive ingredient database,
N/A = not applicable,
SQ = subcutaneous,
IV = intravenous

TABLE 4
Composition of 10XB-101 (Polidocanol) Injection Drug Product, 4.5%

| | Unit Formula | | Composition | | |
|---|---|---|---|---|---|
| Ingredient | (per mL) | | (% w/w) | IID Limit | IID Routes |
| Polidocanol | 45.0 | mg | 4.5 | N/A | N/A |
| Propylene Glycol | 21.0 | mg | 2.1 | 1.4% w/w; 5% w/w | SQ; IV |
| Sodium Phosphate | 2.4 | mg | 0.24 | 0.14% w/w; 55 mg | SQ; IV |
| Dibasic Dihydrate | | | | | |
| Potassium | 0.85 | mg | 0.085 | 8 mg; 1.36% w/w | SQ; IV |
| Phosphate | | | | | |
| Monobasic | | | | | |
| Water for Injection | 930.75 | mg | 93.075 | N/A | N/A |

IID = Inactive ingredient database,
N/A = not applicable,
SQ = subcutaneous,
IV = intravenous

### Repeat Treatment Visits and Dose Modifications

Before each subsequent treatment session, the investigator evaluated (i) the clinical appearance and distribution of SMF and (ii) all AEs with an emphasis on local AEs and injection zone local skin reaction (LSR) associated with prior treatment(s) to assess the need for: a) modification of the number of injections, b) treatment delay or skipping a treatment, or c) termination of further treatment. If reductions in SMF occurred during the study treatment period, the number of injections should be adjusted to effectively treat only those regions with clinically evident residual SMF. In those regions where excessive SMF had resolved, no additional treatment would be administered. Although the number of injections administered may be reduced to match the SMF reduction pattern over time, in no case would the volume of a single injection be modified from 0.2 mL/injection nor would it be diluted in any manner. In the event all excessive SMF had resolved (CSFS=0) at any follow-up treatment visit, no additional treatments would be administered and the subject would be required to complete protocol-specified assessments at Visits 8, 9, and 10. At each follow-up treatment visit (Visits 3, 4, 5, 6 and 7), if local AEs or injection zone LSRs required a delay in the treatment in the opinion of the investigator, the visit may be rescheduled within a 7-14 day period relative to the scheduled day of treatment. If absolutely necessary, a given treatment may also be skipped. If the repeat treatment interval was delayed more than 12 weeks due to continuation of the local AE(s) that caused the delay, the subject will be withdrawn from treatment but would be required to complete Visits 8, 9, and 10 to document the resolution of the AE(s) and complete protocol-specified assessments.

### Warnings, Precautions, and Contraindications

Severe allergic reactions, including anaphylaxis (which in some cases has been fatal), had been reported related to the use of injectable polidocanol for sclerotherapy. Although such reactions were rare, severe reactions were reported more frequently with larger systemic volumes of polidocanol (>3 mL) introduced intravascularly. Investigators and their staff were experienced in the management of severe allergic reactions, including anaphylaxis, and be prepared to treat allergic reactions or even anaphylaxis if it occurred. These test articles were for subcutaneous use only. Intravascular administration could result in tissue ischemia, necrosis, or possibly risk related to embolism. Parenteral drug products were inspected visually for particulate matter and discoloration prior to administration, whenever solution and container permit. The test article was contraindicated in the presence of infection at the injection sites/zones. Subjects with a known allergy or sensitivity to any of the ingredients in the test articles should not participate in this study. The effects of the test article in nursing mothers, pregnant women, and their unborn children were unknown. WOCBP must not be pregnant or planning a pregnancy during the study period.

### Randomization Assignment

Subjects were randomized on a 1:1:1:1 basis to one of 4 treatment groups: treatment group A, 2.0% polidocanol; treatment group B, 3.0% polidocanol; treatment group C, 4.5% polidocanol; and treatment group D, vehicle control. The randomization was blocked by investigational site using a series of consecutively numbered randomization envelopes. The unblinded pharmacist would randomize each subject by opening the envelope with the lowest available randomization number. The randomized treatment group assignment for the subject would be specified on the piece of paper inside the envelope. The unblinded pharmacist would record the subject number who received that randomization, the date and time of randomization, sign and date the form, and kelp it in their secured pharmacy records. Subjects who discontinued from the study would not be replaced.

### Prior and Concomitant Therapies

Use of medications/therapies such as any medications/therapies taken in the 30 days prior to the start of the study (Visit 1/Screening), and medications/therapies that required washout for more than 30 days would be recorded as concomitant medications or concurrent procedures, respectively, with the dose (for medications only) and corresponding indication. The medications to be recorded included prescription and over-the-counter medications; vitamins, minerals, and herbal, holistic, and dietary supplements would only be recorded as concomitant medications if being taken for a therapeutic indication. All medications taken on a regular basis must be recorded on the electronic case report forms (eCRFs). All concomitant medications would be coded with the current version of the WHO Drug Dictionary at the start of the study. Therapies to be recorded included any non-drug therapy used to treat a medical condition. Procedures that occurred prior to Baseline would be recorded as medical history. Any changes in concomitant medications and/or therapies during the study must be recorded. The reason for any changes in concomitant medications and/or therapies was needed and would typically reflect either a baseline medical condition documented in the medical history or in conjunction with an adverse event (AE).

### Prohibited Medications or Therapies

Prior to entry into the study, subjects must not use the medications or procedures/therapies. The following medications or treatments were prohibited during the study: any treatment for SMF (e.g., deoxycholic acid, cryotherapy, liposuction, surgery); botulinum toxin, laser, chemical peel, or dermal filler treatments in the submental area; medications that affected clotting and/or platelet function; or any investigational drug, investigational biologic, or investigational device.

### Allowed Medications or Therapies

Vitamins and mineral supplements were permitted at dosages considered by the investigator as reasonable for maintaining good health and would not be recorded in the eCRFs. Therapies (medication and non-medication therapies, including cold packs) not restricted by the protocol may be used during the study for the treatment or prevention of disease or to maintain good health. Non-prohibited chronic therapies being used at Visit 1/Screening may be continued, but must be recorded.

### Study Procedures

Specific activities for each study visit were listed below. Note: Subject assessments (i.e., PSFS) would be performed prior to any clinical assessments performed by the investigator. Table 5 illustrates a non-limiting example of a study procedure utilized by this example.

TABLE 5
Non-limiting example of schedule of event

| | Screen | Treatment Period¹ | | | | | | |
|---|---|---|---|---|---|---|---|---|
| | Visit | | | | | | | |
| | | | | Visit | | Visit | | Visit |
| | | Visit | | 3.1² | | 4.1² | | 5.1² |
| | Visit | 2 | Visit | Tx #2 | Visit | Tx #3 | Visit | Tx #4 |
| | 1 | Baseline/ | 3 | Follow- | 4 | Follow- | 5 | Follow- |
| | Screening | Tx #1 | Tx #2 | up | Tx #3 | up | Tx #4 | up |
| | Days | | | | | | | |
| | | | | 7 ± 3 | | 7 ± 3 | | 7 ± 3 |
| | −30 | | | after | | after | | after |
| | to −3 | 1 | 29 | V3 | 57 | V4 | 85 | V5 |
| Informed Consent | X | | | | | | | |
| Demographics | X | | | | | | | |
| Inclusion/Exclusion | Review | X | | | | | | |
| Criteria | | | | | | | | |
| Medical History | X | X | | | | | | |
| Con Medications/ | X | X | X | X | X | X | X | X |
| Con | | | | | | | | |
| Procedures/Therapies | | | | | | | | |
| PSFS | X | X | X | | X | | X | |
| CSFS | X | X | X | | X | | X | |
| Submental Skin Laxity | | X | X | | X | | X | |
| Scale (SSLS)⁴ | | | | | | | | |
| Physical Examination⁵ | | X | | | X | | | |
| Vital Signs⁸ | | X | | | | | | |
| Weight | X | X | X | | | | X | |
| ECG⁹ | | X | | | X | | | |
| Clinical Laboratory | X | X | | | X | | | |
| Tests¹⁰ | | | | | | | | |
| UPT¹¹ for WOCBP¹² | | X | X | | X | | X | |
| Photography¹³ | X | X¹⁴ | X | | X | | X | |
| Clinical Assessment of | | X¹⁵ | X¹⁵ | X | X¹⁵ | X | X¹⁵ | X |
| Injection Zone LSRs | | | | | | | | |
| Randomization¹⁶ | | X | | | | | | |
| Inject Test Article | | X | X | | X | | X | |
| Safety Surveillance¹⁷ | | X | X | | X | | X | |
| Adverse Events¹⁸ | X¹⁹ | X | X | X | X | X | X | X |

| | Treatment Period¹ | | | | Post Treatment Follow-up | | |
|---|---|---|---|---|---|---|---|
| | Visit | | | | | | |
| | | | | | | | Visit |
| | | Visit | | Visit | Visit | Visit | 10 |
| | | 6.1² | | 7.1² | 8 | 9 | +24 wks |
| | Visit | Tx #5 | Visit | Tx #6 | +4 wks | +12 wks | after final |
| | 6 | Follow- | 7 | Follow- | after final | after final | treatment/ |
| | Tx #5 | up | Tx #6 | up | treatment | treatment | EOS³ |
| | Days | | | | | | |
| | | 7 ± 3 | | 7 ± 3 | | | |
| | | after | | after | Follow-up | Follow-up | Follow-up |
| | 113 | V6 | 141 | V7 | 29 ± 5 | 85 ± 7 | 169 ± 14 |
| Informed Consent | | | | | | | |
| Demographics | | | | | | | |
| Inclusion/Exclusion | | | | | | | |
| Criteria | | | | | | | |
| Medical History | | | | | | | |
| Con Medications/ | X | X | X | X | X | X | X |
| Con | | | | | | | |
| Procedures/Therapies | | | | | | | |
| PSFS | X | | X | | X | X | X |
| CSFS | X | | X | | X | X | X |
| Submental Skin Laxity | X | | X | | X | X | X |
| Scale (SSLS)⁴ | | | | | | | |
| Physical Examination⁵ | | | X | | X⁶ | | X^{6, 7} |
| Vital Signs⁸ | | | | | | | X^{6, 7} |
| Weight | X | | X | | X | X | X |
| ECG⁹ | X | | X | | X⁶ | X⁶ | X^{6, 7} |
| Clinical Laboratory | X | | X | | X⁶ | X⁶ | X^{6, 7} |
| Tests¹⁰ | | | | | | | |
| UPT¹¹ for WOCBP¹² | X | | X | | X | | X |
| Photography¹³ | X | | X | | X | X | X |
| Clinical Assessment of | X¹⁵ | X | X¹⁵ | X | X | X | X |
| Injection Zone LSRs | | | | | | | |
| Randomization¹⁶ | | | | | | | |
| Inject Test Article | X | | X | | | | |
| Safety Surveillance¹⁷ | X | | X | | | | |
| Adverse Events¹⁸ | X | X | X | X | X | X | X |

¹Follow up treatment visits are scheduled approximately 4 weeks (i.e., 28 ± 5 days) after the prior treatment. The first follow up treatment is scheduled for Visit 3 (Day 29 ± 5): Week 4. If the subject returns per the ideal visit schedule the follow-up treatment visits will occur as follows; Visit 4 (Day 57 ± 5): Week 8, Visit 5 (Day 85 ± 5): Week 12, Visit 6 (Day 113 ± 5): Week 16, Visit 7 (Day 141 ± 5): Week 20.
²This visit will only occur if treatment was administered at the preceding visit.
³Or early termination from the study.
⁴Subjects already enrolled at the time of this amendment will have skin laxity assessed via SSLS; however, subjects without a baseline assessment will be excluded from any summary analysis as described in the SAP.
⁵Assessments will include examination of head and neck, dermatologic (except submental fat), cardiovascular, respiratory, gastrointestinal (abdomen), and gross motor and gait. Clinically significant abnormalities at Visit 2/Baseline will be recorded as medical history. Any new or worsening clinically significant abnormalities at post-Baseline visits will be recorded as AEs.
⁶Only if the preceding results are materially abnormal and clinically significant in the opinion of the investigator, if applicable.
⁷If a subject is undergoing early termination from the study, physical examination, vital signs, ECG, clinical laboratory tests, and a UPT are required.
⁸Assessments will be made after the subject has rested in a seated position for at least 5 minutes. Height will also be measured at Visit 2/Baseline.
⁹12-lead ECGs will be performed after the subject has rested for at least 10 minutes in the supine position. ECGs are to be administered at every other treatment visit.
¹⁰Subjects must be fasting (approximately 8 hours) for Visit 1/Screening and Visit 7, and if possible, for any other visit. For Visit 1/Screening: if fasting samples cannot be collected at the time of other Visit 1/Screening procedures, the subject may return to the clinic for sample collection such that the results of the clinical laboratory tests are available for review prior to Visit 2/Baseline; such testing will still be considered “Visit 1/Screening” labs. If a subject arrives at the clinic for any other visit without fasting for at least 8 hours, laboratory specimens may be collected as non-fasting, at the discretion of the investigator, and with the appropriate fasting status documented on the lab requisition form. Clinical laboratory tests are to be administered at every other treatment visit.
¹¹UPTs must have a minimum sensitivity of 25 mIU β-hCG/mL.
¹²WOCBP include any female who has experienced menarche or is 10 years of age or older and who has not undergone successful surgical sterilization (hysterectomy, bilateral tubal ligation, or bilateral oophorectomy) or is not postmenopausal (defined as amenorrhea >12 consecutive months in women 50 years of age and older).
¹³Photographs will be taken of the submental region as detailed in the photo guidelines provided to the study site.
¹⁴Pre-test article injection photographs will only be taken at this visit if photographs at Screening are deemed unsatisfactory.
¹⁵Pre- and post-injection LSRs will be assessed in the injection zone.
¹⁶Subject will be assigned the next available (lowest) kit number in ascending order.
¹⁷At visits only where treatment is administered, the investigator will observe the subject for systemic and local safety for approximately 15-20 minutes post-injection to assess if any of the following AEs occur. SYSTEMIC: anaphylactic shock, angioedema, deep vein thrombosis, pulmonary embolism, pyrexia, and circulatory collapse; LOCAL: injection site reactions (necrosis and thrombosis, hypertrichosis, ischemia, gangrene, discoloration, allergic dermatitis, neovascularization, and nerve injury).
¹⁸AEs will be classified based on CTCAE, Version 5.0; any subjects with a new onset, systemic Grade ≥2 AE for cardiac and/or blood/lymphatic system categories (or for subjects that enroll with Grade 2 findings then a 1-point increase to Grade 3 will apply) or other systemic or non-systemic (local) Grade ≥3 AE with no clear alternative causative explanation will be discontinued from test article injections, continued in the study, and followed until resolution of the AE or until the event is deemed clinically stable by the investigator.
¹⁹During any wash out and screening periods, any changes in the health of subjects should be recorded as changes in medical history unless an event occurred as a result of a study-related procedure and is unanticipated; in such cases, the event should be recorded as an AE and reported to the Institutional Review Board (IRB) as an “unanticipated problem” in accordance with local procedures.

Visit 1 (Day −30 to −3): Screening. At this visit, the investigator or designee did: obtain a signed, written informed consent; record demographics; review I/E criteria; record medical history; record prior and/or concomitant medications and concurrent procedures/therapies; have the subject perform the PSFS; perform the CSFS according to the instructions found in the SMF Grading Manual; or record the subject's weight; or collect urine and blood samples for clinical laboratory tests (chemistry, hematology, urinalysis, and coagulation parameters). Subjects must be fasting for approximately 8 hours. If fasting samples could not be collected at the time of other Visit 1/Screening procedures, the subject may return to the clinic for sample collection such that the results of the clinical laboratory tests were available for review prior to Visit 2/Baseline; such testing would still be considered “Visit 1/Screening” labs. Photograph the submental region as detailed in the photo guidelines would be provided to the site. Subject was scheduled for the Baseline visit.

Visit 2 (Day 1): Baseline. At this visit, the investigator or designee did: query the subject for any changes in health status since the previous visit, including concomitant medications and concurrent procedures/therapies, and document the findings; review the screening clinical laboratory test results and assess Common Terminology Criteria for Adverse Events (CTCAE) grades for any analytes that are outside the laboratory normal ranges; review and update medical history, if needed; have the subject perform the PSFS according to the instructions found in the SMF Grading Manual; perform the CSFS according to the instructions found in the SMF Grading Manual; assess submental skin laxity scale (SSLS); Perform a brief physical exam; Record clinically significant abnormalities in medical history; measure vital signs such as height and weight; perform electrocardiogram (ECG); or collect urine and blood samples for clinical laboratory tests. If possible, subjects would be fasting for approximately 8 hours. If for any reason, screening labs were not collected after subject was fasting, these labs must be taken after the subject has fasted for at least 8 hours. Other taskes included performing a urine pregnancy tests (UPTs for all WOCBP (the results must be negative for the subject to be enrolled; confirm inclusion or exclusion criteria; if screening photographs were deemed unsatisfactory, photograph the submental region as detailed in the photo guidelines provided to the site prior to test article injections; perform the Clinical Assessment of Injection Zone LSRs just prior to the test article injections; randomize the subject by assigning the next available (lowest) kit number in ascending order; identify the test article injection zone; inject test article; apply cold packs to the treatment area once the last injection is administered for approximately 10 minutes to mitigate any post injection discomfort; observe the subject for systemic and local safety approximately 15-20 minutes post-injection to assess if any of the following occur. SYSTEMIC: anaphylactic shock, angioedema, deep vein thrombosis, pulmonary embolism, pyrexia, and circulatory collapse; LOCAL: injection site reactions (necrosis and thrombosis, hypertrichosis, ischemia, gangrene, injection site discoloration, allergic dermatitis, neovascularization, and nerve injury); record any findings as AEs; photograph the submental region within approximately 30 minutes after test article injection; perform the Clinical Assessment of Injection Zone LSRs within approximately 30 minutes after test article injection (during this period, it was important for the site to periodically check on the subject to be sure they tolerated the procedure well and were not experiencing any allergic-like reactions or other concerns; or schedule a follow-up visit.

Visit 3 (Day 29): Treatment #2 (28+5 days after Treatment #1). At this visit, the investigator or designee did: query the subject for any changes in health status since the previous visit, including concomitant medications and concurrent procedures/therapies, and document the findings; had the subject perform the PSFS according to the instructions found in the SMF Grading Manual; perform the CSFS according to the instructions found in the SMF Grading Manual. If the CSFS score is 0 the subject has completed the treatment phase of the study and this visit will become the first visit of the follow-up phase (i.e., perform Visit 8 procedures); assess SSLS; record the subject's weight; perform a UPT for all WOCBP (the results must be negative for the subject to receive treatment injections. Any positive results must be followed); photograph the submental region as detailed in the photo guidelines provided to the site prior to test article injections; assess the submental region to determine if treatment will be administered (if local AEs or injection zone LSRs required a treatment delay, in the opinion of the investigator, the treatment was rescheduled within 7-14 days and the remainder of the visit assessments was completed at that time. In addition, record the treatment interruption and its associated AE); perform the Clinical Assessment of Injection Zone LSRs prior to the test article injections; inject test article into the test article injection zone identified at the Baseline visit (applied cold packs to the treatment area once the last injection was administered for approximately 10 minutes to mitigate any post injection discomfort); observe the subject for systemic and local safety approximately 15-20 minutes post-injection to assess if any of the following occur: SYSTEMIC: anaphylactic shock, angioedema, deep vein thrombosis, pulmonary embolism, pyrexia, and circulatory collapse; LOCAL: injection site reactions (necrosis and thrombosis, hypertrichosis, ischemia, gangrene, injection site discoloration, allergic dermatitis, neovascularization, and nerve injury); record any findings as AE; photograph the submental region within approximately 30 minutes after test article injection; perform the Clinical Assessment of Injection Zone LSRs within approximately 30 minutes after test article injection. During this period, it is important for the site to periodically check on the subject to be sure they tolerated the procedure well and are not experiencing any allergic-like reactions or other concerns; document any AEs; or schedule the next visit.

Visit 3.1: Treatment #2 Follow-up (7±3 days after Treatment). This visit would only occur if treatment was administered at Visit 3. At this visit, the investigator or designee did: query the subject for any changes in health status since the previous visit, including concomitant medications and concurrent procedures/therapies, and document the findings; perform the Clinical Assessment of Injection Zone LSRs; document any AEs; or schedule the subject for the next visit.

Visit 4 (Day 57): Treatment #3 (28±5 days after Treatment #2). At this visit, the investigator or designee would: query the subject for any changes in health status since the previous visit, including concomitant medications and concurrent procedures/therapies, and document the findings; have the subject perform the PSFS according to the instructions found in the SMF Grading Manual; perform the CSFS according to the instructions found in the SMF Grading Manual (if the CSFS score is 0 the subject had completed the treatment phase of the study and this visit would become the first visit of the follow-up phase, i.e., perform Visit 8 procedures; assess SSLS; perform a brief physical exam; record any new or worsening clinically significant abnormalities as AEs; measure vital signs including weight; perform ECG; collect urine and blood samples for clinical laboratory tests such as chemistry, hematology, or urinalysis (if possible, the subject would be fasting for approximately 8 hours); perform an UPT for all WOCBP (the results must be negative for the subject to receive treatment injections, and any positive results must be followed); photograph the submental region as detailed in the photo guidelines provided to the site prior to test article injections; assess the submental region to determine if treatment will be administered (if local AEs or injection zone LSRs required a treatment delay, in the opinion of the investigator, treatment was rescheduled within 7-14 days, and the remainder of the visit assessments was completed at that time. In addition, the treatment interruption and its associated AE was recorded); perform the Clinical Assessment of Injection Zone LSRs prior to the test article injections; inject test article into the test article injection zone identified at the Baseline visit; apply cold packs to the treatment area once the last injection was administered for approximately 10 minutes to mitigate any post injection discomfort; observe the subject for systemic and local safety approximately 15-20 minutes post-injection to assess if any of the following occur. SYSTEMIC: anaphylactic shock, angioedema, deep vein thrombosis, pulmonary embolism, pyrexia, and circulatory collapse; LOCAL: injection site reactions (necrosis and thrombosis, hypertrichosis, ischemia, gangrene, injection site discoloration, allergic dermatitis, neovascularization, and nerve injury); record any findings as AEs; photograph the submental region within approximately 30 minutes after test article injection; perform the Clinical Assessment of Injection Zone LSRs within approximately 30 minutes after test article injection (during this period, it was important for the site to periodically check on the subject to be sure they tolerated the procedure well and were not experiencing any allergic-like reactions or other concerns); document any AEs; or schedule the next visit.

Visit 4.1: Treatment #3 Follow-up (7±3 days after Treatment). This visit would only occur if treatment was administered at Visit 4. At this visit, the investigator or designee did: query the subject for any changes in health status since the previous visit, including concomitant medications and concurrent procedures/therapies, and document the findings; perform the Clinical Assessment of Injection Zone LSRs; document any AEs; or schedule the subject for the next visit.

Visit 5 (Day 85): Treatment #4 (28±5 days after Treatment #3). At this visit, the investigator or designee did: query the subject for any changes in health status since the previous visit, including concomitant medications and concurrent procedures/therapies, and document the findings; have the subject perform the PSFS according to the instructions found in the SMF Grading Manual; perform the CSFS according to the instructions found in the SMF Grading Manual (if the CSFS score was 0 the subject had completed the treatment phase of the study and this visit wpi; d become the first visit of the follow-up phase. i.e., perform Visit 8 procedures); assess SSLS; record the subject's weight; perform a UPT for all WOCBP (the results must be negative for the subject to receive treatment injections. Any positive results must be followed); photograph the submental region as detailed in the photo guidelines provided to the site prior to test article injections; assess the submental region to determine if treatment would be administered (if local AEs or injection zone LSRs required a treatment delay, in the opinion of the investigator, the treatment was rescheduled within 7-14 days, and the remainder of the visit assessments was completed at that time. In addition, the treatment interruption and its associated AE was recorded); perform the Clinical Assessment of Injection Zone LSRs prior to the test article injections; inject test article into the test article injection zone identified at the Baseline visit; apply cold packs to the treatment area once the last injection was administered for approximately 10 minutes to mitigate any post injection discomfort; observe the subject for systemic and local safety approximately 15-20 minutes post-injection to assess if any of the following occur. SYSTEMIC: anaphylactic shock, angioedema, deep vein thrombosis, pulmonary embolism, pyrexia, and circulatory collapse; LOCAL: injection site reactions (necrosis and thrombosis, hypertrichosis, ischemia, gangrene, injection site discoloration, allergic dermatitis, neovascularization, and nerve injury); record any findings as AEs; photograph the submental region within approximately 30 minutes after test article injection; perform the Clinical Assessment of Injection Zone LSRs within approximately 30 minutes after test article injection (during this period, it was important for the site to periodically check on the subject to be sure they tolerated the procedure well and were not experiencing any allergic-like reactions or other concerns); document any AEs; or schedule the next visit.

Visit 5.1: Treatment #4 Follow-up (7±3 days after Treatment). This visit would only occur if treatment was administered at Visit 5. At this visit, the investigator or designee did: query the subject for any changes in health status since the previous visit, including concomitant medications and concurrent procedures/therapies, and document the findings; perform the Clinical Assessment of Injection Zone LSRs; document any AEs; or schedule the subject for the next visit.

Visit 6 (Day 113): Treatment #5 (28±5 days after Treatment #4). At this visit, the investigator or designee would: query the subject for any changes in health status since the previous visit, including concomitant medications and concurrent procedures/therapies, and document the findings; have the subject perform the PSFS according to the instructions found in the SMF Grading Manual; perform the CSFS according to the instructions found in the SMF Grading Manual (if the CSFS score was 0, the subject had completed the treatment phase of the study and this visit wpi; d become the first visit of the follow-up phase, i.e., perform Visit 8 procedures); assess SSLS; record the subject's weight; perform ECG; collect urine and blood samples for clinical laboratory tests such as chemistry, hematology, and urinalysis (if possible, the subject would be fasting for approximately 8 hours); perform a UPT for all WOCBP (the results must be negative for the subject to receive treatment injections, and any positive results must be followed); photograph the submental region as detailed in the photo guidelines provided to the site prior to test article injections; assess the submental region to determine if treatment would be administered (if local AEs or injection zone LSRs required a treatment delay, in the opinion of the investigator, the treatment was rescheuled within 7-14 days, and the remainder of the visit assessments was completed at that time/In addition, the treatment interruption and its associated AE was recorded); perform the Clinical Assessment of Injection Zone LSRs prior to the test article injections; inject test article into the test article injection zone identified at the Baseline visit; apply cold packs to the treatment area once the last injection was administered for approximately 10 minutes to mitigate any post injection discomfort; observe the subject for systemic and local safety approximately 15-20 minutes post-injection to assess if any of the following occur: SYSTEMIC: anaphylactic shock, angioedema, deep vein thrombosis, pulmonary embolism, pyrexia, and circulatory collapse; LOCAL: injection site reactions such as necrosis and thrombosis, hypertrichosis, ischemia, gangrene, injection site discoloration, allergic dermatitis, neovascularization, and nerve injury; record any findings as AEs; photograph the submental region within approximately 30 minutes after test article injection; perform the Clinical Assessment of Injection Zone LSRs within approximately 30 minutes after test article injection (during this period, it was important for the site to periodically check on the subject to be sure they tolerated the procedure well and were not experiencing any allergic-like reactions or other concerns); document any AEs; or schedule the next visit.

Visit 6.1: Treatment #5 Follow-up (7±3 days after Treatment). This visit would only occur if treatment was administered at Visit 6. At this visit, the investigator or designee did: query the subject for any changes in health status since the previous visit, including concomitant medications and concurrent procedures/therapies, and document the findings; perform the Clinical Assessment of Injection Zone LSRs; document any AEs; or schedule the subject for the next visit

Visit 7 (Day 141): Treatment #6 (28±5 days after Treatment #5). At this visit, the investigator or designee would: query the subject for any changes in health status since the previous visit, including concomitant medications and concurrent procedures/therapies, and document the findings; have the subject perform the PSFS according to the instructions found in the SMF Grading Manual; perform the CSFS according to the instructions found in the SMF Grading Manual (if the CSFS score was 0, the subject had completed the treatment phase of the study and this visit would become the first visit of the follow-up phase, i.e., perform Visit 8 procedures); assess SSLS; perform a brief physical exam; record any new or worsening clinically significant abnormalities as AEs; measure vital signs including weight; perform ECG; collect urine and blood samples for clinical laboratory tests such as chemistry, hematology, and urinalysis (the subject must be fasting for approximately 8 hours); perform a UPT for all WOCBP (the results must be negative for the subject to receive treatment injections, and any positive results must be followed); photograph the submental region as detailed in the photo guidelines provided to the site prior to test article injections; assess the submental region to determine if treatment would be administered (if local AEs or injection zone LSRs required a treatment delay, in the opinion of the investigator, the treatment was rescheduled within 7-14 days and the remainder of the visit assessments was completed at that time. In addition, the treatment interruption and its associated AE was recorded); perform the Clinical Assessment of Injection Zone LSRs prior to the test article injections; inject test article into the test article injection zone identified at the Baseline visit; apply cold packs to the treatment area once the last injection was administered for approximately 10 minutes to mitigate any post injection discomfort; observe the subject for systemic and local safety approximately 15-20 minutes post-injection to assess if any of the following occurred: SYSTEMIC: anaphylactic shock, angioedema, deep vein thrombosis, pulmonary embolism, pyrexia, and circulatory collapse; LOCAL: injection site reactions such as necrosis and thrombosis, hypertrichosis, ischemia, gangrene, injection site discoloration, allergic dermatitis, neovascularization, and nerve injury; record any findings as AEs; photograph the submental region within approximately 30 minutes after test article injection; perform the Clinical Assessment of Injection Zone LSRs within approximately 30 minutes after test article injection (during this period, it was important for the site to periodically check on the subject to be sure they tolerated the procedure well and were not experiencing any allergic-like reactions or other concerns; document any AEs; or schedule the next visit.

Visit 7.1: Treatment #6 Follow-up (7±3 days after Treatment). This visit would only occur if treatment was administered at Visit 7. At this visit, the investigator or designee did: query the subject for any changes in health status since the previous visit, including concomitant medications and concurrent procedures/therapies, and document the findings; perform the Clinical Assessment of Injection Zone LSRs; document any AEs; or schedule the subject for the next visit.

Visit 8 (Follow-up Day 29±5): 4 weeks after final treatment. This visit would occur approximately 4 weeks after the subject's final test article injection. At this visit, the investigator or designee would: query the subject for any changes in health status since the previous visit, including concomitant medications and concurrent procedures/therapies, and document the findings; have the subject perform the PSFS according to the instructions found in the SMF Grading Manual; perform the CSFS according to the instructions found in the SMF Grading Manual; assess SSLS; record the subject's weight; perform the following assessments, as appropriate, if any of the preceding results were materially abnormal and clinically significant: perform a brief physical exam; record any new or worsening clinically significant abnormalities as AEs; measure vital signs; perform ECG; collect urine and blood samples for clinical laboratory tests such as chemistry, hematology, and urinalysis (if possible, the subject would be fasting for approximately 8 hours); perform a UPT for all WOCBP (any positive results must be followed); photograph the submental region as detailed in the photo guidelines provided to the site; perform the Clinical Assessment of Injection Zone LSRs; document any AEs; or schedule the next visit.

Visit 9 (Follow-up Day 85±7): 12 weeks after final treatment. This visit would occur approximately 12 weeks after the subject's final test article injection. At this visit, the investigator or designee did: query the subject for any changes in health status since the previous visit, including concomitant medications and concurrent procedures/therapies, and document the findings; have the subject perform the PSFS according to the instructions found in the SMF Grading Manual; perform the CSFS according to the instructions found in the SMF Grading Manual; assess SSLS; record the subject's weight; perform the following assessments, as appropriate, if any of the preceding results were materially abnormal and clinically significant; perform ECG; collect urine and blood samples for clinical laboratory tests such as chemistry, hematology, and urinalysis (if possible, the subject would be fasting for approximately 8 hours); photograph the submental region as detailed in the photo guidelines provided to the site; perform the Clinical Assessment of Injection Zone LSRs; document any AEs; or schedule the next visit.

Visit 10 (Follow-up Day 169±14): 24 weeks after final treatment or early termination. This visit would occur approximately 24 weeks after the subject's final test article injection. At this visit, the investigator or designee did: query the subject for any changes in health status since the previous visit, including concomitant medications and concurrent procedures/therapies, and document the findings; have the subject perform the PSFS according to the instructions found in the SMF Grading Manual; perform the CSFS according to the instructions found in the SMF Grading Manual; or Assess SSLS; Record the subject's weight. If the subject was undergoing early termination from the study, perform the following assessments: perform a brief physical exam; record any new or worsening clinically significant abnormalities as AEs; measure vital signs; perform ECG; collect urine and blood samples for clinical laboratory tests such as chemistry, hematology, and urinalysis (if possible, the subject would be fasting for approximately 8 hours); or perform a UPT for all WOCBP (any positive results must be followed).

If this is Visit 10, the following assessments, as appropriate, was performed if any of the preceding results were materially abnormal and clinically significant: perform a brief physical exam; record any new or worsening clinically significant abnormalities as AEs; measure vital signs; perform ECG; collect urine and blood samples for clinical laboratory tests such as chemistry, hematology, and urinalysis (if possible, the subject would be fasting for approximately 8 hours); photograph the submental region as detailed in the photo guidelines provided to the site; perform the Clinical Assessment of Injection Zone LSRs; document any AEs; or exit the subject from the study.

### Clinical Evaluations

The following clinical evaluations would be performed. For the CSFS, SSLS, and injection zone LSR assessments, the same expert grader who had received training on how to properly perform the clinical evaluations should ideally complete the evaluations for a given subject throughout the study. If this was not possible (e.g., scheduling conflict), a different expert grader with overlapping experience with the subject and the study may complete the evaluations.

Clinician Submental Fat Scale. The CSFS was a discrete scale based on the investigator's clinical evaluation of the subject as the subject appears on the day of the evaluation. The CSFS score would be based on the investigator's clinical evaluation of the subject, including palpation of the chin and neck area; anterior, oblique, and profile views of the chin and neck; and observation of pronation, supination, and lateral movement of the head. Each site would be provided with the SMF Grading Manual instructing the investigators on how to grade subjects using the CSFS. The investigator or designee grader would select a best grade (0 to 4) on this 5-point static scale that best describes the subject's level of SMF according to provided representative pictures (in the SMF Grading Manual) and the following scale: score of 0 was no submental fat bulge; score of 1 was mild submental fat bulge; score of 2 was moderate submental fat bulge; score of 3 was severe submental fat bulge; and score of 4 was very severe submental fat bulge. FIG. 1 illustrates a visual representation of the scoring of the SMF.

Patient Submental Fat Scale. The PSFS is a single question survey that the subject would be asked to answer based on how the subject feels about their SMF on the day of the evaluation. The subject would position their head in the Frankfort plane and used a mirror to look at the area under their chin to help them answer the survey. Instructions in the SMF Grading Manual would be used by site staff to train the subject on proper head placement and mirror used during their assessment.

Submental Skin Laxity Scale. Submental skin laxity was measured on a 4-point discrete scale from 0-None to 3-Severe based on the investigator's clinical evaluation of the subject as the subject appears on the day of the evaluation. The SSLS did assess skin wrinkling, adherence to underlying neck structures, and redundancy (horizontal and vertical folds). The investigator would assess submental skin laxity, including palpation of the chin and neck area (anterior oblique, and profile views of the chin and neck), as well as observation of pronation, supination, and lateral movement of the head; however, the final determination of the submental skin laxity would be made while the head is in the Frankfort plane posture. The scale in Table 6 would be used.

TABLE 6
SSLS Scoring

| Score | Description |
|---|---|
| 0-None | No or minimal superficial wrinkling |
| | Skin well opposed to deeper neck structures |
| | No skin redundancy (without any vertical or |
| | horizontal folds) |
| 1-Mild | Mild superficial wrinkling |
| | Skin well opposed to deeper neck structures |
| | Minimal skin redundancy (slight vertical |
| | folds with no horizontal folds) |
| 2-Moderate | May have moderate superficial wrinkling |
| | Skin has mild to moderate separation from |
| | deeper neck structures |
| | Moderate skin redundancy (moderate vertical |
| | folds with slight to mild horizontal folds) |
| 3-Severe | Superficial wrinkling present, may be marked |
| | Loose skin separated from deeper neck structures |
| | Marked skin redundancy (severe vertical and |
| | moderate to severe horizontal folds |

### Safety Evaluations

Clinical Assessment of Injection Zone Local Skin Reactions. Per the schedule of events, injection zone LSRs (erythema, edema, tenderness on palpation, bruising, pain, and stinging/burning) would be assessed using a 4-point ordinal scale (0=complete absence, 1-mild, limited involvement, 2-moderate involvement, and 3-severe, extreme involvement). Only LSRs that required medical intervention (e.g., prescription medication), resulted in a postponed or skipped treatment session, or required discontinuation of treatment would be documented as AEs. Any LSRs that were not listed would be recorded as AEs. It was important that the photographs of the injection zones were performed per protocol.

Physical Examination. Brief physical examinations would be performed. Assessments would include examination of head and neck, dermatologic (except submental fat), cardiovascular, respiratory, gastrointestinal (abdomen), and gross motor and gait. Clinically significant abnormalities at Visit 2/Baseline would be recorded as medical history. Any new or worsening clinically significant abnormalities at post-Baseline visits would be recorded as AEs.

Vital Signs. Vital signs including temperature, systolic and diastolic blood pressure, heart rate, respiration rate, would be measured per the Schedule of Events. Assessments would be made after the subject has rested in a seated position for at least 5 minutes. Weight will would also be measured. Height would also be measured at Visit 2/Baseline.

Electrocardiogram. ECGs would be performed. 12-lead ECGs was performed after the subject has rested for at least 10 minutes in the supine position. The investigator must review all the ECG reports in a timely manner. The investigator must complete an appropriate AE form for any new or worsening abnormal test results that are identified as clinically significant after Baseline.

### Photography

Photography documentation was required in this study. Photographs taken as part of this study would be used to document the effects of treatment, AEs, or other findings during the study. The site would be provided with guidelines to assist them in taking standardized photographs. All photographs taken as part of this study were not to be used by the investigator to assist in grading or for any other assessment. Any photos taken would protect subject confidentiality to the extent possible. This may include the use of a photo imaging masking tool to place squares or a bar on the image to mask any identifiable areas (e.g., eyes) or use of an approved mask to cover the eyes during photography. At the election of the Sponsor, an independent separate analysis of the photographs for clinical changes relative to Screening/Baseline may be performed.

### Laboratory Tests

Blood chemistries, hematology, urinalysis, and coagulation parameters. Blood and urine specimens would be collected. Subjects must be fasting (approximately 8 hours) for Visit 1/Screening and Visit 7/Treatment #6. For Visit 1/Screening: if fasting samples could not be collected at the time of other Visit 1/Screening procedures, the subject may return to the clinic for sample collection such that the results of the clinical laboratory tests were available for review prior to Visit 2/Baseline; such testing would still be considered “Visit 1/Screening” labs. If for any reason, screening labs were not collected after subject was fasting, Visit 2/Baseline labs must be taken after the subject has fasted for at least 8 hours. If a subject arrived at the clinic for any other visit without fasting for at least 8 hours (except for Visit 7), laboratory specimens may be collected as non-fasting, at the discretion of the investigator, and with the appropriate fasting status documented on the lab requisition form. Samples taken for clinical laboratory tests at Visit 2/Baseline, Visit 4, Visit 6, Visit 8 (if applicable), Visit 9 (if applicable), and Visit 10 (if applicable) should be taken as fasting if possible. Additionally, for subjects who had an ET visit, every attempt should be made to have fasting lab samples collected. For all lab draws, the appropriate fasting status would be documented on the lab requisition form. Table 7 illustrates non-limiting examples of the laboratory test performed.

TABLE 7
Non-limiting examples of laboratory tests
LABORATORY TESTS

| | | | Coagulation Parameters |
|---|---|---|---|
| Chemistries | Hematology | Urinalysis | (Visit 1/Screening only) |
| Albumin | Hemoglobin | Color | Activated partial |
| | | | thromboplastin time |
| Alkaline phosphatase | Hematocrit | Appearance/Clarity | Prothrombin time |
| ALT (SGPT) | MCV | Bilirubin | |
| AST (SGOT) | MCH | Blood | |
| Bilirubin, total | MCHC | Glucose | |
| Calcium | RBC (Erythrocytes) | Ketones | |
| Carbon Dioxide | WBC (Leucocytes) | Leukocyte Esterase | |
| Chloride | RDW | Nitrite | |
| Creatinine | Differential count | pH | |
| GGT | Basophils | Protein | |
| Glucose, fasting | Eosinophils | Specific gravity | |
| LDH | Lymphocytes | Urobilinogen | |
| Phosphate | Monocytes | Microscopic | |
| | | analysis* | |
| Potassium | Neutrophils | | |
| Protein, total | Platelets | | |
| Sodium | | | |
| Urea (BUN) | | | |
| Uric acid | | | |
| Lipid Panel | | | |
| Cholesterol, total | | | |
| HDL | | | |
| Direct LDL | | | |
| Triglycerides | | | |

*Only if one of the other analytes is positive

Sample collection, handling, labeling, and shipping should be done following the instructions provided by the relevant certified laboratory and the applicable local regulations. The investigator must review all the subject's laboratory reports in a timely manner. The investigator would note, directly on the laboratory report, whether or not any abnormal test results are clinically significant or not clinically significant. Clinical significant laboratory abnormalities that were present at Screening and/or Baseline must be documented in the subject's medical history. In addition, the investigator must complete an appropriate AE form for any new or worsening abnormal test results that were identified as clinically significant or any abnormal test results that meet CTCAE Grade 3 criteria after Baseline. AEs that may be associated with venipuncture and that must be included in the informed consent include: pain; bruising; bleeding at the puncture site; fainting; or inflammation of the vein.

Urine pregnancy test (UPT). The UPTs would be performed at the study site as permitted by applicable local and national health authority laws and regulations. In the United States, the site must be registered and conform to CLIA regulations for such testing (possesses a current valid CLIA Certificate of Waiver or higher), or at an appropriately registered reference laboratory. The investigator would report the UPT results on the eCRFs, in the subject's medical records, and in independent records maintained at the study site. The UPT used must have a minimum sensitivity of 25 mIU of β-hCG/mL.

### End of Study Criteria

At the end of each subject's participation in the study, the investigator completed an end of study disposition form for all completed and discontinued subjects.

Completion of the Study. Subjects who underwent treatment visits as specified in this protocol and complete the end of study (EOS) visit activities were considered to have completed the study.

Subject Discontinuation. A subject may be withdrawn from the study prior to completion for any of the following reasons: AEs; death; lack of efficacy; lost to follow-up; non-compliance with study drug; physician decision; pregnancy; progressive disease; protocol violation; study terminated; withdrawal by subject; or other (e.g., any reason that may affect the outcome of the study or safety of subjects). If a subject withdrew from the study prematurely for any reason, the site should make every effort to have the subject return to the clinic to perform all of the required visit activities. If the subject would not return to the clinic, the site should make every attempt to contact the subject; otherwise the would will be considered lost to follow-up. When a subject was withdrawn from the study for a test article related AE, when possible, the subject should be followed until resolution or stabilization of the AE. If the subject was discontinued from the study due to pregnancy, the pregnancy and its outcome should be followed.

### Study Termination

The study may be terminated by the investigator or the sponsor. If, in the opinion of the investigator, clinical observations made during the study suggested that it may be unwise to continue, he or she may stop the study. A study termination by the investigator would be reported to the sponsor. In addition, a written statement fully documenting the reasons for this action would be submitted to the sponsor by the investigator within 5 working days. In the event that the sponsor chose to discontinue or terminate the study, appropriate notification would be given to the investigator.

### Efficacy Endpoints

The efficacy endpoints would include: “Treatment success” by visit after final treatment where “treatment success” is defined as a ≥1-grade decrease in CSFS score AND a ≥1-grade decrease in PSFS score from Baseline; “Treatment success” by visit after final treatment where “treatment success” is defined as a ≥2-grade decrease in CSFS score AND a ≥2 grade decrease in PSFS score from Baseline; “Treatment success” by visit after final treatment where “treatment success” is defined as a ≥3-grade decrease in CSFS score AND a ≥3 grade decrease in PSFS score from Baseline; change from Baseline in CSFS by visit after final treatment; change from Baseline in PSFS by visit after final treatment; change from Baseline in SSLS by visit; or optional independent photo review.

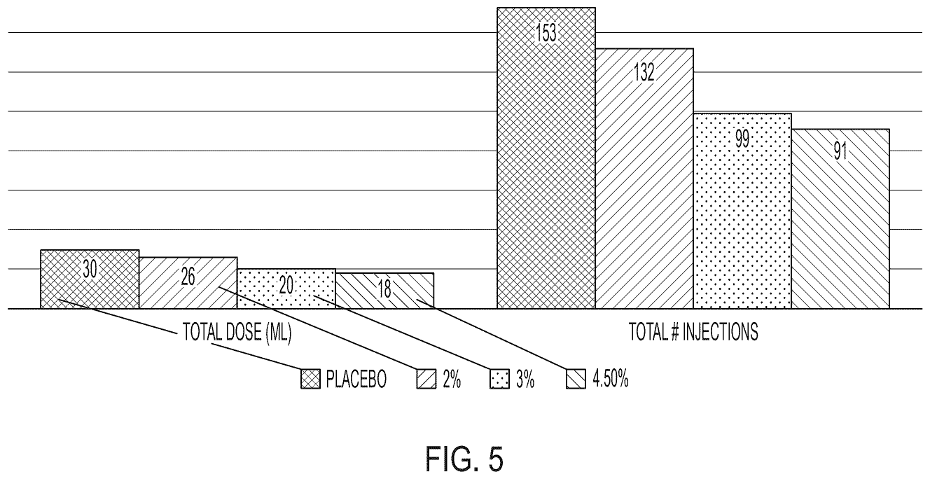

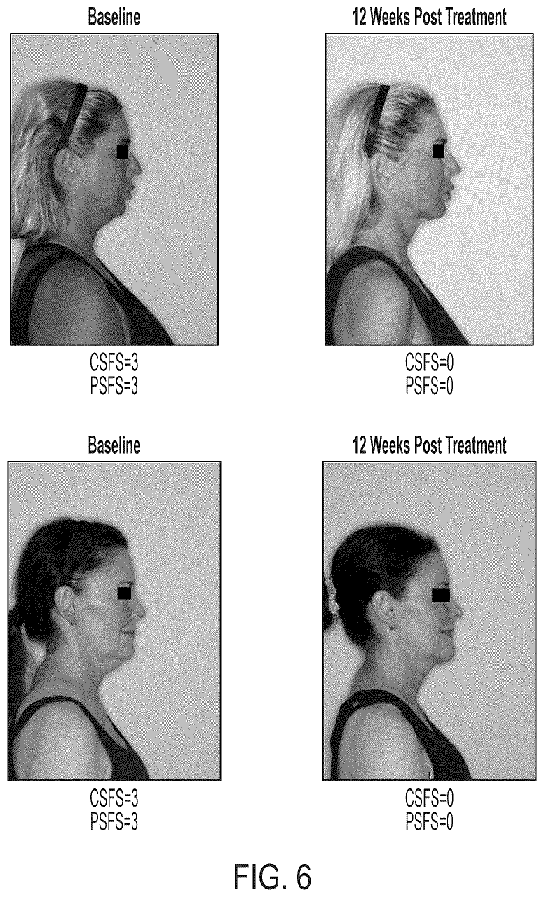

Table 8 illustrates subject disposition (ITT population) subjected to the examination of the protocols of Example 1. FIGS. 2A to 2E illustrate a non-limiting example of submental study based on the protocols of Example 1. FIG. 2A illustrates co-primary endpoint-Composite CSFS/PSFS responders>/=2. CSFS stands for clinician submental fat scale and PSFS stands for patient submental fat scale. FIG. 2A also illustrates comparison with Kybella (deoxycholic acid). Similar to FIG. 2A, Table 9 illustrates grade reduction in CSFS & PSFS (ITT Population, 4 weeks after final treatment) compared to Kybella. FIG. 2B illustrates co-primary endpoint-Composite CSFS/PSFS responders>/=1. Table 10 illustrates that 10XB101 exhibited higher proportion of patients that achieved ≥2 grade improvement in submental fat rating scale (ITT Analysis). FIG. 2C illustrates CSFS/PSFS=3-point improvement. FIG. 2D illustrates proportion of responders clinician submental fat scale (ITT). FIG. 2E illustrates proportion of responders patient submental fat scale (ITT). * denotes statistical significance based on Fisher Exact Test. ** denotes average of Refine 1 and 2 trials. FIG. 3 illustrates 10XB101 showing ≥2-Fold increase in mean CSFS change at same time point versus Kybella (ITT Analysis). Table 11 illustrates 10XB101 exhibiting lower, FDA label included, injection site reaction adverse events related to market acceptance. Table 12 illustrates injection site adverse events lasting >30 days in more than 10% of subjects. FIG. 4 illustrates proportion of responders overtime with CSFS≥2 (ITT). FIG. 5 illustrates greater loss of submental fat during treatment leads to reduced drug volume and injection number. FIG. 6 illustrates submental fat loss after 12-week post treatment accompanied by decreased CSFS and PSFS.

The Safety and intent-to-treat (ITT) populations will include all enrolled subjects who received any injections of test article. The Completers population will include a subset of the ITT population who met the following criteria: (1) had at least 4 treatments or completed treatments per protocol (i.e., treatments did not continue once CSFS=0), (2) completed the 4 weeks or more after final treatment assessments, and (3) does not have any significant protocol deviations that would impact the evaluation of efficacy.

TABLE 8
Subject disposition (ITT population) subjected to
the examination of the protocols of Example 1

| | 10XB-101 | 10XB-101 | 10XB-101 | | |
|---|---|---|---|---|---|
| | 2.0% | 3.0% | 4.5% | Vehicle | Total |
| Randomized | N = 13 | N = 13 | N = 13 | N = 12 | N = 51 |

Completed Study, n (%)

| Yes | 12 | (92.3) | 10 | (76.9) | 9 | (69.2) | 9 | (75.0) | 40 | (78.4) |
|---|---|---|---|---|---|---|---|---|---|---|
| No | 1 | (7.7) | 3 | (23.1) | 4 | (30.8) | 3 | (25.0) | 11 | (21.6) |
| Reason for | | | | | | | | | | |
| Withdrawal, n (%) | | | | | | | | | | |
| Adverse Event | 0 | | 1 | (7.7) | 1 | (7.7) | 0 | | 2 | (3.9) |
| Lost to Follow-up | 1 | (7.7) | 0 | | 0 | | 0 | | 1 | (2.0) |
| Withdrawal by subject | 1 | (7.7) | 1 | (7.7) | 2 | (15.4) | 1 | (8.3) | 5 | (9.8) |

TABLE 9A
Grade reduction in the Composite CSFS & PSFS assessment (ITT Population,
4 weeks after final treatment) compared to Kybella

| | | Kybella Ph 3 | | Kybella Ph 3 | | | | | |
|---|---|---|---|---|---|---|---|---|---|
| | | (Trial 22) | | (Trial 23) | | 10X-B101 Ph 2b | | | |
| | | Kybella | Placebo | Kybella | Placebo | 4.50% | 3.00% | 2.00% | Vehicle |
| | | N = 256 | N = 250 | N = 258 | N = 258 | N = 13 | N = 13 | N = 13 | N = 12 |
| ≥1 grade | (%) | −70.00% | −18.60% | −66.50% | −22.20% | −80.00% | −85.00% | −69.00% | −42.00% |
| reduction | | | | | | | | | |
| ≥2 grade | (%) | −13.40% | (<0.1%) | −18.60% | −3.00% | −54.00% | −77.00% | −46.00% | −8.00% |
| reduction | | | | | | | | | |

TABLE 9B
Grade reduction in the Composite CSFS & PSFS assessment(Completers
Population, 4 weeks after final treatment) compared to Kybella

| | | Kybella Ph 3 | | Kybella Ph 3 | | | | | |
|---|---|---|---|---|---|---|---|---|---|
| | | (Trial 22) | | (Trial 23) | | 10X-B101 Ph 2b | | | |
| | | Kybella | Placebo | Kybella | Placebo | 4.50% | 3.00% | 2.00% | Vehicle |
| | | N = 256 | N = 250 | N = 258 | N = 258 | N = 13 | N = 13 | N = 13 | N = 12 |
| ≥1 grade | (%) | −70.00% | −18.60% | −66.50% | −22.20% | −62.00% | −77.00% | −77.00% | −17.00% |
| reduction | | | | | | | | | |
| ≥2 grade | (%) | −13.40% | (<0.1%) | −18.60% | −3.00% | −54.00% | −62.00% | −39.00% | 0.00% |
| reduction | | | | | | | | | |

TABLE 10
Injection site adverse events lasting
>30 days in more than 10% of subjects

| | 10XB101* | Kybella** |
|---|---|---|
| Numbness | 2% | 42% |
| Edema/Swelling | 4% | 20% |
| Injection Site Pain | 0% | 16% |
| Induration | 0% | 13% |

*LSRs and Spontaneous/Elicited AEs at pre-injection time point (~30 days post prior injection)
**Kybella Label

TABLE 11
10XB101 exhibiting lower, FDA label included, injection
site reaction adverse events related to market acceptance

| AE Proportions | | | | | Kybella (Active)** |
|---|---|---|---|---|---|
| Post Treatment | 4.5% | 3.0% | 2.0% | Vehicle | (1% deoxycholic acid) |
| Pain* | 23% | 8% | 15% | 0% | 70% |
| Bruising* | 54% | 46% | 31% | 69% | 72% |
| Edema/Swelling* | 85% | 77% | 92% | 31% | 87% |
| Burning/Stinging* | 46% | 8% | 8% | 0% | Not Reported |
| Tenderness On | 85% | 62% | 85% | 38% | Not Reported |
| Palpation* | | | | | |
| Erythema* | 8% | 15% | 23% | 15% | 27% |
| Anesthesia | 15% | 0% | 0% | 0% | 66% |
| (Numbness)^{†} | | | | | |
| Induration^{†} | 0% | 0% | 0% | 0% | 23% |
| Paresthesia^{†} | 0% | 0% | 0% | 0% | 14% |
| Nodule^{†} | 0% | 0% | 0% | 0% | 13% |
| Pruritis^{†} | 0% | 0% | 0% | 0% | 12% |
| Nerve Injury^{†} | 0% | 0% | 0% | 0% | 4% |

*10XB101 Local Skin Reaction Measurement (0-4 scale: absent, mild, moderate, severe). Subjects reporting a particular LSR more than once are counted only once for that LSR.
^{†}10XB101 spontaneous or elicited injection site reaction Adverse Events (AE)
**Kybella Label, spontaneous and elicited AEs, 1-week post-injection. Subjects reporting a particular AE more than once are counted only once for that AE.

Table 12 illustrates LSR assessments (all, erythema, edema, tenderness on palpation, bruising, pain, or burning/stinging) in the Safety Population. In subjects receiving active treatment, 79%+ of all LSRs assessed per protocol were grade 0 (none). In subjects receiving active treatment, 98%+ of all LSRs assessed per protocol were grade 0 (none) or grade 1 (mild). Table 12 lists the number of assessments performed during the study and the relative percentage in parentheses for total and individual LSRs for each dose cohort.

TABLE 12
10xB101 LSR Assessments

| All LSRs | | | | | | | | | |
|---|---|---|---|---|---|---|---|---|---|
| Grade | 4.50% | | 3% | | 2% | | Vehicle | | Active Total |
| 0 | 1015 | (77.63%) | 1081 | (82.12%) | 1079 | (79.72%) | 3064 | (82.49%) | (79.81%) |
| 1 | 206 | (20.37%) | 263 | (16.67%) | 221 | (19.4%) | 723 | (16.9%) | (18.83%) |
| 2 | 11 | (2%) | 12 | (0.89%) | 8 | (0.88%) | 48 | (0.61%) | (0.1%) |
| 3 | 4 | (0%) | 0 | (0.32%) | 0 | (0%) | 4 | (0%) | (0.1%) |
| Total | 1236 | (22.37%) | 1356 | (17.88%) | 1308 | (20.28%) | 3839 | (17.51%) | (20.19%) |

| | LSR | | | | | | | | | |
|---|---|---|---|---|---|---|---|---|---|---|
| Grade | 4.50% | | 3% | | 2% | | Vehicle | | Active Total | |
| Erythema | | | | | | | | | | |
| 0 | 164 | (79.23%) | 159 | (77.18%) | 159 | (70.35%) | 162 | (74.31%) | 482 | (75.43%) |
| 1 | 40 | (19.32%) | 43 | (20.87%) | 65 | (28.76%) | 52 | (23.85%) | 148 | (23.16%) |
| 2 | 3 | (1.45%) | 4 | (1.94%) | 2 | (0.88%) | 4 | (1.83%) | 9 | (0%) |
| 3 | 0 | (0%) | 0 | (0%) | 0 | (0%) | 0 | (0%) | 0 | (0%) |
| Total | 207 | (20.77%) | 206 | (22.82%) | 226 | (29.65%) | 218 | (25.69%) | 639 | (24.57%) |
| Edema | | | | | | | | | | |
| 0 | 122 | (58.65%) | 130 | (63.11%) | 134 | (59.29%) | 158 | (72.48%) | 386 | (60.31%) |
| 1 | 72 | (34.62%) | 70 | (33.98%) | 83 | (36.73%) | 59 | (27.06%) | 225 | (35.16%) |
| 2 | 14 | (6.73%) | 6 | (2.91%) | 9 | (3.98%) | 1 | (0.46%) | 29 | (0%) |
| 3 | 0 | (0%) | 0 | (0%) | 0 | (0%) | 0 | (0%) | 0 | (0%) |
| Total | 208 | (41.35%) | 206 | (36.89%) | 226 | (40.71%) | 218 | (27.52%) | 640 | (39.69%) |
| Tenderness on Palpation | | | | | | | | | | |
| 0 | 121 | (58.17%) | 146 | (70.87%) | 151 | (66.81%) | 168 | (77.06%) | 418 | (65.31%) |
| 1 | 81 | (38.94%) | 58 | (28.16%) | 75 | (33.19%) | 50 | (22.94%) | 214 | (33.44%) |
| 2 | 6 | (2.88%) | 1 | (0.49%) | 0 | (0%) | 0 | (0%) | 7 | (0.16%) |
| 3 | 0 | (0%) | 1 | (0.49%) | 0 | (0%) | 0 | (0%) | 1 | (0.16%) |
| Total | 208 | (41.83%) | 206 | (29.13%) | 226 | (33.19%) | 218 | (22.94%) | 640 | (34.69%) |
| Bruising | | | | | | | | | | |
| 0 | 189 | (90.87%) | 190 | (92.23%) | 208 | (92.04%) | 189 | (86.7%) | 587 | (91.72%) |
| 1 | 19 | (9.13%) | 15 | (7.28%) | 18 | (7.96%) | 29 | (13.3%) | 52 | (8.13%) |
| 2 | 0 | (0%) | 0 | (0%) | 0 | (0%) | 0 | (0%) | 0 | (0.16%) |
| 3 | 0 | (0%) | 1 | (0.49%) | 0 | (0%) | 0 | (0%) | 1 | (0.16%) |
| Total | 208 | (9.13%) | 206 | (7.77%) | 226 | (7.96%) | 218 | (13.3%) | 640 | (8.28%) |
| Pain | | | | | | | | | | |
| 0 | 198 | (95.19%) | 202 | (98.06%) | 219 | (96.9%) | 203 | (93.12%) | 619 | (96.72%) |
| 1 | 9 | (4.33%) | 3 | (1.46%) | 7 | (3.1%) | 15 | (6.88%) | 19 | (2.97%) |
| 2 | 1 | (0.48%) | 0 | (0%) | 0 | (0%) | 0 | (0%) | 1 | (0.16%) |
| 3 | 0 | (0%) | 1 | (0.49%) | 0 | (0%) | 0 | (0%) | 1 | (0.16%) |
| Total | 208 | (4.81%) | 206 | (1.94%) | 226 | (3.1%) | 218 | (6.88%) | 640 | (3.28%) |
| Burning/Stinging | | | | | | | | | | |
| 0 | 174 | (83.65%) | 188 | (91.26%) | 210 | (92.92%) | 199 | (91.28%) | 572 | (89.38%) |
| 1 | 33 | (15.87%) | 17 | (8.25%) | 15 | (6.64%) | 16 | (7.34%) | 65 | (10.16%) |
| 2 | 1 | (0.48%) | 0 | (0%) | 1 | (0.44%) | 3 | (1.38%) | 2 | (0.16%) |
| 3 | 0 | (0%) | 1 | (0.49%) | 0 | (0%) | 0 | (0%) | 1 | (0.16%) |
| Total | 208 | (16.35%) | 206 | (8.74%) | 226 | (7.08%) | 218 | (8.72%) | 640 | (10.63%) |

TABLE 13
illustrates the number of injections administered per visit
in the trial, an illustates that fewer average numbers of
injections were necessary in subsequent treatment sessions.
Number of Injections per Visit

| | 2% | 3% | 4.50% | Vehicle |
|---|---|---|---|---|
| VISIT 2-BASELINE-TX 1 | 13 | 13 | 13 | 12 |
| n | | | | |
| Mean (SD) | 28.9 (12.72) | 26.6 (11.92) | 27.0 (15.33) | 28.8 (14.80) |
| Median | 30.0 | 30.0 | 20.0 | 25.5 |
| Min, Max | 12, 50 | 10, 42 | 10, 50 | 10, 47 |
| VISIT 3-TX 2 | 13 | 12 | 11 | 11 |
| n | | | | |
| Mean (SD) | 27.3 (12.81) | 23.7 (13.73) | 22.0 (10.84) | 27.3 (12.07) |
| Median | 25.0 | 20.5 | 18.0 | 25.0 |
| Min, Max | 10, 50 | 7, 47 | 8, 38 | 10, 45 |
| VISIT 4-TX 3 | 11 | 11 | 11 | 12 |
| n | | | | |
| Mean (SD) | 23.1 (11.67) | 16.5 (11.28) | 20.1 (15.39) | 27.6 (13.28) |
| Median | 23.0 | 14.0 | 17.0 | 24.5 |
| Min, Max | 8, 40 | 4, 40 | 3, 48 | 10, 46 |
| VISIT 5-TX 4 | 12 | 10 | 10 | 11 |
| n | | | | |
| Mean (SD) | 22.3 (13.08) | 20.8 (9.41) | 15.5 (10.76) | 26.5 (12.53) |
| Median | 19.5 | 19.0 | 15.0 | 20.0 |
| Min, Max | 4, 45 | 9, 35 | 3, 32 | 13, 45 |
| VISIT 6-TX 5 | 11 | 10 | 10 | 11 |
| n | | | | |
| Mean (SD) | 22.3 (15.03) | 17.8 (8.80) | 11.8 (7.33) | 25.5 (11.32) |
| Median | 20.0 | 13.0 | 12.0 | 20.0 |
| Min, Max | 4, 50 | 10, 35 | 2, 20 | 13, 46 |
| VISIT 7-TX 6 | | | | |
| n | 11 | 8 | 9 | 11 |
| Mean (SD) | 19.5 (15.38) | 11.4 (5.13) | 10.2 (6.20) | 25.5 (12.68) |
| Median | 15.0 | 12.0 | 10.0 | 20.0 |
| Min, Max | 3, 45 | 3, 20 | 3, 20 | 6, 42 |

FIG. 7 illustrates improvements to an injection related side effect(s) when administering polidocanol pharmaceutical formulations according to methods described herein. In FIG. 7, the number of subjects exhibiting a side effect of bruising or edema is shown, along with the grade of the side effect. In FIG. 7, 13 subjects total exhibited bruising, with almost all subjects exhibiting a grade 0 (none) bruising, with only 2 subjects exhibiting grade 1 (mild) bruising, no subjects exhibiting grade 2 bruising, and only 1 subject exhibiting grade 3 bruising. In FIG. 7, 13 subjects total exhibited edema, with most subjects exhibiting a grade 0 (none) edema, with only 5 subjects exhibiting grade 1 (mild) bruising, no subjects exhibiting grade 2 or grade 3 edema. In FIG. 7, it is further shown that fewer subjects exhibit bruising or edema as the trial continued and further treatments were provided at visits 3, 4, 5, and 6. In FIG. 7, it is further shown that a side effect of bruising or edema, if present, generally did not worsen following the treatment and it is further shown that incidences of bruising or edema, if present, generally resolved by the next treatment dose.

FIG. 8 illustrates improvements to an injection related side effect(s), specifically pain, when administering polidocanol pharmaceutical formulations according to methods described herein. In FIG. 8, it is shown that the frequency of pain was generally mild, with the vast majority of subjects exhibiting grade 0 (none) pain, and it is further shown that incidences of pain generally resolved by the next treatment dose.

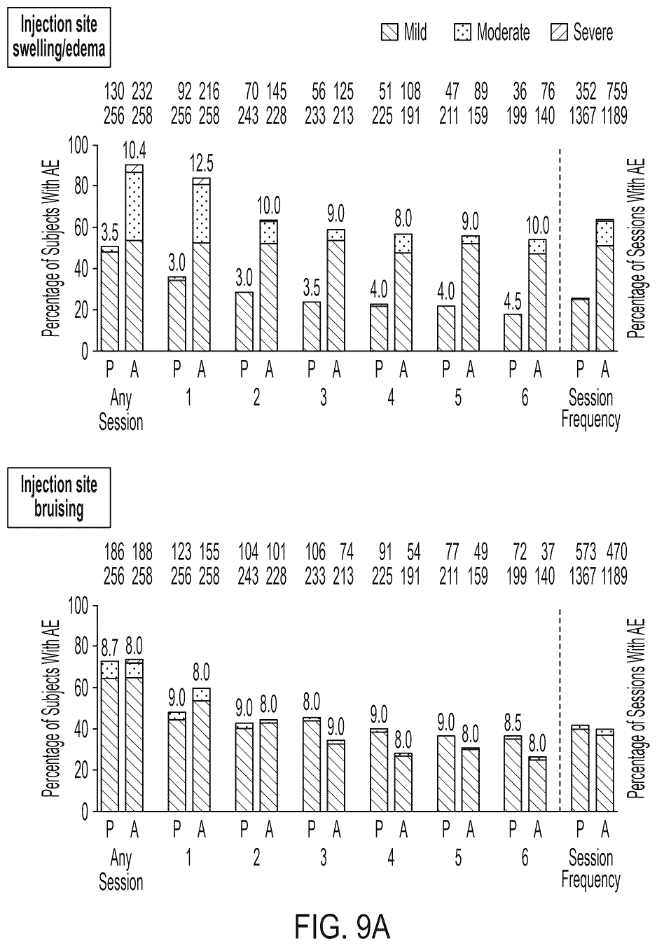

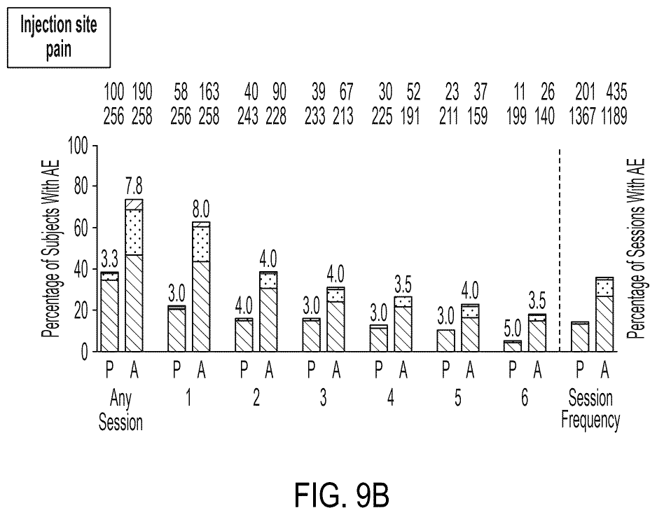

FIGS. 9A and 9B illustrate injection related side effects in subject administered 1% deoxycholic acid (“ATX-101” or “A”), compared to a control (“placebo” or “P”). FIGS. 9A and 9B are graphs illustrating the incidence, severity, and duration of injection-site adverse events (AE) (swelling/edema, bruising, and pain) for individual treatment sessions. At the top of the graphs, the number of patients reporting the AE is listed in the first row; the total number of patients is listed in the second row. The number listed at the top of each bar is the median duration of the AE in a particular session. For session frequency, the data for the percentage of sessions for which the AE was reported are shown. As is shown in FIGS. 9A and 9B, a significantly higher percentage of subjects exhibited an adverse reaction of swelling/edema, bruising, or pain when administered 1% deoxycholic acid.

### Example 2. Flank Body Contouring Study

This example adopted a randomized approach in studying flank body contouring, assigning participants to either right (R) or left (L) flank treatment groups of either receiving the 10XB-101 solution or a placebo (“control” or “vehicle”). This randomization minimized potential confounding variables and enhanced the validity of the study's findings. The study involved up to six treatment sessions, administered at monthly intervals, allowing for a comprehensive evaluation of the treatment's efficacy over time.

The primary outcomes of interest were the Clinician Global Impression of Change (CGIC) and Patient Global Impression of Change (PGIC), assessed at 4 to 12 weeks following the completion of the final treatment session. The assessments utilized a 7-point scale, ranging from “much worse” to “much improved,” providing a nuanced understanding of the participants' subjective experiences and the objective efficacy of the intervention.

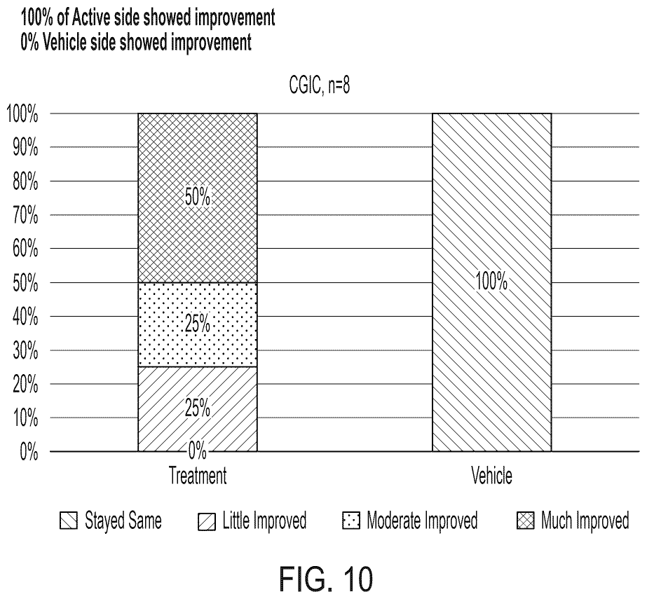

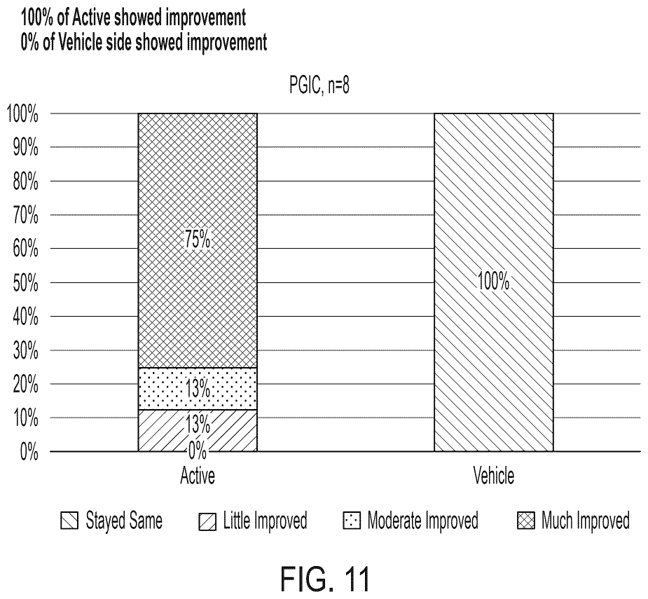

Results from the study indicated that eight participants successfully completed the prescribed treatment regimen and reached either a 4-week or 12-week follow-up period post-treatment. These follow-up intervals allowed for the observation of short-term and medium-term outcomes, providing valuable insights into the durability and sustainability of the treatment effects over time. FIG. 10 and FIG. 11, illustrate the improvement ratings for the active flank treatment side compared to the vehicle (control) side in patients who received more than two treatments, evaluated at 4-12 weeks post-treatment.

FIG. 10, representing the Clinical Global Impression of Change (CGIC), shows a significant contrast between the active flank treatment and the control. On the active treatment side, 50% of patients rated their improvement as “Much Improved,” 25% as “Moderate Improved,” and 25% as “Little Improved.” Notably, no patients reported that their condition stayed the same or worsened, indicating that all participants experienced some degree of improvement with the active treatment.

In contrast, the vehicle side (control) revealed that 100% of patients rated their condition as “Stayed Same,” with no patients reporting any level of improvement. This stark difference underscores the effectiveness of the active flank treatment, as it led to observable improvements in all treated patients, unlike the control, which showed no benefit.

FIG. 11, which depicts the Patient Global Impression of Change (PGIC), presents a similar trend. On the active treatment side, 75% of patients rated their improvement as “Much Improved,” 13% as “Moderate Improved,” and another 13% as “Little Improved.” Again, no patients reported their condition as having stayed the same or worsened, reflecting a unanimous perception of improvement among those receiving the active treatment.

Conversely, the vehicle side in FIG. 11 shows that 100% of patients rated their condition as “Stayed Same,” with no reported improvements, similar to the results of FIG. 10. The consistency in these findings across both the CGIC and PGIC measures strongly suggests that the flank active treatment significantly being more effective than the control in improving the condition being treated. This comprehensive assessment from both clinical and patient perspectives provides robust support of the efficacy of the active flank treatment over the vehicle, highlighting its effectiveness in treating the flank region.

In addition to subjective evaluations, the study incorporated objective measures through photographic assessments to quantify any asymmetry present pre- and post-treatment. Photographic evidence offered a visual representation of the treatment outcomes, enabling the analysis of the changes in body contouring with precision and accuracy.

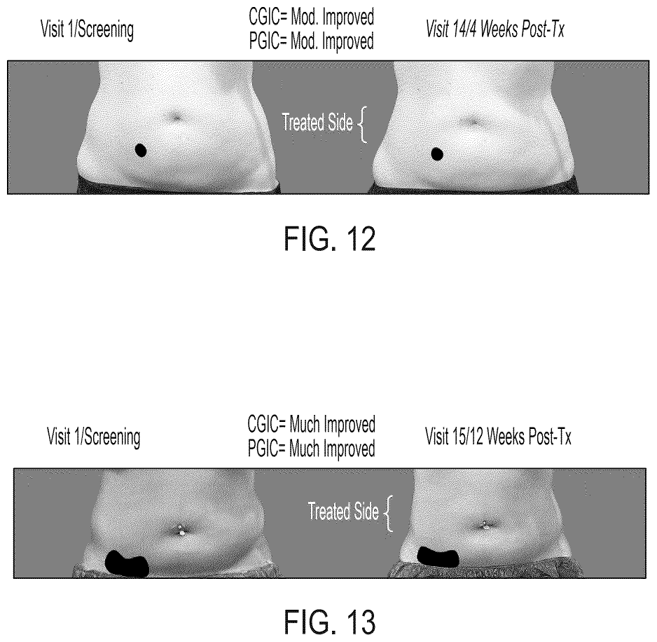

FIG. 12 shows a comparison of a patient's abdominal area before and after the 10XB-101 treatment. The patient, was photographed at two different times: at the initial screening (Visit 1) and four weeks after the treatment (Visit 14). The image on the left, taken during the screening, serves as the baseline. The image on the right, taken four weeks post-treatment, shows the condition of the treated side of the patient's abdomen. Both the Clinical Global Impression of Change (CGIC) and the Patient Global Impression of Change (PGIC) rate the improvement as “Moderately Improved.” This indicates that both clinical evaluators and the patient observed a moderate improvement in the treated area, demonstrating the effectiveness of the treatment.

FIG. 13 shows a comparison of a patient's abdominal area before and after the 10XB-101 treatment. The patient, was photographed at two different times: at the initial screening (Visit 1) and 12 weeks after the treatment (Visit 15). The image on the left, taken during the screening, serves as the baseline. The image on the right, taken 12 weeks post-treatment, shows the condition of the treated side of the patient's abdomen. Both the Clinical Global Impression of Change (CGIC) and the Patient Global Impression of Change (PGIC) rate the improvement as “Much Improved.” This indicates that both clinical evaluators and the patient observed a significant improvement in the treated area, demonstrating the effectiveness of the treatment.

The examples and embodiments described herein were for illustrative purposes only, and various modifications or changes were included within the spirit and purview of this application and scope of the appended claims. All publications, patents, and patent applications cited herein were hereby incorporated by reference for all purposes.

What is claimed is:

1. A method of reducing subcutaneous adipose tissue in the submental region of a subject to achieve at least a two-grade improvement, the method comprising injecting the submental region of the subject with an effective amount of a pharmaceutical composition comprising polidocanol present at 3-4.5% w/w of the pharmaceutical composition, wherein the effective amount is effective to reduce the subcutaneous adipose tissue in the submental region with at least the two-grade improvement as measured via a Patient Submental Fat Scale (PSFS).

2. The method of claim 1, wherein the polidocanol is present at about 4% w/w of the pharmaceutical composition.

3. The method of claim 2, wherein the subject does not exhibit an injection related local skin reaction (LSR) that lasts for more than 30 days and/or the subject exhibits predominantly a mild injection related LSR after injection; wherein the injection related LSR is pain, and whether the subject exhibits an injection related LSR is determined subjectively by the subject using a scale from 0 to 3, wherein grade 0 is no injection related side effect, grade 1 is mild injection related side effect, grade 2 is moderate injection related side effect, and grade 3 is severe injection related side effect.

4. The method of claim 2, wherein the effective amount of the pharmaceutical composition has an anesthetic effect on the subject.

5. The method of claim 2, wherein the effective amount is a total volume of about 3 mL to about 10 mL.

6. The method of claim 5, wherein the total volume is administered in 15 to 50 doses, and each dose has a volume of about 0.2 mL.

7. The method of claim 6, wherein each dose is administered within about 1 cm of another dose.

8. The method of claim 6, wherein the 15 to 50 doses is more than 30 doses and up to 50 doses.

9. The method of claim 2, wherein the pharmaceutical composition comprises propylene glycol at 2% to 5% w/w of the pharmaceutical composition.

10. The method of claim 9, wherein the propylene glycol is present at about 2% w/w of the pharmaceutical composition.

11. The method of claim 2, wherein the effective amount is a first effective amount, and the method further comprises injecting the subject with a second effective amount of the pharmaceutical composition, wherein injecting the subject with the second effective amount of the pharmaceutical composition occurs at least 14 days after injecting the subject with the first effective amount, but not more than 60 days from injecting the subject with the first effective amount.

12. The method of claim 2, wherein the effective amount is effective to reduce the subcutaneous adipose tissue in the submental region with less edema than injecting the subject with 1% deoxycholic acid.

13. The method of claim 2, wherein the effective amount is effective to reduce the subcutaneous adipose tissue in the submental region with less bruising than injecting the subject with 1% deoxycholic acid.

14. The method of claim 2, wherein the subject is not concurrently treated with a laser, chemical peel, or dermal filler in the submental region.

15. A method of reducing subcutaneous adipose tissue in the submental region of a subject with less pain than injecting the subject with 1% deoxycholic acid, the method comprising injecting the submental region of the subject with an effective amount of a pharmaceutical composition comprising polidocanol present at 3-4.5% w/w of the pharmaceutical composition, wherein the effective amount is effective to reduce the subcutaneous adipose tissue in the submental region and has an anesthetic effect on the subject.

16. The method of claim 15, wherein the polidocanol is present at about 4% w/w of the pharmaceutical composition.

17. The method of claim 16, wherein the effective amount is a total volume of about 3 mL to about 10 mL.

18. The method of claim 17, wherein the total volume is administered in 15 to 50 doses, and each dose has a volume of about 0.2 mL.

19. The method of claim 16, wherein the pharmaceutical composition comprises propylene glycol at 2% to 5% w/w of the pharmaceutical composition.

20. The method of claim 19, wherein the propylene glycol is present at about 2% w/w of the pharmaceutical composition.
